(19) United States
(12) Patent Application Publication
(10) Pub. No.: US 2024/0316213 A1
(43) Pub. Date: Sep. 26, 2024

(54) ANTIBODY-PROTAMINE FUSIONS AS TARGETING COMPOUNDS OF A PROTAMINE-BASED NANOPARTICLE

(71) Applicant: Universität Münster, Münster (DE)

(72) Inventors: Sebastian BÄUMER, Münster (DE); Nicole BÄUMER, Münster (DE); Wolfgang BERDEL, Münster (DE); Georg LENZ, Münster (DE); Andreas FAUST, Münster (DE); Lisa WITTMANN, lbbenbüren (DE)

(21) Appl. No.: 18/562,164

(22) PCT Filed: May 20, 2022
(86) PCT No.: PCT/EP2022/063732

§ 371 Date: Nov. 17, 2023

(30) Foreign Application Priority Data
May 21, 2021 (EP) 21175340.5
Oct. 29, 2021 (EP) 21205455.5
Dec. 2, 2021 (EP) 21212002.6

(51) Int. Cl. A61K 47/69; A61K 31/713; A61K 47/64; A61K 47/68

(52) U.S. Cl. CPC A61K 47/6929; A61K 31/713; A61K 47/6455; A61K 47/6849; A61K 47/6883

(57) ABSTRACT

The present invention relates to a method of generating a nanoparticle comprising contacting (a) a fusion protein (A), said fusion protein (A) comprising an antibody (A1) and a positively charged polypeptide (A2); (b) a positively charged polypeptide (B); and (c) a negatively charged molecule (C); thereby forming a nanoparticle. The present invention also relates to a nanoparticle obtainable by a method of the invention, as well as to a nanoparticle comprising (a) a fusion protein (A), said fusion protein (A) comprising an antibody (A1) and a positively charged polypeptide (A2); (b) a positively charged polypeptide (B); and (c) one or more negatively charged molecule(s) (C). The present invention also relates to a composition comprising a nanoparticle of the invention and to a nanoparticle or composition of the invention for use in therapy.

### CROSS-REFERENCE TO RELATED APPLICATIONS

This application claims the benefit of priority of European patent application no. 21175340.5 filed 21 May 2021, European patent application no. 21205455.5 filed 29 Oct. 2021 and of European patent application no. 21212002.6 filed 2 Dec. 2021, the contents of which are being hereby incorporated by reference in their entirety for all purposes.

### FIELD OF THE INVENTION

The present invention relates to a method of generating a nanoparticle comprising contacting (a) a fusion protein (A), said fusion protein (A) comprising an antibody (A1) and a positively charged polypeptide (A2); (b) a positively charged polypeptide (B); and (c) a negatively charged molecule (C); thereby forming a nanoparticle. The present invention also relates to a nanoparticle obtainable by a method of the invention, as well as to a nanoparticle comprising (a) a fusion protein (A), said fusion protein (A) comprising an antibody (A1) and a positively charged polypeptide (A2); (b) a positively charged polypeptide (B); and (c) one or more negatively charged molecule(s) (C). The present invention also relates to a composition comprising a nanoparticle of the invention and to a nanoparticle or composition of the invention for use in therapy.

### BACKGROUND

The principle of RNA inhibition (RNAi) raised high expectations for medical applications and was rewarded with the Nobel prize in 2006. This method shows high efficiency by inactivation of mRNA and subsequent silencing of the expression of virtually any gene by the selection and synthesis of gene-specific siRNA oligonucleotides. While this method revolutionized molecular biology, the translation of this principle to the therapeutic arena proved to be difficult due to a number of specific problems.

The siRNA oligos are attacked by nucleases, show elevated immunogenicity and renal clearance, so the half live and circulation time of “naked siRNAs” are often well below expectations. Consequently, siRNAs have been complexed to stabilizing agents, such as nanoparticles or capsules. With these stabilizing agents, the circulation time and bioavailability of the siRNA was raised, but still lacked target-cell determining structures, that a) target cells with specific surface molecules and deliver the siRNA to these cells and b) enables the target-specific transfer of the anionic siRNA over the anionic cytoplasmic membrane.

Although numerous clinical phase studies I-III have been conducted for the treatment of neurological disorders, viral infections and cancer, till now, only one siRNA has been approved by the FDA. E.g. Patisiran (trade name Onpattro) is a medication for the treatment of polyneuropathy in people with hereditary transthyretin-mediated amyloidosis. Hereditary transthyretin-mediated amyloidosis is a fatal rare disease that is estimated to affect 50,000 people worldwide.

In order to develop a modular therapeutic approach for the treatment of oncological disorders, we developed a system to couple siRNA to antibodies against cancer cell-specific surface molecules and causing internalization upon binding by means of a specific cationic peptide, protamine that delivers siRNA to the intended cancer cells, binds to the respective surface molecules such as receptors and gets internalized in a receptor-dependent fashion.

Protamine is a cationic, nucleic acid-binding peptide transporting a complete set of genomic DNA compressed in the sperm-head. Since it is able to complex nucleic acids and to facilitate the transition of nucleic acids across the cytoplasmic membrane, this attracted numerous researchers to study application in transfection, targeted delivery, and gene therapy (Choi et al., 2009; Chono et al., 2008; Hansen et al., 1979; He et al., 2014; Liu, B., 2007). Protamine was tested as a nucleic acid delivery vehicle and connected to various cell determining targeting moieties. In 2005, Song et al. (Song et al., 2005a) presented a genetic fusion protein connecting a Fab fragment (F105) against a human immunodeficiency virus HIV gp 160 envelope protein and a shortened protamine peptide. The fusion protein complexed siRNAs targeting HIV gag protein, and the conjugate was able to target hard-to-transfect HIV infected T cells and HIV envelope transfected melanoma cells. The siRNA-F105 carrier conjugate inhibited HIV replication in infected T cells.

To verify the targeting principle, the same strategy was applied to the integrin lymphocyte function-associated antigen-1 (Peer et al. 2007) and to target ErbB2 by Erb2 single chain antibody fused to protamine (Yao et al. 2012). With this, the genetic fusion between protamine and cell determinant was followed in a number of high-ranking publications, but this concept never was successfully translated into the clinic. However, in line with the results in the primary publications cited above, the inventors of the present application have found that the previous protamine fusion constructs exhibited only minor, if any siRNA complexing ability (see below).

It is object to the invention to provide further and preferably improved means and methods for delivering anionic molecules to a target cell. It is also object of the invention to provide improved means for complexing siRNA with targeting moieties such as antibodies.

### SUMMARY

The present invention relates to a method of generating a nanoparticle comprising contacting (a) a fusion protein (A), said fusion protein (A) comprising an antibody (A1) and a positively charged polypeptide (A2); (b) a positively charged polypeptide (B); and (c) a negatively charged molecule (C); thereby forming a nanoparticle.

The present invention also relates to a nanoparticle obtainable by a method of the invention.

The present invention also relates to a nanoparticle comprising (a) a fusion protein (A), said fusion protein (A) comprising an antibody (A1) and a positively charged polypeptide (A2); (b) a positively charged polypeptide (B); and (c) one or more negatively charged molecule(s) (C).

The present invention also relates to a composition comprising a nanoparticle of the invention.

The present invention also relates to a nanoparticle of the invention or composition of the invention for use in therapy.

### BRIEF DESCRIPTION OF THE DRAWINGS

FIG. 1: Formation of a multitude of antibody-SMCC-protamine conjugates by the application of the conjugation procedure published in (Bäumer, N. et al., 2016). Without the depletion of the excess sulfo-SMCC after reaction (as in Fehler! Verweisquelle konnte nicht gefunden werden), the residual crosslinker is able to form a multitude of different conjugates from the IgG, the protamine-SMCC and the reactive sulfo-SMCC. Examples of unintended side products, which could be observed by SDS-PAGE are: IgGs that have been internally crosslinked by excess sulfo-SMCC (A and B: anti-EGFR-mAB cetuximab), accompanied by the same crosslinked to protamine. Furthermore, we observed high molecular weight IgG multimers, which are unreducible (A), accompanied with the same crosslinked to protamine, seen in gel B). In extreme, the complexity of unintended side reactions could lead to an appearance of a cloud (B), probably formed by a mixture of all possible conjugates a to d. HC: heavy chain, LC: light chain. Leaving out the depletion of unreacted SMCC could possibly lead to the formation of unwanted side-products, that may interfere with the function of the intended product (c). For instance, reactive SMCC could lead to the crosslinking of light to heavy chain in a given IgG molecule (a) or the crosslinking oft wo IgG molecules forming a IgG dimer (d).

FIG. 2: Modification of the conjugation procedure published in (Bäumer, N. et al., 2016). A: Without the depletion of the excess sulfo-SMCC after coupling of sulfo-SMCC with protamine, the residual crosslinker is able to form a multitude of different conjugates from the IgG, the protamine-SMCC and the reactive sulfo-SMCC. Instead, the antibody-SMCC-protamine conjugate was desalted after the coupling process in the former protocol. This step was omitted in the new protocol. (C: anti-EGFR-antibody cetuximab, D: anti-IGF1R-antibody ImcA12). Examples of unintended side products, which could be observed by SDS-PAGE are highlighted by circles. B: The new conjugation protocol now includes a purification step after coupling of sulfo-SMCC and protamine. The SMCC-protamine conjugate is depleted from unbound sulfo-SMCC using a Zeba-Spin Gel purification column that retains the free sulfo-SMCC and elutes the SMCC-protamine conjugate. As a result, defined conjugation products of SMCC-protamine to heavy (HC) and light (LC) chains of the IgG antibodies cetuximab (Cet; E) and ImcA12 (A12; F) are now formed and can be observed in the Coomassie-stained SDS-PAGE. HC: heavy chain, LC: light chain, P: protamine.

FIG. 3: Antibody-mediated siRNA targeting KRAS in NSCLC. A: Targeting construct between the anti-EGFR-monoclonal antibody (mAB) cetuximab and protamine. B: anti-EGFR-mAB-protamine/free protamine (P/P) complex (α-EGFR-mAB) binds up to 8 mol siRNA/mol of antibody. C: cetuximab (anti-EGFR-mAB-) protamine/free SMCC-protamine (P/P) transports Alexa488-tagged (white dots, upper-left panel) siRNA to endosomes, but not lysosomes, since the Alexa488-positive vesicles do not overlap with the lysosomal marker Lysotracker (white dots, upper-right panel). D: α-EGFR-mAB-protamine/free protamine/siRNA (a, anti; contains P/P) treated NSCLC cells showed silenced KRAS expression by KRAS siRNA, but not control siRNA. E: Cells treated with (α-EGFR-mAB-protamine/free protamine/siRNA (P/P) showed significantly reduced colony formation with KRAS siRNAs targeting wt and G12D mutant allele. F: Systematically applied α-EGFR-mAB-protamine/free protamine in complex with control and KRAS-siRNA was well tolerated in CD1-nude mice with s.c. xenograft-transplanted SKLU1 and A549 cells, α-EGFR-mAB-protamine/free protamine/KRAS-siRNA significantly inhibited tumor growth in SKLU- and A549-tumors. G: A549 tumors were already therapeutically inhibited by cetuximab-P/P carrying control (scrambled) siRNA, but tumors in the KRAS siRNA treatment group were significantly lighter in weight than in any control groups. Excised tumors were presented in H. Statistics: Means+/−Standard Deviations in all experiments except F, here Standard Error Mean (SEM) was chosen. Significance: *p<0.05, 2-sided t-test.

FIG. 4: The proliferation marker Ki67 is less abundant in NSCLC xenografts with KRAS knockdown. Immunofluorescence determination of proliferation marker Ki67 (grey dots in A, C, E and G, I, K) on histological xenograft sections. Compared to the PBS and control siRNA carrier treated groups, the number of Ki67 positive nuclei were massively reduced in KRAS siRNA treated tumor histological sections in A549 (A-F) as well as in SK-LUI (G-L).

FIG. 5: NSCLC xenografts show higher abundance of apoptotic cells. Immunohistological assignment of apoptosis in xenograft tumor sections by TUNEL assay. A-L: A raised rate of apoptosis was seen in KRAS siRNA carrier treated tumors compared to control groups in both xenografted cell lines. M-N: statistics of TUNEL-positive nuclei in sections: The number of TUNEL-positive nuclei was two-fold increased in A549 tumor treated with EGFR-mAB-protamine/free protamine (mAB-P/P) compared to PBS treatment and three-fold increased with tumors treated with KRAS siRNA carrier. In SK-LUI, only EGFR-mAB-protamine/free protamineKRAS siRNA treatment led to a four-fold increase of apoptotic cells. α, anti; cntr, control.

FIG. 6: Rhabdomyosarcoma (RMS) cell lines can be targeted by antibody-siRNA complexes. A: Expression of cell surface receptors EGFR and IGF1R was tested in two RMS cell lines, IGFR1R and EGFR is expressed on both cell lines. B: Cetuximab-protamine (EGFR-mAB-P containing free SMCC-protamine (/P/P)) shuttled Alexa488-marked control siRNA to majority of RD cells (>90% in FACS plot C), while being less effective in RH-30 (FACS not shown). RH-30 in turn were marked by anti IGF1R directed GR11L-protamine (P/P) shuttled Alexa488 control siRNA.

FIG. 7: Targeting of RD (embryonal RMS, ERMS) and RH30 (alveolar RMS, ARMS) cells with cetuximab-protamine/free SMCC-protamine (P/P) mediated siRNA knockdown of cmyc/NRAS and KRAS as well as PAX3 in RH30 reduced colony growth in soft agar assays. Cells were harvested, treated with 30 nM cetuximab-protamine/P (EGFR-mAB-P containing free SMCC-/P) coupled to control (scr) or two siRNAs effective against c-Myc and NRAS as indicated, seeded in 96 plates in soft agar, cultivated for two weeks, stained and counted (A). Combination of two effective siRNA reduced colony numbers from 87% in control group to 63% normalized to PBS controls (B). C: Treatment of RD cells with EGFR-mAB-P/P coupled to either KRAS or NRAS-specific siRNA modestly reduced NRAS expression in the respective cells. P<0.001, 2-sided T-test D: The Sequences GGCCTCTCACCTCAGAATTC (siPF1, SEQ ID NO: 49), GCCTCTCACCTCAGAATTCA (siPF2, SEQ ID NO: 50), CCTCTCACCTCAGAATTCAA (siPF3, SEQ ID NO: 51) show respective positions of siRNAs covering the breakpoint region of the fusion oncogene PAX3-Fork head (FKHR) that is shown by the sequence TGGCCTCTCACCTCAGAATTCAATTCGTC (SEQ ID NO: 48), PAX3 part in light grey, FKHR part in dark grey. E: Cetuximab-protamine/P mediated siRNA knockdown of PAX3-FKHR in RH30 cells reduced colony growth in soft agar assays. Colony growth can be significantly inhibited by application of cetuximab-mediated breakpoint-directed siRNA siPF2 (siRNA walking visualized in D). P<0.05, 2-sided T-test

FIG. 8: Antibody-siRNA-P/P complex formation can be applied to IGF1R targeting. A: Shown here by flow cytometry, A673 Ewing sarcoma cells internalize murine anti-IGF1R antibody GR11L-sulfo-SMCC-protamine/P complexes at 37° C., such as the uncoupled GR11L antibody, which is depicted by a leftward shift in histogram signal compared to the non-internalized 4° C. control. B: Green fluorescent cytoplasmic vesicular structures in A673 cells consisting of Alexa Fluor 488-siRNA were internalized by GR11L-protamine containing free SMCC-P (arrows, right images), but not in the control experiment lacking the antibody conjugate (left images). Internalized Alexa Fluor 488-siRNA can be seen as white vesicular deposits (white arrows). Counterstaining of cell nucleus by Hoechst is shown in grey as kidney-shaped structures. Boxed areas illustrate higher magnifications of the cells indicated. Scale bars, 20 μm. C: The GSP complex was then coupled to siRNA against the mRNA of the oncogenic fusion protein EWS-FLI1, and A673 cells were treated with these complexes. As a result, EWS-FLI1 expression was downregulated as detected here in a western blot of FLI1 expression. EWS-FLI1 (E/F)-specific siRNA 2 reduced EWS-FLI1 protein expression by 80% compared to control siRNAs and the PBS control. Other E/F-specific siRNAs (siRNA 1 and FLI1-esiRNA) proved to be much less effective. EWS-FLI1 travelled as a double band at ˜64 kDa, actin at 43 kDa. Published in (Bäumer, N. et al., 2016).

FIG. 9: Anti-IGF1R-mABs A12 and Tepro for targeting of Ewing sarcoma cells. A: IGF1R-targeting mABs A12 (cixutumumab) and Tepro (teprotumumab) are expressed and purified in our lab and can be conjugated to protamine/P to enable siRNA binding and transport. IgG-protamine/P conjugates exhibit a decent molecular weight shift (arrows). HC=heavy chain, LC=light chain, −P=SMCC-protamine B: Bandshift assay using anti-IGF1R-mABs-protamine and different ratios of siRNA. C: Anti-IGF1R-mABs-protamine (containing free SMCC-P) shuttled Alexa488-marked control siRNA to SKNM-C Ewing cells (white dots).

FIG. 10: FIG. 10: Breakpoint siRNA significantly reduced colony formation in Ewing SKNM-C cells compared to control. SKNM-C cells were treated with protamine/P-conjugated A12 (A) or Tepro (B) and the indicated siRNA and subjected to colony formation assays. E/F-siRNA is an siRNA interfering with the mRNA of the driving Ewing's sarcoma EWS-Fli1. BCL2, siRNA against BCL2. P<0.05, 2-sided T-test.

FIG. 11: Illustration of a cross section through an example of a nanoparticle-like structure fulfilling those conditions for an effective antibody-SMCC-protamine/P-siRNA or -SM-1/RF carrier complex deduced from our experiments. Electrostatic binding bridges are formed between mAB, with some protamines coupled to the targeting antibody and the respective anionic cargo, which includes the siRNA (A) as well as the anionic small molecule such as SM-1/RF (B) or both (C).

FIG. 12: Antibody-protamine/free protamine conjugates can bind single-stranded antisense oligonucleotides (ASOs). The Bandshift-Assay reveals that one mol EGFR-antibody-protamine-conjugate binds 8-32 mol ASOs.

FIG. 13: Description of the molecular composition of the effective siRNA binder. Anti-CD20 mAB was conjugated to SMCC-protamine with the molar excess over the mAB as indicated. The resulting conjugate mixture was then tested for its ability to complex siRNAs. Independent from the molar excess of offered protamine-SMCC, the resulting ability to complex siRNA did not markedly differ and ranged around 16 mol siRNA per mol of carrier binder.

FIG. 14: CD20-mAB rituximab-protamine/P conjugates bind 8 mol siRNA. A: Coomassie-stained SDS-PAGE showing anti-CD20-mAB, anti-CD20-mAB coupled with 30×SMCC-protamine and molecular marker (M); HC=heavy chain, LC=light chain, −P=SMCC-protamine B: Bandshift assay using CD20-mAB-protamine/P and different ratios of siRNA.

FIG. 15: Targeting of DLBCL cell lines with antibody-P/P-siRNA complexes. Top panel: A selection of DLBCL cell lines was tested for their expression regarding CD20 and CD33 by FACS analysis. Middle panel (white dots): Alexa488-tagged siRNA was bound to protamine-conjugated CD20 (rituximab) and CD33 (gemtuzumab) monoclonal Abs (both containing free SMCC-protamine) and respective cell lines treated with the composition overnight. siRNA was internalized via the respective antibody targeting and condensed in cytoplasmic vesicular structures. Both targeting mABs (left: anti-CD20, right: anti-CD33) have been shown to transport siRNA to the respective cell lines. Lower panel: DLBCL cell lines were seeded into methylcellulose and treated with antibody-protamine/P-siRNA conjugates as indicated. Although CD33 is highly expressed in all cell lines, and rituximab transports siRNA to intracellular vesicles, the response to crucial gene knockdown as detected by colony formation capacity is low, which points towards a problematic endosomal release. In contrast, the lower expressed CD33 targeting gemtuzumab shows a much better response to gene-knockdown: Significance *p<0.01. Here, especially HBL-1 cells show a good reaction towards knockdown of BTK kinase, as well as cytoplasmic kinase SYK and further components of the B-cell receptor signalling pathway such as CARD11b, CD79B and MYD88.

FIG. 16: Synthesis of a polyanionic small molecule (SM) derivative for electrostatic transportation by monoclonal antibodies. SM-1 was conjugated to the poly-anionic red fluorescent chromophore (RF) to form a low molecular weight (1.44 kDa) poly-anion.

FIG. 17: CD20-mAB rituximab-protamine/P conjugates and EGFR-mAB cetuximab-protamine/P conjugates bind SM-1/RF. A: Bandshift assay using CD20-mAB-protamine/P and EGFR-mAB-protamine/P using different ratios of SM-1/RF up to 1:32. B: Bandshift assay using CD20-mAB-protamine/P and EGFR-mAB-protamine/P using different molecular excess of SM-1/RF up to 1:200. At least 100 mol SM-1/RF can be complexed by antibody-protamine conjugates containing also free SMCC-protamine.

FIG. 18: CD20-mAB rituximab-protamine/P conjugates and EGFR-mAB cetuximab-protamine/P conjugates transport SM-1/RF. A: CD20-positive HBL-1 DLBCL cells internalize CD20-mAB-protamine/P/SM-1/RF (containing free SMCC-P) complexes (grey shadows, left-hand side). B: EGFR-positive A549 NSCLC cells internalize EGFR-mAB-protamine/P/SM-1/RF/P complexes (white dots, left-hand side).

FIG. 19: EGFR-mAB cetuximab-protamine conjugates do not bind siRNA efficiently after depletion of free SMCC-protamine by HPLC. A: Coomassie-stained SDS-PAGE showing anti-EGFR-mAB, anti-EGFR-mAB coupled with 32×SMCC-protamine and HPLC-fractions 25-31 of anti-EGFR-mAB coupled with 32×SMCC-protamine upon depletion of unbound SMCC-protamine; HC=heavy chain, LC=light chain, −P=SMCC-protamine. B: Bandshift assays.

FIG. 20: Colony-formation assays in soft agar of NSCLC cells treated with different carriers of siRNA with and without free protamine. A: A549 cells treated with EGFR-mAB-protamine/P-KRAS-siRNA form significantly less colonies in soft agar than cells treated with EGFR-mAB-protamine/P/contr (scr)-siRNA. B: SK-LUI cells. No differences compared to PBS treated cells in colony formation can be observed when A549 (A) or SK-LUI (B) cells were treated with EGFR-mAB-protamine conjugates without free protamine (see FIG. 19A, fraction 29/30) or when using the same amount of SMCC-protamine only. Shown here are photos of colony assays and mean of three independent experiments+/−SD. Asterisks indicate significant differences (P<0.05, 2-sided T-test).

FIG. 21: CD33-mAB gemtuzumab-protamine conjugates do not bind siRNA efficiently after depletion of free SMCC-protamine by HPLC. A: Coomassie-stained SDS-PAGE showing anti-CD33-mAB, anti-CD33-mAB coupled with 32×SMCC-protamine and HPLC-fractions 24-30 of anti-CD33-mAB coupled with 32×SMCC-protamine upon depletion of unbound SMCC-protamine; HC=heavy chain, LC=light chain, −P=SMCC-protamine B: Bandshift assays. C: Colony formation assays. OCI-AML2 cells treated with CD33-mAB-protamine/P-DNMT3A-siRNA (containing free SMCC-P) form significantly less colonies in soft agar than cells treated with CD33-mAB-protamine/P-contr (scr)-siRNA (containing free SMCC-P). No differences compared to PBS treated cells in colony formation can be observed when OCI-AML2 cells were treated with CD33-mAB-protamine conjugates without free SMCC-protamine (see A+B, fraction 30). Shown here mean of three independent experiments±SD. *P<0.033, 2-sided T-test.

FIG. 22: CD20-mAB rituximab-protamine conjugates do not bind SM-1/RF efficiently after depletion of free SMCC-protamine by HPLC. A: Coomassie-stained SDS-PAGE showing anti-CD20-mAB, anti-CD20-mAB coupled with 32×SMCC-protamine and HPLC-fractions 19 25/26 of anti-CD20-mAB coupled with 32×SMCC-protamine upon depletion of unbound SMCC-protamine; HC=heavy chain, LC=light chain, −P=SMCC-protamine B: Bandshift assays with protamine-depleted (left) and protamine containing CD20-mAB preparations (right). Not SMCC-protamine depleted CD20-mAB-preparations bind >32 mol of SM-1/RF.

FIG. 23: anti-IGF1R monoclonal AB IMCA-12 (A12)-protamine conjugates do not bind siRNA efficiently after depletion of free SMCC-protamine by HPLC. A: Coomassie-stained SDS-PAGE showing anti-IGF1R, coupled with 32×SMCC-protamine and HPLC-fractions 15-21 of anti-IGF1R-mAB coupled with 32×SMCC-protamine upon depletion of unbound SMCC-protamine; HC=heavy chain, LC=light chain, −P=SMCC-protamine. B: Bandshift assays with protamine-depleted (lower part; fraction 20; see A) and SMCC-protamine containing IGF1R-mAB preparations (upper part). Not SMCC-protamine depleted IGF1R-mAB-preparations bind 8 mol of siRNA.

FIG. 24: Colony-formation assays in soft agar of SKNM-C Ewing sarcoma cells treated with A12 carrier with and without free protamine. SKNM-C cells treated with IGF1R (A12)-mAB-protamine/P-EWS-FLI1-siRNA containing free SMCC-P form significantly less colonies in soft agar than cells treated with IGF1R (A12)-mAB-protamine/P-contr (scr)-siRNA containing free SMCC-P. No differences in colony formation can be observed when SKNM-C cells were treated with EGFR-mAB-protamine conjugates without free protamine. Shown here are mean of three independent experiments+/−SD. Asterisk indicates significant differences (*P<, 2-sided T-test).

FIG. 25: Colony-formation assays in soft agar of SKNM-C Ewing sarcoma cells treated with different carriers of siRNA with and without free SMCC-protamine. SKNM-C cells treated with IGF1R (A12)-mAB-protamine/P/EWS-FLI1 (E/F)-siRNA containing free SMCC-P form significantly less colonies in soft agar than cells treated with IGF1R (A12)-mAB-protamine/P/contr (scr)-siRNA containing free SMCC-P. No differences compared to scr-siRNA (scr, scrambled) treated cells in colony formation can be observed when SKNM-C cells were treated with EGFR-mAB-protamine conjugates with or without free SMCC-protamine (see FIG. 19, fraction 29/30) or when using the same amount of SMCC-protamine only. Shown here are and means of 3 independent experiments+/−SD. Asterisk indicates significant difference (P<0.05, 2-sided T-test).

FIG. 26: Colony-formation assays in soft agar of SKNM-C Ewing sarcoma, OCI-AML-2 leukemia and A549 NSCLC cells treated with non-depleted A12 anti IGF1R mAB versus non-antibody-bound SMCC-protamine in the same concentration as in the presumed IgG-protamine-SMCC-protamine/free protamine/siRNA complex. A: SKNM-C cells treated with IGF1R (A12)-mAB-protamine/EWS-FLI1-siRNA/free SMCC-P form significantly less colonies in soft agar than cells treated with IGF1R (A12)-mAB-protamine/contr (scr)-siRNA/free SMCC-P. By contrast, if the effective EWS-FLI1 (E/F)-siRNAs are complexed to only SMCC-protamine in 1800 nM concentration, this proved to be ineffective (A, right). B: The SMCC-protamine was also used in AML cell line OCI-AML2 in conjunction with the effective DNMT3a siRNA, without targeting antibodies and showed no inhibition of colony formation. C: Last, the same setup was tested in A549 with effective KRAS siRNA bound to free SMCC-protamine with no effect and no difference to control siRNAs. Shown here are means of 3 independent experiments+/−SD. Asterisks indicate significant differences (P<0.05, 2-sided T-test).

FIG. 27: Vesicular tracking in A549 NSCLC cells. Cells treated with EGFR-mAB-protamine/free SMCC-P-Alexa488 siRNA were subjected to Lysotracker red staining. The vesicles containing Alexa488 (white dots, right panel) rarely colocalized with lysotracker (grey dots, middle panel) staining.

FIG. 28: FIG. 28: Internalization of different anti-EGFR-mAB (cetuximab) preparations in EGFR-positive NSCLC cells SK-LUI treated with different complexes. SK-LUI cells treated with EGFR-mAB-protamine/P/Alexa488-control-siRNA with (white dots in upper panel) and without free SMCC-protamine (lower panel).

FIG. 29: Internalization of different anti-EGFR-mAB (cetuximab) preparations in EGFR-positive NSCLC cells A549 treated with different complexes and free SMCC-protamine. A549 cells treated with EGFR-mAB-protamine/P/Alexa488-control-siRNA with (nuclear staining in A and siRNA as white dots in D) and without free SMCC-protamine (B and E) and free SMCC-protamine (C and F). A-C: Nuclear staining using Hoechst, D-F: Green channel (white dots) depicting the same cells as in A-C for Alexa488-siRNA internalized vesicles.

FIG. 30: Internalization of different anti-CD33-mAB (gemtuzumab) preparations in CD33-positive AML cells OCI-AML2 treated with different complexes. OCI-AML2 cells treated with anti-CD33-mAB-protamine/P/Alexa488-control-siRNA with (A and D) and without free SMCC-protamine (B and E) and free SMCC-protamine (C and F). A-C. Nuclear staining using Hoechst (grey dots), D-F. Green channel (white dots in D) depicting the same cells as in A-C for Alexa488-siRNA internalized vesicles.

FIG. 31: Internalization of different anti-IGF1R-mAB (ImcA12) preparations in IGF1R-positive Ewing's sarcoma cells SKNM-C treated with different complexes. SKNM-C cells treated with anti-IGF1R-mAB-protamine/P/Alexa488-control-siRNA with (A and D) and without free SMCC-protamine (B and E) and free SMCC-protamine (C and F). A-C: Nuclear staining using Hoechst, D-F: Green channel (white dots in D) depicting the same cells as in A-C for Alexa488-siRNA internalized vesicles.

FIG. 32: Presence of different anti-EGFR-mAB (cetuximab) preparations in EGFR-negative SKNM-C-cell cultures. Lower panels depict higher magnifications of insets of upper panels as indicated by white frames. A: Uncoupled cetuximab does not transport Alexa488-control siRNA into SKNMC-cells. B: Protamine-coupled cetuximab does not transport Alexa488-control siRNA into SKNMC-cells, Alexa488-positive vesicular structures only occur next to the cells (white dots, arrow in upper panel) and in cell-free areas of the culture (arrow in lower panel). C: Protamine-coupled cetuximab without free SMCC-protamine (which was removed by HPLC) does not form Alexa488-positive vesicular structures anymore.

FIG. 33: EGFR-mAB cetuximab-protamine conjugates in DLS measurements. Upper panel: Coomassie-stained PAGE gel depicting the different complexes isolated or used for the lower panel measurement. Lower panel: EGFR-mAB-P with and without free SMCC-protamine and SMCC-protamine alone were incubated for 2 hrs at room temperature and then measured via dynamic light scattering (DLS) on a zeta-counter (MALVERN). The different peaks represent particles of different size in nm. While the highest peak with EGFR-mAB-P with free SMCC-protamine/siRNA occurs at around 427 nm, the same without free SMCC-protamine only produces a peak at around 3.2 nm and free SMCC-protamine/siRNA at 5.7 nm.

FIG. 34: EGFR-mAB cetuximab-protamine/P conjugates in DLS measurements during 0 to 24 hrs incubation at room temperature. The siRNA monomers (appr. 1.92 nm) assembled by the cetuximab-protamine/unbound protamine carrier system to larger structures after mixing, which stabilize and further assemble to much larger macrostructures (˜500 nm). After 24 h in unprotected environment in PBS, the macrostructures start to partially dis-assemble again. Numbers below indicate the measured size of particles in nm.

FIG. 35: Antibody-protamine/free SMCC-P (P/P) conjugates with fluorescent Alexa488-siRNA (white dots) in cell-free incubation overnight on chamber slides. A: EGFR-mAB-P/P, B: CD20-mAB-P/P, C: CD33-mAB-P/P, D: IGF1R-mAB-P/P at 40× magnification, bars=10 μm. E-H: Higher magnifications of A-D, bars still 10 μm.

FIG. 36: Antibody-protamine conjugates with and without free SMCC-protamine and SMCC-protamine only with fluorescent Alexa488-siRNA (white dots) in cell-free incubation overnight on chamber slides. Antibody complexes with free SMCC-protamine: A-D. A. EGFR-mAB-P, B. CD20-mAB-P, C. CD33-mAB-P, D. IGF1R-mAB-P. E. free SMCC-protamines. Antibody complexes without free SMCC-protamine: F-I. F. EGFR-mAB-P, G. CD20-mAB-P, H. CD33-mAB-P, I. IGF1R-mAB-P. All at 40× magnification.

FIG. 37: FIG. 37: Fluorescence light microscopy (A and B) and laser scan microscopy (LSM) photographs on one confocal optical section (C and D) of antibody complexes. Formation of cetuximab anti-EGFR-mAB-protamine/free SMCC_P conjugates (A and C) and anti-CD20-mAB-protamine/free SMCC-P (B and D) with fluorescent Alexa488-siRNA (white dots) in cell-free incubation overnight on chamber slides.

FIG. 38: Anti-EGFR-antibody-protamine conjugates with free SMCC-protamine with fluorescent Alexa488-siRNA (white dots) in cell-free incubation overnight on chamber slides at different temperatures as indicated.

FIG. 39: Conjugation of cetuximab with different ratios of antibody to SMCC-protamine. A: Detailed formulation of each conjugation process. B: Coomassie-stained SDS-PAGE showing uncoupled anti-EGFR-antibody cetuximab compared to the conjugation products that were coupled as depicted in A.

FIG. 40: Functional analysis of the conjugation products of cetuximab with different ratios of antibody to SMCC-protamine. A-F: Bandshift assays using the different conjugation products as introduced in Figure. G-L: Formation of vesicles without cells on slides when the different conjugation products were incubated with Alexa488-siRNA (white dots, especially in J). M-R: Internalisation of the different cetuximab-SMCC-protamine/P/Alexa488-siRNA complexes into EGFR-positive A549 cells (white dots, arrows). S-X: Colony formation of A549 cells treated with the different cetuximab-SMCC-protamine/P conjugations containing free protamine in complex with control (“scr”) siRNA or anti-KRAS siRNA (“KRAS”). When more than 50×SMCC-protamine is used, the conjugate is unspecifically toxic. Shown here are means of 3 independent experiments+/−SD. Asterisk indicates significant differences (P<0.009, 2-sided T-test).

FIG. 41: Functional analysis of the vesicle formation of anti-EGFR-mAB-protamine depleted from free SMCC-protamine with different ratios of supplemented SMCC-protamine or protamine alone. Formation of vesicles without cells on slides upon incubation with Alexa488-siRNA. The anti-EGFR-mAB-protamine without free SMCC-protamine does not form vesicles as shown in Figure (white dots). When free SMCC-protamine was added stepwise, vesicle formation does not occur with 1×SMCC-protamine (A), to a low extent with 10×SMCC-protamine (B) and to a high extent with 32×SMCC-protamine (C). When free protamine that was not coupled to sulfo-SMCC was added stepwise, vesicle formation does not occur with 1×SMCC-protamine (D), to a low extent with 10×SMCC-protamine (E) and to a high extent with 32×SMCC-protamine (white dots in F).

FIG. 42: Formation of anti-CD20-mAB-protamine/free SMCC-P conjugates with fluorescent Alexa488-siRNA and/or SM-1/RF in cell-free incubation o/n on chamber slides. A.-C. green fluorescence channel (white dots), D.-F. red fluorescence channel (grey dots). A. and D. anti-CD20-mAB-P/P with Alexa488-siRNA, B. and E. anti-CD20-mAB-P/P with red fluorescent SM-1/RF, C. and F. anti-CD20-mAB-P/P with Alexa488-siRNA and red fluorescent SM-1/RF, at 40× magnification, bars=10 μm.

FIG. 43: Formation of anti-EGFR-mAB-protamine/free SMCC-P conjugates with fluorescent Alexa488-siRNA and/or SM-1/RF in cell-free incubation o/n on chamber slides. A-D: green fluorescence channel (white dots), E-H: red fluorescence channel (grey dots). A. and E. EGFR-mAB-P/P with Alexa488-siRNA, B. and F. EGFR-mAB-P/P with red fluorescent SM-1/RF, C. and G. EGFR-mAB-P/P with non-fluorescent control-siRNA and red fluorescent SM-1/RF, D. and H. EGFR-mAB-P/P with green fluorescent Alexa488-siRNA and red fluorescent SM-1/RF, at 40× magnification, bars=10 μm.

FIG. 44: Formation of rituximab anti-CD20-mAB-protamine/free SMCC-P conjugates with fluorescent Alexa488-siRNA combined with SM-1/RF in cell-free incubation o/n on chamber slides. A-C: 40× magnifications green (white dots) and red fluorescence (grey dots) channel, D-L: Equal magnifications of details from A-C, bars=10 μm. In D, G and L: grey rings depict Alexa488-siRNA fluorescence (arrows). In E, H and K: Grey circle depict red-fluorescence of SM-1/RF (arrows). In F, I, and L green-fluorescent (grey) rim (Alexa488-siRNA, arrows) and red internal fluorescence (SM-1/RF) can be discriminated.

FIG. 45: Formation of cetuximab anti-EGFR-mAB-protamine/free SMCC-P conjugates with green-fluorescent Alexa488-siRNA combined with red-fluorescent SM-1/RF in cell-free incubation o/n on chamber slides. A-C: 40× magnifications green (white and rings in A and C) and red fluorescence channel (grey circles in B and C). D: Magnifications of detail from A-C, bars=10 μm.

FIG. 46: Large micellar structures formed by anti-CD20-mAB/P/free SMCC-/P (grey rings in A), SM-1/RF (grey circles in B) and Alexa488-siRNA are visible in light microscopy in phase contrast (B and C). bars=5 μm.

FIG. 47: LSM photographs on one confocal optical section and Z-stacks of antibody complexes. A: Formation of anti-EGFR-mAB (cetuximab)-protamine/free SMCC-P conjugates with fluorescent Alexa488-siRNA (white dots) combined with SM-1/RF in cell-free incubation o/n on chamber slides. a: one level across a vesicle, b and c: Z-stacks reconstituting the 3D-structure of the vesicle on both axes. B: Formation of anti-CD20-mAB (rituximab)-protamine/P conjugates with fluorescent Alexa488-siRNA (white rings and dots) combined with SM-1/RF (grey shadows) in cell-free incubation o/n on chamber slides. d: one level across a vesicle, e and f: Z-stacks reconstituting the 3D-structure of the vesicle on both axes.

FIG. 48: Properties of a genetic fusion of gemtuzumab-heavy chain and human protamine as a siRNA carrier: An illustrative SDS-PAGE of αCD33-mAB gemtuzumab chemically conjugated in two different molar ratios of protamine-SMCC to gemtuzumab compared to the genetic fusion, where one protamine is C-terminally fused to the heavy chain HC. Although the resulting fusion protein is 100% protamine decorated on the HC, the fusion does not bind siRNA (C). By contrast, the regular chemical conjugate 1:30 excess SMCC-protamine over gemtuzumab binds siRNA in a 1:8 ratio (B). αCD33-mAB-P, anti-CD33-monoclonal antibody gemtuzumab chemically coupled to protamine; αCD33-mAB-hPRM1 fusion, anti-CD33-monoclonal antibody gemtuzumab genetically fused to human protamine 1 (PRM1) cDNA; HC, heavy chain, LC, light chain, HC-P, heavy chain with protamine; LC-P, light chain with protamine.

FIG. 49: Properties of a genetic fusion of gemtuzumab-heavy chain and human protamine as a siRNA carrier: When the αCD33-mAB-hPRM1 fusion protein was supplemented with different amounts of free protamine-sulfate, siRNA binding was restored as detected by band-shift assays (A: 20×, B: 25×, C: 30×molar excess of free protamine-sulfate).

FIG. 50: Properties of a genetic fusion of gemtuzumab-heavy chain and human protamine as a siRNA complexer: Reconstitution of the siRNA binding properties by adding free protamine-sulfate. The purified gemtuzumab-protamine genetic fusion was modified by adding non-conjugated protamine as an additional complexer comparable to the excess SMCC-chemical conjugation. The addition of free protamine-sulfate reconstituted the ability of the genetic fusion to complex Alexa488-siRNA to nanoparticles. Whereas a 10× excess of protamine was not enough to fulfil the requirements for complexing Alexa488, the samples with higher protamine-sulfate content were suitable as siRNA carriers and formed regular micellar structures. Upper panels: fluorescent fotographs in 40× magnification, lower panels: fluorescent fotographs in 400× magnification. Grey dots represent green fluorescent Alexa488-siRNA signal observed as vesicular nano-structures. αCD33-mAB-hPRM1 fusion, anti-CD33-monoclonal antibody gemtuzumab genetically fused to human protamine 1 (hPRM1) cDNA; αCD33-mAB-P, anti-CD33-monoclonal antibody gemtuzumab chemically coupled to SMCC-protamine; 32×SMCC-P, 32×excess of free SMCC-coupled protamine.

FIG. 51: Schematic depiction of an exemplary gemtuzumab-protamine fusion protein. A. The gemtuzumab (anti-CD33-mAB)-heavy chain was fused to a linker and subsequently to the human protamine 1 (hPRM1) cDNA, another linker and an endosomal escape domain (sequence: GFWFG; “EED1”). An illustrative example of an expression vector for this fusion protein is shown in SEQ ID NO: 65. B. The teprotumumab (anti-IGF1R-mAB)-heavy chain was fused to a linker and subsequently to the human protamine (PRM1) cDNA. An illustrative example of an expression vector for this fusion protein is shown in SEQ ID NO: 78. C. The teprotumumab (anti-IGF1R-mAB)-light chain and heavy chain were fused to a linker and subsequently to the human protamine (PRM1) cDNA. An illustrative example of an expression vector for this fusion protein is shown in SEQ ID NO: 80. D. The teprotumumab (anti-IGF1R-mAB)-light chain was fused to a linker and subsequently to the human protamine (PRM1) cDNA. An illustrative example of an expression vector for this fusion protein is shown in SEQ ID NO: 82.

FIG. 52: Schematic depiction of an exemplary cetuximab-protamine fusion protein. A. The cetuximab (anti-EGFR-mAB)-heavy chain was fused to a linker and subsequently to the human protamine (PRM1) cDNA. An illustrative example of an expression vector for this fusion protein is shown in SEQ ID NO: 70. B. The cetuximab (anti-EGFR-mAB)-light chain as well as the heavy chain was fused to a linker and subsequently to the human protamine (PRM1) cDNA. An illustrative example of an expression vector for this fusion protein is shown in SEQ ID NO: 72. C. The cetuximab (anti-EGFR-mAB)-light chain was fused to a linker and human protamine 1 (PRM1) cDNA and cetuximab light chain was fused to a linker and to the human protamine 2 (PRM2) cDNA. An illustrative example of an expression vector for this fusion protein is shown in SEQ ID NO: 74. D. The cetuximab (anti-EGFR-mAB)-light chain was fused to a linker and subsequently to the human protamine (PRM1) cDNA. An illustrative example of an expression vector for this fusion protein is shown in SEQ ID NO: 76. E. The cetuximab (anti-EGFR-mAB)-light chain was fused to a linker and subsequently to the human protamine (PRM2) cDNA. An illustrative example of an expression vector for this fusion protein is shown in SEQ ID NO: 77.

FIG. 53: Schematic depiction of exemplary antibody-protamine fusion proteins. Construct 1: The heavy chain of an antibody is fused to a linker and subsequently to the human protamine (PRM) cDNA. In construct 2, PRM-cDNA is fused to the light chain (LC) of the antibody. Construct 3: PRM can also be fused via a linker to the heavy chain (HC) as well as the light chain (HC) of antibody. PRM can be human PRM1 or PRM2 or protamine coding sequences of other species.

FIG. 54: αCD33-mAB gemtuzumab-protamine fusion reduces colony formation in presence of free protamine-sulfate. Colony formation assays. OCI-AML2 cells treated with αCD33-mAB-PRM1/DNMT3A-siRNA (D3A) in presence of 20×, 25× or 30× free protamine-sulfate, respectively, form significantly less colonies in soft agar than cells treated with αCD33-mAB-protamine/contr (scr)-siRNA with the same amounts of free protamine-sulfate. No differences compared to PBS treated cells in colony formation can be observed when OCI-AML2 cells were treated with αCD33-mAB-PRM1 fusion proteins without free protamine-sulfate. Shown here mean of three independent experiments±SD. *P<0.05, 2-sided T-test. α, anti.

FIG. 55: A: Visualization of a molecular weight shift of the cetuximab heavy chain caused by genetic fusion of linker-hPRM-1 in SDS-PAGE. B: Cetuximab-hPRM-1 fusion protein itself is unable to bind siRNA in significant amounts. C: The addition of a 20× molar excess of free protamine to the preparation from A and B enables a binding of 8 mol siRNA/mol of cetuximab-fusion. D and E: Rising the molar excess of protamine to 25× and 30×, respectively, enables more siRNA to be bound to the resulting complex. α, anti.

FIG. 56: Deciphering optimal conditions for the formation of nanocomplexes from siRNA, free protamine and αEGFR-(cetuximab)-hPRM1-fusion. Here, rising excess concentrations of free protamine, siRNA and cetuximab-hPRM1-fusion were allowed to form nanocomplexes in a cell-free environment. Complexes form in an optimal way between 32 and 50×molar excess of free protamine. Below: phase contrast microscopy. α, anti.

FIG. 57: Proposed model of an idealized nanocomplex consisting of siRNA, free protamine and anti-receptor-IgG-hPRM1-fusion. It is observed that IgG-hPRM-1-fusion forms a shell structure framing certain and self-stabilizing amounts of siRNA and free protamine in a self-organized fashion. The spheroid structures have an approximate diameter with 50 to 500 nm being the most prevalent fraction and forming in a time-dependent manner.

FIG. 58: Internalization of Alexa488-marked siRNA to A549 NSCLC cells driven by cetuximab-(αEGFR)-hPRM-1 fusion protein combined by rising excess concentrations of free protamine. Upper panels (A-D): Nuclei stained with Hoechst counterstain. E-H: Green fluorescence, representing Alexa488-siRNA. Here, the preparation lacking free protamine-sulfate (E)) was unable to transport Alexa488-siRNA to A549 cells, whereas rising the protamine-sulfate concentration in the mixture gave rise to increasing number of internalized intracellular vesicles filled with Alexa488-siRNA (white dots in F, G and H), the optimal molar excess was around 30× free protamine-sulfate per mol of cetuximab-hPRM-1 fusion protein in conjunction with Alexa488-siRNA.

FIG. 59: αEGFR-mAB cetuximab-protamine fusion reduces colony formation in presence of free protamine-sulfate. Colony formation assays. A549 cells treated with αEGFR-mAB-PRM1/KRAS-siRNA in presence of 20× or 30× free protamine-sulfate, respectively, form significantly less colonies in soft agar than cells treated with αEGFR-mAB-protamine/contr (scr)-siRNA with the same amounts of free protamine-sulfate. No differences compared to PBS treated cells in colony formation can be observed when A549 cells were treated with αEGFR-mAB-PRM1 fusion proteins without free protamine-sulfate. Shown here mean of three independent experiments±SD. *P<0.05, 2-sided T-test. α, anti.

FIG. 60: Synthesis of a polyanionic ibrutinib derivative for electrostatic transportation by monoclonal antibodies. Ibrutinib was conjugated to the Cy3.5 chromophore to form a low molecular weight (1.44 kDa) polyanion. Chemical structure of the anionic ibrutinib-Cy3.5 (Cy3.5-RMA561) which builds with the cationic protamine-linked mAB stable vesicles upon electrostatic interaction.

FIG. 61: High resolution mass spectrometry of Cy3.5-RMA561. The sample was ionized and fragmented by electron beam in a mass spectrometer, resulting fragments were analysed by their mass-to-charge (m/z) ratio according to their specific deflection. HRMS (ESI, CH₃CN/H₂O):
m/z calc. for C₆₄ H₆₂ N₉ O₁₅ S₄³⁻ [M-H] (z=3): 441.44216; found: 441.44160;
m/z calc. for C₆₄ H₆₂ N₉ O₁₅ S₄H²⁻ [M-H] (z=2): 662.66687; found: 662.66630;
m/z calc. for (C₆₄ H₆₂ N₉ O₁₅ S₄H)₂⁴⁻ +[M-H] (z=4): 662.66687; found: 662.66630.

FIG. 62: αCD20-mAB rituximab-protamine/free protamine-SMCC conjugates and αEGFR-mAB cetuximab-protamine/free protamine-SMCC conjugates bind ibrutinib-Cy3.5. A. Bandshift assay using αCD20-mAB-protamine/P and αEGFR-mAB-protamine/P using different ratios of ibrutinib-Cy3.5 up to 1:32. B. Bandshift assay using αCD20-mAB-protamine/free protamine-SMCC and αEGFR-mAB-protamine/free protamine-SMCC using different molecular excess of ibrutinib-Cy3.5 up to 1:200. At least 100 mol ibrutinib-Cy3.5 can be complexed by antibody-protamine conjugates. α, anti.

FIG. 63: αCD20-mAB rituximab-protamine/free protamine-SMCC/ibrutinib-Cy3.5 conjugates and αEGFR-mAB cetuximab-protamin/free protamine-SMCC conjugates internalize ibrutinib-Cy3.5. A. CD20-positive HBL-1 DLBCL cells internalize αCD20-mAB-protamine/free protamine-SMCC/ibrutinib-Cy3.5 complexes. B. EGFRpositive A549 NSCLC cells internalize αEGFR-mAB-protamine/free protamine-SMCC/ibrutinib-Cy3.5 complexes. α, anti.

FIG. 64: Covalent labelling of BTK kinase in vitro by ibrutinib-Cy3.5 conjugate transported by rituximab-protamine/free protamine-SMCC as a carrier molecule (αCD20-mAB-P/ibrutinib-Cy3.5). 10⁵ cells were treated with the indicated concentrations of compounds overnight, lysed in loading dye and run over the gel. The gel was exposed to UV light on a SYBR Gold filter (left) for Cy3.5 emission on an INTAS gel imager, then blotted and incubated with anti-BTK-mAB for identification (right). Intracellular BTK bound free ibrutinib-Cy3.5 as well as antibody-protamine-complexed ibrutinib-Cy3.5. RTX, rituximab.

FIG. 65: αCD20-mAB (rituximab)-protamine/free protamine conjugates and αEGFR-mAB cetuximab-protamine/free protamine conjugates transport ibrutinib-Cy3.5 and inhibit colony formation more effectively than ibrutinib-Cy3.5 or the antibody alone. A. and B. Colony formation assays. HBL-1 cells (A) treated with αCD20-mAB (rituximab)-protamine/free protamine-SMCC/ibrutinib-Cy3.5 and (B) A549 cells treated with αEGFR mAB-(cetuximab) protamine/free protamine-SMCC/ibrutinib-Cy3.5 form significantly less colonies in methylcellulose than cells treated with PBS, uncomplexed ibrutinib-Cy3.5, or treated with αCD20 mAB-(rituximab) protamine/free protamine-SMCC/ibrutinib-Cy3.5. Shown here are means of 3 independent experiments±SD. Asterisks show significant differences, (p-values<0.05, 2-sided t-test). α, anti.

FIG. 66: αCD20-mAB rituximab-protamine conjugates do not bind ibrutinib-Cy3.5 efficiently after depletion of free protamine-SMCC by HPLC. A. Coomassie-stained SDS-PAGE showing αCD20-mAB, αCD20-mAB coupled with 32×protamine-SMCC and HPLC-fractions 19 25/26 of αCD20-mAB coupled with 32×protamine-SMCC upon depletion of unbound protamine-SMCC; HC=heavy chain, LC=light chain, −P=protamine-SMCC B. Bandshift assays with protamine-depleted (left) and protamine containing αCD20-mAB preparations (right). Not protamine-SMCC depleted αCD20-mAB-preparations bind >32 mol of ibrutinib-Cy3.5. C. Colony formation assays. HBL-1 cells treated with αCD20-mAB-protamine/free protamine-SMCC/ibrutinib-Cy3.5 form significantly less colonies in soft agar than cells treated with uncoupled ibrutinib-Cy3.5 or uncoupled αCD20-mAB alone. No differences compared to PBS treated cells in colony formation can be observed when HLB-1 cells were treated with αCD20-mAB-protamine conjugates without free protamine-SMCC (see A+B, fraction 25). Shown here are means of three independent experiments±SD. *P<0.0003, 2-sided T-test. α, anti.

FIG. 67: αCD20-mAB rituximab-protamine/free protamine-SMCC conjugates efficiently coordinate and transport ibrutinib-Cy3.5 to the tumor site in vivo and reduce tumor growth significantly. A. Tumor growth and treatment regimen of the NSG-HBL1 xenograft model. After transplantation, tumors were grown to 200 mm³, before treatment was started twice a week intraperitoneally (i.p.). B. Treatment with rituximab-protamine/free protamine/ibrutinib-Cy3.5 1:20 complex (4 mg/kg mouse weight, or 0.625 nmol rituximab-protamine and/or 12.5 nmol ibrutinib derivatives per single dose) (in the picture=rituximab-ibrutinib-Cy3.5 (C) or rituximab-P/P/ibrutinib-Cy3.5 (B)) significantly reduced tumor volumes and growth. On every treatment day, twice a week, tumor volumes were assessed by caliper measurements. Whereas in the rituximab-protamine/free protamine/ibrutinib-Cy3.5 1:20 complex treated group, the tumor volume was limited to well below 1,000 mm³ and after three treatments started to shrink to 600 mm³, all other groups showed fast tumor growth and had to be sacrificed early by predefined legal regulations. C. Survival curves of the treatment versus control groups. Groups of 10 mice each were treated with PBS, rituximab, ibrutinib standard, ibrutinib-Cy3.5 and rituximab-protamine/free protamine/ibrutinib-Cy3.5 1:20 complex. Ibrutinib-Cy3.5 did not reduce tumor growth eight days after treatment start, all mice had to be sacrificed because of predefined criteria, whereas PBS and rituximab treated mice lived insignificantly longer up to day 16. The last of 10 ibrutinib treated mice had to be sacrificed at day 20, whereas 5 of 10 rituximab-protamine/free protamine/ibrutinib-Cy3.5 1:20 complex treated mice survived until day 16 and four to day 20 post treatment start. The difference between the rituximab-protamine/free protamine/ibrutinib-Cy3.5 treatment group and the controls was evaluated as p≤0.03 (ANOVA). α, anti, RTX, rituximab.

FIG. 68: Tumors of HBL-1 cells xenografted in NSG mice show a marked enrichment of Cy3.5 fluorescence signals in rituximab-protamine/free protamine-SMCC/ibrutinib-Cy3.5 treated mice. Xenograft mice from the experiment shown in FIG. 55 were sacrificed after reaching intolerable tumor sizes, organs as well as tumors prepared and exposed to ex vivo fluorescence detection for Cy3.5 signals at 530 nm excitation and 600 nm emission. Tumors from the rituximab-P/free P/ibrutinib (in the picture=Rtx/ibrutinib-Cy3.5) treated group (lower row) showed marked enrichment of Cy3.5-dependent fluorescence signals as compared to non-targeted ibrutinib-Cy3.5 and standard ibrutinib over all parts of the tumor tissue, while in control organs, only necrotic foci showing auto-fluorescence could be detected. The diameters of the tumor preparations shown were similar in all cases, but differ in the fluorescence area. Scale represents arbitrary units of fluorescence. Dotted lines represent outer limits of each tumor. Numbers refer to individual mouse identifiers.

FIG. 69: Overview about different mouse organs analysed for Cy3.5 fluorescence from NSG mice xenografted with HBL1 mice. Xenograft mice from the experiment shown in FIGS. 55 and 56 were sacrificed after reaching intolerable tumor sizes, organs as well as tumors prepared and exposed to ex vivo fluorescence detection for Cy3.5 signals at 530 nm excitation and 600 nm emission. Tumors from the rituximab-P/free P/ibrutinib (in the picture=Rtx/ibrutinib-Cy3.5) treated group (lower rows) showed marked enrichment of Cy3.5-dependent fluorescence signals as compared to non-targeted ibrutinib-Cy3.5. Scales represents arbitrary units of fluorescence. Organs are always arranged in the same orientation (as depicted in the scheme on the right side) in bright field (upper panels) and red (Cy3.5) fluorescence (lower panels).

FIG. 70: Formation of rituximab αCD20-mAB-protamine/free SMCC-protamine nano-vesicles with green-fluorescent Alexa488-siRNA and/or red-fluorescent ibrutinib-Cy3.5 in cell-free incubation overnight (o/n) on chamber slides. A.-C. green fluorescence channel, D.-F. red fluorescence channel. A. and D. αCD20-mAB-P/free P with green Alexa488-siRNA, B. and E. αCD20-mAB-P/free P with red fluorescent ibrutinib-Cy3.5, C. and F. αCD20-mAB-P/free P with green Alexa488-siRNA and red ibrutinib-Cy3.5, at 40× magnification, bars=10 μm. All rituximab-protamine preparations contain unbound protamine-SMCC. α, anti.

FIG. 71: Formation of cetuximab αEGFR-mAB-protamine/free protamine-SMCC conjugates with green-fluorescent Alexa488-siRNA and/or red-fluorescent ibrutinib-Cy3.5 in cell-free incubation overnight (o/n) on chamber slides. A.-D. green fluorescence channel, E-H. red fluorescence channel A. and E. Cetuximab αEGFRmAB-P/free protamine-SMCC with green-fluorescent Alexa488-siRNA, B. and F. cetuximab αEGFR-mAB-P/free protamine-SMCC with red-fluorescent ibrutinib-Cy3.5, C. and G. αEGFR-mAB-P with non-fluorescent control siRNA (scr, scrambled) and red-fluorescent ibrutinib-Cy3.5, D. and H. αEGFR-mAB-P with non-fluorescent Alexa488-siRNA and red-fluorescent ibrutinib-Cy3.5, at 40× magnification, bars=10 μm. All cetuximab protamine preparations contain unbound protamine-SMCC. α, anti.

FIG. 72: Formation of cetuximab αEGFR-mAB-protamine/free protamine-SMCC (A-B) and rituximab anti-CD20-mAB-protamine/free protamine-SMCC (C-D) conjugates with green-fluorescent Alexa488-siRNA combined with red-fluorescent ibrutinib-Cy3.5 in cell-free incubation overnight (o/n) on chamber slides. 40× magnifications of green- and red-fluorescence channels, In A and C, green-fluorescent rim (Alexa488-siRNA) is visible, while in B and D, red internal fluorescence (ibrutinib-Cy3.5) can be seen. All antibody-protamine preparations contain unbound protamine-SMCC.

FIG. 73: Determination of particle sizes in different complex formations of αCD20-mAB rituximab-protamine/free protamine-SMCC with siRNA and ibrutinib-Cy3.5. A. Graphic illustration of the mean diameter (nm) shown in B-E. Zetaview measurements of the indicated complexes were performed at 1 h and 2 h after incubation start. Shown here is the mean vesicle size (nm) that is determined by the mean diameter of each particle depicted in the histograms in B-E. All rituximab-protamine preparations contain unbound protamine-SMCC. α, anti.

FIG. 74: A: αCD20-mAB rituximab-protamine/free protamine-SMCC (αCD20-mAB-P/P) conjugates bind ibrutinib-Alexa488. Bandshift assay using αCD20-mAB-protamine/P using different ratios of ibrutinib-Alexa488 up to 1:2. α, anti. B: Due to the limited anionic charge of the Alexa488 molecule of −2 (arrows), the interactions between the polycationic protamine fusions and Alexa488 were found to be less intense than those with Cy3.5, which has a net charge of −4. With Alexa488-conjugated ibrutinib and protamine conjugates, coupling ratios of only 2:1 were realized. However, complexation of ibrutinib-Alexa488 with αCD20-mAB rituximab-protamine/free protamine-SMCC (αCD20-mAB-P/P) was still successful. C—H: Stability after 1 h-auto-assembly of αCD20-mAB-protamine, free protamine and ibrutinib-Cy3.5 in a 1:20 ratio and subsequent incubation for 24 h in PBS (C, D), and in challenging conditions such as cell culture medium RPMI/10% FCS (E, F) and PBS/50% FCS (G, H). C, E, G: Cy3.5 fluorescence, D, F, H: phase contrast. α, anti.

FIG. 75: Charged ibrutinib-Cy3.5, but not uncharged ibrutinib (trade name: imbruvica) forms stable nanoparticles with different protamine-conjugated mABs. The respective antibody carriers, conjugated via SMCC to protamine and containing free SMCC-protamine were loaded with charged ibrutinib-Cy3.5 in comparison to uncharged ibrutinib. Only those ibrutinib samples conjugated with Cy3.5 showed a dense formation of nanoparticles, but not uncharged ibrutinib. Tested were anti-EGFR antibody (A-D), anti-CD33 antibody (E-H) and anti-IGF1R antibody (I-L) in Cy3.5 dependent fluorescence micrographs (top) and phase contrast (lower). α, anti.

FIG. 76: Charged ibrutinib-Cy3.5, but not uncharged ibrutinib (trade name: imbruvica) forms stable nanoparticles with different protamine-fused mABs. Here, we used hPRM1-protamine-fusions of anti-EGFR (A-D) as well as hPRM1-fusions with anti-CD33 to complex charged ibrutinib-Cy3.5. Stable nanoparticles were formed with ibrutinib-Cy3.5, but not with uncharged ibrutinib. Tested were anti-EGFR antibody (αEGFR-mAB-PRM1: A-D) and anti-CD33 antibody (αCD33-mAB-PRM1: E-H) in Cy3.5 dependent fluorescent micrographs (top) and phase contrast (lower). α, anti.

FIG. 77: Illustration of a cross section through an ideal example of a nanoparticle-like structure fulfilling those conditions for an effective antibody-protamine-protamine-siRNA or -ibrutinib-Cy3.5 carrier complex deduced from our experiments. Illustrations not to scale. Electrostatic binding bridges are formed between mAB, with some protamines coupled to the targeting antibody and the respective anionic cargo, which includes the siRNA (A) as well as the ibrutinib-Cy 3.5 (B) or both (C).

FIG. 78: Electrostatic nanoparticle formation by αCD20-mAB-protamine/free protamine-ibrutinib-Cy3.5. The carrier antibody-protamine conjugate was loaded with anionic ibrutinib-Cy3.5 in 1:20 ratio and applied to cell-culture treated glass slides for fluorescence microscopy (A, B) or copper grids for phospho-Wolfram negative stained electron microscopy (C). Here, the electrostatic loading led to the formation of numerous aggregates, where the larger aggregates showed intense Cy3.5 fluorescence (A) and were visible in light microscopy using emboss dynamic filter to illustrate 3D structures through contrast enhancement (B). In transmission electron microscopy (C), negative staining led to roughly the same range of particle sizes but revealed the presence of a plethora of smaller vesicles (C) undetectable in light microscopy. α, anti.

FIG. 79: Cellular targeting of Bruton's kinase BTK by αCD20-mAB-P/P-complexed ibrutinib-Cy3.5. A-F: Fluorescence microscopy of HBL1 DLBCL cells treated with targeting conjugates and controls showing a marked intracellular enrichment of Cy 3.5-signals. G: lysates from cells treated for 72 h with targeting conjugates and controls were subjected to SDS PAGE and illuminated for Cy 3.5 signals. Here, a clear band of 70 kDa, identified as BTK by parallel immunoblot, was covalently marked by ibrutinib-Cy3.5, indicating binding and thus functionality of the ibrutinib-Cy3.5 derivate. H-P: fluorescence microscopy of HBL1 DLBCL cells pre-treated with ibrutinib-bodipy (green, N and P) do not show intracellular enrichment of Cy3.5-signals after αCD20-mAB-P/P-ibrutinib-Cy3.5 treatment (M, compared to L). α, anti.

FIG. 80: Physiological and functional consequences of BTK-inactivation by αCD20-mAB-protamine/free protamine-ibrutinib-Cy3.5 treatment in DLBCL cell lines. A: HBL1 cells were treated by the respective conjugates shown for 72 hrs, lysed and subjected to SDS-PAGE and Western blotting for phospho-BTK (pBTK), total BTK (tBTK), phospho-ERK (p-ERK), total-ERK (t-ERK) and actin as a loading control. Here, untargeted ibrutinib-Cy3.5 inhibited the phosphorylation of BTK a bit less than &CD20-mAB-protamine-ibrutinib-Cy3.5, the difference of expected downstream phosphorylation targets such as ERK was more pronounced: Here, only αCD20-mAB-P/P-mediated ibrutinib-Cy3.5 treatment was able to reduce ERK phosphorylation. B: In colony formation assays, untargeted ibrutinib-Cy3.5 modestly reduced colony growth of HBL1 cells, while the specific targeting of ibrutinib-Cy3.5 by αCD20-mAB-P/P boosted the colony growth reduction to below 30%. In order to demonstrate the significance of the free protamine in the conjugate construct, we depleted it from the conjugate mixture, the application of this combination revealed no more colony forming reduction than the single application of ibrutinib-Cy3.5, so the antibody conjugate has lost its targeting ability (B, rightmost bar). α, anti.

FIG. 81: Induction of apoptosis of BTK targeting by αCD20-mAB-P/P complexed ibrutinib-Cy3.5 treatment in the DLBCL cell line HBL1. HBL1 cells were treated by the respective conjugates shown for 72 hrs and subjected to Annexin V-staining. Apoptotic cells were detected by AnnexinV-expression (upper panel, X-Axis) by flow cytometry, while increased internalized ibrutinib-Cy 3.5 fluorescence is seen by fluorescence in Y-axis (upper panel) especially in the αCD20-mAB-P/P complexed ibrutinib-Cy3.5 treated cells. Values from upper right and lower right gates were counted. Lower panel: Annexin V-positive cells in three independent experiments were summarized. P<0.05, 2-sided T-test. α, anti.

FIG. 82: Ewing sarcoma xenograft tumor growth is inhibited upon knockdown of oncogenic EWS-FLI1 translocation product through systemic therapy with αIGF1R-mAB-protamine/free protamine-siRNA-protamine nano-carriers. A. Treatment scheme of the in vivo experiments. Nanoparticles were given intraperitoneally as indicated. B-C. Results of systemic in vivo application of targeted nano-carriers on SK-N-MC xenograft tumors. B. Tumor growth curves SK-N-MC treated with αIGF1R-mAB teprotumumab (“Tepro”)-protamine/PsiRNA nanoparticles (means+/−SEM; 2-sided t-test, * p<0.05). C. Weight statistics of the excised tumors at the end of the experiment (mean+SD. 2-sided t-test, *p<0.05). α, anti.

FIG. 83: Nanoparticles formed by carrier antibodies-protamine/free protamine and siRNA expose an almost neutral surface charge. Nanoparticles were formed as described elsewhere for 2 hrs and subjected to dynamic light scattering (DLS) analysis (Malvern Zeta-sizer). Particle sizes ranged between 350 to 750 nm with indicated deviations, depending on the different antibody conjugation preparations. More importantly, the zeta-potential of the particle surface was only slightly negative to neutral.

FIG. 84: Deciphering preconditions for effective nanoparticle formation between anti-EGFR-mAB-SMCC-protamine conjugate, free SMCC-protamine and siRNA. A-G. Vesicle formation with 60 nM αEGFR-mAB-P in presence of 32×SMCC-protamine and rising (1:0.6-1:40) molar ratios of Alexa488-control-siRNAs compared to the antibody concentration. Vesicle formation can be observed at 5 to 10×molar excess of siRNA (D-E). Upper panels: Fluorescence microscopy of Alexa488-siRNA positive vesicle. Lower panels: Phase contrast of the same preparations as in upper panels. α, anti.

FIG. 85: Nanoparticles formed by αEGFR-protamine/free protamine-Alexa488-siRNA are stable in serum-containing conditions. A-B. Stability after 2 h-auto-assembly of αEGFR-mAB-protamine, free protamine and Alexa488-siRNA in a 1:10 ratio and subsequent incubation for 24 h in PBS (A) and in PBS/50% FCS (B) for the 24 h. α, anti.

FIG. 86: Serum stability of the αCD20-mAB-protamine/free P-ibrutinib-Cy3.5 nanocarrier. A-F. Stability after 2 h-auto-assembly of αCD20-mAB-protamine, free protamine and ibrutinib-Cy3.5 in a 1:20 ratio and subsequent incubation for 24 h (A-C) or 72 h (D-F) in PBS (A, D), and in challenging conditions such as cell culture medium RPMI/10% FCS (B, E) and PBS/50% FCS (C, F). A-F: Cy3.5 fluorescence microscopy, α, anti.

FIG. 87: pH stability of siRNA nanocarriers constructed with three different targeting antibodies. The nanocarriers formed with αEGFR-mAB-protamine/free protamine (upper panels), αIGF1R-mAB-protamine/free protamine (middle panels) and αCD33-mAB-protamine/free protamine (lower panels), each with 10-fold molar excess of siRNA were formed under standard conditions for 2 hrs at RT and then diluted in 30-fold volume of the respective buffer for each pH stability test for 24 hrs in chamber slides. Next, the slides were washed, mounted and subjected to fluorescence microscopy. The nanocarriers were shown to be stable at pH values between 5.2 and 8.0, with a tendency of agglomeration at lower pH. α, anti.

FIG. 88: pH stability of nanocarriers constructed with αCD20-mAB-protamine/free protamine and ibrutinib-Cy3.5. The nanocarriers formed with αCD20-mAB-protamine/free protamine with 20-fold molar excess of ibrutinib-Cy3.5 were formed under standard conditions for 2 hrs at RT and then diluted in 30-fold volume of the respective buffer for each pH stability test for 24 hrs in chamber slides. Next, the slides were washed, mounted and subjected to fluorescence microscopy. The nanocarriers were shown to be stable at pH values between 5.8 and 8.0, with a tendency of disintegration at lower pH. α, anti.

FIG. 89: Immunolabeling of targeting IgG antibodies in αEGFR-mAB-P/free protamine-siRNA nanocarriers. Nanocarriers were formed by auto-assembly for 2 hrs (αEGFR-P/free protamine plus Alexa488-siRNA (green in A and D)), immobilized o/n on treated glass surface (A, E), stained with αhIgG-Alexa647 (A-C), rinsed with PBS, mounted with DAKO fluo mounting medium and subjected to fluorescence microscopy. Nanocarrier structures show prominent staining of Alexa647 of the targeting αEGFR-antibodies only on surface regions (B-C). F. Schematic overview about staining procedure. α, anti.

FIG. 90: Immunolabeling of targeting IgG antibodies in αIGF1R-mAB-P/free protamine siRNA nanocarriers. Nanocarriers were formed by auto-assembly for 2 hrs (teprotumumab-protamine plus Alexa488-siRNA (green)), immobilized o/n on treated glass surface (A, D), stained with αhIgG-Alexa647 (A-C), rinsed with PBS, mounted with DAKO fluo mounting medium and subjected to fluorescence microscopy. Nanocarrier structures show prominent staining of Alexa647 of the targeting teprotumumab antibodies only on surface regions (B-C). F. Schematic overview about staining procedure. α, anti.

FIG. 91: Visualisation of the free protamine in the nanocarrier complex. Here, an αEGFR-mAB-protamine preparation depleted for free protamine by size exclusion chromatography and reconstituted this preparation with free protamine was used, tagged with the Cy3 chromophor. A: protamine was conjugated with Cy3-NHS ester according to the manufacturers recommendations and purified by spin-columns. The resulting protamine-Cy3 exhibited strong Cy3-dependent fluorescence and was concentrated in a comparable way than the unconjugated material, therefore it was reconstituted to the antibody-protamine in the usual 32fold molar excess (B). siRNA nanocarriers formed by this reconstituted material with non-tagged siRNA exhibited strong Cy3-dependent fluorescence signals of the protamine in the lumen of the nanostructures (C). By contrast, the same nanostructures, stained for a human IgG signals with αhIgG-Alexa 647 antibody exposed a rim structure stained positive for antibody location (D). E: Merge and higher magnification of highlighted part in C and D. F: Higher magnification of highlighted part in E. α, anti.

FIG. 92: Synthesis of cyanine-dye conjugated inhibitors gefitinib, gemcitabine and venetoclax.

FIG. 93: Expanding the concept to easier and cheaper polyanionic molecular moieties.

### DETAILED DESCRIPTION

The inventors of the present application have surprisingly found that fusion proteins of an antibody and protamine, as e.g., described by Yao et al. 2012 only perform poorly for the complexation of negatively charged cargo molecules. However, it was surprisingly found that addition of free protamine dramatically increased the fusion protein's capacity of complexing negatively charged cargo molecules, such as siRNA. Addition of free protamine results in the formation of nanoparticles comprising the antibody-protamine fusion, free protamine and a negatively charged cargo molecule to be delivered to a target.

It was also surprisingly found that a modification of the conjugation protocol for antibody-protamine conjugates as described Bäumer, N. et al., 2016 Nat. Protoc. 11, 22-36 results in the formation of nanoparticles comprising the antibody-protamine conjugate, free protamine and a negatively charged cargo molecule to be delivered to a target.

Formation of a complex nanoparticle comprising a targeting antibody fused to protamine, free protamine and a negatively charge cargo molecule such as a siRNA can be used for cell-type specific therapeutic delivery of siRNA and other effector drugs which can selectively block oncogenic pathways. The same applies to nanoparticles comprising a targeting moiety chemically conjugated to protamine, free protamine and a negatively charge cargo molecule.

In a protocol of the disclosure, for the step of loading siRNA onto the antibody-protamine fusion protein, the antibody-protamine fusion protein and the siRNA are contacted with each other in the presence of a certain amount of free protamine. Surprisingly, nanoparticles comprising antibody-protamine fusion proteins, free protamine, and the siRNA are formed with this modified protocol.

The inventors of the present application have further surprisingly found that such nanoparticles provide a more efficient binding and transport of an siRNA, as compared to antibody fusion proteins that were generated following protocols of the prior art.

The nanostructures generated by the method of the invention is by far bigger than the linear single antibody-protamine-siRNA complex and can be detected as a vesicular structure by light microscopy. The inventors of the present application have found out that a certain amount of unbound protamine is needed to form these nanoparticles and to target the respective cells efficiently. It was observed that a knockdown of the intended target (onco-) genes can then be performed specifically. A positively charged nanostructure (micelle) can also serve as carrier for other negatively charged small molecules as shown in Example 6 and 15, in which a small molecule was also transported in a targeted and efficient way into the respective cells. With this approach the therapeutic molecule, such as a siRNA, is not only operative to form the electrostatic nanostructure, but is also encapsuled inside the nanostructure.

Since most cancer types in an advanced and metastasized stage are not effectively curable when the present invention is made, there is a strong need for new, more effective and better tolerable therapy options. The nanoparticle comprising of a targeting moiety such as a cancer cell-specific antibody fused to protamine and free protamine is able to transport negatively charged molecules such as siRNA and other small molecules that are otherwise not taken up by eukaryotic cells. Because especially siRNAs can be defined against any gene, this system is potentially applicable to a large number of diseases including cancer, neurodegeneration, and viral infections.

The present application thus relates to a method of generating a nanoparticle comprising contacting (a) a fusion protein (A), said fusion protein (A) comprising an antibody (A1) and a positively charged polypeptide (A2); (b) a positively charged polypeptide (B); and (c) a negatively charged molecule (C); thereby forming a nanoparticle.

In the method of the present disclosure, the fusion protein (A), a positively charged polypeptide (B), and a negative charged molecule (C) are contacted with each other. Without wishing to be bound by theory, it is observed that the afore-mentioned three components self-assemble to form a nanoparticle.

When referring to a positively charged polypeptide (B), the expression preferably refers to a free positively charged polypeptide, that is not comprised in a fusion protein with a targeting moiety, such as an antibody. The positively charged polypeptide (B) may however comprise post-translational and/or chemical modifications. The term positively charged polypeptide (B) also encompasses mixtures of different positively charged polypeptides, including mixtures of different types of positively charged polypeptides.

Preferred positively charged polypeptides in the context of step (d) include, but are not limited to, a protamine, a histone subunit, or mixtures thereof with a protamine being preferred.

In some methods of the disclosure, the positively charged polypeptide is preferably in molar excess as compared to the fusion protein (A), which means that there are preferably more positively charged polypeptide (B) molecules than fusion protein (A) molecules. In some embodiments the molar ratio between the positively charged polypeptide (B) and the fusion protein (A) is at least about 10:1, preferably at least about 15:1, preferably at least about 20:1. In some embodiments, the molar ratio between the positively charged polypeptide (B) and fusion protein (A) is up to about 70:1, preferably up to about 60:1, preferably up to about 50:1. In some embodiments, the molar ratio between the positively charged polypeptide (B) and the fusion protein (A) is in the range of about 10:1 to 50:1, preferably about 15:1 to about 50:1, preferably about 20:1 to about 50:1. Preferred embodiments, the molar ratio between the positively charged polypeptide (B) and the fusion protein (A) is about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, and/or about 50:1.

In some methods of the disclosure, the negative charged molecule (C) is preferably in molar excess as compared to the fusion protein (A), which means that there are preferably more negatively charged molecules (C) than fusion protein (A) molecules. In some embodiments the molar ratio between the negatively charged molecule (C) and the fusion protein (A) is at least about 1:1, is at least about 2:1, is at least about 3:1, is at least about 4:1, is at least about 5:1, is at least about 6:1, is at least about 7:1, is at least about 8:1, is at least about 9:1, is at least about 10:1, at least about 15:1, at least about 20:1, at least about 25:1, at least about 30:1, at least about 40:1, at least about 50:1, at least about 70:1, at least about 100:1, at least about 150:1, at least about 200:1, at least about 250:1, at least about 300:1, at least about 400:1, or at least about 500:1.

In some methods of the present disclosure, the negative charged molecule (C) is preferably equimolar or in molar excess as compared to the positively charged polypeptide (B), which means that there are preferably about the same number or more negatively charged molecules (B) compared with positively charged polypeptide molecules (B). In some embodiments the molar ratio between the negatively charged molecule (C) and the positively charged polypeptide (B) is at least about 1:1, at least about 2:1, at least about 3:1, at least about 4:1, at least about 5:1, at least about 6:1, at least about 7:1, at least about 8:1, at least about 9:1, at least about 100:1, at least about 20:1, at least about 30:1, at least about 40:1, at least about 50:1, at least about 60:1, at least about 70:1, at least about 80:1, at least about 90:1, or at least about 100:1.

The method of the disclosure can be carried out at a large range of temperatures. The Examples of the present application show that nanoparticles can be formed at about 4° C., at room temperature, as well as at 37° C. Accordingly, it is envisioned that the method of the present disclosure, including can be carried out at a temperature of from about 1° C. to about 60° C., preferably from about 2° C. to about 50° C., preferably from about 3° C. to about 40° C., more preferably be from about 4° C. to about 37° C. Thus, at least a part of the method of the disclosure, such as the step of contacting a fusion protein (A), a positively charged polypeptide (B), and a negatively charged molecule (C) be carried out at a temperature of from about 1° C. to about 60° C., preferably from about 2° C. to about 50°, preferably from about 3° C. to about 40° C., more preferably be from about 4° C. to about 37° C.

It is envisioned the step of contacting a fusion protein (A), a positively charged polypeptide (B), and a negatively charged molecule (C) preferably comprises an incubation step to allow formation of nanoparticles. The incubation step is preferably carried out for at least about 1 h, preferably at least about 1.5 h, preferably at least about 2 h. The incubation step is preferably carried out for up to about 48 h, preferably up to about 24 h, preferably up to about 18 h, preferably up to about 12 h, preferably up to about 10 h, preferably up to about 9 h, preferably up to about 8 h, preferably up to about 7 h, preferably up to about 6 h. The incubation step is preferably carried out for about 1 h to about 48 h, preferably for about 1 h to about 24 h, preferably from about 1 h to about 18 h, preferably from about 1 h to about 12 h, preferably from about 1 h to about 10 h, preferably from about 1 h to about 9 h, preferably from about 1.5 h to about 8 h, preferably from about 1.5 to about 7 h, preferably from about 2 h to about 6 h. Without wishing to be bound by theory, it is believed that optimal results can be achieved within incubation for about 2 hours to about 6 hours. Thus, a preferred embodiments, step comprises an incubation step from about 2 h to about 6 h, including about 2 h, about 2.1 h, about 2.2 h, about 2.3 h, about 2.4 h, about 2.5 h, about 2.6 h, about 2.7 h, about 2.8 h, about 2.9 h, about 3 h, about 3.1 h, about 3.2 h, about 3.3 h, about 3.4 h, about 3.5 h, about 3.6 h, about 3.7 h, about 3.8 h, about 3.9 h, about 4 h, about 4.1 h, about 4.2 h, about 4.3 h, about 4.4 h, about 4.5 h, about 4.6 h, about 4.7 h, about 4.8 h, about 4.9 h, about 5 h, about 5.1 h, about 5.2 h, about 5.3 h, about 5.4 h, about 5.5 h, about 5.6 h, about 5.7 h, about 5.8 h, about 5.9 h, and about 6 h.

The term “conjugation” or “conjugate” as used herein refer to the joining together of two or more molecules, through all forms of covalent linkage, by means including, but not limited to chemical conjugation. So, the conjugation may include conjugation of at least one portion of a linker to a polypeptide. This connection can be achieved via different reactive groups or via the same reactive groups.

As used interchangeably herein, the terms “fuse” or “fusion” refer to the joining together of two or more subunits, by means including genetic fusion. The term “fusion polypeptide” or “fusion protein” as used herein interchangeably refers to a polypeptide or protein comprising two or more subunits. Within the fusion polypeptide, these subunits may be linked by covalent or non-covalent linkage. Preferably, the fusion polypeptide is a translational fusion between the two or more subunits. The translational fusion may be generated by genetically engineering the coding sequence for one subunit in a reading frame with the coding sequence of a further subunit. Both subunits may be interspersed by a nucleotide sequence encoding a linker. The subunits forming the fusion polypeptide or fusion protein are typically linked to each other as follows: C-terminus of one subunit to N-terminus of another subunit or N-terminus of one subunit to C-terminus of another subunit. The subunits of the fusion polypeptide can be linked in any order and may include more than one of any of the constituent subunits. If one or more of the subunits is part of a protein (complex) that consists of more than one polypeptide chain, such as an antibody, the term “fusion polypeptide” may also refer to the polypeptide comprising the fused sequences and all other polypeptide chain(s) of the protein (complex).

The methods of the present disclosure may further comprise a step of separating and/or recovering a nanoparticle from a component of its production environment. Preferably, after the separation and/or recovery, the nanoparticle is free or substantially free of association with all other components from its production environment. Contaminant components of its production environment, are materials that would typically interfere with the uses, in particular therapeutic uses for the nanoparticle, and may include free fusion proteins (A) (i.e., fusion proteins (A) not comprised in a nanoparticle), free positively charged polypeptides (B) (i.e., not comprised in a nanoparticle), or free negatively charged molecules (C) (i.e., not comprised in a nanoparticle). The nanoparticles may e.g constitute at least about 5%, at least about 10%, at least about 15%, at least about 20%, at least about 25%, at least about 30%, at least about 35%, at least about 40%, at least about 45% by weight of the total protein in a given sample. In preferred embodiments, the nanoparticles constitute at least about 50% at least about 55%, at least about 60%, at least about 65%, at least about 70%, at least about 75%, at least about 80%, at least about 85% by weight of the total protein in a given sample. In preferred embodiments, the nanoparticles constitute at least about 90% at least about 91%, at least about 92%, at least about 93%, at least about 94%, at least about 95%, at least about 96%, at least about 97%, at least about 98%, or at least about 99% by weight of the total protein in a given sample. It is understood that the isolated nanoparticle may constitute from about 5% to about 99.9% or about 100% by weight of the total protein content, depending on the circumstances.

The term “polypeptide” as used herein refers to a compound made up of a single chain of amino acid residues linked by peptide bonds. The term “protein” as used herein may be synonymous with the term “polypeptide” or may refer, in addition, to a complex of two or more polypeptides. A polypeptide as used herein may comprise at least about 10, at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500, at least about 600, at least about 700 or even more amino acids.

A polypeptide as used herein preferably consists of naturally occurring and/or proteogenic amino acids. However, peptidomimetics wherein amino acid(s) and/or peptide bond(s) have been replaced by functional analogues are also encompassed by the invention. The term polypeptide also refers to, and does not exclude, modifications of the polypeptide, e.g., glycosylation, acetylation, phosphorylation and the like. Such modifications are well described in basic texts and in more detailed monographs, as well as in the research literature.

The term “positively charged polypeptide” means a polypeptide having a net positive charge at or near physiological pH (e.g., in solutions having a pH between 4 to 10, between 5 to 9, or between 6 to 8) and that is preferably capable of binding a nucleic acid or a negatively charged small molecule through electrostatic interactions. Carriers of this class include, but are not limited to a protamine, a histone or histone subunit. Preferably, such a positively charged polypeptide has a net charge of at least 2+, preferably at least 3+, preferably at least 4+, preferably at least 5+, preferably at least 6+ preferably at least 7+, preferably at least 8+, preferably at least 9+, preferably at least 10+, preferably at least 11+, preferably at least 12+, preferably at least 13+, preferably at least 14+, preferably at least 15+, preferably at least 16+, preferably at least 17+, preferably at least 18+, preferably at least 19+, preferably at least 20+. The term “positively charged polypeptide” may encompass both, a free (i.e. unconjugated) polypeptide, as well as conjugated polypeptides, or a polypeptide comprised in a fusion protein.

A preferred positively charged polypeptide according to the disclosure comprises a protamine. A protamine refers to small, strongly basic proteins, the positively charged amino acid groups of which (especially arginines) are usually arranged in groups and neutralize the negative charges of nucleic acids because of their polycationic nature. The term “protamine” as used herein are meant to comprise any protamine amino acid sequence obtained or derived from native or biological sources including fragments thereof and multimeric forms of said amino acid sequence or fragment thereof. Protamines may be of natural origin or produced by recombinant methods. Use of recombinant methods allows multiple copies of the protamine to be produced or modifications may be made in the molecular size and amino acid sequence of the protamine. Corresponding compounds may also be chemically synthesized. When an artificial protamine is synthesized, the procedure used may include, for example, replacing amino acid residues which have functions in the natural protamine that are undesirable for the transporting function (e.g., the condensation of DNA) with other suitable amino acids. Generally, a protamine according to the disclosure can be of any species or derived from any species. A protamine of the disclosure can be from a mammal, a bird, an amphibian, a reptile, or a fish. A protamine of the disclosure can be of any species or derived from any species selected from the group consisting of a human, dog, cat, mouse, rat, horse, cattle, pig, goat, chicken, sheep, donkey, rabbit, alpaca, llama, goose, ox, turkey, salmon, or the like, preferably human or salmon. A protamine of the disclosure may also be a mixture of different protamines. Preferred protamines include salmon protamine. Preferred protamines include human protamine. A preferred protamine comprises or preferably consists of a sequence that has at least about 80%, preferably at least about 85%, preferably at least about 90%, preferably at least about 95%, sequence identity to salmon protamine as shown in SEQ ID NO: 53. A preferred protamine comprises or preferably consists of salmon protamine as shown in SEQ ID NO: 53. A preferred protamine comprises or preferably consists of a sequence that has at least about 80%, preferably at least about 85%, preferably at least about 90%, preferably at least about 95%, sequence identity to human protamine 1 as shown in SEQ ID NO: 55. A preferred protamine comprises or preferably consists of human protamine 1 as shown in SEQ ID NO: 55.

As described above, protamine is a strong positively charged protein that also interacts immediately with negatively charged nucleic acids such as siRNA. Without wishing to be bound by theory it is believed that the uptake of the complex into the cell is mediated via receptor-mediated endocytosis. It is further believed that the antibody binds to the receptor and the nanoparticle is internalized via endocytosis in clathrin coated pits. It is further believed that the vesicles are transported into the cell where the siRNA is released from the nanoparticle and can enter the RNAi pathway.

A further preferred positively charged polypeptide according to the disclosure comprises a histone or histone subunit. Histones refer to small DNA-binding proteins present in the chromatin having a high pro-portion of positively charged amino acids (lysine and arginine) which enable them to bind to DNA independently of the nucleotide sequence and fold it into nucleosomes. The term “histone” as used herein are meant to comprise any histone amino acid sequence obtained or derived from native or biological sources including histone subunits, fragments thereof and multimeric forms of said amino acid sequence or fragment thereof. The histones H2, H3 and H4 are particularly suitable. Generally, a histone according to the disclosure can be of any species or derived from any species. A histone of the disclosure can be from a mammal, a bird, an amphibian, a reptile, or a fish. A histone of the disclosure can be of any species or derived from any species selected from the group consisting of a human, dog, cat, mouse, rat, horse, cattle, pig, goat, chicken, sheep, donkey, rabbit, alpaca, llama, goose, ox, turkey, salmon, or the like, preferably human. A histone of the disclosure may also be a mixture of different histones or histone subunits. Preferred histones include a human histone. A preferred histone comprises or preferably consists of a sequence that has at least about 80%, preferably at least about 85%, preferably at least about 90%, preferably at least about 95%, preferably at least about 98%, preferably at least about 99% sequence identity to human histone H2 as shown in SEQ ID NO: 56. A preferred histone comprises or preferably consists of human histone H2 as shown in SEQ ID NO: 56. A preferred histone comprises or preferably consists of a sequence that has at least about 80%, preferably at least about 85%, preferably at least about 90%, preferably at least about 95% sequence identity to the human histone H2 derived peptide as shown in SEQ ID NO: 57. A preferred histone comprises or preferably consists of the human histone H2 derived peptide as shown in SEQ ID NO: 57. A preferred histone comprises or preferably consists of a sequence that has at least about 80%, preferably at least about 85%, preferably at least about 90%, preferably at least about 95% sequence identity to the human histone H2 derived peptide as shown in SEQ ID NO: 58. A preferred histone comprises or preferably consists of the human histone H2 derived peptide as shown in SEQ ID NO: 58.

In the methods or nanoparticles of the disclosure, a positively charged polypeptide (B) may be the same positively charged polypeptide as the positively charged polypeptide (A2) that is comprised in the fusion protein (A). In the methods or nanoparticles of the disclosure, a positively charged polypeptide (B) may be different from the positively charged polypeptide (A2) that is comprised in the fusion protein (A). In the methods or nanoparticles of the disclosure, a positively charged polypeptide (B) may comprise the same amino acid sequence as the positively charged polypeptide (A2) that is comprised in the fusion protein (A). In the methods or nanoparticles of the disclosure, a positively charged polypeptide (B) may comprise a different amino acid sequence as the positively charged polypeptide (A2) that is comprised in the fusion protein (A).

A “linker” as used herein that may be comprised by a fusion protein or polypeptide of the present disclosure joins together two or more subunits of a fusion polypeptide as described herein. For example, the antibody (A1) and the positively charged polypeptide (A2) can be fused via a linker. The linkage can be covalent or non-covalent. A preferred covalent linkage is via a peptide bond, such as a peptide bond between amino acids. A preferred linker is a peptide linker. A preferred linker is an unstructured linker. Accordingly, in a preferred embodiment, said linker comprises one or more amino acids, such as 1, 2, 3, 4, 5, 6, 7, 8, 9, 10, 11, 12, 13, 14, 15, 16, 17, 18, 19, 20 or more amino acids. Preferred peptide linkers are described herein, including glycine-serine (GS) linkers, glycosylated GS linkers, and proline-alanine-serine polymer (PAS) linkers. In some preferred embodiments, a GS linker is a (G2S)4 linker as described in SEQ ID NO: 67 and is used to join together the subunits of a fusion polypeptide. A linker may also comprise functional elements, such as an endosomal escape domain, such as EED1 as comprised in the linker of SEQ ID NO: 69.

The definition of the term “antibody” includes embodiments such as monoclonal, chimeric, single chain, humanized and human antibodies. In addition to full-length antibodies, the definition also includes antibody derivatives and antibody fragments, like, inter alia, Fab fragments. Antibody fragments or derivatives further comprise F(ab′)2, Fv, scFv fragments or single domain antibodies such as domain antibodies or nanobodies, single variable domain antibodies or immunoglobulin single variable domain comprising merely one variable domain, which might be VHH, VH or VL, that specifically bind an antigen or epitope independently of other V regions or domains. Said term also includes diabodies or Dual-Affinity Re-Targeting (DART) antibodies. Further envisaged are (bispecific) single chain diabody, tandem diabody (Tandab), “minibodies” exemplified by a structure which is as follows: (VH-VL-CH3)₂, (scFv-CH3)₂ or (scFv-CH3-scFv)₂, “Fc DART” and “IgG DART”, multibodies such as triabodies. Immunoglobulin single variable domains encompass not only an isolated antibody single variable domain polypeptide, but also larger polypeptides that comprise one or more monomers of an antibody single variable domain polypeptide sequence.

Furthermore, the term “antibody” as employed herein also relates to derivatives or variants of the antibodies described herein which display the same specificity as the described antibodies. Examples of “antibody variants” include humanized variants of non-human antibodies, “affinity matured” antibodies and antibody mutants with altered effector function(s) (see, e.g., U.S. Pat. No. 5,648,260).

The term “antibody” also comprises immunoglobulins (Ig's) of different classes (i.e. IgA, IgG, IgM, IgD and IgE) and subclasses (such as IgG1, IgG2 etc.). Derivatives of antibodies, which also fall under the definition of the term antibody in the meaning of the invention, include modifications of such molecules as for example glycosylation, acetylation, phosphorylation, disulfide bond formation, farnesylation, hydroxylation, methylation or esterification.

A functional fragment of an antibody includes the domain of a F(ab′)₂ fragment, a Fab fragment, scFv or constructs comprising single immunoglobulin variable domains or single domain antibody polypeptides, e.g. single heavy chain variable domains or single light chain variable domains as well as other antibody fragments as described herein above. The F(ab′)₂ or Fab may be engineered to minimize or completely remove the intermolecular disulphide interactions that occur between the CH1 and CL domains.

The term “human” antibody as used herein is to be understood as meaning that the antibody or its functional fragment, comprises (an) amino acid sequence(s) contained in the human germline antibody repertoire. For the purposes of definition herein, an antibody, or its fragment, may therefore be considered human if it consists of such (a) human germline amino acid sequence(s), i.e. if the amino acid sequence(s) of the antibody in question or functional fragment thereof is (are) identical to (an) expressed human germline amino acid sequence(s). An antibody or functional fragment thereof may also be regarded as human if it consists of (a) sequence(s) that deviate(s) from its (their) closest human germline sequence(s) by no more than would be expected due to the imprint of somatic hypermutation. Additionally, the antibodies of many non-human mammals, for example rodents such as mice and rats, comprise VH CDR3 amino acid sequences which one may expect to exist in the expressed human antibody repertoire as well. Any such sequence(s) of human or non-human origin which may be expected to exist in the expressed human repertoire would also be considered “human” for the purposes of the present invention. The term “human antibody” hence includes antibodies having variable and constant regions corresponding substantially to human germline immunoglobulin sequences known in the art, including, for example, those described by Kabat et al (Kabat et al., (1991) ‘Sequences of Proteins of Immunological Interest, 5th Ed.’, National Institutes of Health).

The human antibodies of the disclosure may include amino acid residues not encoded by human germline immunoglobulin sequences (e.g., mutations introduced by random or site-specific mutagenesis in vitro or by somatic mutation in vivo), for example in the CDRs, and in particular, CDR3. The human antibody can have at least one, two, three, four, five, or more positions replaced with an amino acid residue that is not encoded by the human germline immunoglobulin sequence.

The non-human and human antibodies or functional fragments thereof are preferably monoclonal. It is particularly difficult to prepare human antibodies which are monoclonal. In contrast to fusions of murine B cells with immortalized cell lines, fusions of human B cells with immortalized cell lines are not viable. Thus, the human monoclonal antibodies are the result of overcoming significant technical hurdles generally acknowledged to exist in the field of antibody technology. The monoclonal nature of the antibodies makes them particularly well suited for use as therapeutic agents, since such antibodies will exist as a single, homogeneous molecular species which can be well-characterized and reproducibly made and purified. These factors result in products whose biological activities can be predicted with a high level of precision, very important if such molecules are going to gain regulatory approval for therapeutic administration in humans. The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256: 495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352: 624-628 (1991) and Marks et al., J. Mol. Biol., 222: 581-597 (1991), for example.

It is especially preferred that the monoclonal antibodies or corresponding functional fragments be human antibodies or corresponding functional fragments. In contemplating antibody agents intended for therapeutic administration to humans, it is highly advantageous that the antibodies are of human origin. Following administration to a human patient, a human antibody or functional fragment thereof will most probably not elicit a strong immunogenic response by the patient's immune system, i.e. will not be recognized as being a foreign that is non-human protein. This means that no host, i.e. patient, antibodies will be generated against the therapeutic antibody which would otherwise block the therapeutic antibody's activity and/or accelerate the therapeutic antibody's elimination from the body of the patient, thus preventing it from exerting its desired therapeutic effect.

According to a further embodiment of the invention, the antibody may be an immunoglobulin. According to a further embodiment of the invention, the antibody may be an IgG antibody. An IgG isotype comprises not only the variable antibody regions of the heavy and light chains responsible for the highly discriminative antigen recognition and binding, but also the constant regions of the heavy and light antibody polypeptide chains normally present in “naturally” produced antibodies and, in some cases, even modification at one or more sites with carbohydrates. Such glycosylation is generally a hallmark of the IgG format, and located in the constant regions comprising the so called Fc region of a full antibody which is known to elicit various effector functions in vivo. In addition, the Fc region mediates binding of IgG to Fc receptor, as well as facilitating homing of the IgG to locations with increased Fc receptor presence-inflamed tissue, for example. Advantageously, the IgG antibody is an IgG1 antibody or an IgG4 antibody, formats which are preferred since their mechanism of action in vivo is particularly well understood and characterized. This is especially the case for IgG1 antibodies.

According to a further embodiment of the invention, the functional fragment of the antibody may preferably be an scFv, a single domain antibody, an Fv, a VHH antibody, a diabody, a tandem diabody, a Fab, a Fab′ or a F(ab)2. These formats may generally be divided into two subclasses, namely those which consist of a single polypeptide chain, and those which comprise at least two polypeptide chains. Members of the former subclass include a scFv (comprising one VH region and one VL region joined into a single polypeptide chain via a polypeptide linker); a single domain antibody (comprising a single antibody variable region) such as a VHH antibody (comprising a single VH region). Members of the latter subclass include an Fv (comprising one VH region and one VL region as separate polypeptide chains which are non-covalently associated with one another); a diabody (comprising two non-covalently associated polypeptide chains, each of which comprises two antibody variable regions-normally one VH and one VL per polypeptide chain—the two polypeptide chains being arranged in a head-to-tail conformation so that a bivalent antibody molecule results); a tandem diabody (bispecific single-chain Fv antibodies comprising four covalently linked immunoglobulin variable-VH and VL-regions of two different specificities, forming a homodimer that is twice as large as the diabody described above); a Fab (comprising as one polypeptide chain an entire antibody light chain, itself comprising a VL region and the entire light chain constant region and, as another polypeptide chain, a part of an antibody heavy chain comprising a complete VH region and part of the heavy chain constant region, said two polypeptide chains being intermolecularly connected via an interchain disulfide bond); a Fab′ (as a Fab, above, except with additional reduced disulfide bonds comprised on the antibody heavy chain); and a F(ab)2 (comprising two Fab′ molecules, each Fab′ molecule being linked to the respective other Fab′ molecule via interchain disulfide bonds). In general, functional antibody fragments of the type described hereinabove allow great flexibility in tailoring, for example, the pharmacokinetic properties of an antibody desired for therapeutic administration to the particular exigencies at hand. For example, it may be desirable to reduce the size of the antibody administered in order to increase the degree of tissue penetration when treating tissues known to be poorly vascularized (for example, joints). Under some circumstances, it may also be desirable to increase the rate at which the therapeutic antibody is eliminated from the body, said rate generally being accelerable by decreasing the size of the antibody administered. An antibody fragment is defined as a functional antibody fragment in the context of the invention as long as the fragment maintains the specific binding characteristics for the epitope/target of the parent antibody, e.g. as long as it specifically binds to CD33, EGFR, IGF1R, or CD20, or antibodies targeted to other cell surface structures a with the ability to internalize upon antibody binding.

According to a further embodiment of the invention, said antibody may comprise a CL domain. According to a further embodiment of the invention, said antibody may comprise a CH1 domain. According to a further embodiment of the invention, said antibody may comprise a CH2 domain. According to a further embodiment of the invention, said antibody may comprise a CH3 domain. According to a further embodiment of the invention, said antibody may comprise an entire light chain. According to a further embodiment of the invention, said antibody may comprise an entire heavy chain.

According to a further embodiment of the invention, said antibody or functional fragment thereof may be present in monovalent monospecific; multivalent monospecific, in particular bivalent monospecific; or multivalent multispecific, in particular bivalent bispecific forms. In general, a multivalent monospecific, in particular bivalent monospecific antibody such as a full human IgG as described hereinabove may bring with it the therapeutic advantage that the neutralization effected by such an antibody is potentiated by avidity effects, i.e. binding by the same antibody to multiple molecules of the same antigen, here e.g. CD33, EGFR, IGF1R, or CD20. Several monovalent monospecific forms of fragments of antibodies have been described above (for example, a scFv, an Fv, a VHH or a single domain antibody).

The antibodies or functional fragments thereof may be derivatized, for example with an organic polymer, for example with one or more molecules of polyethylene glycol (“PEG”) and/or polyvinyl pyrrolidone (“PVP”). As is known in the art, such derivatization can be advantageous in modulating the pharmacodynamic properties of antibodies or functional fragments thereof. Especially preferred are PEG molecules derivatized as PEG-maleimide, enabling conjugation with the antibody or functional fragment thereof in a site-specific manner via the sulfhydryl group of a cysteine amino acid. Of these, especially preferred are 20 kD and/or 40 kD PEG-maleimide, in either branched or straight-chain form. It may be especially advantageous to increase the effective molecular weight of smaller human antibody fragments such as scFv fragments by coupling the latter to one or more molecules of PEG, especially PEG-maleimide.

The antibodies of the present disclosure also include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) of mostly human sequences, which contain minimal sequence derived from non-human immunoglobulin. For the most part, humanized antibodies are human immunoglobulins (recipient antibody) in which residues from a hypervariable region (also CDR) of the recipient are replaced by residues from a hypervariable region of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody optimally also will comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Preferred antibodies are those which bind a cell surface molecule, preferably a cell surface domain, which is preferably internalized in a receptor-dependent manner, e.g. anti-CD33 antibodies, anti-EGFR antibodies, anti-IGF1R antibodies, or anti-CD20 antibodies. A preferred antibody is selected from the group consisting of cetuximab, gemtuzumab, cixutumumab, teprotumumab, GR11L, and rituximab. Further antibodies that bind cell surface domains and are internalized in a receptor-dependent manner are disclosed in LU 92353 A, which is incorporated by reference.

A preferred antibody, comprises a VH domain comprising the following three heavy chain CDRs and a VL domain comprising the following three light chain CDRs: a CDR-H1 having the sequence GFSLTNYG (SEQ ID NO: 1), a CDR-H2 having the sequence IWSGGNT (SEQ ID NO: 2), a CDR-H3 having the sequence ARALTYYDYEFAY (SEQ ID NO: 3), a CDR-L1 having the sequence QSIGTN (SEQ ID NO: 4), a CDR-L2 having the sequence YAS, and a CDR-L3 having the sequence QQNNNWPTT (SEQ ID NO: 5). A preferred antibody comprises a VH domain having the sequence set forth in SEQ ID NO 6 and a VL domain having a sequence set forth in SEQ ID NO: 7. A preferred antibody comprises a heavy chain having the sequence set forth in SEQ ID NO: 8 and a light chain having the sequence set forth in SEQ ID NO: 9.

A preferred antibody, comprises a VH domain comprising the following three heavy chain CDRs and a VL domain comprising the following three light chain CDRs: a CDR-H1 having the sequence GYTITDSN (SEQ ID NO: 10), a CDR-H2 having the sequence IYPYNGGT (SEQ ID NO: 11), a CDR-H3 having the sequence VNGNPWLAY (SEQ ID NO: 12), a CDR-L1 having the sequence ESLDNYGIRF (SEQ ID NO: 13), a CDR-L2 having the sequence AAS, and a CDR-L3 having the sequence QQTKEVPWS (SEQ ID NO: 14). A preferred antibody comprises a VH domain having the sequence set forth in SEQ ID NO 15 and a VL domain having a sequence set forth in SEQ ID NO: 16. A preferred antibody comprises a heavy chain having the sequence set forth in SEQ ID NO: 17 and a light chain having the sequence set forth in SEQ ID NO: 18. A preferred antibody comprises a heavy chain having the sequence set forth in SEQ ID NO: 66 and a light chain having the sequence set forth in SEQ ID NO: 18.

A preferred antibody, comprises a VH domain comprising the following three heavy chain CDRs and a VL domain comprising the following three light chain CDRs: a CDR-H1 having the sequence GGTFSSYAIS (SEQ ID NO: 19), a CDR-H2 having the sequence GIIPIFGTANYAQKFQ (SEQ ID NO: 20), a CDR-H3 having the sequence APLRFLEWSTQDHYYYYYMDV (SEQ ID NO: 21), a CDR-L1 having the sequence QGDSLRSYYAT (SEQ ID NO: 22), a CDR-L2 having the sequence GENKRPS (SEQ ID NO: 23), and a CDR-L3 having the sequence KSRDGSGQHLV (SEQ ID NO: 24). A preferred antibody comprises a VH domain having the sequence set forth in SEQ ID NO 25 and a VL domain having a sequence set forth in SEQ ID NO: 26. A preferred antibody comprises a heavy chain having the sequence set forth in SEQ ID NO: 27 and a light chain having the sequence set forth in SEQ ID NO: 28.

A preferred antibody comprises a VH domain comprising the following three heavy chain CDRs and a VL domain comprising the following three light chain CDRs: a CDR-H1 having the sequence GFTFSSYG (SEQ ID NO: 29), a CDR-H2 having the sequence IWFDGSST (SEQ ID NO: 30), a CDR-H3 having the sequence ARELGRRYFDL (SEQ ID NO: 31), a CDR-L1 having the sequence QSVSSY (SEQ ID NO: 32), a CDR-L2 having the sequence IWFDGSST (SEQ ID NO: 33), and a CDR-L3 having the sequence QQRSKWPPWT (SEQ ID NO: 34). A preferred antibody comprises a VH domain having the sequence set forth in SEQ ID NO 35 and a VL domain having a sequence set forth in SEQ ID NO: 36. A preferred antibody comprises a heavy chain having the sequence set forth in SEQ ID NO: 37 and a light chain having the sequence set forth in SEQ ID NO: 38.

A preferred antibody comprises a VH domain comprising the following three heavy chain CDRs and a VL domain comprising the following three light chain CDRs: a CDR-H1 having the sequence GYTFTSYN (SEQ ID NO: 39), a CDR-H2 having the sequence IYPGNGDT (SEQ ID NO: 40), a CDR-H3 having the sequence CARSTYYGGDWYFNV (SEQ ID NO: 41), a CDR-L1 having the sequence SSVSYI (SEQ ID NO: 42), a CDR-L2 having the sequence ATS, and a CDR-L3 having the sequence QQWTSNPPT (SEQ ID NO: 43). A preferred antibody comprises a VH domain having the sequence set forth in SEQ ID NO 44 and a VL domain having a sequence set forth in SEQ ID NO: 45. A preferred antibody comprises a heavy chain having the sequence set forth in SEQ ID NO: 46 and a light chain having the sequence set forth in SEQ ID NO: 47.

A “cell surface domain” as used herein means any protein on the cell surface. The cell surface domain also includes a cell surface antigen. It additionally includes any epitope that can be recognized on the cell surface of a cell. Preferably, the epitope or protein is cell type-specific as it only is present in a certain cell type. In one embodiment, the cell surface domain is present on a cancer cell. Potential cell surface targets include CD19, CD20, CD22, CD25, CD30, CD33, CD40, CD56, CD64, CD70, CD74, CD79, CD105, CD138, CD174, CD205, CD227, CD326, CD340, MUC16, GPNMB, PSMA, Cripto, ED-B, TMEFF2, EphB2, EphA2, FAP Av, integrin, Mesothelin, EGFR, TAG-72, GD2, CAIX and/or 5T4. Other potential cell surface domains include CD52, CD3, CD117, CD99, CD34, CD44, CD117, CA15-3, CA-125, CA27-29, EpCAM, Carcinoembryonic antigen, melanoma antigen recognized by T-cells 1 (MART1), trophoblast glycoprotein (TPBG). A cell surface molecule according to the invention is one that is preferably expressed on a cell which is susceptible to therapeutic treatment by the negatively charged molecule.

Preferably such a cell surface domain is CD33, EGFR, IGF1R, CD20. The cell surface domain can also provide for an epitope to which an antibody in accordance with the present invention can bind.

A preferred fusion protein (A) may comprise a polypeptide chain comprise an amino acid sequence having at least about 80%, preferably at least about 85%, preferably at least about 90%, preferably at least about 95%, preferably at least about 98%, preferably at least about 99% sequence identity or being identical to SEQ ID NO: 65. A preferred fusion protein (A) may comprise (a) polypeptide chain(s) which comprise (an) amino acid sequence(s) having at least about 80%, preferably at least about 85%, preferably at least about 90%, preferably at least about 95%, preferably at least about 98%, preferably at least about 99% sequence identity or being identical to SEQ ID NOs: 65 and 18.

A preferred fusion protein (A) may comprise a polypeptide chain comprising an amino acid sequence having at least about 80%, preferably at least about 85%, preferably at least about 90%, preferably at least about 95%, preferably at least about 98%, preferably at least about 99% sequence identity or being identical to SEQ ID NO: 71. A preferred fusion protein (A) may comprise (a) polypeptide chain(s) comprising (an) amino acid sequence(s) having at least about 80%, preferably at least about 85%, preferably at least about 90%, preferably at least about 95%, preferably at least about 98%, preferably at least about 99% sequence identity or being identical to SEQ ID NOs: 71 and 9. A preferred fusion protein (A) may comprise (a) polypeptide chain(s) comprising (an) amino acid sequence(s) having at least about 80%, preferably at least about 85%, preferably at least about 90%, preferably at least about 95%, preferably at least about 98%, preferably at least about 99% sequence identity or being identical to SEQ ID NOs: 71 and 73. A preferred fusion protein (A) may comprise (a) polypeptide chain(s) comprising (an) amino acid sequence(s) having at least about 80%, preferably at least about 85%, preferably at least about 90%, preferably at least about 95%, preferably at least about 98%, preferably at least about 99% sequence identity or being identical to SEQ ID NOs: 71 and 75.

A preferred fusion protein (A) may comprise a polypeptide chain comprising an amino acid sequence having at least about 80%, preferably at least about 85%, preferably at least about 90%, preferably at least about 95%, preferably at least about 98%, preferably at least about 99% sequence identity or being identical to SEQ ID NO: 73. A preferred fusion protein (A) may comprise (a) polypeptide chain(s) comprising (an) amino acid sequence(s) having at least about 80%, preferably at least about 85%, preferably at least about 90%, preferably at least about 95%, preferably at least about 98%, preferably at least about 99% sequence identity or being identical to SEQ ID NOs: 8 and 73.

A preferred fusion protein (A) may comprise a polypeptide chain comprising an amino acid sequence having at least about 80%, preferably at least about 85%, preferably at least about 90%, preferably at least about 95%, preferably at least about 98%, preferably at least about 99% sequence identity or being identical to SEQ ID NO: 75. A preferred fusion protein (A) may comprise (a) polypeptide chain(s) comprising (an) amino acid sequence(s) having at least about 80%, preferably at least about 85%, preferably at least about 90%, preferably at least about 95%, preferably at least about 98%, preferably at least about 99% sequence identity or being identical to SEQ ID NOs: 8 and 75.

A preferred fusion protein (A) may comprise a polypeptide chain comprising an amino acid sequence having at least about 80%, preferably at least about 85%, preferably at least about 90%, preferably at least about 95%, preferably at least about 98%, preferably at least about 99% sequence identity or being identical to SEQ ID NO: 79. A preferred fusion protein (A) may comprise (a) polypeptide chain(s) comprising (an) amino acid sequence(s) having at least about 80%, preferably at least about 85%, preferably at least about 90%, preferably at least about 95%, preferably at least about 98%, preferably at least about 99% sequence identity or being identical to SEQ ID NOs: 79 and 38. A preferred fusion protein (A) may comprise (a) polypeptide chain(s) comprising (an) amino acid sequence(s) having at least about 80%, preferably at least about 85%, preferably at least about 90%, preferably at least about 95%, preferably at least about 98%, preferably at least about 99% sequence identity or being identical to SEQ ID NOs: 79 and 81.

A preferred fusion protein (A) may comprise a polypeptide chain comprising an amino acid sequence having at least about 80%, preferably at least about 85%, preferably at least about 90%, preferably at least about 95%, preferably at least about 98%, preferably at least about 99% sequence identity or being identical to SEQ ID NO: 81. A preferred fusion protein (A) may comprise (a) polypeptide chain(s) which comprising (an) amino acid sequence(s) having at least about 80%, preferably at least about 85%, preferably at least about 90%, preferably at least about 95%, preferably at least about 98%, preferably at least about 99% sequence identity or being identical to SEQ ID NOs: 37 and 81.

In line with the above, the term “epitope” defines an antigenic determinant, which is specifically bound/identified by an antibody as defined herein. The antibody may specifically bind to/interact with conformational or continuous epitopes, which are unique for the target structure.

In preferred embodiment, the antibody of the disclosure is specific for a cancer associated antigen. As used herein, “cancer associated antigen” or “tumor associated antigen”, which can be used interchangeably herein, generally refers to any antigen that is associated with cancer or tumor cells, ie, occurs to the same or a greater extent compared to normal cells. Such antigens can be relatively tumor specific and their expression on the surface of malignant cells is limited, but they can also be found in non-malignant cells. In one embodiment, the antibody of the disclosure binds to a cancer-associated antigen.

The term “internalized” as used in the present invention means endocytosis, in which molecules such as proteins are engulfed by the cell membrane and drawn into the cell. In particular, the cell surface domain to which the binding domain binds is internalized. A method of how this internalization can be measured is disclosed in the examples of the present application. Otherwise, for example such a process may be observed by time-laps microscopy, where the receptor of interest and the cell membrane are double stained. Preferably, the nanoparticle comprising an antibody capable of binding to a cell surface molecule is internalized upon binding to the cell surface molecule.

A “fusion protein (A)” as used herein refers to a fusion protein comprising or preferably consisting of an antibody disclosed herein, a positively charged polypeptide disclosed herein and preferably a linker disclosed herein. Here, the positively charged polypeptide and the antibody are preferably interconnected via the (peptidic) linker.

Generally, an antibody (A1) and a positively charged polypeptide (A2) at any position suitable for such a fusion, e.g., at any N or C terminus of the antibody and/or positively charged fusion polypeptide. In some fusion proteins (A1), a positively charged polypeptide (A2) is fused to a C terminus of a heavy chain of the antibody (A1). In some fusion proteins (A1), a positively charged polypeptide (A2) is fused to a C terminus of a light chain of the antibody (A1). In some preferred fusion proteins, a positively charged polypeptide (A2) is fused to the C terminus of each of the heavy chains of the antibody (A1). In some preferred fusion proteins, a positively charged polypeptide (A2) is fused to the C terminus of each of the light chains of the antibody (A1). In some preferred fusion proteins, a positively charged polypeptide (A2) is fused to the C terminus of each of the heavy chains and each of the light chains of the antibody (A1). A fusion is optionally via a linker disclosed herein, such as a glycine serine linker.

The term “negatively charged molecule” refers to a molecule having a net positive charge at or near physiological pH (e.g., in solutions having a pH between 4 to 10, between 5 to 9, or between 6 to 8) and that is preferably capable of binding a positively charged polypeptide, such as a protamine or a histone through electrostatic interactions. Preferred negatively charged molecules are nucleic acids and negatively charged small molecules. Preferably, such a negatively charged molecule has a net charge of at least 2-, preferably at least 3-, preferably at least 4-, preferably at least 5-, preferably at least 6-, preferably at least 7-, preferably at least 8-, preferably at least 9-, or preferably at least 10-.

When referred to herein the terms “nucleotide sequence(s)”, “polynucleotide(s)”, “nucleic acid(s)”, “nucleic acid molecule” are used interchangeably and refer to nucleotides, either ribonucleotides or deoxyribonucleotides or a combination of both, in a polymeric unbranched form of any length. Nucleic acid sequences include DNA, cDNA, genomic DNA, RNA such as e.g. mRNA, siRNA, synthetic forms and mixed polymers, both sense and antisense strands, or may contain non-natural or derivatives nucleotide bases, as will be readily appreciated by those skilled in the art. Further, non-limiting examples of such nucleic acids include but are not limited to any type of RNA interfering (RNAi), whether single stranded or double stranded, that perform gene cessation and/or gene knockdown, including gene knockdown of message (mRNA) by degradation or translational arrest of the mRNA, inhibition of tRNA and rRNA functions or epigenetic effects; short (or small) interfering RNA (siRNA), short hairpin RNA (shRNA), endoribonuclease-prepared siRNAs (esiRNA), antisense oligonucleotides, microRNA and non-coding RNA or the like, short RNAs activity on DNA, and Dicer-substrate siRNAs. Preferred nucleic acids are siRNA, esiRNA antisense oligonucleotides, or miRNA, with siRNA being most preferred. In some embodiments, a nucleic acid has about 18 to about 25 bp, preferably if the nucleic acid is double stranded. In some embodiments, a nucleic acid has about 18 to about 25 nt, preferably if the nucleic acid is single stranded.

The nucleic acid utilized by the present invention effects a target cell. E.g. via provision of the nucleic acid molecule the expression of a specific molecule or protein is reduced or increased in a target cell. Preferably, the expression of a specific molecule protein is reduced by utilization of a nucleic acid molecule.

The nucleic acids according to the disclosure include siRNA molecules that are designed to target and suppress or block the expression of a gene or protein associated with cancer or is involved in the development and/or progression of cancer.

Preferred nucleic acid molecules are selected from siRNA, esiRNA, antisense oligonucleotides, or miRNA that is/are preferably specific for KRAS, BRAF, PIK3CA, PAX3-FKHR, EWS-FLI1, c-MYC, TP53, DNMT3A, IDH1, NPM1, or FLT3. Even more preferred is a siRNA specific for KRAS, BRAF, PIK3CA, PAX3-FKHR, EWS-FLI1, c-MYC, TP53, DNMT3A, IDH1, NPM1, or FLT3. Such siRNA are known to the skilled person and illustrative examples for such siRNA are shown in the following table.

| Target | siRNA sequence |
|---|---|
| KRAS | UUC UGC UUG UGA CAU UAA AAA |
| | (SEQ ID NO: 59) |
| PIK3CA | AAA CUU GGC UGA AGU UUA AAA |
| | (SEQ ID NO: 60) |
| PAX3-FKHR | UGA AUU CUG AGG UGA GAG GCTT |
| | (SEQ ID NO: 61) |
| EWS-FLI1 | GGC AGC AGA ACC CUU CUU AUU |
| | (SEQ ID NO: 62) |
| c-MYC | ACA CAA ACU UGA ACA GCU ATT |
| | (SEQ ID NO: 63) |
| TP53 | GAA AUG UUC UUG CAG UUA ATT |
| | (SEQ ID NO: 64) |

Preferred nucleic acids of the disclosure also include a mixture of different siRNA that are directed against one or more targets, preferably one target. For example, nucleic acids of the disclosure may comprise a mixture of siRNA that are specific for a target selected from the group consisting of KRAS, BRAF, PIK3CA, PAX3-FKHR, EWS-FLI1, c-MYC, TP53, DNMT3A, IDH1, NPM1, and FLT3.

A negatively charged molecule according to the invention may also be a molecule that is not a nucleic acid. Such a molecule may be a small molecule, or preferably, a small organic molecule. A negatively charged molecule may have a molecular weight of about 20 kDa or less, preferably about 15 kDa or less, or about 10 kDa or less. A negatively charged molecule may also have a molecular weight of about 9 kDa or less, about 8 kDa or less, about 7 kDa or less, about 6 kDa or less, about 5 kDa or less, about 4 kDa or less, about 3 kDa or less, or about 2 kDa or less. As an illustrative example, the negatively charged molecule can be a drug and/or prodrug. The drug and/or prodrug can be conjugated with a negatively charged moiety. For example, the drug may be ibrutinib that is conjugated to a negatively charged moiety, e.g. Cy 3.5 or Alexa488. However, the drug may also be conjugated to another negatively charged moieties. Suitable negatively charged moieties for conjugation are known to the skilled person. Illustrative examples of suitable negatively charged moieties include (poly)sulfonated aryls (e.g. as co-ligands for transition metals), (poly)sulfonated dyes (e.g. canine dyes), mono/di/triphosphates, (poly)sulfates of monosaccharides or branched oligosaccharides, oligopeptides from glutamic or aspartic acid. The drug and/or prodrug can have a negative net charge without being conjugated to any additional moiety. As an illustrative example, a drug having a negative net charge is remdesivir triphosphate. The skilled person will understand that the aforementioned examples are for illustration purposes only and that many other negatively charged molecules can be used within the context of the present invention.

As used herein “ibrutinib” (IUPAC name: 1-[(3R)-3-[4-amino-3-(4-phenoxyphenyl)pyrazolo[3,4-d]pyrimidin-1-yl]piperidin-1-yl]prop-2-en-1-one, CAS number: 936563-96-1) relates to a molecule having (in its free form) the following structure.

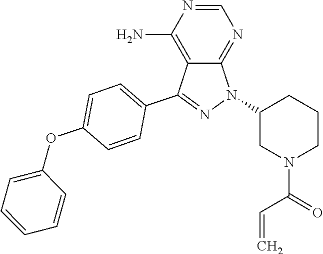

As used herein “remdesivir triphosphate” (IUPAC name [[(2R,3S,4R,5R)-5-(4-aminopyrrolo[2,1-f][1,2,4]triazin-7-yl)-5-cyano-3,4-dihydroxyoxolan-2-yl]methoxy-hydroxyphosphoryl] phosphono hydrogen phosphate, CAS number: 1355149-45-9) relates to a molecule having the following structure.

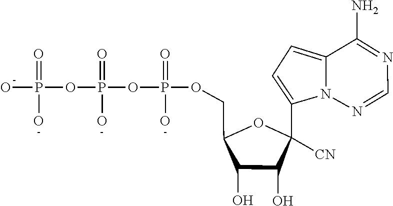

The present application also relates to a nanoparticle. Such a nanoparticle is preferably obtainable by a method described herein. A nanoparticle of the invention may comprise (a) a fusion protein (A), preferably as disclosed herein, said fusion protein (A) comprising an antibody (A1) and a positively charged polypeptide (A2); (b) a positively charged polypeptide (B), preferably as disclosed herein; and (c) one or more negatively charged molecule(s) (C), preferably as disclosed herein.

Without wishing to be bound by theory, it is believed that the positively charged polypeptide (B), the fusion protein (A), and the one or more negatively charged molecule(s) (C), are not homogeneously distributed within the particle. Instead, in some embodiments, the fusion protein (A) is enriched in the outer portion of the nanoparticle. In some embodiments, the one or more negatively charged molecule(s) (C) is/are enriched in the inner portion of the nanoparticle. In some embodiments, the positively charged polypeptide (B) is enriched in the outer portion of the nanoparticle. In some embodiments, the positively charged polypeptide (B) is enriched in the inner portion of the nanoparticle. Thus, the nanoparticle of the invention may form a vesicle-like structure, in which the fusion protein (A) is predominantly present in the outer portion, while the negatively charged molecule (C) is enriched or encapsuled in the inner portion of the nanoparticle. Without wishing to be bound by theory, it is believed that such a structure protects the negatively charged molecules (C) and thus increase their stability. It is further believed that at least a part or even the entire nanoparticle can be internalized upon binding to a cell via the fusion protein (A).

In some embodiment, the nanoparticles of the disclosure have a mean diameter that is at least about 0.05 μm. In some embodiment, the nanoparticles of the disclosure have a mean diameter that is at least about 0.1 μm. In some embodiment, the mean diameter of the nanoparticle is at least about 0.2 μm. In some embodiments, the nanoparticles of the disclosure have a mean diameter in the range from about 0.05 μm to about 10 μm, preferably from about 0.1 μm to about 10 μm, preferably from about 0.2 μm to about 5 μm. The mean diameter of the nanoparticles of the disclosure may also be in a range of about 0.3 μm to about 4 μm, about 0.4 μm to about 3 μm, or about 0.5 μm to about 2 μm. The mean diameter of the nanoparticles may be determined by any method suitable for the determination of particle sizes, including dynamic light scattering and microscopic analysis. The preferred method for the determination of particle sizes is by microscopic analysis, preferably by transmission light microscopy.

The present invention further relates to a composition comprising the nanoparticle of the invention and/or the nanoparticle obtainable by the method of the present invention.

The present invention further relates to a pharmaceutical composition comprising the nanoparticle of the present invention and/or the nanoparticle obtainable by the method of the present invention.

In a composition of the disclosure, including a pharmaceutical composition of the disclosure, at least about 10% of the fusion proteins (A) that are comprised in the composition may be comprised in a nanoparticle. In preferred embodiments, at least 20%, preferably at least 30%, preferably at least 40%, preferably at least 50%, preferably at least 60%, preferably at least 70%, preferably at least 80%, preferably at least 90%, preferably at least 95%, preferably at least 98%, preferably at least 99% of the fusion proteins (A) that are comprised in the composition are comprised in a nanoparticle. In preferred embodiments, the composition is essentially free of fusion proteins (A) that are not comprised in a nanoparticle.

In a composition of the disclosure, including a pharmaceutical composition of the disclosure, at least about 10% of the positively charged polypeptides (B) that are comprised in the composition may be comprised in a nanoparticle. In preferred embodiments, at least 20%, preferably at least 30%, preferably at least 40%, preferably at least 50%, preferably at least 60%, preferably at least 70%, preferably at least 80%, preferably at least 90%, preferably at least 95%, preferably at least 98%, preferably at least 99% of the positively charged polypeptides (B) that are comprised in the composition are comprised in a nanoparticle. In preferred embodiments, the composition is essentially free of positively charged polypeptides (B) that are not comprised in a nanoparticle.

In a composition of the disclosure, including a pharmaceutical composition of the disclosure, at least about 10% of the negatively charged molecules (C) that are comprised in the composition may be comprised in a nanoparticle. In preferred embodiments, at least 20%, preferably at least 30%, preferably at least 40%, preferably at least 50%, preferably at least 60%, preferably at least 70%, preferably at least 80%, preferably at least 90%, preferably at least 95%, preferably at least 98%, preferably at least 99% of the negatively charged molecules (C) that are comprised in the composition are comprised in a nanoparticle. In preferred embodiments, the composition is essentially free of negatively charged molecules that are not comprised in a nanoparticle.

The term “pharmaceutical composition” relates to a composition for administration to a patient, preferably a human patient. Pharmaceutical compositions or formulations are usually in such a form as to allow the biological activity of the active ingredient to be effective and may therefore be administered to a subject for therapeutic use as described herein. Usually, a pharmaceutical composition comprises suitable (i.e. pharmaceutically acceptable) formulations of carriers, stabilizers and/or excipients. Examples of suitable pharmaceutical carriers are described in “Remington's Pharmaceutical Sciences” by E. W. Martin. Such compositions will contain a therapeutically effective amount of the aforementioned molecules, preferably in purified form, together with a suitable amount of carrier so as to provide the form for proper administration to the patient. The formulation should suit the mode of administration.

In one embodiment, the pharmaceutical composition is a composition for parenteral, trans-dermal, intra-luminal, intra-arterial, intrathecal and/or intranasal administration or for direct injection into tissue. It is in particular envisaged that said composition is administered to a patient via infusion or injection. Administration of the suitable compositions may be effected by different ways, e.g., by intravenous, intra-peritoneal, subcutaneous, intra-muscular, topical or intra-dermal administration. The composition of the present invention may further comprise a pharmaceutically acceptable carrier. Examples of suitable pharmaceutical carriers are well known in the art and include buffered saline solutions, water, emulsions, such as oil/water emulsions, various types of wetting agents, sterile solutions, liposomes, etc. Compositions comprising such carriers can be formulated by well-known conventional methods.

In accordance with the present embodiments, the term “therapeutically effective amount” refers to an amount of the molecules of the present invention and/or the molecule obtainable by the method of the present invention that is effective for the treatment of diseases associated with cancer. Preferred dosages and preferred methods of administration are such that after administration the molecules of the present invention and/or the molecule obtainable by the method of the present invention is present in the blood in effective dosages. The administration schedule can be adjusted by observing the disease conditions and analysing serum levels of the molecule decreasing the expression of target molecules in laboratory tests followed by either extending the administration interval e.g. from twice per week or once per week to once per two weeks, once per three weeks, once per four weeks, and the like, or, alternatively, reducing the administration interval correspondingly. In the case of cancer, the therapeutically effective amount of the molecules or compositions disclosed herein may reduce the number of cancer cells; reduce the tumor size; inhibit (i.e., slow and/or stop) cancer cell infiltration into peripheral organs; inhibit (i.e., slow and/or stop) tumor metastasis; inhibit tumor growth; and/or relieve one or more of the symptoms associated with the cancer.

In another embodiment, the pharmaceutical composition is suitable to be administered in combination with an additional drug, i.e. as part of a co-therapy. In said co-therapy, an active agent may be optionally included in the same pharmaceutical composition as the molecule of the invention, or may be included in a separate pharmaceutical composition. In this latter case, said separate pharmaceutical composition is suitable for administration prior to, simultaneously as or following administration of said pharmaceutical composition comprising the molecule of the invention. The additional drug or pharmaceutical composition may be a non-proteinaceous compound or a proteinaceous compound. In the case that the additional drug is a proteinaceous compound, it is advantageous that the proteinaceous compound be capable of providing an activation signal for immune effector cells. Preferably, said proteinaceous compound or non-proteinaceous compound may be administered simultaneously or non-simultaneously with the molecule (or preparation) of the invention as defined hereinabove, a vector as defined hereinabove, or a host as defined hereinabove.

The pharmaceutical compositions can be administered to the subject at a suitable dose. The dosage regimen will be determined by the attending physician and by clinical factors. As is well known in the medical arts, dosages for any one patient depend upon many factors, including the patient's size, body surface area, age, the particular compound to be administered, sex, time and route of administration, general health, and other drugs being administered concurrently.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, inert gases and the like. In addition, the pharmaceutical composition in accordance with the present invention might comprise proteinaceous carriers, like, e.g., serum albumin or immunoglobulin, preferably of human origin. It is envisaged that the pharmaceutical composition in accordance with the invention might comprise, in addition to the above described molecules further biologically active agents, depending on the intended use of the pharmaceutical composition. Such agents might be drugs acting on the gastro-intestinal system, drugs acting as cytostatica, drugs preventing hyperurikemia, drugs inhibiting immunoreactions (e.g. corticosteroids), drugs modulating the inflammatory response, drugs acting on the circulatory system and/or agents such as cytokines known in the art.

To analyse the effect of the nanoparticles of the present invention and/or the nanoparticles obtainable by the method of the present invention for example in cancer therapy, outcome measures can be selected e.g. from pharmacokinetics, immunogenicity, and the potential to decrease the size of a cancer by e.g. MRI imaging as well as patient reported outcomes.

Another major challenge in the development of drugs such as the pharmaceutical composition in accordance with the invention is the predictable modulation of pharmacokinetic properties. To this end, a pharmacokinetic profile of the drug candidate, i.e. a profile of the pharmacokinetic parameters that affect the ability of a particular drug to treat a given condition, is established. Pharmacokinetic parameters of the drug influencing the ability of a drug for treating a certain disease entity include, but are not limited to: half-life, volume of distribution, hepatic first-pass metabolism and the degree of blood serum binding. The efficacy of a given drug agent can be influenced by each of the parameters mentioned above. “Half-life” means the time where 50% of an administered drug are eliminated through biological processes, e.g. metabolism, excretion, etc. By “hepatic first-pass metabolism” is meant the propensity of a drug to be metabolized upon first contact with the liver, i.e. during its first pass through the liver. “Volume of distribution” means the degree of retention of a drug throughout the various compartments of the body, like e.g. intracellular and extracellular spaces, tissues and organs, etc. and the distribution of the drug within these compartments.

“Degree of blood serum binding” means the propensity of a drug to interact with and bind to blood serum proteins, such as albumin, leading to a reduction or loss of biological activity of the drug.

Pharmacokinetic parameters also include bioavailability, lag time (Tlag), Tmax, absorption rates and/or Cmax for a given amount of drug administered. “Bioavailability” means the amount of a drug in the blood compartment. “Lag time” means the time delay between the administration of the drug and its detection and measurability in blood or plasma. “Tmax” is the time after which maximal blood concentration of the drug is reached, the absorption is defined as the movement of a drug from the site of administration into the systemic circulation, and “Cmax” is the blood concentration maximally obtained with a given drug. The time to reach a blood or tissue concentration of the drug which is required for its biological effect is influenced by all parameters.

The term “toxicity” as used herein refers to the toxic effects of a drug manifested in adverse events or severe adverse events. These side events might refer to a lack of tolerability of the drug in general and/or a lack of local tolerance after administration. Toxicity could also include teratogenic or carcinogenic effects caused by the drug.

The terms “safety”, “in vivo safety” or “tolerability” as used herein define the administration of a drug without inducing severe adverse events directly after administration (local tolerance) and during a longer period of application of the drug. “Safety”, “in vivo safety” or “tolerability” can be evaluated e.g. at regular intervals during the treatment and follow-up period. Measurements include clinical evaluation, e.g. organ manifestations, and screening of laboratory abnormalities. Clinical evaluation may be carried out and deviating to normal findings recorded/coded according to NCI-CTC and/or MedDRA standards. Organ manifestations may include criteria such as allergy/immunology, blood/bone marrow, cardiac arrhythmia, coagulation and the like, as set forth e.g. in the Common Terminology Criteria for adverse events v 3.0 (CTCAE). Laboratory parameters which may be tested include for instance haematology, clinical chemistry, coagulation profile and urine analysis and examination of other body fluids such as serum, plasma, lymphoid or spinal fluid, liquor and the like. Safety can thus be assessed e.g. by physical examination, imaging techniques (i.e. ultrasound, x-ray, CT scans, Magnetic Resonance Imaging (MRI), other measures with technical devices (i.e. electrocardiogram), vital signs, by measuring laboratory parameters and recording adverse events. The term “effective and non-toxic dose” as used herein refers to a tolerable dose of the molecules of the present invention and/or the molecule obtainable by the method of the present invention, preferably the antibody as defined herein, which is high enough to cure or stabilize the disease of interest without or essentially without major toxic effects. Such effective and non-toxic doses may be determined e.g., by dose escalation studies described in the art and should be below the dose inducing severe adverse side events (dose limiting toxicity, DLT).

The pharmaceutical composition of the present invention may have different formulations. The formulation (sometimes also referred to herein as “composition of matter”; “composition”, or “solution”) may preferably be in various physical states such as liquid, frozen, lyophilized, freeze-dried, spray-dried and reconstituted formulations, with liquid and frozen being preferred.

“Liquid formulation” as used herein refers to a composition of matter that is found as a liquid, characterized by free movement of the constituent molecules among themselves but without the tendency to separate at room temperature. Liquid formulations include aqueous and non-aqueous liquid, with aqueous formulations being preferred. An aqueous formulation is a formulation in which the solvent or main solvent is water, preferably water for injection (WFI). The dissolution of the molecules of the present invention and/or the molecule obtainable by the method of the present invention in the formulation may be homogenous or heterogeneous, with homogenous being preferred as described above.

Any suitable non-aqueous liquid may be employed provided that it provides stability to the formulation of the invention. Preferably, the non-aqueous liquid is a hydrophilic liquid. Illustrative examples of suitable non-aqueous liquids include: glycerol; dimethyl sulfoxide (DMSO); polydimethylsiloxane (PMS); ethylene glycols, such as ethylene glycol, diethylene glycol, triethylene glycol, polyethylene glycol (“PEG”) 200, PEG 300, and PEG 400; and propylene glycols, such as dipropylene glycol, tripropylene glycol, polypropylene glycol (“PPG”) 425 and PPG 725.

“Mixed aqueous/non-aqueous liquid formulation” as used herein refers to a liquid formulation that contains a mixture of water, preferably WFI, and an additional liquid composition.

When used herein a “formulation” or “composition” is a mixture of the molecules of the present invention and/or the molecule obtainable by the method of the present invention (i.e., the active drug/substance) and further chemical substances and/or additives required for a medicinal product which is preferably in a liquid state. A formulation of the invention includes a pharmaceutical formulation.

The preparation of the formulation includes the process in which different chemical substances, including the active drug, are combined to produce a final medicinal product such as a pharmaceutical composition. The active drug of the formulation of the invention is the nanoparticle of the present invention and/or the nanoparticle obtainable by the method of the present invention.

In certain embodiments, the nanoparticle of the present invention and/or the nanoparticle obtainable by the method of the present invention can be formulated essentially pure and/or essentially homogeneous (i.e., substantially free from contaminating substances, e.g. proteins, etc. which can be product-related and/or process-related impurities). The term “essentially pure” means a composition comprising at least about 80%, preferably about 90% by weight of the compound, preferably at least about 95% by weight of the compound, more preferably at least about 97% by weight of the compound or most preferably at least about 98% by weight of the compound. The term “essentially homogeneous” means a composition comprising at least about 99% by weight of the compound, preferably of the compound in a monomeric state, excluding the mass of various stabilizers and water in solution.

A “stable” formulation is one in which the molecules of the present invention and/or the molecule obtainable by the method of the present invention therein essentially retains its physical stability and/or chemical stability and/or biological activity upon storage and/or does not show substantial signs of aggregation, precipitation, fragmentation, degradation and/or denaturation compared to a control sample, preferably upon visual examination of colour and/or clarity, or as measured by UV light scattering or by size exclusion chromatography. Various further analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example.

“During storage,” as used herein, means a formulation that once prepared, is not immediately used; rather, following its preparation, it is packaged for storage, either in a liquid form, in a frozen state, or in a dried form for later reconstitution into a liquid form or other form.

A “subject” in accordance with the present invention is a vertebrate, preferably a mammal, more preferably a human subject.

A “vertebrate” includes vertebrate fish, birds, amphibians, reptiles and mammals.

A “mammal” includes dogs, cats, horses, rats, mice, apes, rabbits, cows, pigs, sheep, and preferably humans. To a human can also be referred to by the term patient.

The present invention further relates to the nanoparticle obtainable by the method of the present invention or the nanoparticle of the present invention and/or the pharmaceutical composition of the present invention for use in therapy. The use in therapy is preferably in a method for treating cancer in a subject.

The term “cancer” and “cancerous” as used by the present invention means a condition in vertebrates, preferably mammals, more preferably humans that is typically characterized by unregulated cell growth.

Cancers are classified by the type of cell that the tumor cells resemble and are therefore presumed to be the origin of the tumor. These types include carcinoma, sarcoma, blood cancer, germ cell tumors, and blastoma.

A “carcinoma” when referred to herein can include cancers derived from epithelial cells.

A “sarcoma” when referred to herein can include a cancer that arises from cells of mesenchymal (connective tissue) origin.

A “blood cancer” when referred to herein can include classes of cancer arising from hematopoietic (blood-forming) cells that leave the marrow and tend to mature in the lymph nodes and blood, respectively. When referred herein to leukemia, bone marrow-derived cells that normally mature in the bloodstream can be included. When referring herein to a lymphoma, bone marrow-derived cells that normally mature in the lymphatic system can be included.

A “germ cell tumor” when referred to herein can include cancers derived from pluripotent cells, most often presenting in the testicle or the ovary.

A “blastoma” when referred to herein can include cancers derived from immature “precursor” cells or embryonic tissue.

The molecule obtainable by a process of the present invention or the molecules of the present invention and/or the molecule obtainable by the method of the present invention may be used in a method for treating cancer. The cancer may be a solid tumor. The cancer may be selected from the group consisting of lung cancer, such as non small cell lung cancer, sarcoma, such as rhabdomyosarcoma or Ewing's sarcoma, colorectal cancer, blood cancer, such as leukemia or lymphoma, such as acute myeloid leukemia (AML) or diffuse large B-cell lymphoma (DLBLC).

The term “treatment” as used herein, means to alleviate, reduce, stabilize, or inhibit progression of a disease or disorder, such as cancer.

The present invention further relates to the nanoparticle obtainable by a process of the present invention or the nanoparticle of the present invention or the pharmaceutical composition of the present invention which is used in a method for inhibiting and/or controlling tumor growth in a subject.

A “tumor” or “neoplasm” is an abnormal mass of tissue as a result of abnormal growth or division of cells. The growth of neoplastic cells exceeds, and is not coordinated with that of the normal tissues around it. However, a tumor in the sense of the present invention does also include leukemia, and carcinoma in situ. A tumor can be benign, pre-malignant, or malignant. In a preferred embodiment the tumors are pre-malignant or malignant. Most preferably, the tumor is malignant.

The present invention further relates to the nanoparticle obtainable by a method of the present invention or the nanoparticle of the present invention or the (pharmaceutical) composition of the present invention for delivering a nucleic acid molecule to the site of a tumor in a subject.

In one embodiment of the present invention, the nanoparticle obtainable by a process of the present invention or the nanoparticle of the present invention or the pharmaceutical composition of the present invention for use of the present invention includes a siRNA selected from the group consisting of KRAS, BRAF, PIK3CA, PAX3-FKHR, EWS-FLI1, c-MYC, TP53, DNMT3A, IDH1, NPM1, and FLT3 siRNA. An siRNA of the present invention can target KRAS, BRAF, PIK3CA, PAX3-FKHR, EWS-FLI1, c-MYC, TP53, DNMT3A, IDH1, NPM1, or FLT3. Preferably the siRNA reduces the expression of KRAS, BRAF, PIK3CA, PAX3-FKHR, EWS-FLI1, c-MYC, TP53, DNMT3A, IDH1, NPM1, or FLT3 of a cell. Preferably, the expression of these targets is decreased.

A “siRNA targeting” means the target which is recognized by a specific siRNA. siRNAs can be constructed in different ways. For example, a siRNA can be targeting mRNA.

In general, the design of a siRNA is known to the skilled artesian. See for example Reynolds et al., (Reynolds et al., (2004) “Rational siRNA design for RNA interference” Nature Biotechnology 22, 326-330) or Judge et al., (Judge et al., 2006) “Design of Noninflammatory Synthetic siRNA Mediating Potent Gene Silencing in Vivo” Molecular Therapy (2006) 13, 494-505) or Sioud and Leirdal (Sioud and Leirdal (2004) “Potential design rules and enzymatic synthesis of siRNAs” Methods Mol Biol. 2004; 252:457-69).

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product. The mRNA is then translated into peptide/polypeptide chains, which are ultimately folded into the final peptide/polypeptides/proteins. Protein expression is commonly used by proteomics researchers to denote the measurement of the presence and abundance of one or more proteins in a particular cell or tissue. The expression of a protein of a cell can be measured by various means. For example, with immunohistochemistry or western blot analysis. Here, the obtained results should be evaluated in comparison, to a healthy cell, or control standard. A lower expressing cell shows a staining, which is decreased e.g. in intensity, when compared to a control cell. A higher expressing cell shows a staining, which is increased e.g. in intensity, when compared to a control cell in the same setting. Also, the expression of the mRNA can be measured e.g. by RT-PCR. Here, a lower expressing cell shows e.g. a higher number of amplification cycles to overt a detectable signal when compared to a control cell in the same setting. The person skilled in the art knows different techniques, how to determine the expression of a protein, mRNA of a cell.

For example, the cell can be present in the blood, liver, stomach, mouth, skin, lung, lymphatic system, spleen, bladder, pancreas, bone marrow, brain, kidneys, intestines, gallbladder, brain, larynx or pharynx of the subject.

In one embodiment, the nanoparticle obtainable by a method of the present invention or the nanoparticle of the present invention or the pharmaceutical composition of the present invention is used according to the present invention, wherein the subject is mammal, preferably a human being.

The present invention also relates to a kit comprising one or more coupling buffer/reagents and protocol suitable for performing the method of the present invention.

In one embodiment, the kit comprises one or more coupling buffer/reagents and protocol suitable for performing the method of the present invention.

The present invention relates to a kit comprising buffer/reagents and protocol suitable for performing the method of the present invention and optionally means to purify or enrich for e.g. molecules of the present invention or molecules obtained by the method of the present invention and/or means to wash said molecules and/or means to store said molecules. Said molecules and the additional means are thereby preferably packaged together in one sealed package or kit.

The present invention also relates to a kit that comprises the nanoparticle of the present invention and/or the nanoparticle obtainable by a method of the present invention.

The present invention relates to a kit comprising the nanoparticle of the present invention and/or the nanoparticle obtainable by a method of the present invention and/or optionally means to purify or enrich said molecules and/or means to wash said molecules and/or means to store said molecules. Said molecules and the additional means are thereby preferably packaged together in one sealed package or kit.

Parts of the kit (or the “kit of parts”) of the invention can be packaged individually in vials or bottles or in combination in containers or multicontainer units. The manufacture of the kits follows preferably standard procedures, which are known to the person skilled in the art.

The kit of the present invention may comprise one or more container(s), optionally with a label. Suitable containers include, for example, bottles, vials, and test tubes. The containers may be formed from a variety of materials such as glass or plastic, and are preferably sterilized. The container holds a composition having an active ingredient or comprising a buffer which is effective for the method of the present invention. Further container may hold suitable buffers (for example reaction buffers), which allow the specific reactions to take place. It is also envisaged that containers are included which hold diverse buffers, for example reaction buffers and/or buffers for the purification of the molecules of the present invention and/or the molecule obtainable by the method of the present invention etc. The active agent in the composition is preferably the molecule obtainable by the method of the present invention or the molecule of the present invention or the pharmaceutical composition of the present invention.

The kit may also comprise written instructions for performing the method of the present invention in accordance with the methods and uses of the present invention. Said kit may further comprise a label or imprint indicating that the contents can be used for the augmentation of the nanoparticle in accordance with the present invention and/or for said nanoparticle of the present invention.

It is also envisaged that the kit of the present invention, further comprises for example buffers, vials, control(s), stabilizer(s), written instructions which aid the skilled person in the preparation or use of the nanoparticle of the present invention.

In addition, the present invention also relates to the use of the nanoparticle of the present invention or the nanoparticle obtainable by the method of the present invention or the pharmaceutical composition of the present invention in therapy, preferably in the treatment of cancer in a subject.

The present invention also relates to a method of treating cancer in a subject, comprising administering a therapeutically effective amount of the nanoparticle of the present invention or the nanoparticle obtainable by the method of the present invention or the pharmaceutical composition of the present invention to said subject.

The term “administration” means administering of a therapeutically or diagnostically effective dose of the aforementioned nanoparticle of the present invention to a subject. Different routes of administration are possible and are described above.

The present invention also relates to the use of the nanoparticle of the present invention or the nanoparticle obtainable by the method of the present invention or the pharmaceutical composition of the present invention, for the preparation of a medicament. For example, for a medicament effective in the treatment of cancer.

Unless otherwise stated, the following terms used in this document, including the description and claims, have the definitions given below.

Those skilled in the art will recognize, or be able to ascertain, using not more than routine experimentation, many equivalents to the specific embodiments of the invention described herein. Such equivalents are intended to be encompassed by the present invention.

It is to be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein, the term “comprising” can be substituted with the term “containing” or “including” or sometimes when used herein with the term “having”.

When used herein, “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms. Any such replacement is envisioned by the present disclosure.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All documents cited throughout the text of this specification (including all patents, patent publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The invention is further illustrated by following items:

Item 1. A method of generating a nanoparticle comprising contacting
a) a fusion protein (A), said fusion protein (A) comprising an antibody (A1) and a positively charged polypeptide (A2);
b) a positively charged polypeptide (B); and
c) a negatively charged molecule (C);
thereby forming a nanoparticle.

Item 2. The method of item 1, wherein the method further comprises recovery of the nanoparticle.

Item 3. The method of item 1 or 2, wherein the positively charged polypeptide (B) is in molar excess compared to the fusion protein (A).

Item 4. The method of any one of the preceding items, wherein the negatively charged molecule (C) is in molar excess compared to the fusion protein (A).

Item 5. The method of any one of the preceding items, wherein the negatively charged molecule (C) is in molar excess compared to the positively charged polypeptide (B).

Item 6. The method of any one of the preceding items, wherein the molar ratio between the positively charged polypeptide (B) and the fusion protein (A) is at least about 10:1.

Item 7. The method of any one of the preceding items, wherein the molar ratio between the positively charged polypeptide (B) and the fusion protein (A) is from about 20:1 to about 50:1.

Item 8. The method of any one of the preceding items, wherein the molar ratio between the negatively charged molecule (C) and the fusion protein (A) is at least about 1:1.

Item 9. The method of any one of the preceding items, wherein the nanoparticle is formed by self-assembly.

Item 10. The method of any one of the preceding items, wherein the method comprises incubation at about 0-37° C.

Item 11. The method of any one of the preceding items, wherein the method comprises incubation for at least about 1 h.

Item 12. The method of any one of the preceding items, wherein in the fusion protein (A) the antibody (A1) and the positively charged polypeptide (A2) are fused via a linker.

Item 13. The method of item 12, wherein the linker is an unstructured linker.

Item 14. The method of item 12 or 13, wherein the linker is a glycine-serine linker.

Item 15. The method of any one of the preceding items, wherein the antibody (A1) comprises a heavy chain and a light chain.

Item 16. The method of any one of the preceding items, wherein in the fusion protein (A) the positively charged polypeptide (A2) is fused to the C terminus of a heavy chain of the antibody (A1) and/or the C terminus of a light chain of the antibody (A1).

Item 17. The method of any one of the preceding items, wherein in the fusion protein (A) a positively charged polypeptide (A2) is fused to the C terminus of each heavy chain of the antibody (A1) and/or to the C terminus of each light chain of the antibody (A1).

Item 18. The method of any one of the preceding items, wherein the antibody (A1) is specific for a cell surface molecule.

Item 19. The method of item 18, wherein cell surface molecule is capable of internalization upon binding of the antibody.

Item 20. The method of item 18 or 19, wherein the cell surface molecule is expressed on a cell which is susceptible to therapeutic treatment by the negatively charged molecule.

Item 21. The method of any one of the preceding items, wherein the antibody (A1) is specific for a cancer-associated antigen.

Item 22. The method of any one of the preceding items, wherein the antibody (A1) is specific for CD33, EGFR, IGF1R, or CD20.

Item 23. The method of any one of the preceding items, wherein the antibody (A1) is gemtuzumab, cetuximab, cixutumumab, teprotumumab, GR11L, rituximab.

Item 24. The method of any one of the preceding items, wherein the antibody (A1) has the CDR sequences selected form the group consisting of:

a.
CDR-H1:
(SEQ ID NO: 1)
GFSLTNYG,
CDR-H2:
(SEQ ID NO: 2)
IWSGGNT,
CDR-H3:
(SEQ ID NO: 3)
ARALTYYDYEFAY,
CDR-L1:
(SEQ ID NO: 4)
QSIGTN,
CDR-L2:
YAS,
and
CDR-L3:
(SEQ ID NO: 5)
QQNNNWPTT;
b.
CDR-H1:
(SEQ ID NO: 10)
GYTITDSN,
CDR-H2:
(SEQ ID NO: 11)
IYPYNGGT,
CDR-H3:
(SEQ ID NO: 12)
VNGNPWLAY,
CDR-L1:
(SEQ ID NO: 13)
ESLDNYGIRF,
CDR-L2:
AAS,
and
CDR-L3:
(SEQ ID NO: 14)
QQTKEVPWS;
c.
CDR-H1:
(SEQ ID NO: 19)
GGTFSSYAIS,
CDR-H2:
(SEQ ID NO: 20)
GIIPIFGTANYAQKFQ,
CDR-H3:
(SEQ ID NO: 21)
APLRFLEWSTQDHYYYYYMDV,
CDR-L1:
(SEQ ID NO: 22)
QGDSLRSYYAT,
CDR-L2:
(SEQ ID NO: 23)
GENKRPS,
and
CDR-L3:
(SEQ ID NO: 24)
KSRDGSGQHLV;
d.
CDR-H1:
(SEQ ID NO: 29)
GFTFSSYG,
CDR-H2:
(SEQ ID NO: 30)
IWFDGSST,
CDR-H3:
(SEQ ID NO: 31)
ARELGRRYFDL,
CDR-L1:
(SEQ ID NO: 32)
QSVSSY,
CDR-L2:
(SEQ ID NO: 33)
IWFDGSST,
and
CDR-L3:
(SEQ ID NO: 34)
QQRSKWPPWT;
and
e.
CDR-H1:
(SEQ ID NO: 39)
GYTFTSYN,
CDR-H2:
(SEQ ID NO: 40)
IYPGNGDT,
CDR-H3:
(SEQ ID NO: 41)
CARSTYYGGDWYFNV,
CDR-L1:
(SEQ ID NO: 42)
SSVSYI,
CDR-L2:
ATS,
and
CDR-L3:
(SEQ ID NO: 43)
QQWTSNPPT.

Item 25. The method of any one of the preceding items, wherein the antibody (A1) has the VH and VL sequences selected form the group consisting of:
a. SEQ ID NO: 6 and 7;
b. SEQ ID NO: 15 and 16;
c. SEQ ID NO: 25 and 26;
d. SEQ ID NO: 35 and 36; and
e. SEQ ID NO: 44 and 45.

Item 26. The method of any one of the preceding items, wherein the antibody (A1) has the heavy chain and light chain sequences selected form the group consisting of: a. SEQ ID NO: 8 and 9; b. SEQ ID NO: 17 and 18; c. SEQ ID NO: 27 and 28; d. SEQ ID NO: 37 and 38; e. SEQ ID NO: 46 and 47; and f. SEQ ID NO: 66 and 18.

Item 27. The method of any one of the preceding items, wherein the negatively charged molecule (C) is a nucleic acid.

Item 28. The method of item 27, wherein the nucleic acid is a double stranded nucleic acid.

Item 29. The method of item 27, wherein the nucleic acid is a single stranded nucleic acid.

Item 30. The method of any one of items 27-29, wherein the nucleic acid has about 18 to about 25 bp.

Item 31. The method of any one of items 27-29, wherein the nucleic acid has about 18 to about 25 nt.

Item 32. The method of any one of the preceding items, wherein the negatively charged molecule (C) is a DNA or RNA.

Item 33. The method of any one of the preceding items, wherein the negatively charged molecule (C) is siRNA, esiRNA, antisense oligonucleotide, or miRNA.

Item 34. The method of any one of the preceding items, wherein the negatively charged molecule (C) is a siRNA specific for KRAS, BRAF, PIK3CA, PAX3-FKHR, EWS-FLI1, c-MYC, TP53, DNMT3A, IDH1, NPM1, or FLT3.

Item 35. The method of any one of the preceding items, wherein the negatively charged molecule (C) is a mixture of siRNAs specific for one or more targets preferably selected from the group consisting of KRAS, BRAF, PIK3CA, PAX3-FKHR, EWS-FLI1, c-MYC, TP53, DNMT3A, IDH1, NPM1, and FLT3.

Item 36. The method of any one of the preceding items, wherein the negatively charged molecule (C) has a molecular weight of about 20 kDa or less.

Item 37. The method of any one of the preceding items, wherein the negatively charged molecule (C) has a charge of at least 2−.

Item 38. The method of any one of the preceding items, wherein the positively charged polypeptide (B) comprises the same amino acid sequence as the positively charged polypeptide (A2) that is comprised in the fusion protein (A).

Item 39. The method of any one of the preceding items, wherein the positively charged polypeptide (B) is a protamine or histone.

Item 40. The method of any one of the preceding items, wherein the positively charged polypeptide (A2) comprised in the fusion protein (A) is a protamine or histone.

Item 41. A nanoparticle obtainable by a method of any one of the preceding items.

Item 42. A nanoparticle comprising:
a) a fusion protein (A), said fusion protein (A) comprising an antibody (A1) and a positively charged polypeptide (A2);
b) a positively charged polypeptide (B); and
c) one or more negatively charged molecule(s) (C).

Item 43. The nanoparticle of item 41 or 42, wherein the positively charged polypeptide (B) is enriched in the outer and/or inner portion of the nanoparticle.

Item 44. The nanoparticle of any one of items 41-43, wherein the fusion protein (A) is enriched in the outer portion of the nanoparticle.

Item 45. The nanoparticle of any one of items 41-44, wherein the one or more negatively charged molecules (C) are enriched in the inner portion of the nanoparticle.

Item 46. The nanoparticle of any one of items 41-45, wherein the nanoparticle has a mean diameter of about 0.05 μm to about 10 μm.

Item 47. The nanoparticle of any one of items 42-47, wherein in the fusion protein (A) the antibody (A1) and the positively charged polypeptide (A2) are fused via a linker.

Item 48. The nanoparticle of item 47, wherein the linker is an unstructured linker.

Item 49. The nanoparticle of item 47 or 48, wherein the linker is a glycine-serine linker.

Item 50. The nanoparticle of any one of items 41-49, wherein the antibody (A1) comprises a heavy chain and a light chain.

Item 51. The nanoparticle of any one of items 41-50, wherein the antibody (A1) is specific for a cell surface molecule.

Item 52. The nanoparticle of item 51, wherein cell surface molecule is capable of internalization upon binding of the antibody.

Item 53. The nanoparticle of item 51 or 52, wherein the cell surface molecule is expressed on a cell which is susceptible to therapeutic treatment by the negatively charged molecule.

Item 54. The nanoparticle of any one of items 41-53, wherein the antibody (A1) is specific for a cancer-associated antigen.

Item 55. The nanoparticle of any one of items 41-54, wherein the antibody (A1) is specific for CD33, EGFR, IGF1R, or CD20.

Item 56. The nanoparticle of any one of items 41-55, wherein the antibody (A1) is gemtuzumab, cetuximab, cixutumumab, teprotumumab, GR11L, rituximab.

Item 57. The nanoparticle of any one of items 41-56, wherein the antibody (A1) has the CDR sequences selected form the group consisting of:

a.
CDR-H1:
(SEQ ID NO: 1)
GFSLTNYG,
CDR-H2:
(SEQ ID NO: 2)
IWSGGNT,
CDR-H3:
(SEQ ID NO: 3)
ARALTYYDYEFAY,
CDR-L1:
(SEQ ID NO: 4)
QSIGTN,
CDR-L2:
YAS,
and
CDR-L3:
(SEQ ID NO: 5)
QQNNNWPTT;
b.
CDR-H1:
(SEQ ID NO: 10)
GYTITDSN,
CDR-H2:
(SEQ ID NO: 11)
TYPYNGGT,
CDR-H3:
(SEQ ID NO: 12)
VNGNPWLAY,
CDR-L1:
(SEQ ID NO: 13)
ESLDNYGIRF,
CDR-L2:
AAS,
and
CDR-L3:
(SEQ ID NO: 14)
QQTKEVPWS;
c.
CDR-H1:
(SEQ ID NO: 19)
GGTFSSYAIS,
CDR-H2:
(SEQ ID NO: 20)
GIIPIFGTANYAQKFQ,
CDR-H3:
(SEQ ID NO: 21)
APLRFLEWSTQDHYYYYYMDV,
CDR-L1:
(SEQ ID NO: 22)
QGDSLRSYYAT,
CDR-L2:
(SEQ ID NO: 23)
GENKRPS,
and
CDR-L3:
(SEQ ID NO: 24)
KSRDGSGQHLV;
d.
CDR-H1:
(SEQ ID NO: 29)
GFTFSSYG,
CDR-H2:
(SEQ ID NO: 30)
IWFDGSST,
CDR-H3:
(SEQ ID NO: 31)
ARELGRRYFDL,
CDR-L1:
(SEQ ID NO: 32)
QSVSSY,
CDR-L2:
(SEQ ID NO: 33)
IWFDGSST,
and
CDR-L3:
(SEQ ID NO: 34)
QQRSKWPPWT;
and
e.
CDR-H1:
(SEQ ID NO: 39)
GYTFTSYN,
CDR-H2:
(SEQ ID NO: 40)
IYPGNGDT,
CDR-H3:
(SEQ ID NO: 41)
CARSTYYGGDWYFNV,
CDR-L1:
(SEQ ID NO: 42)
SSVSYI,
CDR-L2:
ATS,
and
CDR-L3:
(SEQ ID NO: 43)
QQWTSNPPT.

Item 58. The nanoparticle of any one of items 41-57, wherein the antibody (A1) has the VH and VL sequences selected form the group consisting of: a. SEQ ID NO: 6 and 7; b. SEQ ID NO: 15 and 16; c. SEQ ID NO: 25 and 26; d. SEQ ID NO: 35 and 36; and e. SEQ ID NO: 44 and 45.

Item 59. The nanoparticle of any one of items 41-58, wherein the antibody (A1) has the heavy chain and light chain sequences selected form the group consisting of: a. SEQ ID NO: 8 and 9; b. SEQ ID NO: 17 and 18; c. SEQ ID NO: 27 and 28; d. SEQ ID NO: 37 and 38; e. SEQ ID NO: 46 and 47; f. SEQ ID NO: 66 and 18.

Item 60. The nanoparticle of any one of items 41-59, wherein negatively charged molecule (C) is a nucleic acid.

Item 61. The nanoparticle of item 60, wherein the nucleic acid is a double stranded nucleic acid.

Item 62. The nanoparticle of item 60, wherein the nucleic acid is a single stranded nucleic acid.

Item 63. The nanoparticle of any one of items 60-62, wherein the nucleic acid has about 18 to about 25 bp.

Item 64. The nanoparticle of any one of items 60-63, wherein the single stranded nucleic acid has about 18 to about 25 nt.

Item 65. The nanoparticle of any one of items 41-64, wherein the negatively charged molecule (C) is a DNA or RNA.

Item 66. The nanoparticle of any one of items 41-65, wherein the negatively charged molecule (C) is siRNA, esiRNA, antisense oligonucleotide, or miRNA.

Item 67. The nanoparticle of any one of items 41-66, wherein the negatively charged molecule (C) is siRNA specific for KRAS, BRAF, PIK3CA, PAX3-FKHR, EWS-FLI1, c-MYC, TP53, DNMT3A, IDH1, NPM1, or FLT3.

Item 68. The nanoparticle of any one of items 41-67, wherein the negatively charged molecule (C) is a mixture of siRNAs specific for one or more targets preferably selected from the group consisting of KRAS, BRAF, PIK3CA, PAX3-FKHR, EWS-FLI1, c-MYC, TP53, DNMT3A, IDH1, NPM1, and FLT3.

Item 69. The nanoparticle of any one of items 41-68, wherein the negatively charged molecule (C) has a molecular weight of about 20 kDa or less.

Item 70. The nanoparticle of any one of items 41-69, wherein the negatively charged molecule (C) has a charge of at least 2−.

Item 71. The nanoparticle of any one of items 41-70, wherein the positively charged polypeptide (B) comprises the same amino acid sequence as the positively charged polypeptide (A2) that is comprised in the fusion protein (A).

Item 72. The nanoparticle of any one of items 41-71, wherein the positively charged polypeptide (B) is a protamine or histone.

Item 73. The nanoparticle of any one of items 41-72, wherein the positively charged polypeptide (A2) comprised in the fusion protein (A) is a protamine or histone.

Item 74. A composition comprising a nanoparticle of any one of items 41-73.

Item 75. The composition of item 74, wherein the composition is a pharmaceutical composition.

Item 76. A nanoparticle of any one of items 41-73 or a composition of item 74 or 75 for use in therapy.

Item 77. The nanoparticle or composition for the use of item 76, wherein the use is in the treatment of cancer.

Item 78. The nanoparticle or composition for the use of item 76, wherein the use is in the treatment of a solid tumor.

Item 79. The nanoparticle or composition for the use of item 76, wherein the use is in the treatment of a cancer selected from the group consisting of lung cancer, sarcoma, colorectal cancer, blood cancer.

Item 80. A kit comprising a nanoparticle of any one of items 41-73 or a composition of item 74 or 75.

Item 81. The method of any one of items 1-40 or the nanoparticle of any one of items 41-73, wherein the negatively charged molecule is a drug and/or prodrug, such as remdesivir triphosphate.

Item 82. The method of any one of items 1-40 or the nanoparticle of any one of items 41-73, wherein the negatively charged molecule is a drug and/or prodrug, such as ibrutinib, conjugated to a moiety having a negative net charge, such as Cy 3.5 or Alexa488.

### EXAMPLES

The following examples illustrate the invention. These examples should not be construed as to limit the scope of this invention. The examples are included for purposes of illustration and the present invention is limited only by the claims.

### Example 1: Modification of the Conjugation Protocol

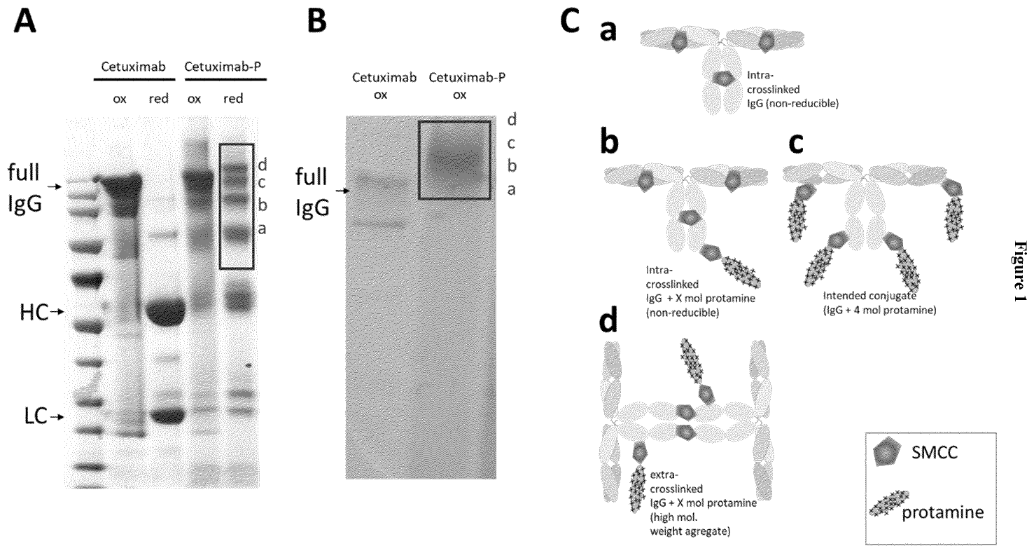

While we performed the chemical conjugations between the chosen carrier antibodies, sulfo-SMCC and protamine as published in Bäumer, N. et al., 2016; Bäumer, N. et al., 2018; Bäumer, S. et al., 2015, we were puzzled by resulting conjugates, that had unintended properties in SDS-PAGE electrophoresis: For instance, we frequently observed the phenomenon of IgG conjugates, that prove to be no longer reducible by reducing agents such as DTT, DTE, beta-mercapto-ethanol or TCEP (FIG. 1 BRIEF DESCRIPTION OF THE DRAWINGS

Figure, A and B, see gel a for illustration). Next, we observed conjugates exhibiting a much higher molecular weight than intended, representing dimerized or multimers of IgG crosslinked to each other, some with additional protamine, some without (FIG. 1 BRIEF DESCRIPTION OF THE DRAWINGS

Figure, C, see gel B for illustration). In extreme cases, the complexity of all of those side reactions lead to a cloud-like appearance of the resulting conjugates, probably caused by a mixture of all of these conjugates a-d in FIG. 1B.

Conversely, the intended conjugate was the formulation marked with C in FIG. 1 BRIEF DESCRIPTION OF THE DRAWINGS

Figure, a molecule which retains its natural disulphide bonds intra- and extrapeptide HC and LC, without additional internal crosslinking manipulations, but with a manifold of crosslinked protamine to light (LC) and heavy chain (HC).

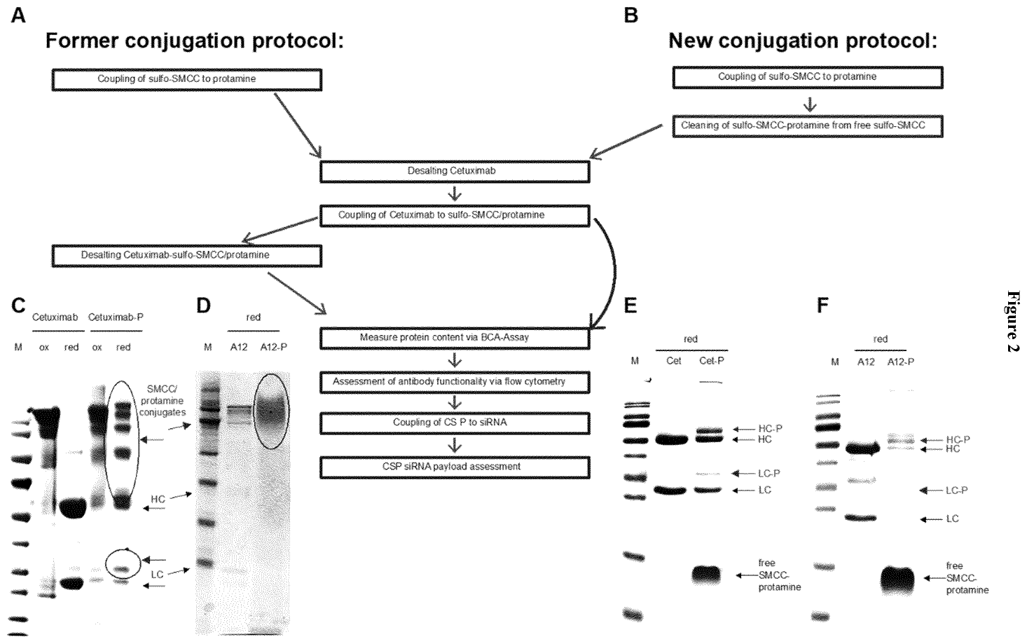

As a consequence, we have modified the conjugation protocol by introducing an additional purification step after the amino-terminal activation of the protamine peptide with sulfo-SMCC. The resulting products were purified by gel chromatography, separating the activated SMCC-protamine from the still active excess educt sulfo-SMCC. From this step on, the antibody conjugation was performed by a homogeneous solution of pure SMCC-protamine, without any contaminations of residual crosslinker (FIG. 2).

As a consequence, all experiments shown in the following Examples were performed by following the “new” SMCC-depletion protocol. The resulting complexes improved enormously regarding their electrophoretical homogeneity, targeting performance and functional effectivity.

So, all findings shown here were performed with a therapeutic agent synthesized by a new production process, not with the formulations published in Bäumer, N. et al., 2016; Bäumer, N. et al., 2018; Bäumer, S. et al., 2015.

### Example 2: Oncogene Inactivation in Non-Small Cell Lung Cancer Using the Unpublished Modified Conjugation Protocol

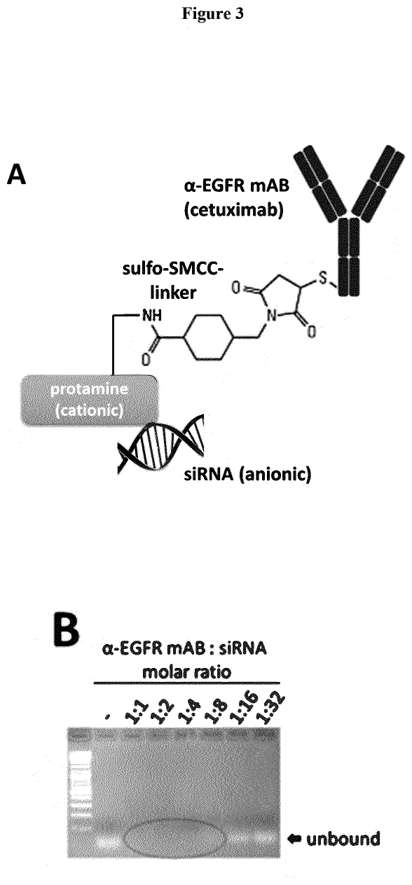

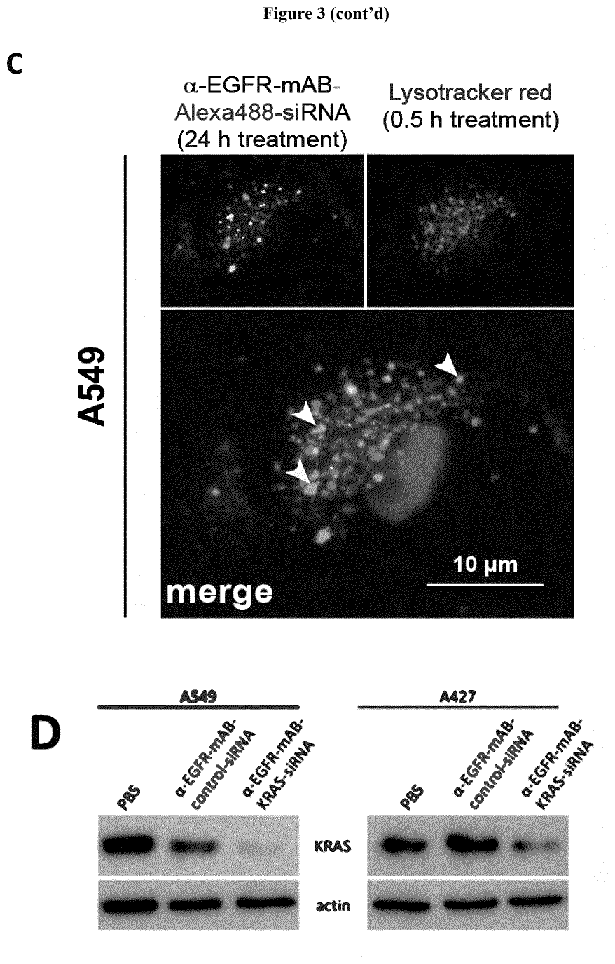

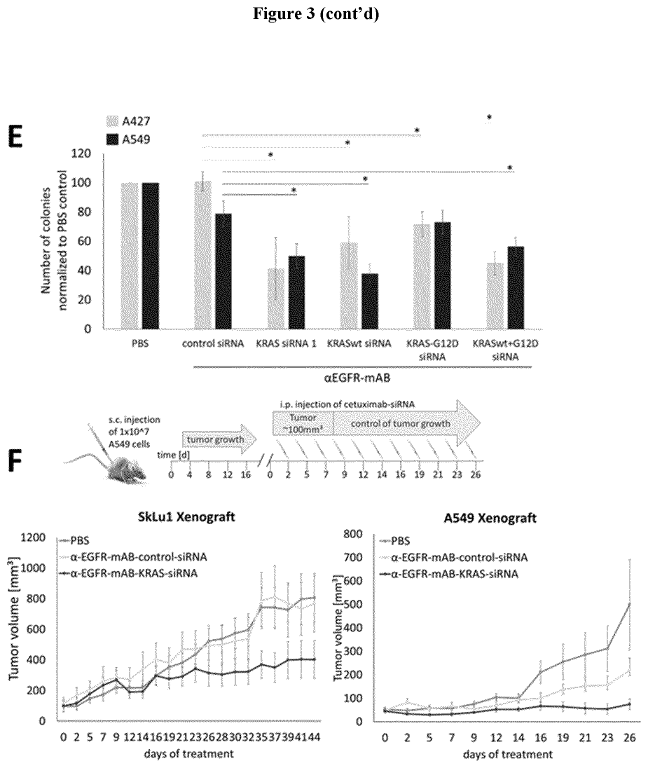

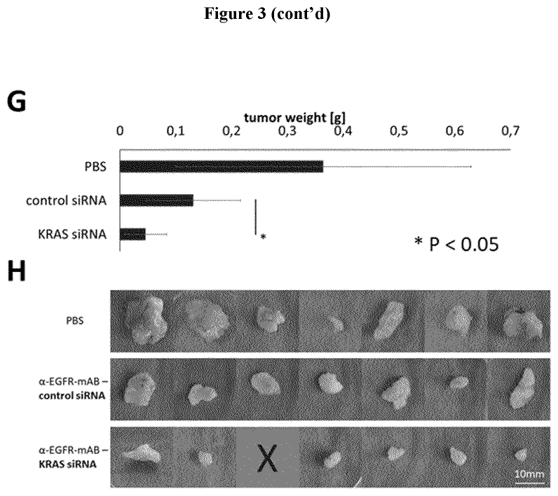

Next, we targeted EGFR-expressing non-small cell lung cancer cell lines (NSCLC) with cetuximab-protamine. Here, cetuximab-protamine was able to bind 8 mol of siRNA per mol of cetuximab-protamine (FIG. 3A, B) and delivered siRNA in a receptor-dependent manner to early endosomes (FIG. 3 C). KRAS was silenced effectively in in vitro-treated NSCLC cell lines after treatment with anti-EGFR-mAB-protamine complexed with siRNA against KRAS (FIG. 3 FIG. D). In cetuximab-sensitive A549 cells, conjugated cetuximab loaded with control siRNA had a small effect on cell growth, colony formation, tumor growth, and tumor weight in CD1 nude mice, which is in line with randomized clinical trials using cetuximab in NSCLC patients. But this effect was significantly amplified by use of KRAS siRNA, see FIGS. 3 E and F (right panel). The cetuximab-resistant SK-LUI cells tolerated cetuximab-control siRNA much better, but also here, colony- and tumor growth was significantly inhibited by KRAS siRNA (FIGS. 3 E and F, left panel).

Next, we investigated the effect of the systemic anti-EGFR-mAB-siRNA treatment of the NSCLC xenograft tumors on proliferation marker Ki67 expression in immunofluorescence on cryo-sections (A549 tumors) and paraffin sections (SK-LU-1 tumors). In PBS or anti-EGFR-mAB-control-siRNA treated A549 (FIG. 4A-D) and SK-LU-1 tumors (FIG. 4 G-J), Ki67 staining was widespread amongst Hoechst-stained nuclei of the respective tumor cells. On the contrary, tumors treated with anti-EGFR-mAB-KRAS-siRNA exhibited much less proliferative cells exhibiting Ki67 staining (FIGS. 4 E-F and 2 K-L).

Tumor growth retardation by systemic anti-EGFR-mAB-siRNA application can be induced not only by a reduced proliferation of the tumor tissue, but also by increased apoptosis. Moreover, the induction of apoptosis is of course a desirable effect of a potential cancer therapeutic agent to be able to actively reduce tumor size. We aimed to investigate the abundance of apoptotic cells by TUNEL (terminal deoxynucleotidyl transferase dUTP nick end labeling) staining of the respective tumor tissues ex vivo that reveals DNA fragmentation in nuclei as a sign for apoptosis. After peroxidase-staining development, control treated tumor sections exhibited a doubling in TUNEL-positive nuclei in A549 tumors treated with anti-EGFR-mAB-control siRNA (FIG. 5 C-D) compared to PBS treatment (FIG. 5A-B) and a further doubling when the carrier contained the KRAS siRNA (FIG. 5 E-F for illustration and FIG. 5 FIG. M for statistics). By means of the apoptosis signal, the SK-LUI tumors treated with PBS (FIG. 5 G-H) and anti-EGFR-mAB-control siRNA (FIG. 5 I-J) were indistinguishable, but the number of TUNEL-positive nuclei was 4-fold increased, if KRAS-siRNA was conjugated to the antibody carrier and applied to the xenograft tumor (FIG. 5 K-L and FIG. 5 N).

Taken together, the marked and significant reduction of the tumor size by treatment with anti-EGFR-mAB-KRAS siRNA can be explained by a combination of reduced proliferation and increased apoptosis in the respective tumors.

Moreover, we also exploit the fact that EGFR is surface-expressed also in sarcomas (see (Herrmann et al., 2010) and next chapter. The observation that cetuximab was ineffective as a single agent in a first clinical trial in sarcoma (Ha et al., 2013) is not relevant for our purposes, since our system relies on the antibody not as an active anti-cancer agent but as a component of the shuttle system carrying the oncogene-specific effector siRNA.

### Example 3: Targeting Oncogenes in Rhabdomyosarcoma

Rhabdomyosarcomas (RMS) are aggressive soft tissue sarcomas that originate from immature myoblasts and mainly occur in children and young adults. Pediatric RMS are divided into two major categories according to their histological appearance: approximately ⅔ represent embryonal RMS (ERMS), which have a more favorable prognosis, and ⅓ represent the more aggressive alveolar RMS (ARMS) (Stevens, 2005). So far, no common genetic lesions of diagnostic value have been found in ERMS, except an accumulation of loss of heterozygosity in 11p15 (Chen et al., 2013). Targeting critical drivers of ARMS is more likely to have a therapeutic impact. Genetic lesions characterizing alveolar rhabdomysarcoma (ARMS) are the PAX3-FKHR or PAX7-FKHR fusions by chromosomal translocation of t(1; 13) or t(2; 13). Thus, targeting of PAX-FKHR fusion genes and their transcripts could be a specific and effective means to inhibit malignant growth and induce apoptosis of ARMS cells. Inhibition of oncogenic fusion proteins or mutated proteins was found to be challenging. Either the proteins were stated “undruggable”, or drug treatment led to the selection of a resistant version of the protein and to a more aggressive relapse (Verdine and Walensky, 2007).

Downregulation of fusion proteins by RNAi is intended to overcome these problems, since the expression is inhibited on the mRNA level. Specifically, downregulation of expression of the PAX3-FKHR fusion protein by RNAi in RMS cells had a direct effect on the malignant phenotype. Silencing PAX3-FKHR fusion by siRNA against PAX3 or PAX3-FKHR reduced proliferation, mobility and colony formation of RMS cell lines (Kikuchi et al., 2008; Liu, L. et al., 2012). Therefore, we hoped that effective downregulation of ARMS-specific fusion proteins by RNAi carried to the tumor cells by a stable and specific method by our established antibody-protamine carrier system in vivo leads to a therapeutic effect.

In RMS, IGF1R and epithelial growth factor receptor (EGFR) are candidate targets for our modular carrier. Our technique allows to distinguish tumor cells from other cells by two independent characteristics and thus provides a dual layer of specificity: a) The cell surface receptor decoration and b) the cellular oncogenic equipment. We and others (Herrmann et al., 2010) identified EGFR as well as IGF1R high-density surface expression on different alveolar (and embryonic) RMS cell lines. Both cell surface receptors can serve as target components of our system.

To check targeting efficiency of our antibody constructs in RMS cell lines, we treated the ERMS EGFR⁺ cell line RD with anti-EGFR antibody (cetuximab)-siRNA complexes and the ARMS IGF1R⁺ cell line RH-30 with anti-IGF1R-siRNA complexes (FIG. 6). Both cell lines express IGF1R and EGFR to variable levels (FIG. 6A), and according to their highest expression, RD cells internalize preferentially anti-EGFR-Alexa488-siRNA (FIGS. 6 B and C, upper panels) whereas RH-30 cells internalize preferentially anti-IGF1R-Alexa488 siRNA complexes (FIG. 6 B, lower panel). Alexa488-siRNA could be internalized into up to 90% of all EGFR⁺ RD cells by cetuximab-protamine (FIG. 6 C, middle panel, whereas anti-IGF1R antibody performed less effective in IGF1R⁺ RH-30 cells (FIG. 6 B, lower panel).

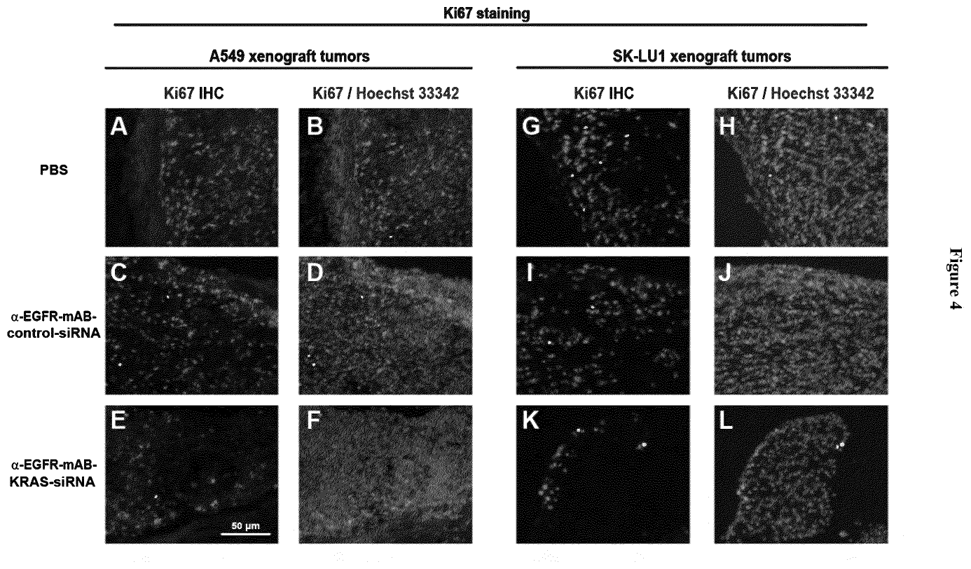

To perform a proof-of-principle experiment for a specific targeting of RMS-typical PAX3-Forkhead fusion oncogene, we designed various siRNAs spanning the breakpoint region (FIG. 7 D) of PAX3-FKHR and subjected ARMS RH-30 cells to treatment with those breakpoint spanning siRNAs coupled to cetuximab-protamine in a colony formation assay. Breakpoint siRNA significantly reduced colony formation in RH-30 compared to control (FIG. 7 E), whereas ERMS type cells RD colony formation was compromised by transport of NRAS combined with cMyc siRNA (FIGS. 7A and B), both target genes representing well known oncogenes in ERMS. Consequently, oncogene expression of NRAS was reduced in Western blot after treatment with anti-EGFR/NRAS siRNA in RD cells (middle row FIG. 7 C).

These results illustrate that we can target RMS cell lines using our modular antibody-siRNA system with at least 2 different monoclonal antibodies, depending on receptor decoration of RMS tumors to specifically transport nucleic acids of our choice to elicit oncogene inactivation.

### Example 4: Targeting Oncogenes in Ewing's Sarcoma

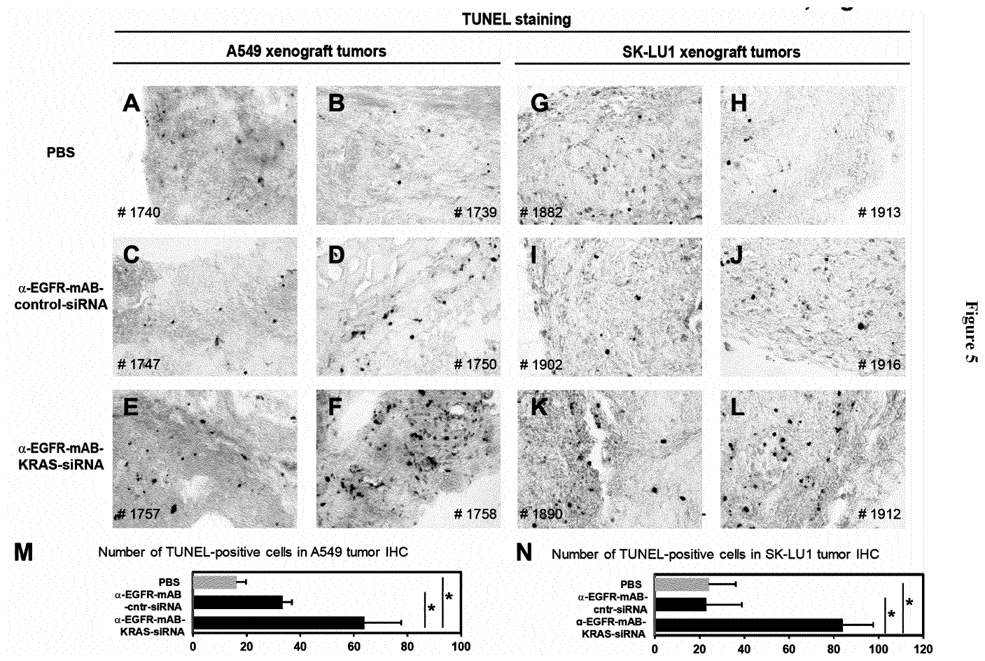

Ewing-sarcomas are bone tumors in children and young adults. With less than 35% long-term complete remissions in the metastasized stage, there is a high need for better therapeutic options (Paulussen et al., 1998; Paulussen et al., 2008). A central genetic event is the occurrence of the chromosomal translocation t(11; 22) that results in the formation of the fusion protein EWS-FLI1 in these tumor cells (Arvand and Denny, 2001). Ewing sarcoma cells express high amounts of IGF1R on their surface. We therefore intended to apply our modular therapy using the anti-IGF1R-antibody like the clone ImcA12 (cixutumumab) or teprotumumab as SMCC-protamine conjugate to transport EWS-FLI1 breakpoint-specific siRNA. As a proof-of-principle, we used the commercial anti-IGF1R murine antibody GR11L (Merck) and showed targeting of Ewing cells. These results were published in (Bäumer, N. et al., 2016) and are depicted in FIG. 8 Figure.

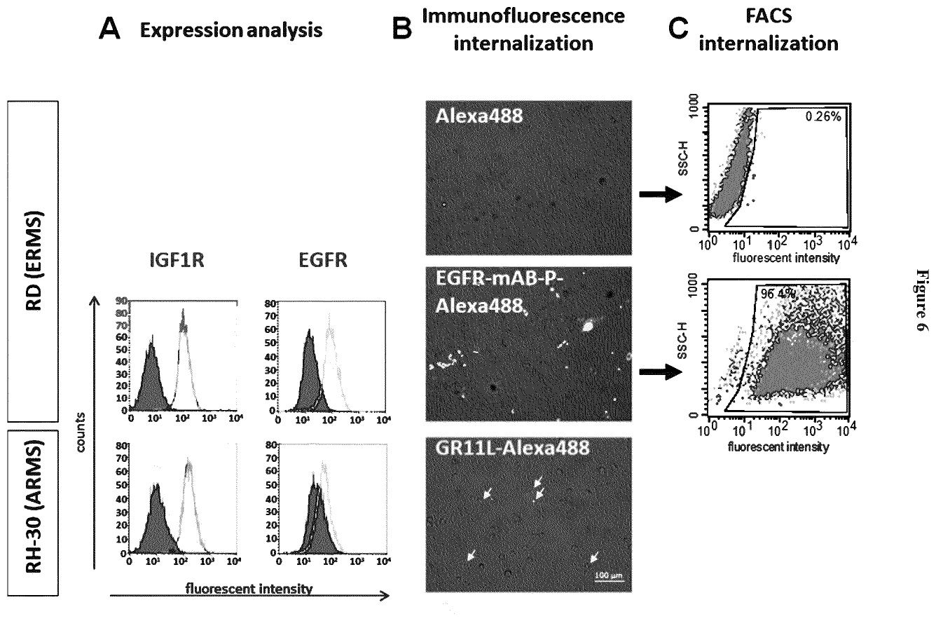

To be able to use this therapy option, we cloned and expressed two different IGF1R-antibodies in CHO-S cells and purified them using HPLC. These were the clones cixutumumab (here referred to as “A12”) and teprotumumab (here referred to as “Tepro”). Both were produced in sufficient amounts and coupled to SMCC-protamine (FIG. 9A), both bind siRNA (FIG. 9 B) and transport siRNA into IGF1R-positive cells SKNM-C (FIG. 9 C).

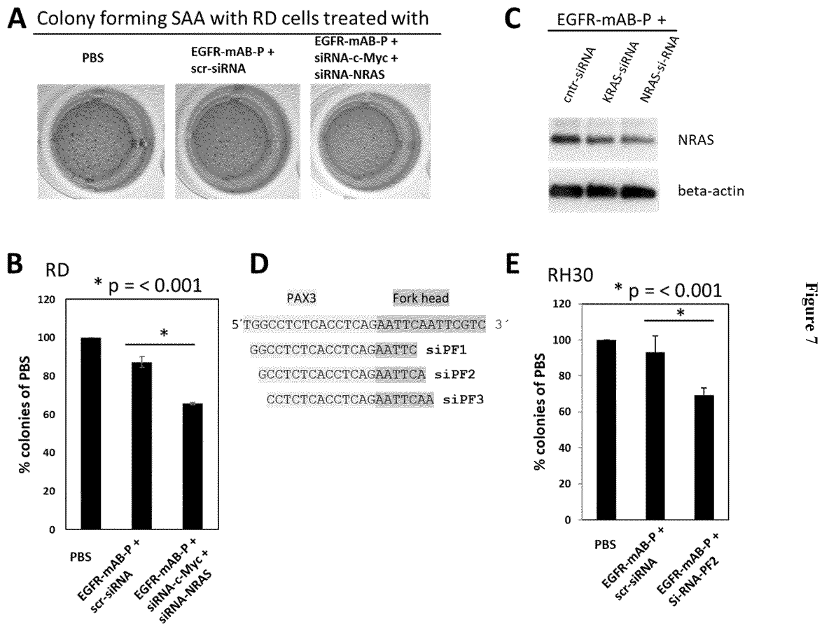

When cells were incubated with these IGF1R-mAB-protamine conjugates in complex with siRNA against EWS-FLI1 and seeded in semisolid soft-agar, colony formation was significantly reduced (FIGS. 10A and B).

We therefore conclude that our modular system can also be applied for the use of anti-IGF1R antibodies and sarcoma cells and especially for the knockdown of fusion proteins specific siRNA such as the EWS-FLI1-siRNAs.

### Example 5: Oncogene Targeting in Lymphoma Models

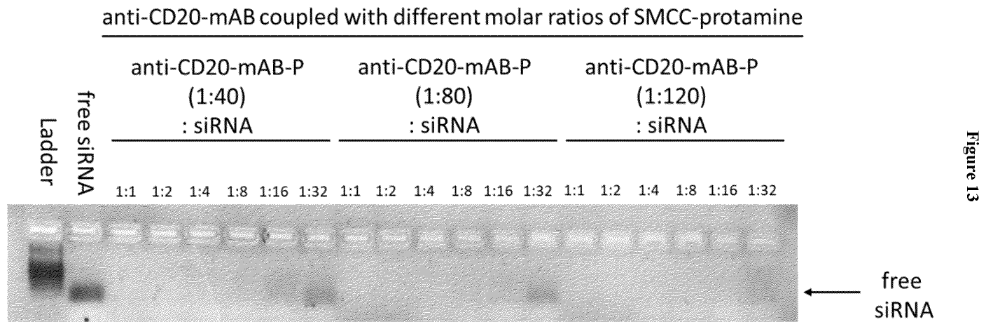

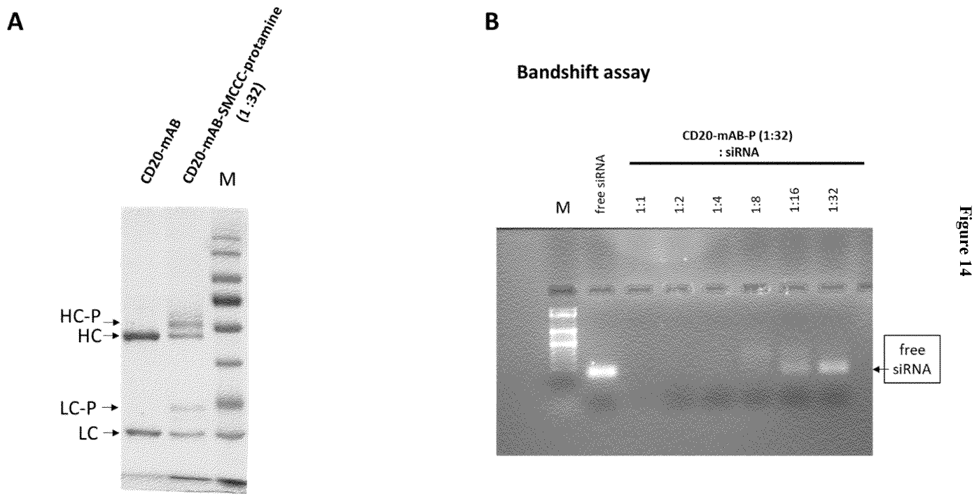

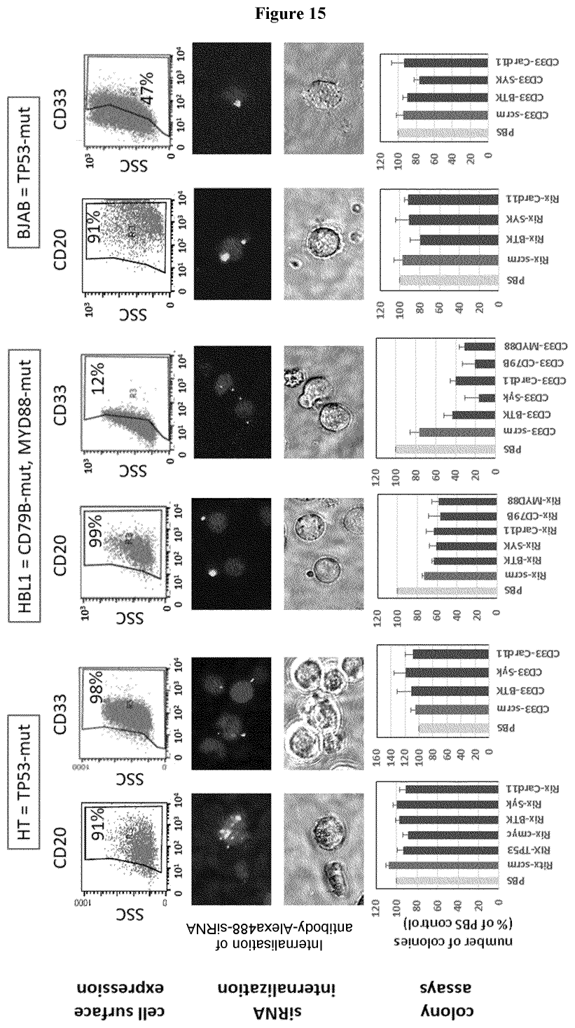

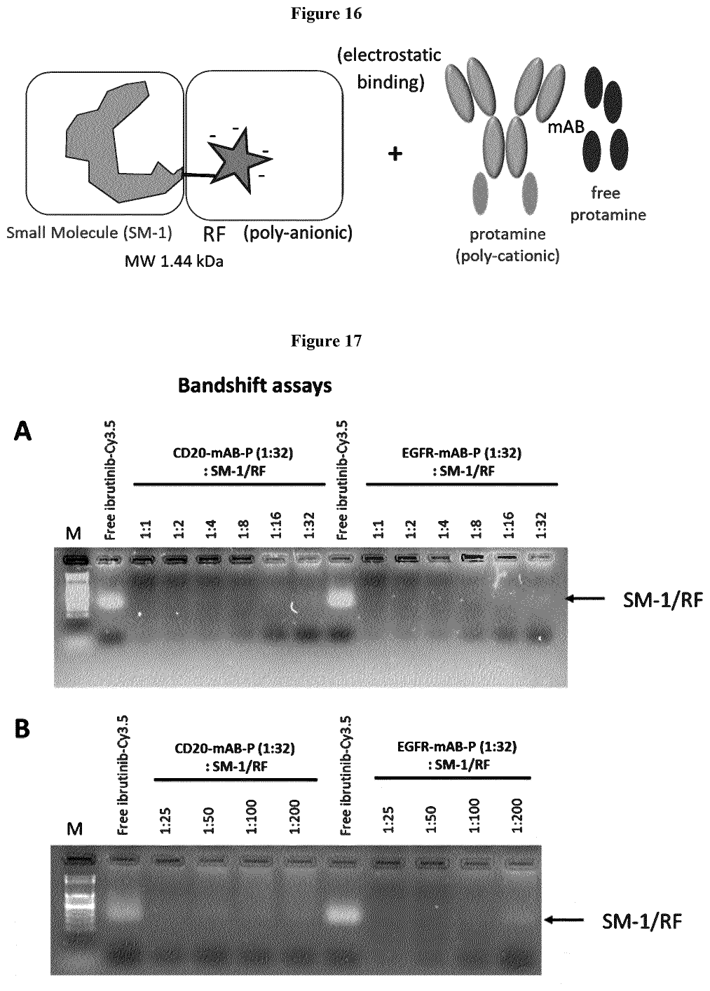

Diffuse large B-cell lymphoma (DLBCL) represents a frequent lymphoma subtype. DLBCL cells express CD20 on their surface. The standard first-line therapy for affected patients is a combination of chemotherapy and the anti-CD20 antibody rituximab. Rituximab binds and blocks the CD20 molecule and leads to antibody-dependent cellular cytotoxicity (ADCC). By this approach, roughly 65% of patients can be cured. Patients who are refractory to first-line treatment or who relapse after initial response are characterized by extremely poor survival indicating that novel therapeutic approaches are urgently warranted. Therefore, we aimed to combine the first line drug rituximab as a cell-targeting antibody with siRNAs against different oncogenes identified in the genetically quite diverse lymphoma cells (FIG. 15 Figure).

We first chemically coupled the CD20-antibody rituximab with different ratios of SMCC-protamine to compare the effectivity of each complex to bind siRNA (FIG. 13). Interestingly, despite the high excess of unbound SMCC-protamine of 80 or 120 mol, the binding of siRNA was almost identical as with coupling one mol antibody with 40 mol SMCC-protamine (FIG. 13). We therefore concluded that the least amount of SMCC-protamine is sufficient and went back to our routine ratio of antibody:SMCC-protamine of 1:32 (FIG. 14). We checked the binding capacity of rituximab-protamine first with siRNA, as done before, and observed similar coordination of siRNA and the carrier system (about 8 mol/mol) as found with other carrier antibodies (FIG. 14 B).

Subsequently, different DLBCL cell lines were tested positive for surface expression of CD20 and CD33 (FIG. 15, upper panel). Consequently, internalisation studies were carried out by anti-CD20 mAB rituximab and anti-CD33-mAB gemtuzumab.

DLBCL cell lines were subjected to a B-cell receptor-axis molecular screen by means of antibody-mediated siRNA knockdown to give rise to further target molecules besides BTK (FIG. 15 Figure), which lead to significant colony growth inhibition especially in HBL1 cells using anti-CD33-antibody-siRNA targeting (FIG. 15, lower panel).

### Example 6: Complexes Formed by Carrier Antibodies-Protamine and Small Molecular Weight Poly-Anionic Drugs with Chemical Structures Different from siRNA

In another project, we hypothesized that therapeutic monoclonal antibodies such as rituximab or gemtuzumab could be harnessed as carrier molecules for low molecular weight (lmw) drugs. This strategy would be of importance in the clinic, because the pharmacodynamics and safety of a number of lmw drugs, such as the group of kinase inhibitors, could possibly be improved by the targeted carrier over the untargeted form. Thus, we hypothesized for the use of such antibody-inhibitor conjugates that i) they can be applied in lower dosage, because the antibody helps to enrich the inhibitor in the intended target cells and ii) the antibody inhibits the inhibitor to be taken up by non-intended cells, where it may induce unintended toxic reactions.

In a first set of experiments, we synthesized negatively charged small molecule having the charge of 4—that is a derivative of a known proliferation inhibitor, here referred to as Small Molecule 1 (SM-1). Therefore, we converted the uncharged small-molecule-inhibitor to the strongly anionic compound by adding a negative charged and emitting red-fluorescent light (here referred to as “SM-1/RF”), which allowed to bind it by means of electrostatic force to our protamine-based carrier system in order to form an antibody-inhibitor-conjugate.

Besides the strong polyanionic charge of the red fluorescent dye, the conjugate had the advantage of being easily traceable in vitro and in vivo in form of a red fluorescence.

Subsequently, we performed the same experiments for the binding between SM-1/RF and two carrier systems, rituximab (CD20)-protamine and the reliable cetuximab (EGFR)-protamine. Both carriers showed a strong co-assembly of the SM-1/RF and, due to its low molecular weight as compared to siRNA (appr. 13 kDa), an extremely high mol/mol ratio of electrostatic saturation, exceeding 100 mol SM-1/RF per mol of carrier mAB (FIG. 17).

The corresponding conjugates rituximab- and cetuximab-protamine loaded with a sub-critical 20× excess of SM-1/RF were incubated with CD20 and EGFR-expressing cell lines and analysed for intracellular enrichment of SM-1/RF. In both cases, intracellular enrichment of fluorescent signals at the typical red fluorescent excitation/emission wavelength combination could be documented (FIG. 18). Thus, the modified SM-1/RF compound is being internalized by cells and consequently can get to action.

### Example 7: Surprising Features of Effective Antibody-Protamine-siRNA Formulations

The exact conjugation procedure of all our used antibodies can be divided in two steps: First, the amino-terminal conjugation of the protamine to sulfo-SMCC, then a size exclusion process to remove the excess of conjugation cross-linker, and second the cysteine-directed conjugation of the activated protamine-SMCC to the IgG backbone. The resulting bioconjugate exhibits a significant molecular weight shift as seen from SDS-PAGE electrophoresis both, in the heavy chain and in the light chain of the IgG. About 60-80% of the IgG is converted to contain protamine-tags, and residual amount of the excess protamine was always visible.

As shown in FIG. 19A for the EGFR-mAB cetuximab-protamine conjugates, we depleted unbound protamine from the reaction mixture by protein G interaction chromatography. The protamine-conjugated antibody was bound to the protein G matrix, the unbound protamine eluted early and was followed by the purified IgG-protamine complex without protamine, see fractions 29-31. To our surprise, this material, although protamine-conjugated, was not able to bind siRNA in a classic band-shift assay, see right half of FIG. 19 B, whereas the unpurified mAB-protamine complex was binding siRNA in the usual 1:16 molar ratio.

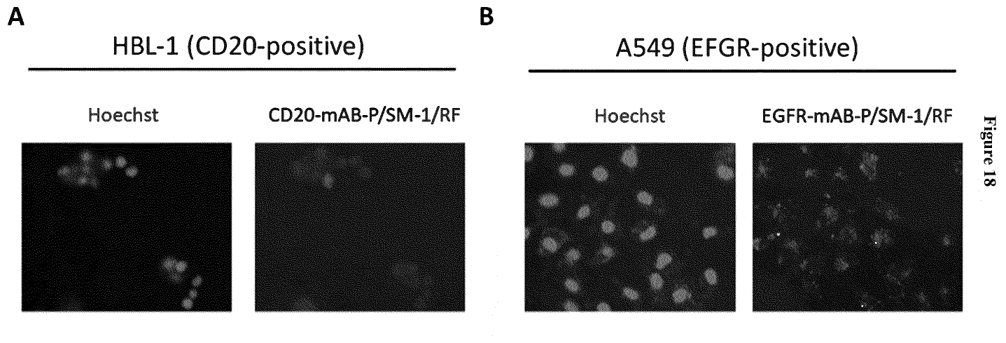

To further examine the effectivity of the protamine-containing and protamine-depleted preparations, we treated EGFR-expressing and KRAS-dependent A549 and SK-LUI NSCLC cell lines with both preparations transporting KRAS-siRNA and control-siRNA. Only those preparations containing free protamine (see FIGS. 19A and B, “anti-EGFR-mAB coupled with 32×SMCC-protamine”) effectively reduced colony growth of the cell lines with KRAS-siRNA, as would have been expected for their oncogene addiction (FIGS. 20A and B).

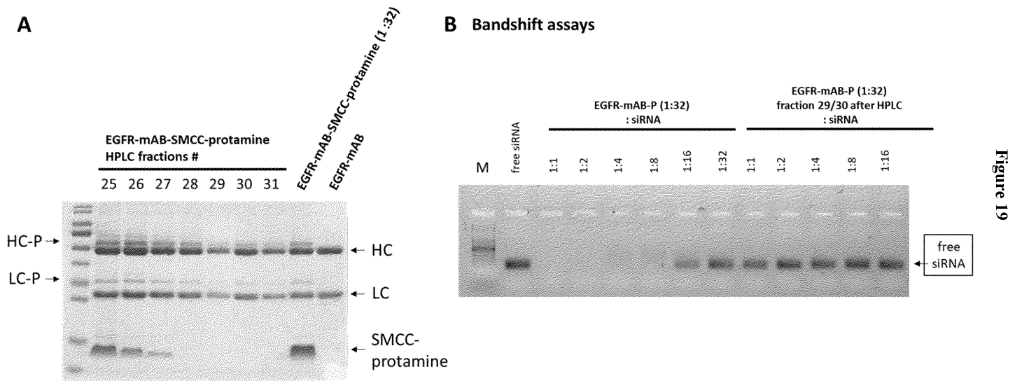

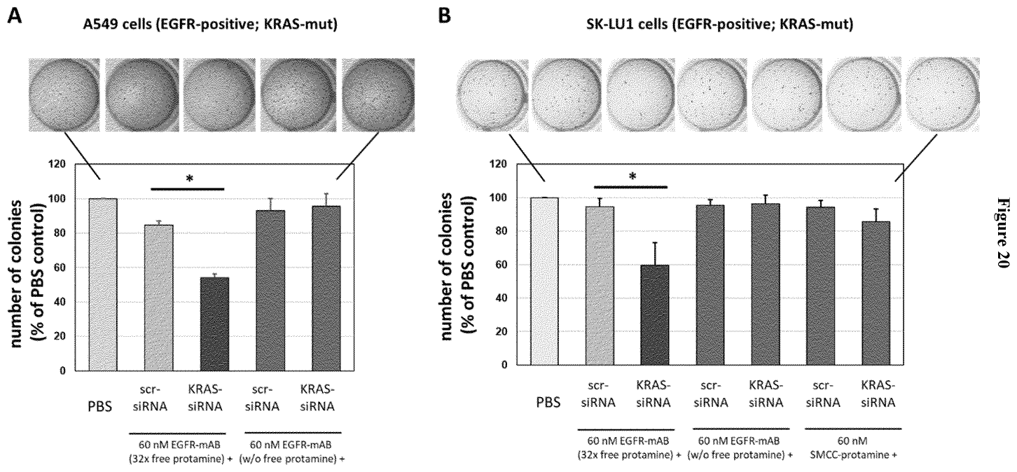

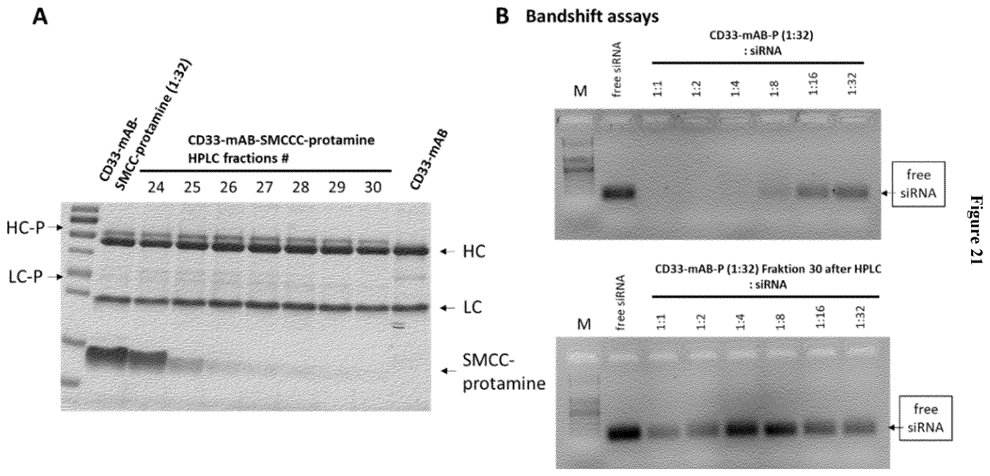

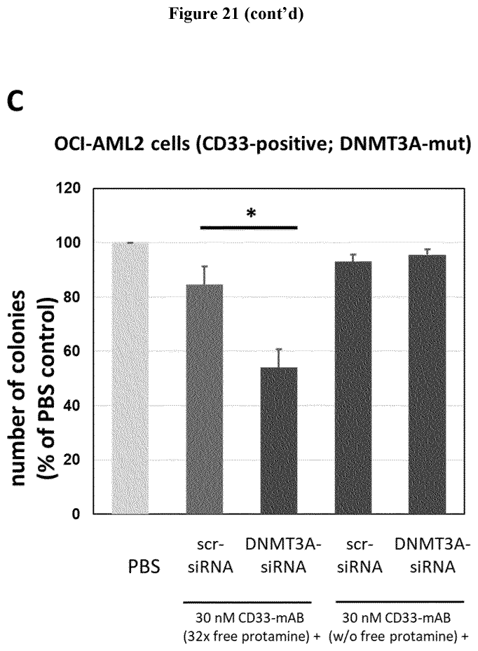

We performed exactly the same purification procedures to the anti-CD33 mAB gemtuzumab, which we used for experimental treatment of AML and observed the same effect: conjugate preparations with unbound protamine depleted by HPLC did not bind siRNA to a desirable amount (FIG. 21 B, lower panel), while unpurified did exactly that (FIG. 21 B, upper panel).

Furthermore, in contrast to non-purified material, the carrier without unbound SMCC-protamine had no remaining inhibitory efficacy of anti-DNMT3A-siRNA to OCI-AML2 cells in a colony formation assay (FIG. 21 C). This observation was not compatible with our previous molecular assembly hypothesis of the carrier system (FIG. 3A), and challenged our previous hypothesis in general.

To find an explanation for these puzzling and unexpected observations, we performed multiple experiments.

Taking the results from FIG. 19 into account, we hypothesized, that the presence of unbound SMCC-protamine in the CD20-mAB-protamine-SM-1/RF-protamine adduct could be as important as in the siRNA adducts, so we depleted protamine from the rituximab-CD20 mAB preparation by affinity chromatography as done before. As hypothesized, the depleted preparation (FIG. 22A, fraction 25) was not able to bind and coordinate polyanionic SM-1/RF to the same extent (FIG. 22 B, left) as the SMCC-protamine containing preparation (FIG. 22 B, right).

Identical findings were made by using the anti-IGF1R monoclonal AB IMCA-12 conjugates (FIG. 23). SMCC-protamine depleted antibody-protamine conjugates were not able to electrostatically bind siRNA.

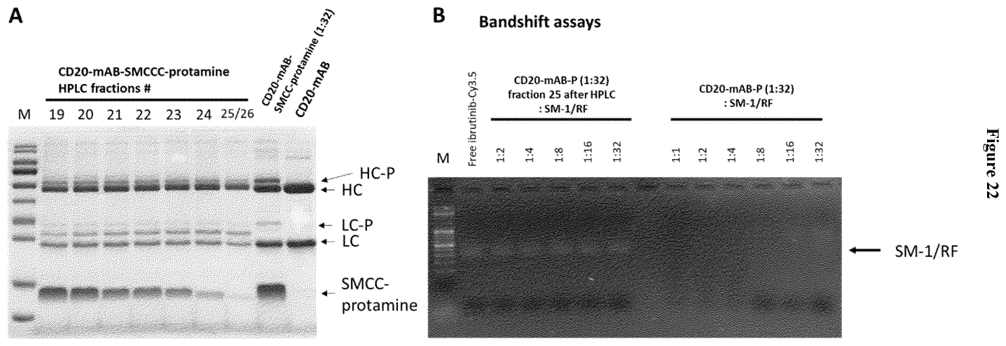

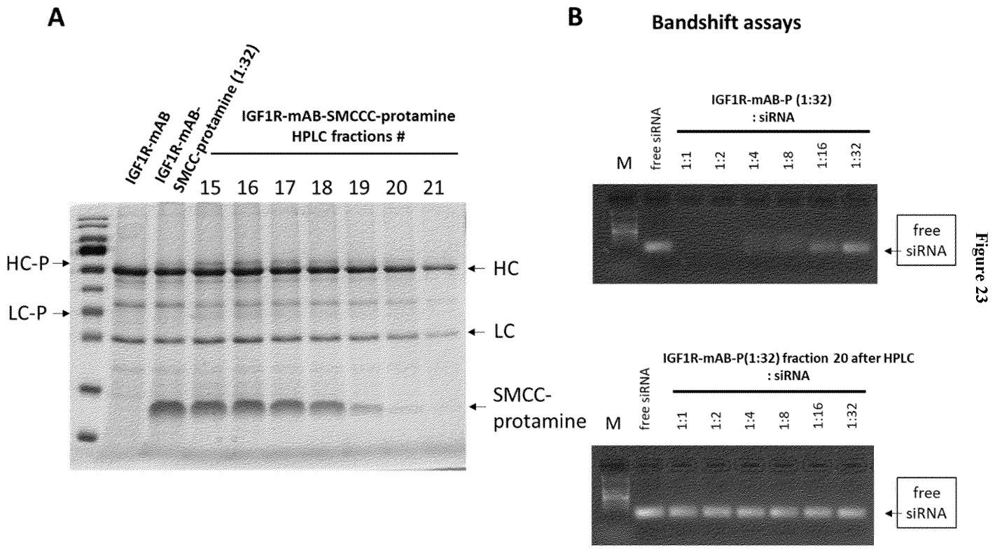

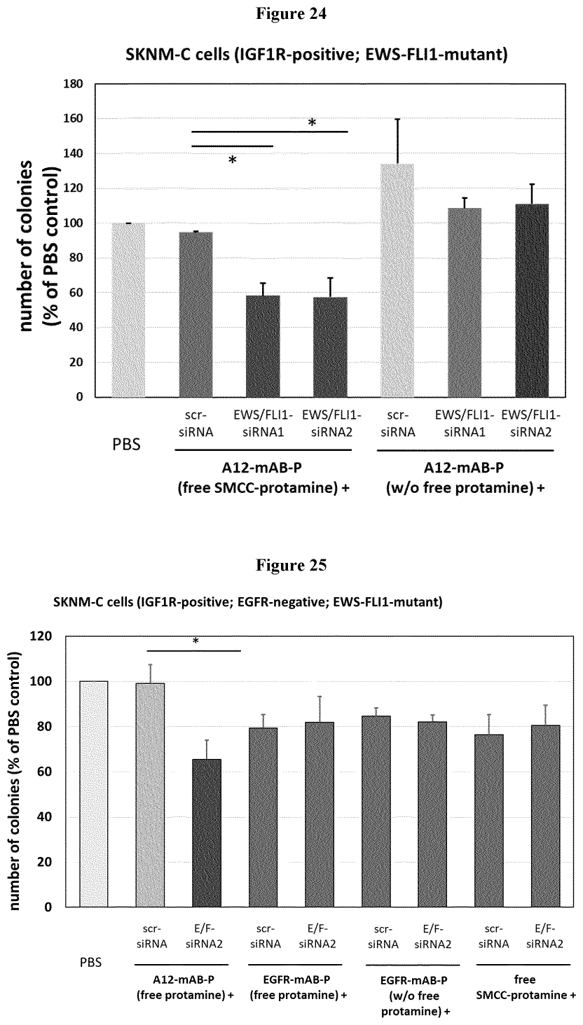

In FIG. 24, we tested the ability of SMCC-protamine depleted A12 carrier antibody (FIG. 23) to effectively deliver oncogene-inactivating siRNA in SKNM-C Ewing sarcoma cells. While non-depleted A12-SMCC-protamine loaded with effective siRNA reduced colony growth, the depleted preparations (see FIG. 23 Figure, Fractions 19-21) did not reduce colony growth.

### Example 8: Deciphering the Role of Free SMCC-Protamine within the Antibody-SMCC-Protamine/Free SMCC-Protamine Complexes

According to the former results, we found out that our targeting does not work if free SMCC-protamine is absent. We therefore wanted to find out which role the free SMCC-protamine plays within the complex. Of course we had to rule out that the main function was performed by the free SMCC-protamine. We therefore performed a series of experiments with free SMCC-protamine and other negative controls.

First, we performed colony formation assays with the IGF1R-positive, but EGFR-negative Ewing's sarcoma cell line SKNM-C that is dependent on the EWS-FLI1-translocation product (FIG. 25). As a positive control, we inhibited the EWS-FLI1-translocation product by treating the cells with the anti-IGF1R-mAB-protamine complex conjugated to anti-EWS-FLI1 (E/F)-siRNA, which lead to significant reduction in colony growth of SKNM-C cells (FIG. 25). On the contrary, transport of the anti-EWS-FLI1-siRNA using the anti-EGFR-mAB-protamine as a carrier with and without free SMCC-protamine did not lead to decreased colony formation compared to the control siRNA (FIG. 25), indicating that the IGF1R-mediated targeting was specific and not due to the free SMCC-protamine. Moreover, also SMCC-protamine alone in the same concentration as the anti-EGFR-antibody (60 nM) was not able to induce inhibition of colony formation (FIG. 25).

Next, we confronted SKNM-C Ewing's sarcoma cells with the regular targeting mixture including the anti-IGF1R antibody conjugated to SMCC-protamine, free SMCC-protamine in the same amount as present in the anti-IGF1R-mAB-protamine complex (60 nM carrier conjugated with 30-fold excess of SMCC-protamine equals ≤1800 nM of potentially free SMCC-protamine) and effective anti-EWS-FLI1-siRNA (FIG. 26A). When we omitted the targeting anti-IGF1R-mAB A12 and treated the SKNM-C cells only with the same concentration of free SMCC-protamine as in the complete mixture plus effective siRNA, no reduction of colony growth as compared to the control scr-siRNA could be observed (FIG. 26A). We tested this strategy also in the AML cell line OCI-AML-2 (FIG. 26 B) and in A549 NSCLC cells (FIG. 26 C), always omitting the targeting mAB (here: anti-CD33 or anti-EGFR, respectively), but giving normally effective siRNAs. As in the SKNM-C, free SMCC-protamine loaded with effective siRNA remained without any effect as the control siRNAs.

This lead us to the assumption that while the correct antibody is needed for the detection of the intended target cell surface molecules, two additional preconditions to effectively bind and complex siRNA and target it to the oncogenic molecules for therapeutic efficacy must be fulfilled: a) protamine conjugated to the targeting antibody and b) a sufficient residual amount of unbound SMCC-protamine present in the mixture. Free SMCC-protamine without the antibody-coupled carrier could not fulfil these requirements.

The next question to be answered was which modified assembly structure of our carrier system could explain without contradiction all in vitro and in vivo efficacy results observed in our studies.

### Example 9: Detection and Visualization of an Unexpected Electrostatic Macrostructure Forming a Stable Nanoparticle

The intracellular antibody-protamine-siRNA complexes are easily identified in treated cell culture samples, if the corresponding cell surface receptor or molecule is expressed, such as depicted in FIG. 27 Figure: here, cetuximab (anti-EGFR)-SMCC-protamine with bound siRNA is internalized into EGFR expressing NSCLC cells, and is internally processed to early endosomes (white dots, left panel), but not lysosomes (grey dots, middle panel).

### Example 10: No Internalisation of Antibody-Protamine-siRNA Complexes without Free SMCC-Protamine or of Free SMCC-Protamine-siRNA Alone in Target Cells

Interestingly, typical internalized vesicular structures were only seen, if residual SMCC-protamine from the conjugation protocol was left in the mixture (FIG. 28, right hand), but not, if the protamine-SMCC was depleted by means of affinity chromatography (FIG. 28, left hand, see also FIG. 19 Figure for chromatography results). So, also visually, the existence of unbound protamine-SMCC is critical to the efficiency of the conjugate.

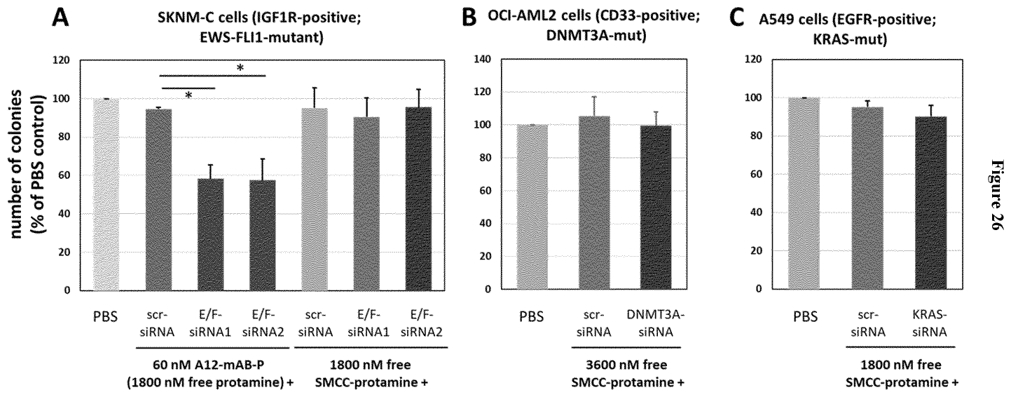

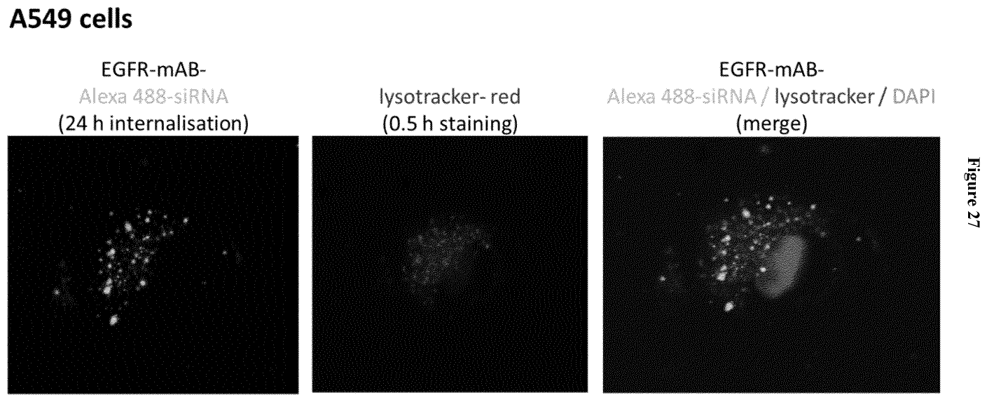

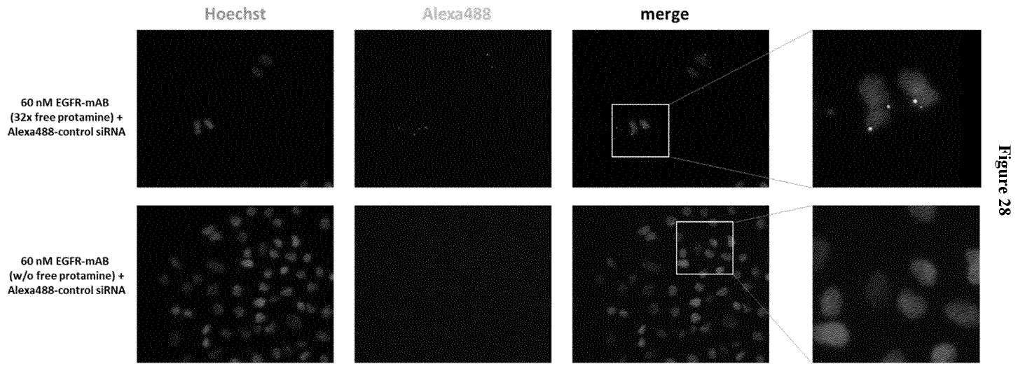

We performed this also with other antibody-SMCC-protamine complexes in comparison to their counterparts that were depleted from free SMCC-protamine via HPLC and with free SMCC-protamine: no internalisation could be observed in any cell line with antibody-protamine-siRNA complexes without free SMCC-protamine nor with free SMCC-protamine only (FIG. 29, FIG. 30, and FIG. 31).

### Example 11: Formation of Vesicular Structures In Vitro by Antibody-Protamine-siRNA Complexes

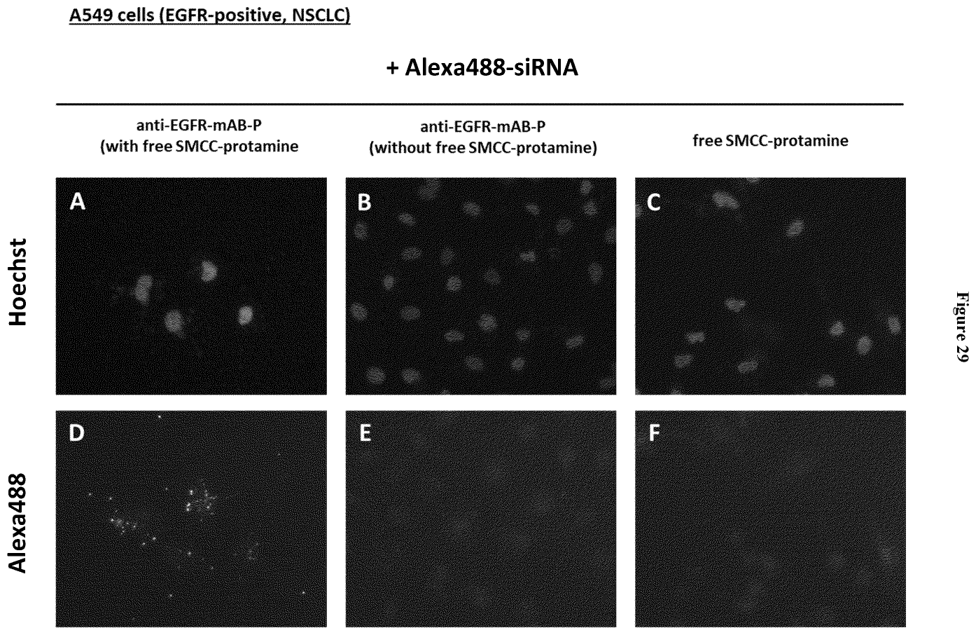

Whenever we performed negative control experiments, i.e. treating cells NOT expressing the EGF receptor with effective conjugates targeting the EGF receptor, we consequently observed extremely low cellular targeting efficiencies (FIG. 32 B), but we were intrigued by the accumulation of fluorescent micellar structures OUTSIDE the cells. These accumulations, probably attached to matrix-like structures on the treated dish surface, were only seen, if there was no target found on the cells to bind and internalize the conjugates. In addition, these accumulations were all of a similar size and, they had to be much larger than the approx. 10-20 nm, which would account for a monomer of one IgG plus several protamines plus eight attached siRNAs, because this size would not be detectable as a particle in fluorescent light microscopy, which approximately has a resolution of half of the emission wavelength, giving rise to particle sizes larger than 200 nm.

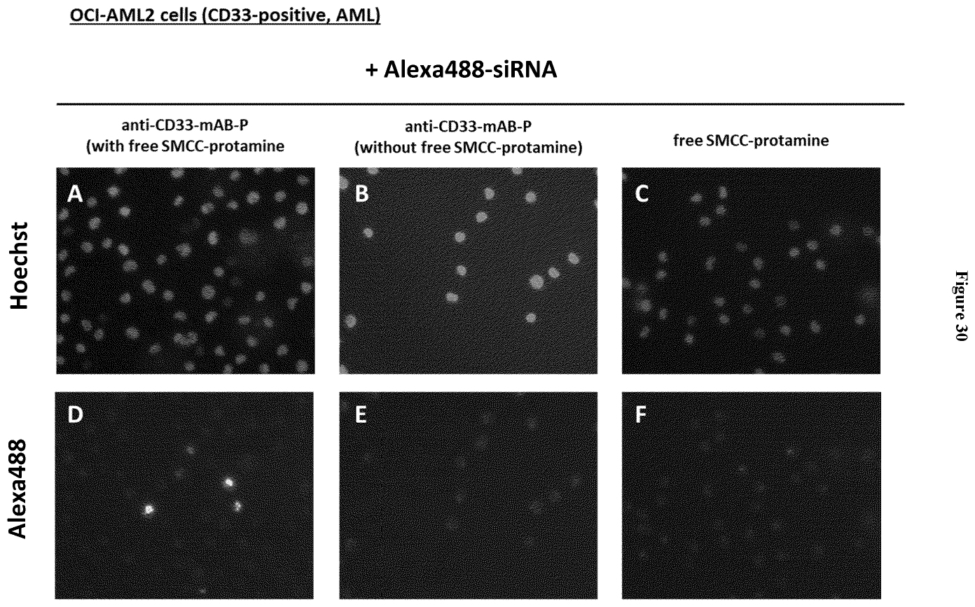

Therefore, we assumed the 3 components 1. cetuximab-SMCC-protamine, 2. siRNA and 3. unbound SMCC-protamine to form a kind of stable macrostructure necessary for activity. To verify the existence of such a macrostructure that is responsible for the efficiency of the IgG-protamine-siRNA, we applied particle size detection by means of dynamic light scattering (DLS) on a zeta-counter (MALVERN), which correlates light diffusion caused by particles in a solution. The results were intriguing: As a dynamic process, the components, which are of completely plausible sizes (22 nm for the IgG-protamine-protamine-siRNA monomer) spontaneously assemble to nanostructures greater 400 nm in size after a few hours, while this process is not detectable in protamine-depleted preparations (FIG. 33).

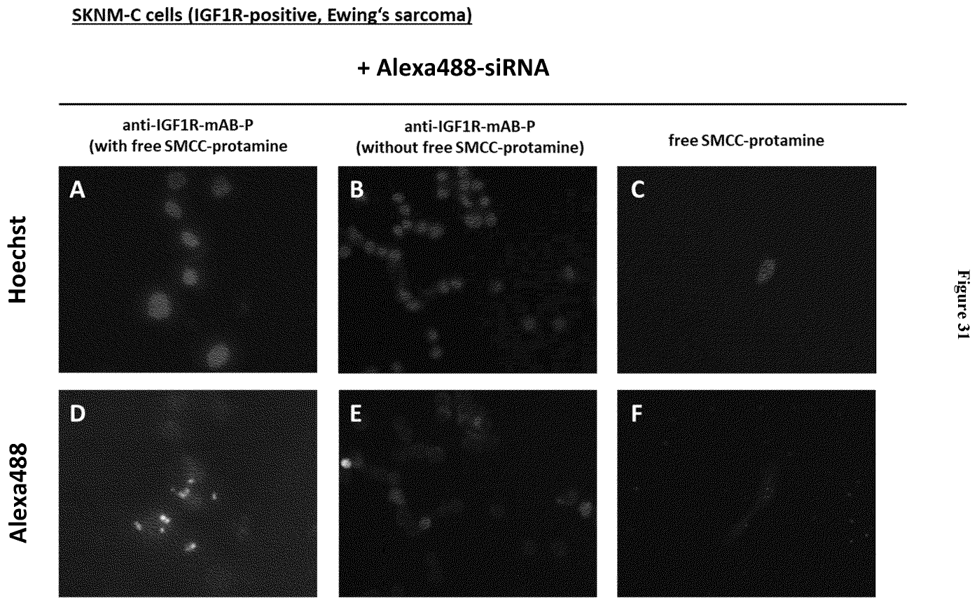

This process is time-dependent and the larger structures only form after certain times of incubation (FIG. 34 Figure). Time-spans between 2 and 6 hours are sufficient to form those macrostructures, which again start to partially disassemble after 24 h in unprotected PBS surroundings at room temperature.

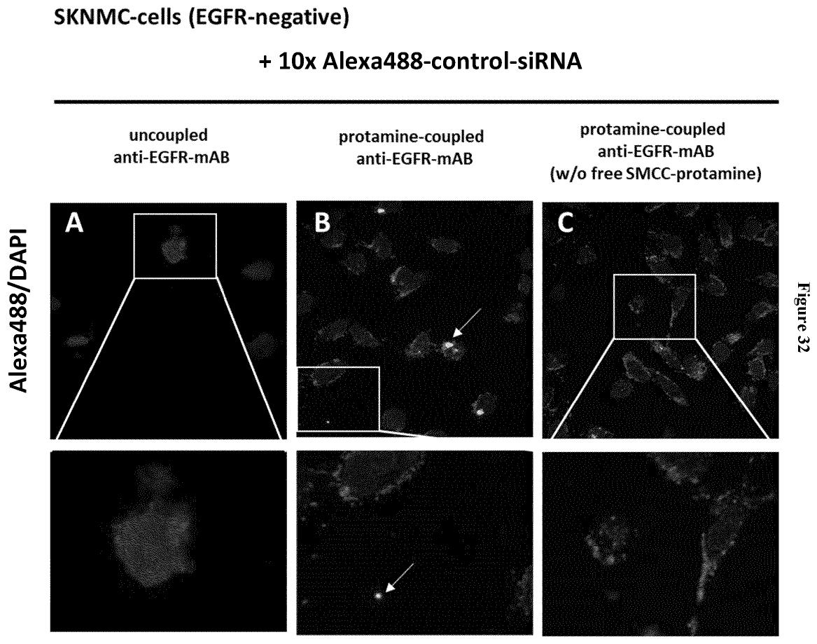

Based upon these measurements, we hypothesized that the antibody-protamine/free SMCC-protamine/siRNA complexes form outside cells and independently of a cellular context. To visualize these complexes, we incubated different complex compositions without cells using Alexa488-siRNA on coated chamber slides overnight and fixed the developed structures at next day. Fluorescence microscopy revealed that vesicular structures only form when the 3 components find each other: 1. antibody-SMCC-protamine, 2. free SMCC-protamine, 3. siRNA (see FIG. 35).

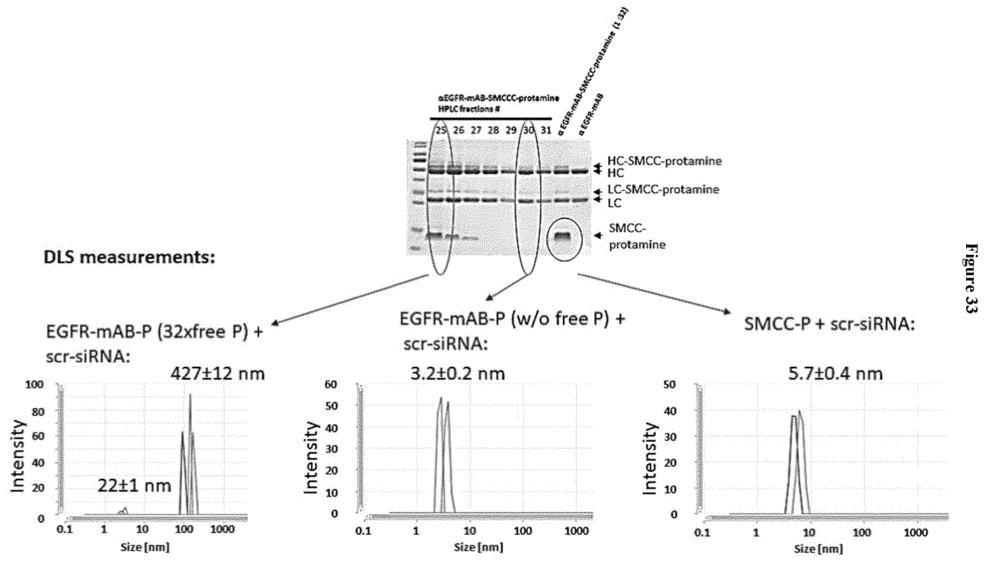

All antibody-protamine complexes with free SMCC-protamine do form vesicular nanostructures, whereas without free SMCC-protamine or SMCC-protamine alone did not form any nanostructure (FIG. 36).

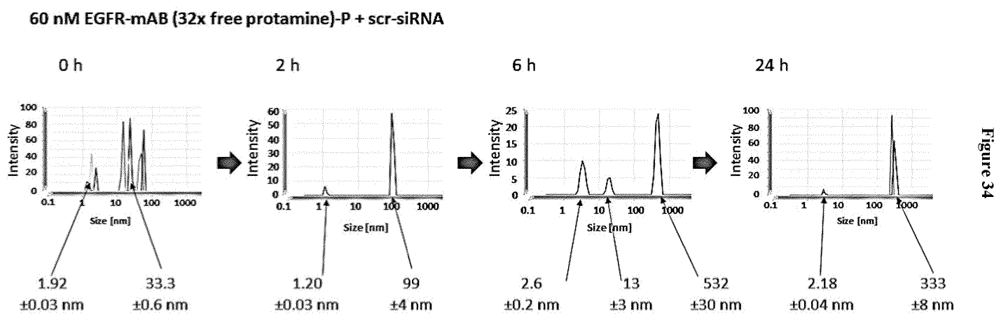

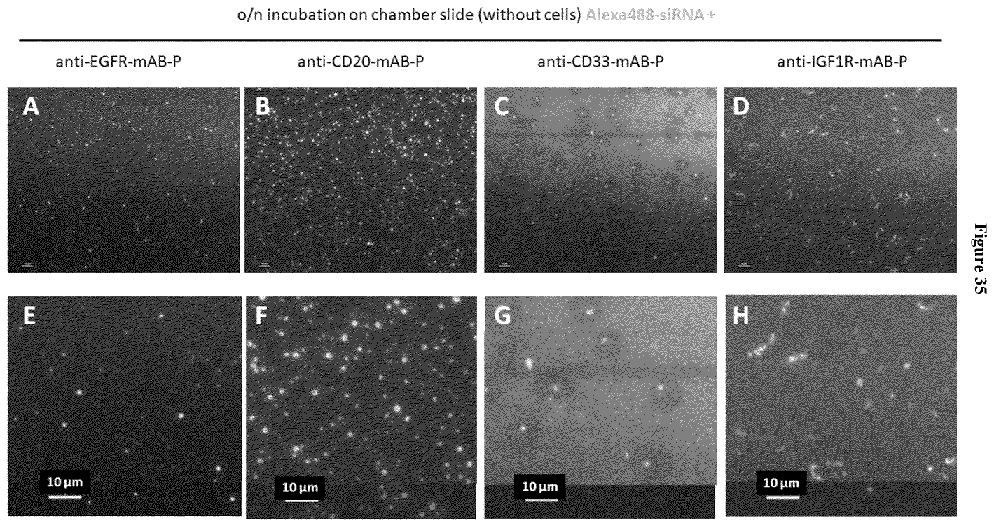

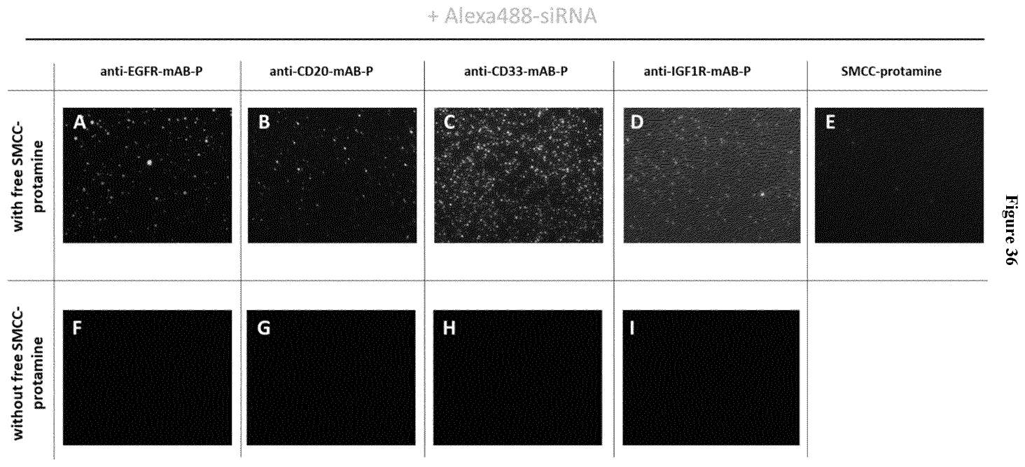

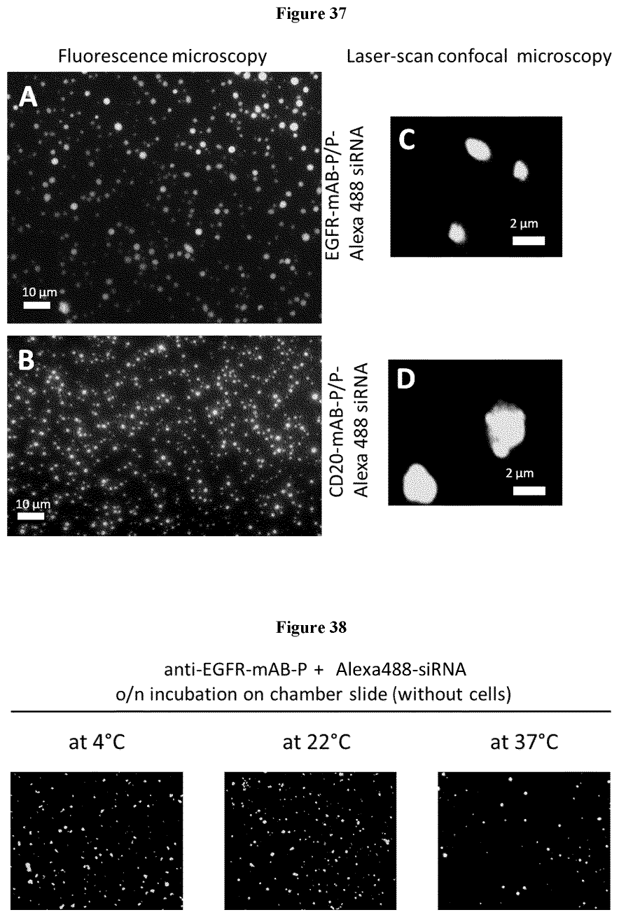

In detail, the nanostructures resemble a spheroid shape of micrometre size, formed by the three components mAB-SMCC-protamine, unconjugated SMCC-protamine and the Alexa488-labeled siRNA (FIG. 37). The structures are verified by fluorescence microscopy and laser scan confocal microscopy. It became evident that the spheroid structures are completely filled with Alexa488 signals from the siRNA compound.

The results are summarized in the following Table.

| | Dynamic light | Microscopic | |
|---|---|---|---|
| Sample | scattering | analysis | synopsis |
| Cetuximab-SMCC- | 512 ± 22 nm | >0.5 μm, <2 μm | >0.2 μm < 5 μm |
| protamine + siRNA | | | |
| CD20-SMCC- | 607 ± 31 nm | >0.5 μm < 2 μm | >0.2 μm < 5 μm |
| protamine + siRNA | | | |
| CD20-SMCC-protamine | | No visible | |
| (SMCC-protamine | | vesicles | |
| depleted) + siRNA | | | |
| CD20-SMCC- | | No visible | |
| protamine | | vesicles | |

### Example 12: Vesicular Structures In Vitro Occurs at Different Temperatures

We also tested if the formation of vesicular structures is dependent on a certain temperature. In fact, they are formed at 4° C., at room temperature (˜22° C.) and at 37° C. (FIG. 38)

### Example 13: The Formation of Functional Vesicular Structures In Vitro by Antibody-Protamine-siRNA Complexes Depends on the Amount of Free SMCC-Protamine

To further elucidate the function of SMCC-protamine coupled to the antibody and as free molecule within the complex, we titrated the amount of SMCC-protamine added to the antibody, here: the anti-EGFR-antibody cetuximab. We used a constant amount of cetuximab and added molar ratios of 1:1 up to 1:100 of SMCC-protamine (for details see (FIG. 39A). We then checked the conjugation efficiency on a Coomassie-stained SDS-PAGE and found out that the coupling of the light (LC) and heavy chain (HC) of the antibody appeared suboptimal when 1:1 up to 1:10 antibody:SMCC-protamine ratios were incubated (FIG. 39 B). The conjugation process appeared saturated at the molar antibody:SMCC-protamine ratio of 1:32 with no more enhancement at 1:50 and 1:100 (FIG. 39 B).

We analysed the siRNA binding capacity of these different conjugations by our routine bandshift assays (FIG. 40A-F). The binding of siRNA was detectable at the conjugation ratios of 1:32 to 1:100 (FIG. 40 D-F), at lower ratios no efficient siRNA binding was detectable (FIG. 40A-C).

We further analysed the properties of the different conjugation products according to their capacity to form vesicular-structures without cells when these conjugation products were incubated with Alexa488-control-siRNA (FIG. 40 G-L) as well as with EGFR-positive A459 cells (FIG. 40 M-R). When cetuximab was conjugated with SMCC-protamine at ratios of 1:1 up to 1:10, no efficient cell-free vesicle formation could be observed (FIG. 40 G-I), while at the ratio 1:32, the vesicle formation was very abundant FIG. 40 J) and decreased at ratios 1:50 and 1:100 (FIG. 40 K-L). The internalisation capacity of these complexes was highest upon conjugation 1:32 (FIG. 40 P), decrease upon conjugation 1:10 (FIG. 40 O). No internalisation was observed at the ration 1:1 (FIG. 40 M), 1:3.2 (FIG. 40 N) or 1:50 to 1:100 (FIG. 40 Q-R). This hinted at an optimal complex formation of cetuximab with SMCC-protamine of 1:32, since this lead to the most efficient conjugation, vesicle formation without cells and internalisation into cells.

As a functional assay, we compared the efficiency of the different conjugation products to inhibit the colony formation of A549 cells upon knockdown of the oncogene KRAS. We also compared this to the equal amount of free SMCC-protamine without conjugated cetuximab to elucidate the impact of the unbound SMCC-protamine alone (FIG. 40 S-X). Surprisingly, only the conjugation cetuximab: SMCC-protamine 1:32 complexed to KRAS-siRNA leads to a significant decrease of colony formation in comparison to scrambled control-siRNA (FIG. 40 V). SMCC-protamine alone cannot induce inhibition of colony formation via KRAS knockdown at any concentration (FIG. 40 S-X). We only observed a striking toxicity of SMCC-protamine in complex with scr-siRNA as well as KRAS-siRNA at the highest concentrations (FIG. 40 W-X).

### Example 14: Free SMCC-Protamine can be Re-Added to the SMCC-Protamine-Depleted Antibody-Protamine Conjugates to Form Vesicular Structures In Vitro and Free SMCC-Protamine can be Substituted by Free Protamine

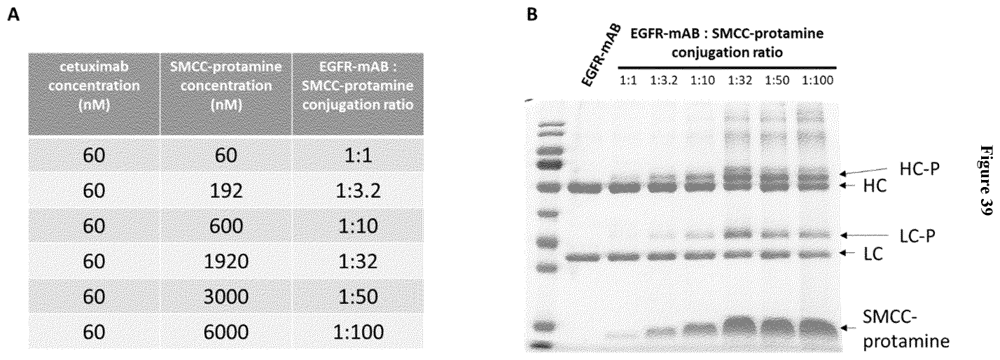

To further elucidate the function of SMCC-protamine coupled to the antibody and as free molecule within the complex, we titrated the amount of SMCC-protamine added to the antibody-protamine-conjugates that were depleted from free SMCC-protamine by HPLC. These antibody-protamine conjugates are not able to form vesicular structures with fluorescent siRNA as depicted in FIG. 36 F. We therefore wondered if free SMCC-protamine can be re-added to fulfil this function and if SMCC-protamine can be substituted by protamine without sulfo-SMCC. We added different amounts of free SMCC-protamine and protamine alone to anti-EGFR-mAB-P (FIG. 41). The addition of 1×SMCC-protamine or 10×SMCC-protamine in relation to the antibody were not effective to form vesicular structures (FIGS. 41A and B), while the addition of 32×SMCC-protamine lead to a very effective formation of vesicles (FIG. 41 C). According to this assay, free SMCC-protamine can be substituted by protamine without chemically coupled sulfo-SMCC (FIG. 41 F).

### Example 15: Formation of Vesicular Structures In Vitro by Antibody-Protamine-siRNA and/or SM-1/RF Complexes

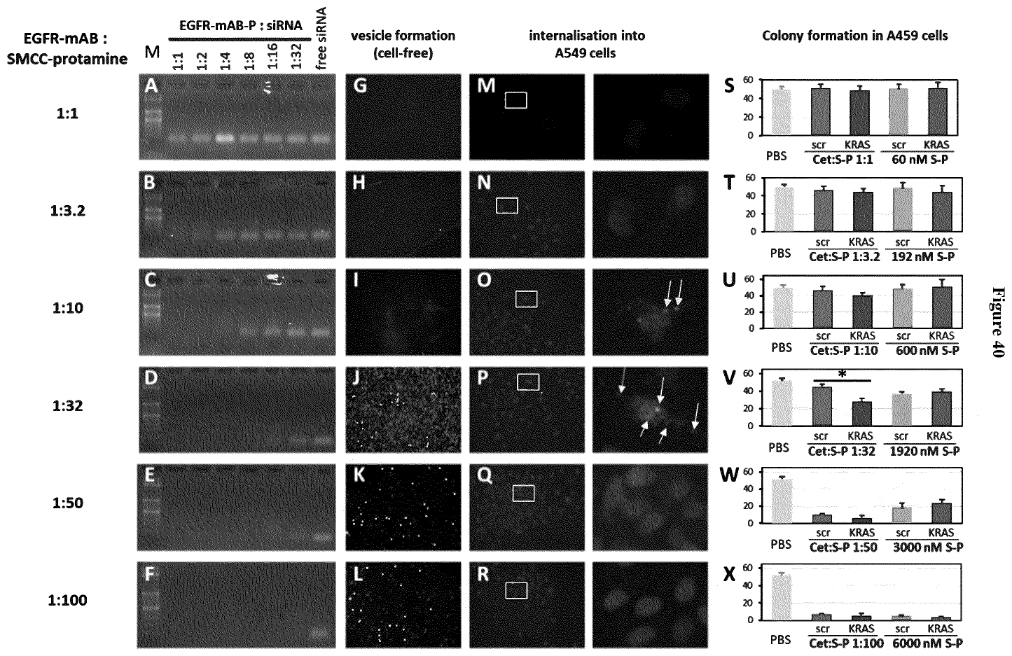

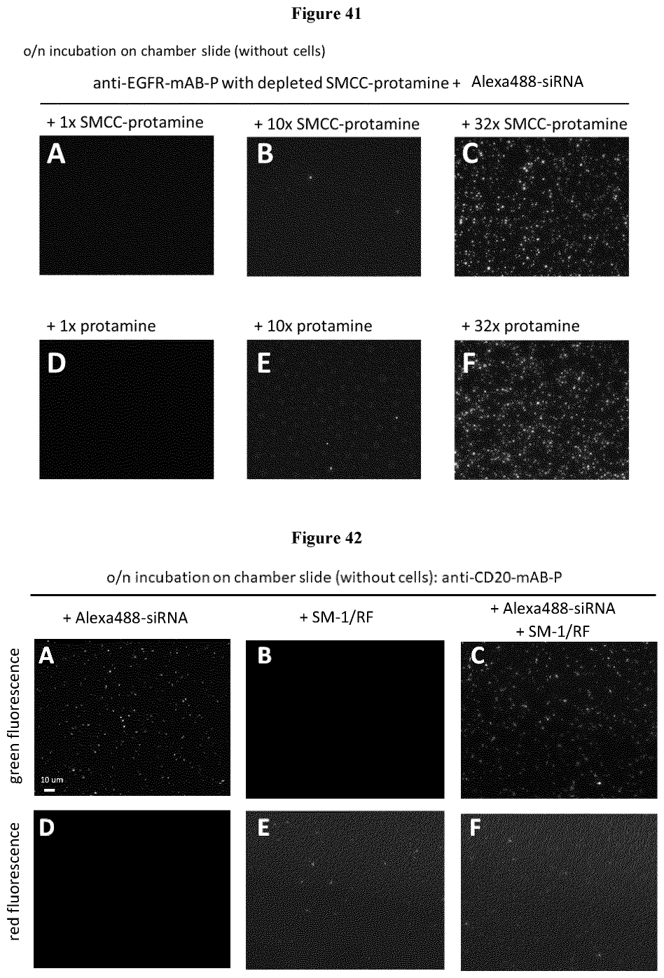

To test this new and unexpected nanostructure model we used the structurally completely different, but electrostatically equally charged molecule SM-1/RF (see Example 8) and complexed it to antibody-protamine conjugates (FIG. 42-FIG. 45).

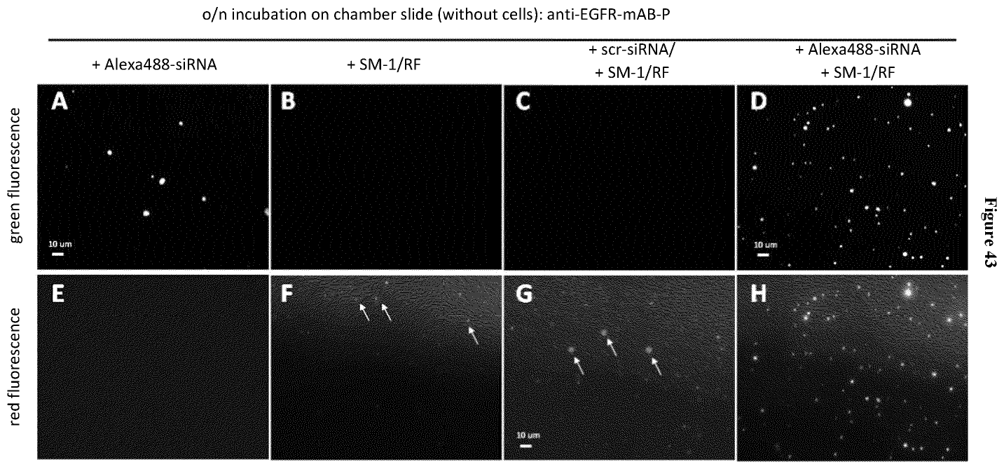

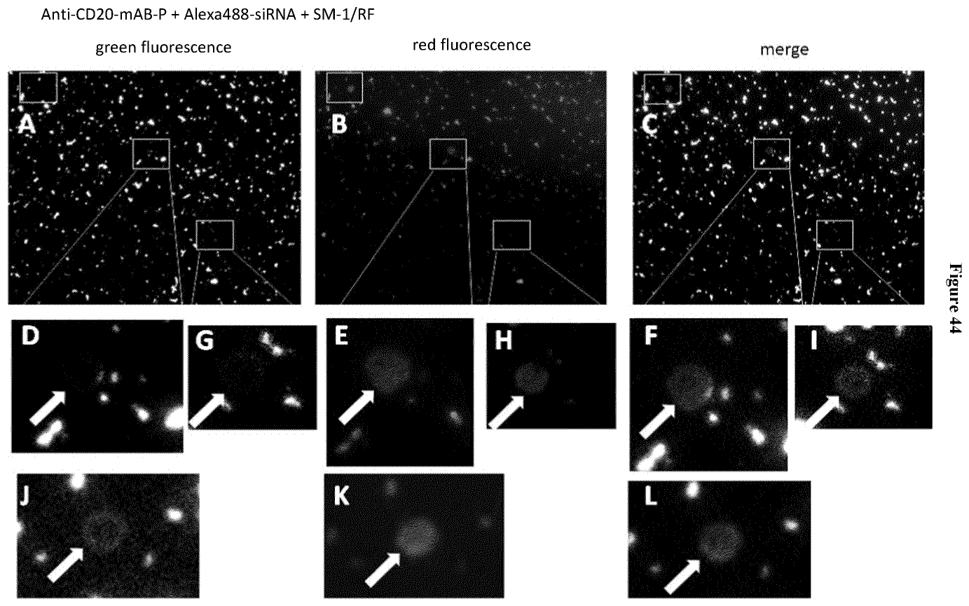

During the inspection of the results of the cell-free assembly of mAB-protamine-SM-1/RF-conjugates with and without additional siRNA, we observed marked differences in the quantity and size of the respective nanostructures: Those assembled by siRNA AND the SM-1/RF complexed by mAB-protamine plus free SMCC-protamine were considerably larger and more frequent than without siRNA (FIGS. 42 C and F and FIGS. 43 D and H). We explain this phenomenon by a hypothesis of a mixed particle consisting of all 4 components forming a stable nanostructure. In detailed fluorescence micrographs, the nanostructure of the largest particles can be revealed as siRNA forming the boundaries of a spheroid micelle, whereas the SM-1/RF fills the lumen of this sphere (FIG. 44 and FIG. 45, see magnifications).

This phenomenon that mixed antibody-protamine particles are more frequent and much larger than those with only SM-1/RF complexed to the mAB-protamine could be seen both with anti-CD20-mAB rituximab (FIG. 44) and anti-EGFR-mAB cetuximab as carrier antibodies (FIG. 45).

This means that the negatively charged SM-1/RF can form small vesicles with antibody-protamine complexes (FIG. 42 E and FIG. 43 F), but siRNA, which is a linear and highly negatively charged molecule, might serve as a kind of electrostatic “glue” between the antibody-protamine/free SMCC-protamine complexes. This can be observed as a circular shining in extraordinary large vesicles that seem to be filled with red fluorescent SM-1/RF (FIG. 44 and FIG. 45).

The large micellar structures as seen in FIGS. 44 and 37 are also visible in regular light microscopy analysis under phase-contrast conditions (see FIG. 46).

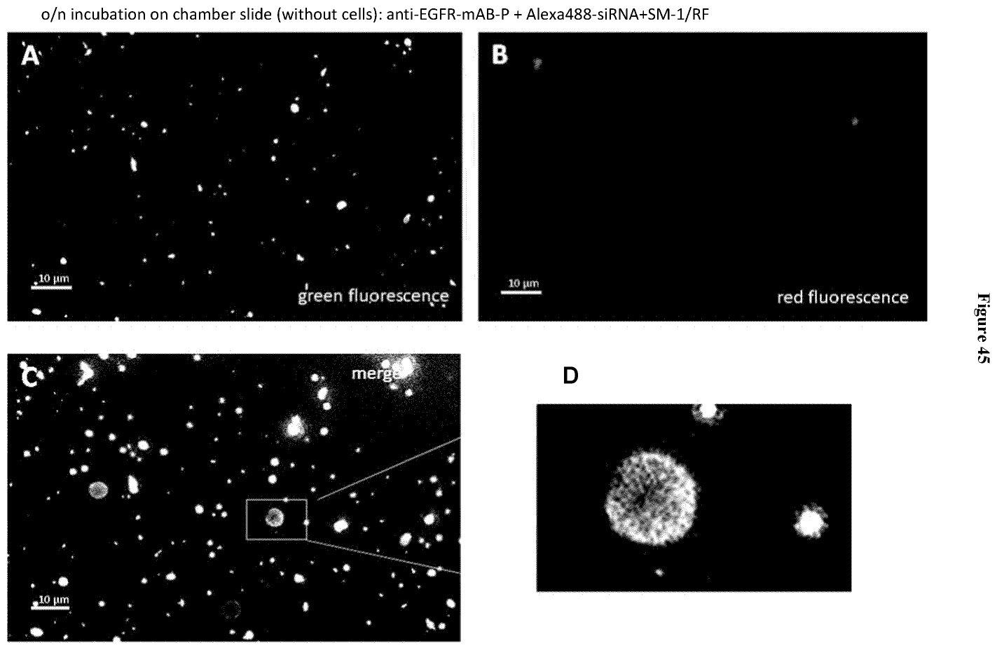

We further confirmed this observation by laser scan microscopy (LSM, FIG. 47 Figure). Here, confocal analysis of vesicles formed without cells showed that green fluorescent siRNA forms a ring (FIG. 47A a-c and FIG. 47 Figure B d-f) and for extraordinary huge vesicles like formed with anti-CD20-mAB-P, one can see that the lumen of this vesicle is filled with the red fluorescent SM-1/RF (FIG. 47 B d and f).

### Example 16: Proposed Model

Taken together, the principle of binding anionic cargo molecules by a carrier-antibody-protamine plus unbound protamine can be applied also to cargos other than siRNA-nucleic acids. Here, it is important to modify the cargo molecule to give it a polyanionic character, and to leave unbound protamine-SMCC in the preparation to enable a strong electrostatic coordination and self-assembly of the reactants.

This observation strongly supports the new and unexpected macromolecular nanostructure as being potentially necessary and sufficient for the in vitro and in vivo pharmacodynamic efficacy of our carrier system.

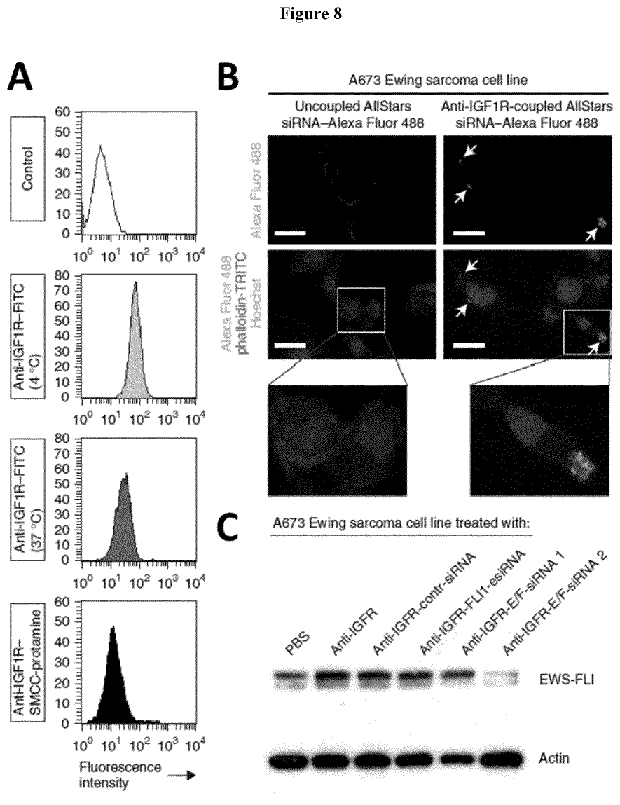

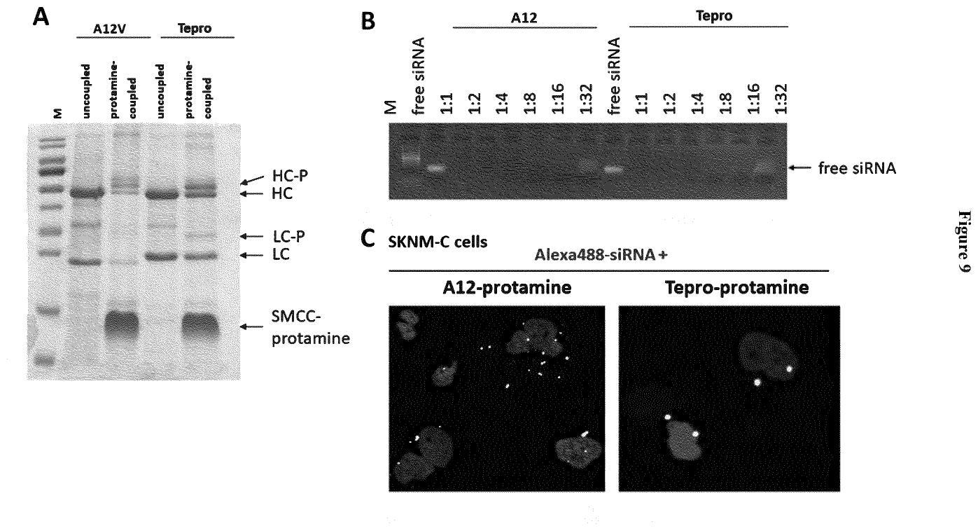

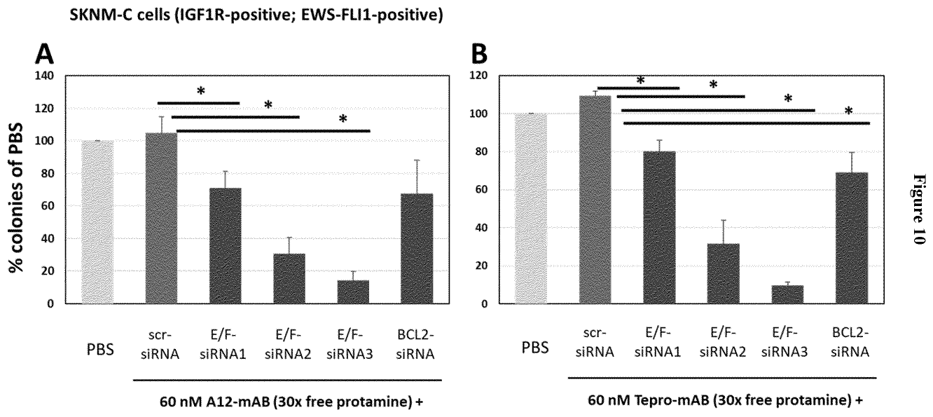

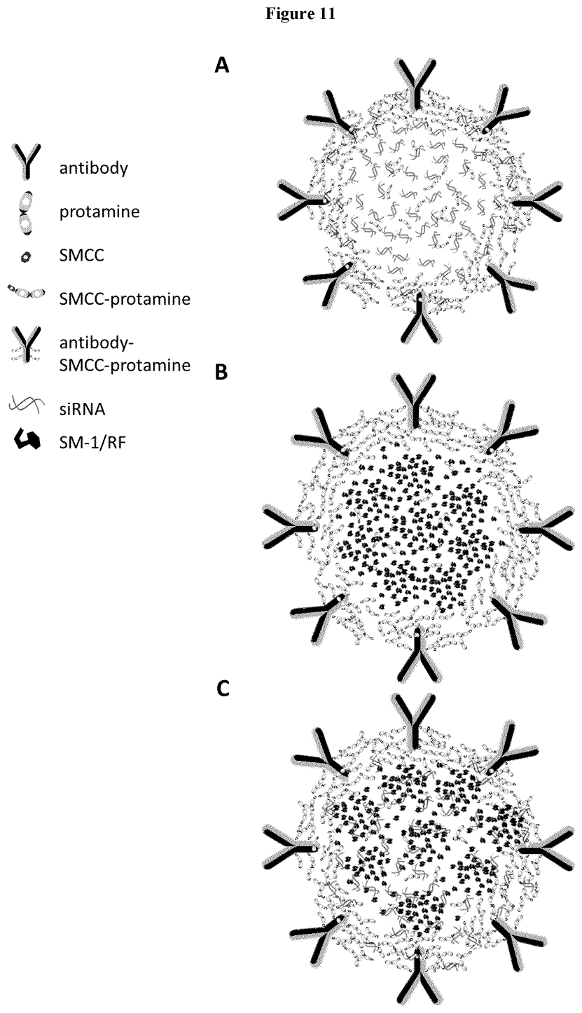

Therefore, we hypothesize the combination of the components 1. antibody-protamine, 2. siRNA/anionic small molecule inhibitor and 3. unbound (SMCC-)protamine to form a nanoparticle-like macrostructure, which is responsible for the stability of siRNA and can effectivity deliver siRNA and/or anionic small molecule inhibitors to the intended cells, which is a totally unexpected observation. An illustrative and idealized model of this nanostructure assembly is shown in FIG. 11.

In conclusion, experiments using various chemically different effector pay loads with the minimal common denominator requirement of being poly-anionic and no other structural similarity, lend experimental evidence for our new and unexpected nanostructure model as being the basis for the in vitro and in vivo pharmacodynamic characteristics of our antibody-SMCC linker-protamine siRNA carrier and our antibody-SM-1/RF system.

This modular nanostructure system with dual specificity, 1. for siRNA/anionic small molecule transport and specific delivery to target cells and 2. for specific intracellular oncogene inactivation can be used for various disease groups including cancer.

### Example 17: Coupling Antisense Oligonucleotides to Antibody-Protamine Conjugate

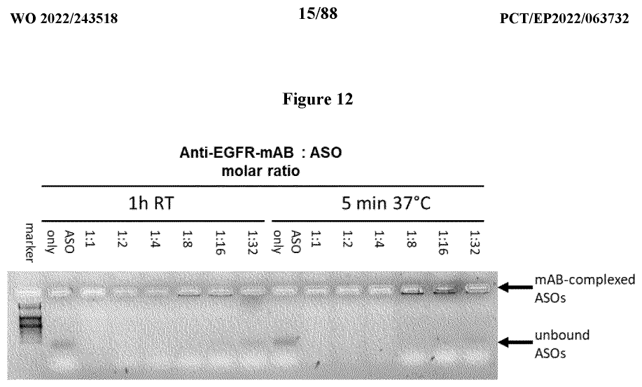

To check if the antibody-protamine nanoparticle can also be used to transport single-stranded oligonucleotides, which are currently used as an alternative tool to knockdown gene expression. These synthetic antisense single-stranded oligonucleotides (“ASO”) are as short as siRNA and it is hypothesized that they bind to the αEGFR-mAB-protamine conjugate analogous to siRNA. We therefore performed a band shift assay using a control ASO and saw that 1 mol αEGFR-mAB-protamine conjugate can bind at least 8 to 32 mol of ASO when incubated for one hour at room temperature or five minutes at 37° C. (FIG. 12).

### Example 18: Investigating the Potential of Antibody-Protamine Fusion Proteins

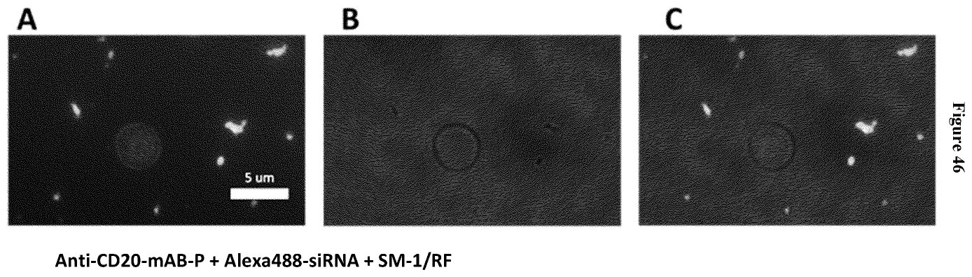

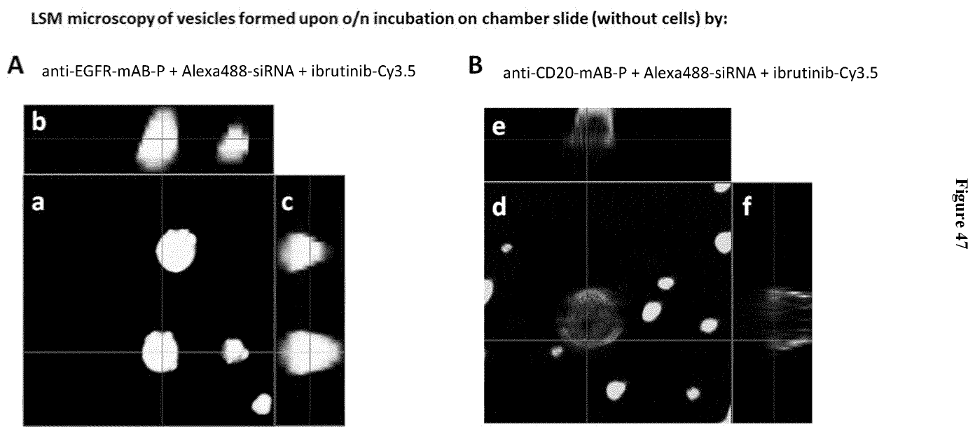

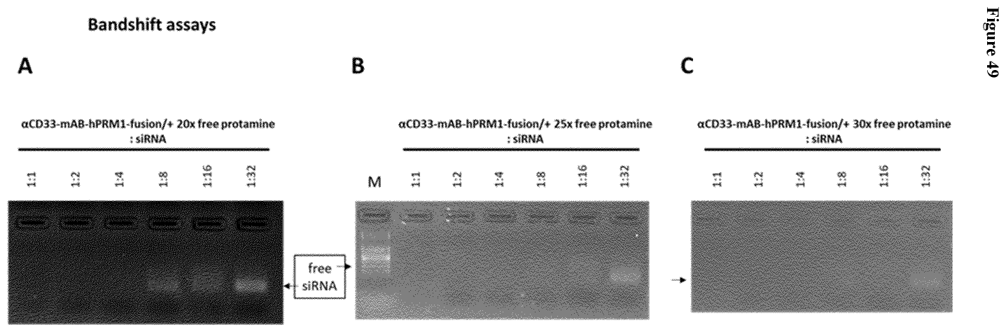

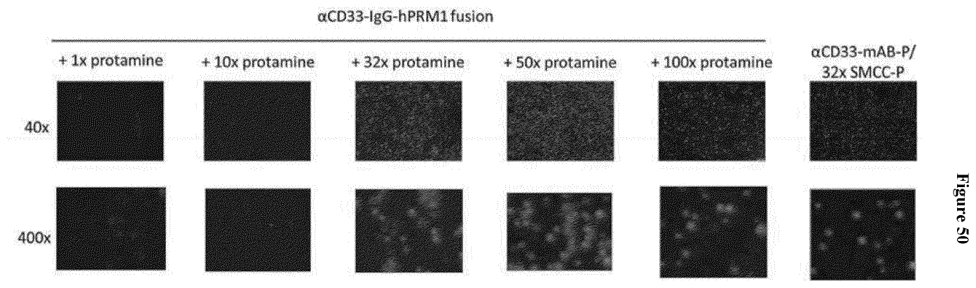

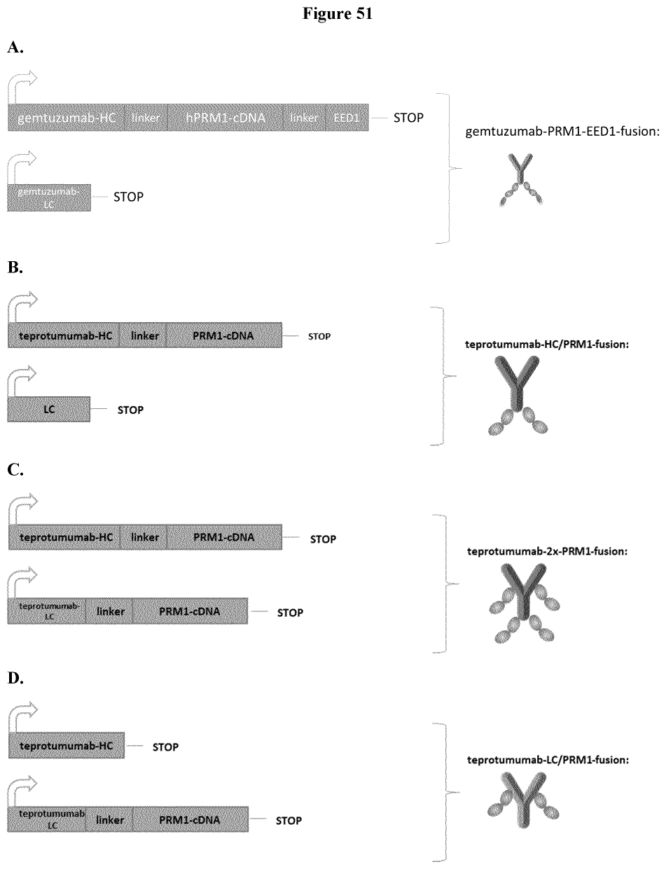

The principle of genetic fusion between cell-determining targeting moiety and the electrostatic siRNA binder protamine such as presented in Song et al. was evaluated by applying this strategy to in a variation to target e.g. leukemic blasts (FIG. 48). The well-known anti-CD33 receptor antibody gemtuzumab was expressed with (FIG. 51) and without a direct genetic fusion to protamine as a siRNA carrier. To test the ability of this construct to bind and carry siRNA, electro-mobility shift assays were performed (“Bandshift assay”), and surprisingly, the heavy chain fusion between gemtuzumab and protamine alone does not enable the conjugate to bind siRNA (FIG. 48 C). As a control, chemically conjugated pure gemtuzumab IgG by means of sulfo-SMCC cross-linker to protamine as described herein were used (FIG. 48A), The chemically conjugated molecules comprised protamine attachments both at heavy and light chain and further comprised a certain amount of unbound protamine-SMCC because the cross-linker has been introduced in excess. This last conjugate preparation easily complexed up to eight mol of siRNA per mol of monoclonal antibody (mAB, FIG. 48 B), see disappearance of bands because of complexation of siRNA. When certain amounts of free protamine-sulfate was added to the αCD33-mAB-PRM1-fusion protein, Bandshift show that these complexes can bind siRNA again (FIG. 49).

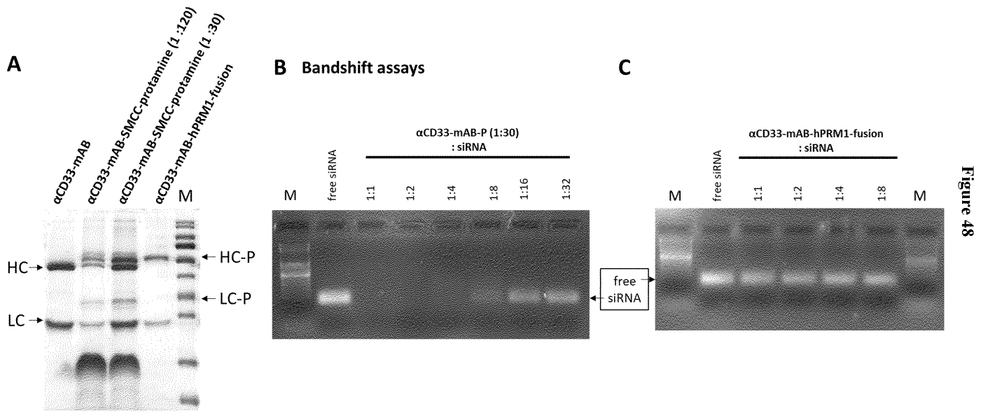

Moreover, when the ineffective anti-CD33-human-protamine fusion protein from FIG. 48 was taken and complemented this with certain amounts of free protamine, it was possible to complex Alexa488-tagged siRNA in form of large nanoparticles seen in fluorescent microscopy in a cell free environment (FIG. 50), whereas non-complemented CD33-protamine fusion was unable to form any complex structures. Those structures have been shown in further experiments to be critical for therapeutic efficacy.

In addition, these nanocarriers proved to be effective in knockdown DNMT3A in DNMT3A-dependent OCI-AML2 cells (see also FIG. 54), since they mediate decreased colony formation in those cells upon addition of different formulations of free protamine and DNMT3A-siRNA.

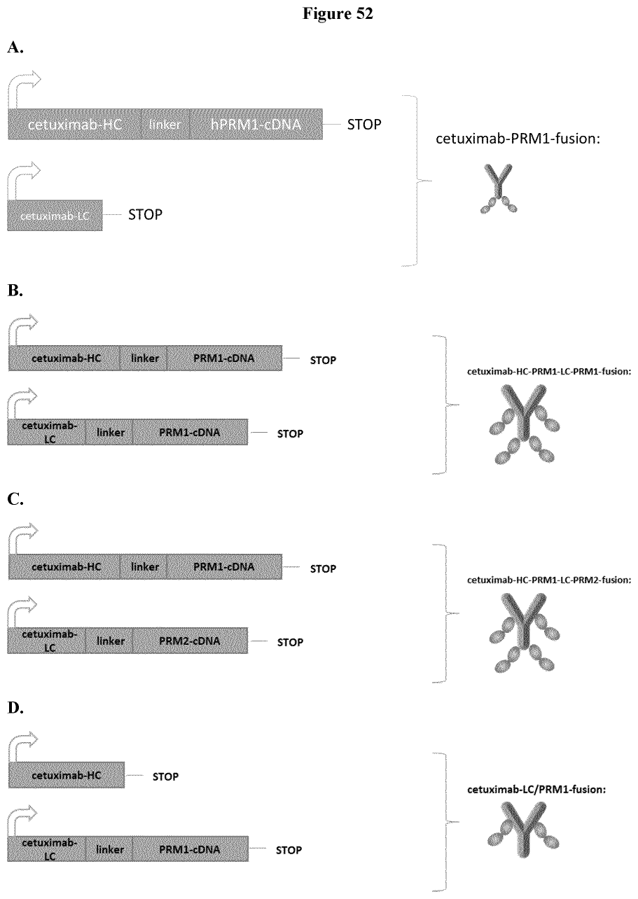

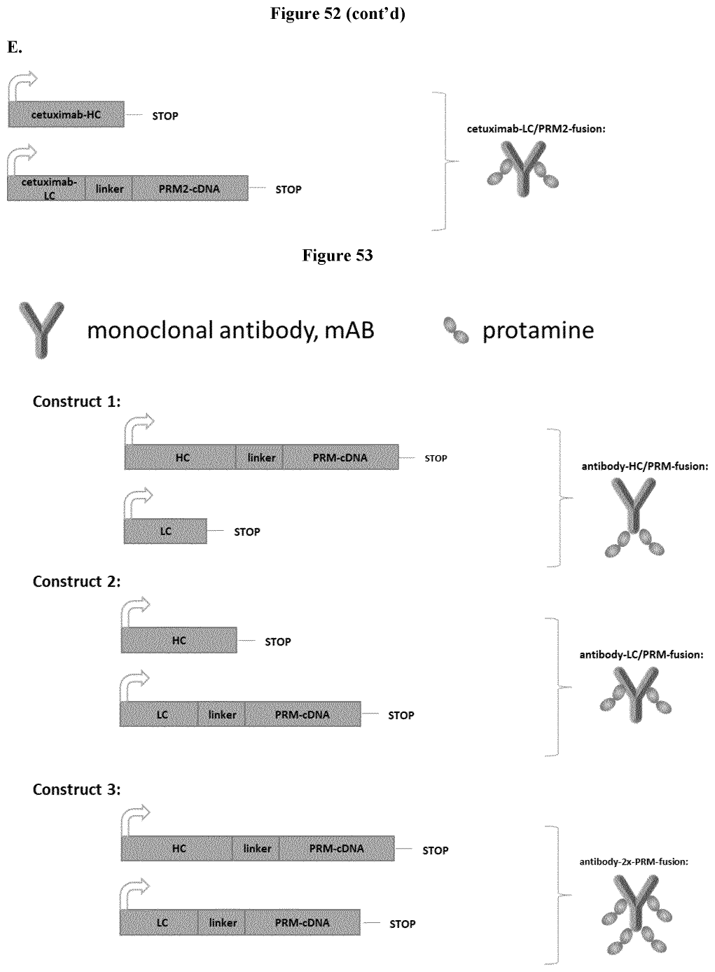

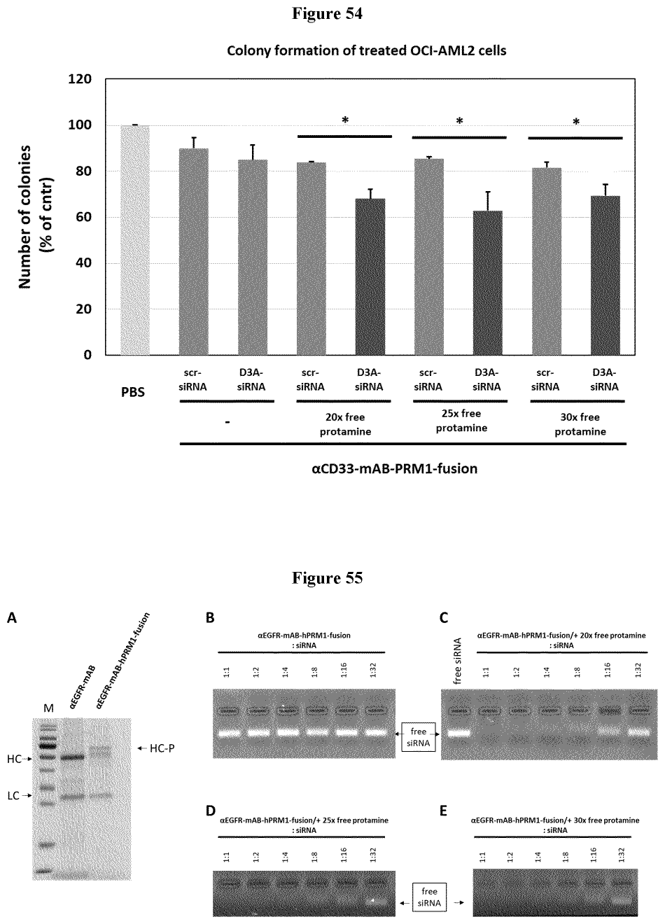

As a second example, we cloned and expressed a cetuximab IgG preparation generated by a genetic fusion between the cetuximab heavy chain and human protamine, as exemplified in FIG. 52 (see FIG. 55 for Coomassie-stained PAGE). This αEGFR-mAB-PRM1-fusion protein alone was shown to be unable to bind siRNA (FIG. 55 B), whereas the stepwise addition of protamine to the preparation restored the ability of the IgG-protamine fusion to bind siRNA in the indicated ratios (FIG. 55 C-E)

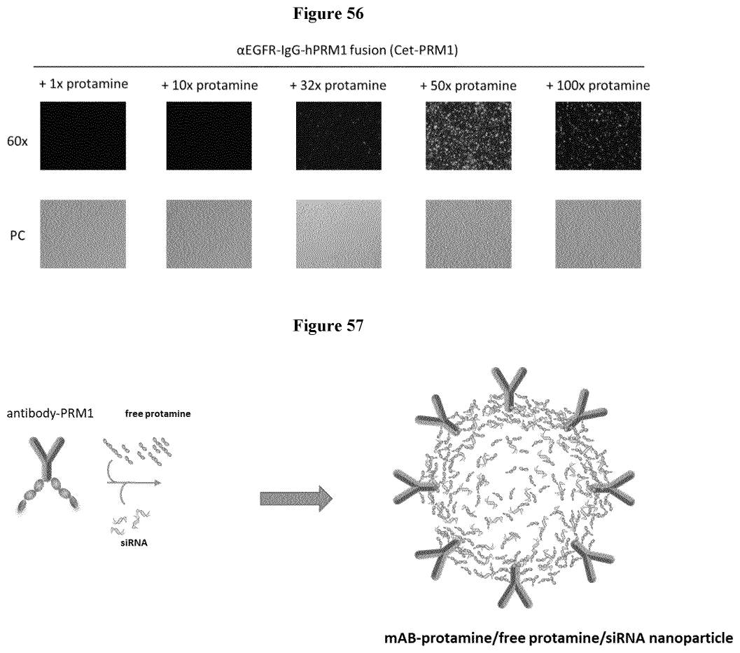

A cetuximab IgG preparation generated by a genetic fusion between the cetuximab heavy chain and human protamine alone was shown to be unable to form stable nanoparticles in cell free environment with Alexa488-marked siRNA, whereas the stepwise addition of protamine to the preparation restored the ability of the IgG-protamine fusion to form stable nanoparticles. The process was depending on the molar excess of free protamine over the IgG-protamine fusion, where molar excess of 50× free protamine over the fusion protein proved to be optimal for the nanoparticle stability. (FIG. 56).

Here, we present a proposed model of an idealized nanocomplex consisting of from siRNA, free protamine and receptor-IgG-hPRM1-fusion. It is believed that IgG-hPRM-1-fusion forms a shell structure framing certain and self-stabilizing amounts of siRNA and free protamine in a self-organized fashion. The spheroid structures form in a time-dependent manner and have an approximate diameter with 50 to 500 nm being the most prevalent fraction (FIG. 57).

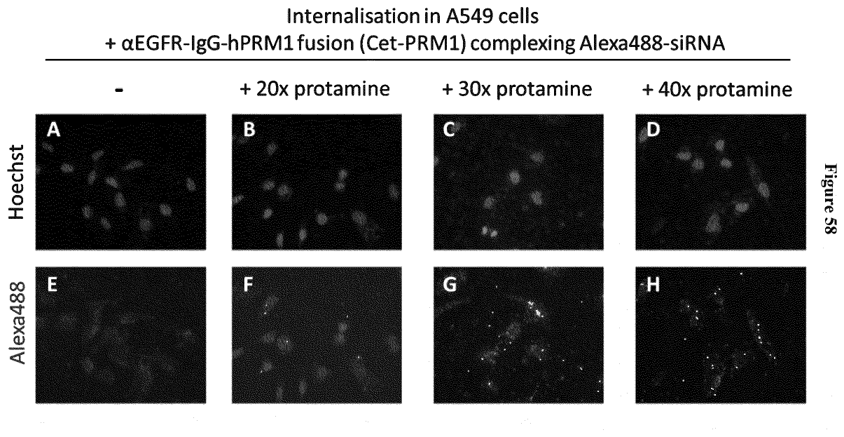

The internalization of Alexa488-marked siRNA to A549 NSCLC cells driven by cetuximab-hPRM-1 fusion protein was tested combined by rising excess concentrations of free protamine-sulfate. Here, the preparation lacking free protamine-sulfate (left) was unable to transport Alexa488-siRNA to A549 cells, whereas rising the protamine-sulfate concentration in the mixture gave rise to increasing number of internalized intracellular vesicles filled with Alexa488-siRNA, the optimal molar excess was around 30× free protamine per mol of cetuximab-hPRM1 fusion protein in conjunction with Alexa488-siRNA (FIG. 58).

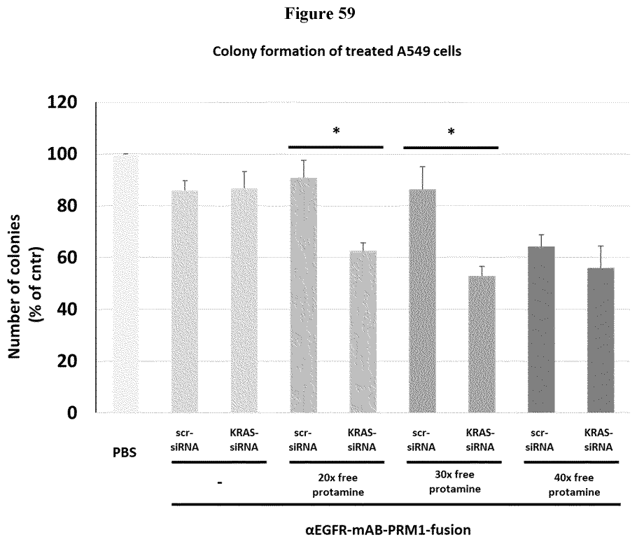

Here, we found that αEGFR-mAB cetuximab-protamine fusion reduces colony formation in presence of free protamine-sulfate in colony formation assays. A549 cells treated with αEGFR-mAB-PRM1/KRAS-siRNA in presence of 20× or 30× free protamine-sulfate, respectively, form significantly less colonies in soft agar than cells treated with αEGFR-mAB-protamine/contr (scr)-siRNA with the same amounts of free protamine-sulfate (FIG. 59). No differences compared to PBS treated cells in colony formation can be observed when A549 cells were treated with αEGFR-mAB-PRM1 fusion proteins without free protamine-sulfate. Shown here mean of three independent experiments±SD. *P<0.05, 2-sided T-test. α, anti

### Example 19: Synthesis of the Small Molecular Weight Poly-Anionic Drug Ibrutinib-Cy3.5 (“RMA561”)

Ibrutinib is a covalent binder of the Bruton's kinase. Ibrutinib is used in some lymphoma subtypes and blocks signal transduction downstream of the B-cell receptor by covalent addition to a cysteine in an ATP binding pocket in the soluble Bruton's kinase. Ibrutinib can have severe side effects such as infections, pneumonitis, or arrhythmias as ibrutinib does not only target lymphoma cells, but also BTK in normal cells (Wilson et al. 2015). The latter leads to higher dosage, interception by irrelevant cells and in addition to adverse effects, which could be partly due to the bystander effects on targets other than BTK (Byrd et al. 2013). Next, prolonged ibrutinib dosage can lead to development of resistance (Lenz 2017). Here, we first tried to conjugate ibrutinib by means of advanced linker chemistry to a suitable carrier antibody, rituximab which targets CD20 and is part of the standard therapy in DLBCL. The conjugation was successful, but it changed the solubility of the conjugate and thus proved to be not further exploitable.

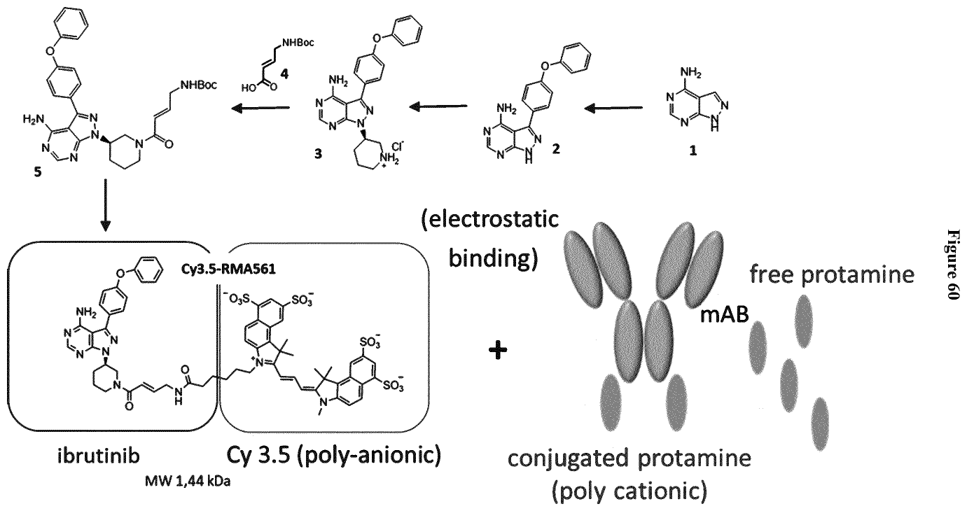

Therefore, we converted the uncharged ibrutinib to the strongly anionic compound Cy3.5-RMA561, herein referred to as ibrutinib-Cy3.5, which allowed to bind it by means of electrostatic force to our protamine-based carrier system in order to form an antibody-inhibitor-complex The cyanine dye Cy3.5 exhibits strong anionic character by exposing four sulfonic acid groups as potential binders (FIG. 60). From our point of view, it is preferred to concentrate the anionic charges concentrated on one site of the molecule and to have an overall linear shape to form the nano-carrier. In addition, the cyanine dye allows for the possibility of using a fluorescent read out in all stages of evaluation. According to the published data (Kim et al. 2015; Turetsky et al. 2014), we synthesized an amino-functionalized ibrutinib-derivative 5 starting with the commercially available pyrazolopyrimidine 1 which was subsequently iodinated and substituted with 4-phenyloxybenzene boronic acid via Suzuki-coupling to form the main part 2 of the ibrutinib core structure. Important for high binding affinity (S)—N-Boc-3-hydroxypiperidine was installed via stereocontrolled MITSUNOBU reaction forming compound 3. After deprotection of the piperidine moiety an α,β-unsaturated linker 4 (MICHAEL acceptor) was introduced for irreversible binding to the target. The resulting Boc-protected amine 5 represents the lead structure for labelling with different anionic moieties such as the cyanine dye Cy3.5 (Lumiprobe) which yields the corresponding amide ibrutinib-Cy3.5 (Cy3.5-RMA561) under basic conditions. The final product was purified by C18-SPE cartridge (purity >98% (HPLC)) and verified by high resolution mass spectrometry.

The Boc-protected derivative 5 (8.2 mg, 0.014 mmol, 1.05 eq.) was dissolved in 0.5 mL dry dichloromethane (dried on mol. sieves 4A), hydrogen chloride 4M solution in dioxane (41 μL, 0.166 mmol, 12 eq.) was added and reaction mixture was stirred at room temperature until complete conversion of 5 into the free amine (tracking by TLC: silica, solvent: 10% MeOH/EtOAc, detection: UV254 and ninhydrin staining). The reaction mixture was evaporated on vacuum with heating to 35° C. The residual white solid was dissolved in 0.5 mL anhydrous dimethylformamide and to that solution was added the NHS ester of Cy3.5 (“sulfo-Cy3.5 NHS ester” by Lumiprobe; 15 mg, 0.014 mmol, 1.0 eq.) dissolved in 0.5 mL anhydrous dimethylformamide and N,N-diisopropylethylamine (72 μL, 0.414 mmol, 30 eq.). The reaction mixture was stirred protected from light at room temperature until completion of reaction, controlled by TLC analysis (RP C-18, solvent: MeOH/H2O/AcOH 10/0.5/0.2 v/v/v, detection: UV-VIS and ninhydrin staining). The reaction mixture was evaporated on vacuum with heating to 35° C. and the residue was triturated with pentane, diethyl ether, ethyl acetate and dried in vacuum at room temperature to give 21 mg of crude product (Cy3.5-RMA561) in form of violet solid.

Analytically pure sample was prepared by chromatographic purification of crude product on 12 g C18 SPE cartridge. Cartridge was preconditioned by washing with water (10 mL). Crude product was divided in two parts, each dissolved in 0.5 mL water, loaded on the cartridge and then washed with water (10 mL) and further with acetonitrile (10 mL) to remove impurities and side products of the reaction. After that, product was eluted with mixture ACN/H2O 1:1 (v/v) in a few fractions containing exclusively pure Cy3.5-RMA561. After lyophilisation 2×8 mg of pure product Cy3.5-RMA561 as violet solid was obtained.

Besides the strong polyanionic character of the Cy3.5 dye, the conjugate had the advantage of being easily traceable in vitro and in vivo in form of a red fluorescence.

### Example 20: Analysis of Antibody-Protamine/Protamine Complex Formation with Ibrutinib-Cy3.5 In Vitro

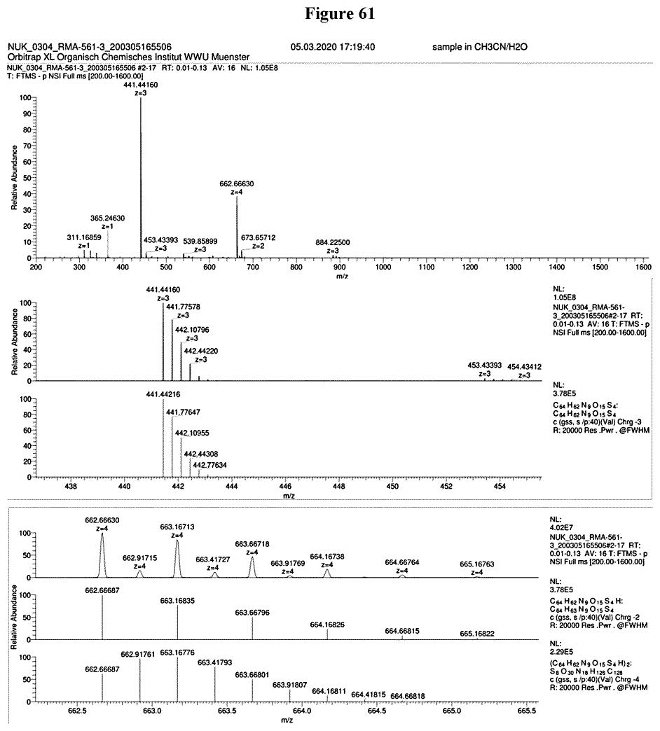

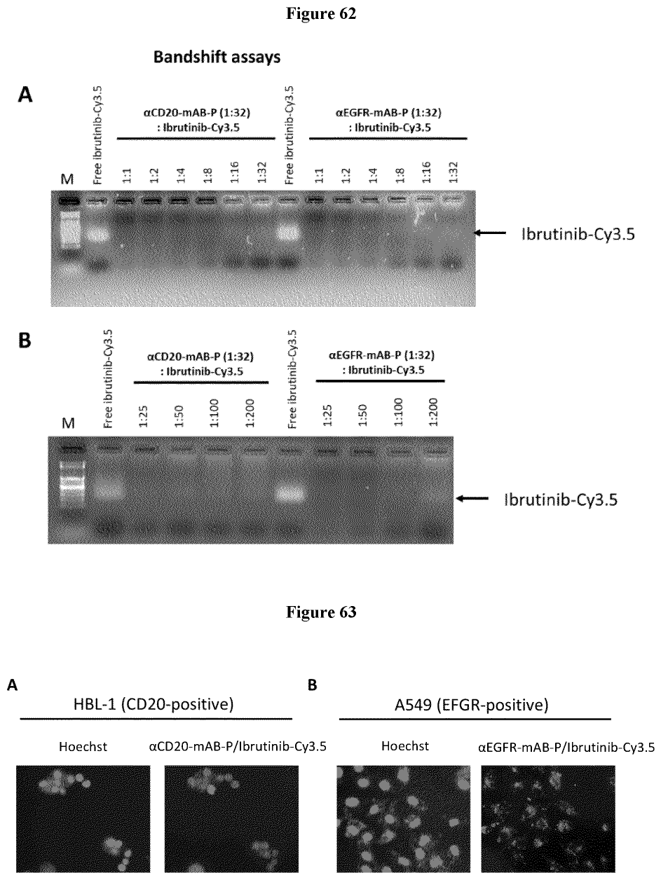

First, we performed experiments to characterize the binding between ibrutinib-Cy3.5 and two carrier systems, the both antibodies (rituximab and cetuximab) chemically conjugated by the bifunctional cross-linker sulfo-SMCC to protamine, namely rituximab (anti-CD20-mAB)-protamine, containing unbound protamine-SMCC and the cetuximab (anti-EGFR-mAB)-protamine, containing unbound protamine-SMCC in bandshift assays. Both carrier-conjugates containing unbound protamine-SMCC showed a strong co-assembly of the ibrutinib-Cy3.5 derivate and, due to its low molecular weight as compared to siRNA (appr. 13 kDa), an extremely high mol/mol ratio of electrostatic saturation, exceeding 100 mol ibrutinib-Cy3.5 per mol of carrier mAB (FIG. 62). In all further experiments, the composition of the chemically conjugated antibodies-to-protamine remains unchanged, meaning that the resulting product still contains unbound protamine-SMCC besides the antibody-protamine conjugate.

The corresponding conjugates rituximab- and cetuximab-protamine, both containing unbound protamine-SMCC loaded with a sub-critical 20× excess of ibrutinib-Cy3.5 were incubated with CD20 and EGFR-expressing cell lines and analysed for intracellular enrichment of ibrutinib-Cy3.5. In both cases, intracellular enrichment of fluorescence signals at the typical Cy3.5 excitation/emission wavelength combination could be documented (FIG. 63). Thus, the modified ibrutinib-Cy3.5 compound is still being internalized by cells and consequently can get to action, provided it still binds to its target BTK.

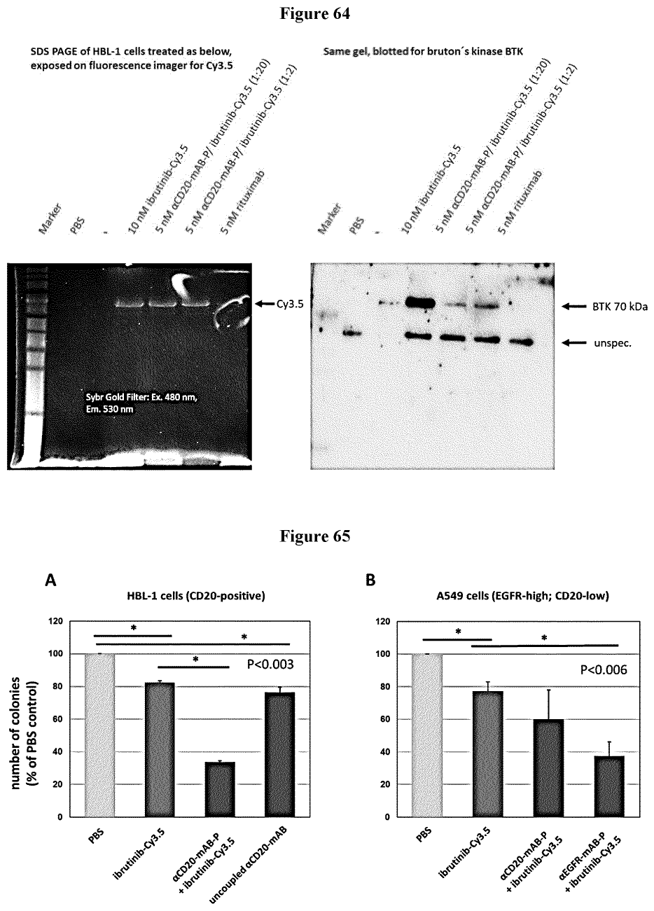

Next, we confronted DLBCL cell lines with the ibrutinib-Cy3.5 derivative coupled to rituximab carrier antibody, lysed the cells and subjected the lysate to SDS PAGE analysis. The gel was then UV-illuminated and scanned for emission from Cy3.5 chromophore, and indeed, there was a single protein band at 70 kDa emitting Cy3.5 fluorescence, which later was identified for being Bruton's kinase BTK by western bot analysis (FIG. 64).

In conclusion, the chemical modification of the ibrutinib core structure to a polyanionic derivative does not alter the efficiency of the conjugate to bind Bruton's kinase BTK.

Next, we planned functional assays for identifying the effectivity of the antibody-inhibitor-complexes. DLBCL tumor cells were seeded in methylcellulose to form anchorage-free colony growth as a substitute marker for tumorigenicity. Assays were treated with combinations of antibody-inhibitor complexes and compared with appropriate control groups. It became evident, that HBL-1 cells, high in CD20-expression, formed only 30% of colonies when treated with rituximab-protamine/protamine with ibrutinib-Cy3.5 as compared to the control groups without the carrier mAB. In contrast, unconjugated rituximab had only a mild effect on colony growth. Additionally, in A549 NSCLC cells, high in EGFR-expression but low in CD20-expression (FIG. 65 B), the cetuximab carrier performed significantly better than the rituximab carrier, revealing a receptor-specific uptake mechanism as intended.

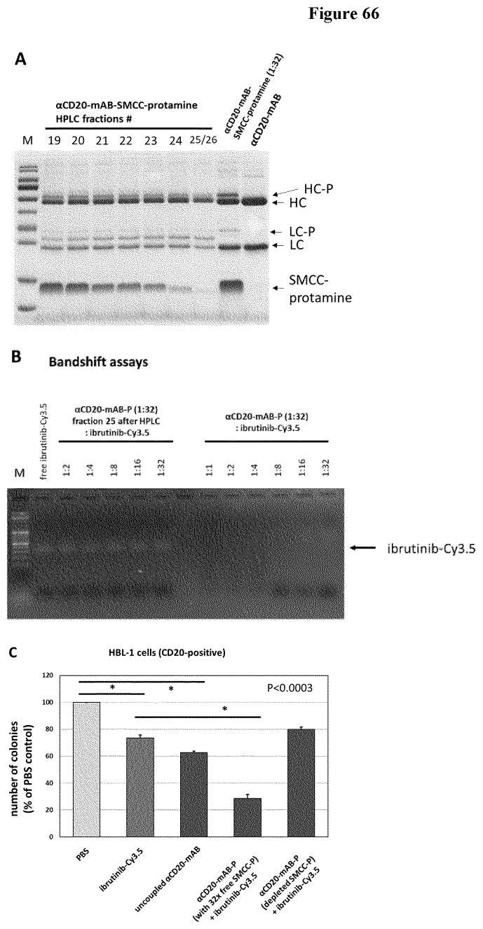

As seen from results of our antibody-protamine/free protamine-SMCC/siRNA conjugation experiments, we hypothesized, that the presence of unbound/free protamine-SMCC in the αCD20-mAB-protamine-ibrutinib-Cy3.5-protamine adduct could be as important as in the siRNA adducts, so we depleted free protamine-SMCC from the rituximab-αCD20 mAB preparation by affinity chromatography as done before. As expected, the depleted preparation (FIG. 66A, fraction 25) was not able to bind and coordinate polyanionic ibrutinib-Cy3.5 to the same extent (FIG. 66 B, left) as the protamine-SMCC containing preparation (FIG. 66 B, right). Comparable to αCD20-mAB-protamine/free protamine-SMCC complexes after depletion of free protamine-SMCC, CD20-mAB-P without free protamine-SMCC does not confer inhibition of colony formation when complexed to ibrutinib-Cy3.5 (FIG. 66 C).

### Example 21: Analysis of Antibody-Protamine/Free Protamine-SMCC Complex Formation with Ibrutinib-Cy3.5 In Vivo

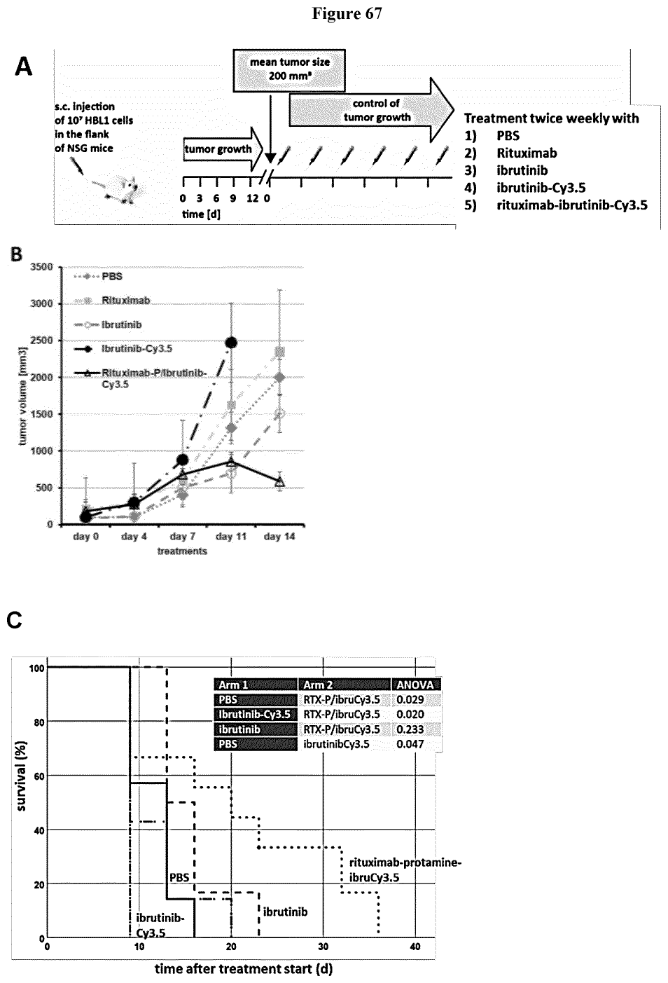

In order to further characterize the therapeutic in vivo efficacy of rituximab-protamine/free protamine/ibrutinib-Cy3.5 carrier in comparison to all necessary component controls, we performed in vivo treatment experiments as depicted (see FIG. 67A). To this end, we subcutaneously transplanted 107 HBL-1 DLBCL-cells to immune-deficient NSG mice, observed tumor growth up to a mean size of 200 mm³, sorted the mice to groups of ten mice each, and started treatment with a standard concentration of 4 mg/kg body weight, calculated for rituximab, corresponding to 0.625 nmol rituximab conjugate per single dose, rituximab-protamine/free protamine-SMCC/ibrutinib-Cy3.5 (1:20) corresponding to 0.625 nmol rituximab conjugate per single dose plus 18 μg or 12.5 nmol of ibrutinib-Cy3.5. and equivalents of uncoordinated ibrutinib (12.5 nmol) and ibrutinib-Cy3.5 (12.5 nmol), further controlled by PBS. The therapy with ibrutinib-Cy3.5 showed no therapeutic effect on tumor growth, whereas application of the equivalent amount of ibrutinib-Cy3.5, bound into the rituximab-protamine carrier yielded significantly slower growth of tumors compared to all other groups (FIG. 67 C). This translated also into the survival analysis of the animals (FIG. 67 B).

Therefore, rituximab-protamine/free protamine/ibrutinib-Cy3.5 1:20 complex in an in vivo model, showed significantly superior targeting and therapeutic profile in comparison to all appropriate component controls. Furthermore, the applied single doses of ibrutinib were in the range of twentyfold lower (12.5 nmol of ibrutinib corresponds to 0.720 mg/kg mouse, the standard dose is 12 mg/kg in Nod-SCID mice (Chen et al. 2016; Zhang et al. 2017).

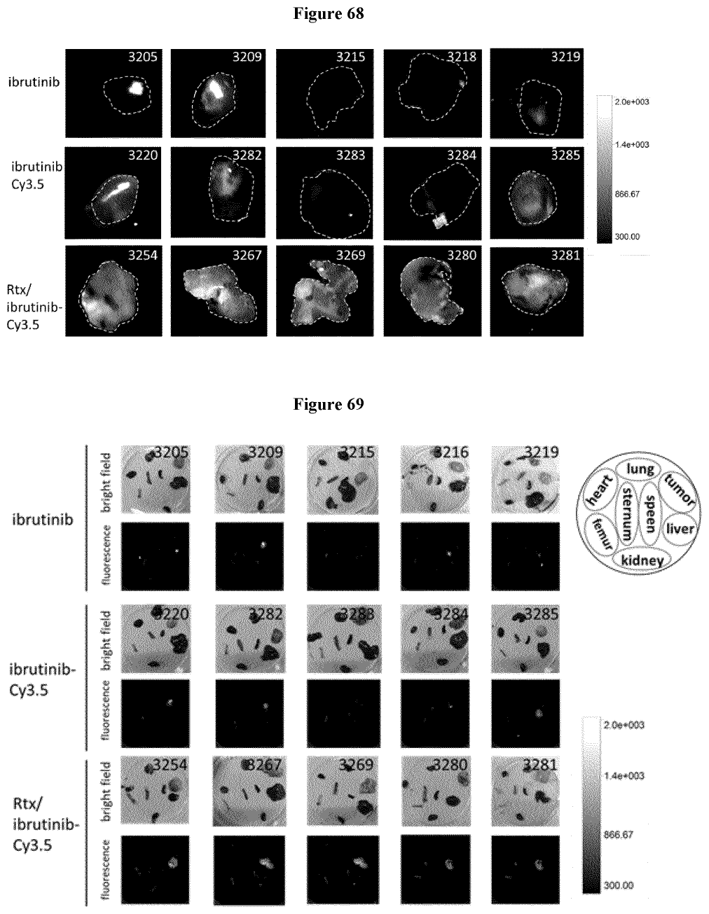

In order to prove this hypothesis, we prepared organs from sacrificed mice and subjected them to ex vivo fluorescence detection of incorporated ibrutinib-Cy3.5 (FIG. 68). As a result, tumors from rituximab-protamine/ibrutinib-Cy3.5 treated mice showed a marked accumulation of Cy3.5-originated fluorescence signals, increasing with treatment cycles. In contrast to this finding, there was rarely a signal detectable in tumors from mice treated with ibrutinib-Cy3.5 that was given non-coordinated by rituximab (FIG. 68), whereas in this group, there was a tendency of a diffuse background signal seen in most organs (FIG. 69).

Moreover, no specific fluorescence was detected in the organs analyzed (FIG. 69). We conclude that the rituximab-protamine/free protamine-SMCC/ibrutinib-Cy3.5 conjugates are enriched specifically in the CD20-positive tumors.

### Example 22: Formation of Vesicular Structures In Vitro by Antibody-Protamine/Free Protamine-siRNA and/or Ibrutinib-Cy3.5 Complexes

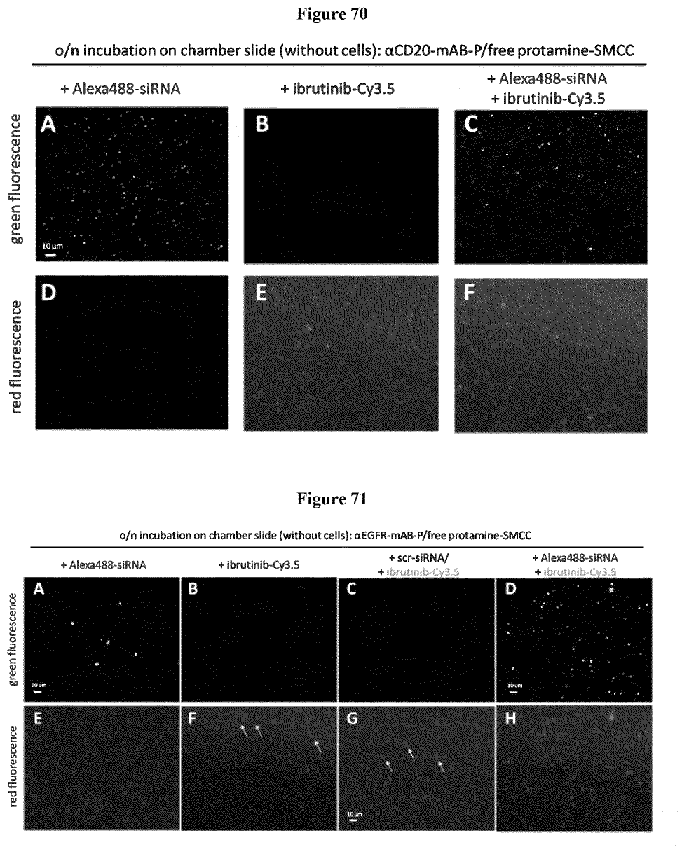

To further characterize the new nanostructure, we used electrostatically charged green-fluorescent siRNAs and red-fluorescent ibrutinib-Cy3.5 and complexed it to antibody-protamine/free protamine-SMCC conjugates (FIG. 70-FIG. 72).

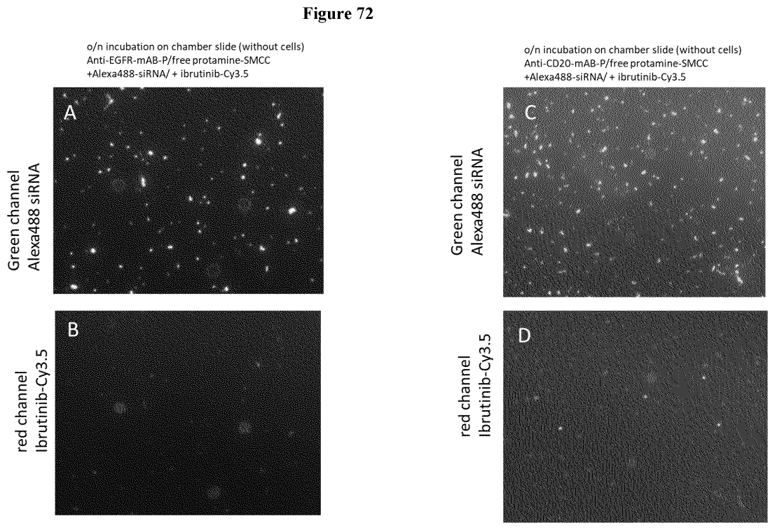

During the inspection of the results of the cell-free assembly of mAB-protamine/free protamine/ibrutinib-Cy3.5-conjugates with and without additional siRNA, we observed marked differences in the quantity and size of the respective nanostructures: those assembled by siRNA AND the ibrutinib-Cy3.5 complexed by mAB-protamine plus free protamine-SMCC were considerably larger and more frequent than without siRNA (FIGS. 70 C and F and FIGS. 71 D and H). We explain this phenomenon by a hypothesis of a mixed particle consisting of all 4 components forming a stable nanostructure. In detailed fluorescence micrographs, the nanostructure of the largest particles can be revealed as siRNA forming the outer rim of a spheroid micelle, whereas the ibrutinib-Cy3.5 fills more the lumen of this nanostructure (FIG. 72).

This phenomenon that mixed antibody-protamine particles are more frequent and much larger than those with only ibrutinib-Cy3.5 complexed to the mAB-protamine/protamine carrier could be seen both with αCD20-mAB rituximab (FIG. 72 C-D) and anti-EGFR-mAB cetuximab as carrier antibodies (FIGS. 72A and B).

This means that the negatively charged ibrutinib-Cy3.5 can form small vesicles with antibody-protamine complexes (FIG. 70 E and FIG. 71 F), but siRNA, which is a linear and highly negatively charged molecule, might serve as a kind of electrostatic “glue” between the antibody-protamine/free protamine-SMCC complexes. This can be observed as a circular shining in extraordinary large vesicles that seem to be filled with red fluorescent ibrutinib-Cy3.5 (FIG. 72).

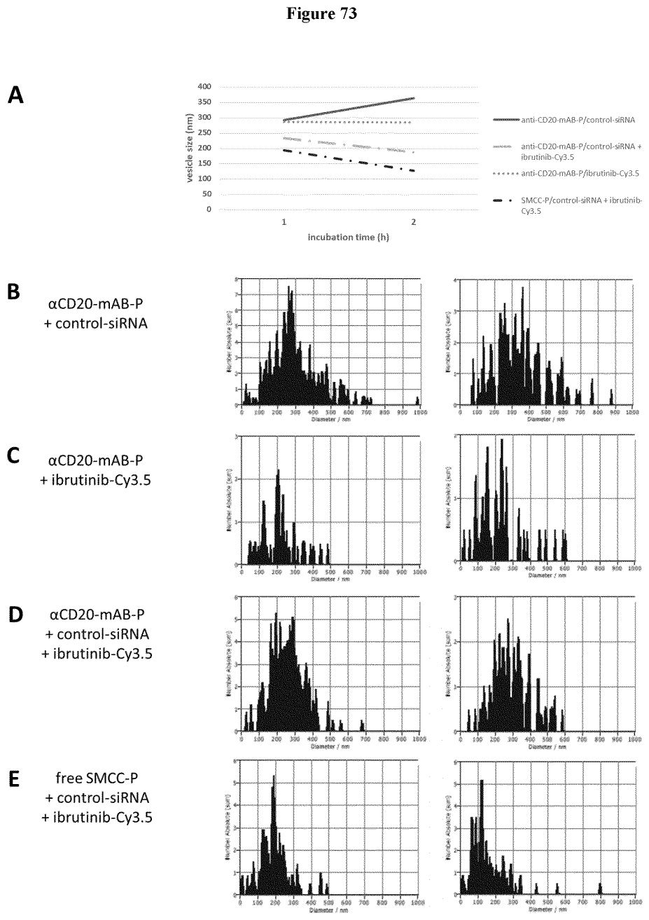

To further characterize this structure, we performed measurements of particle sizes using a Zeta View® nanoparticle tracking video-microscope. Here, particles between 1 and 1000 nm were detected and analysed for their size and number (FIG. 73). We detected the stable and largest particles 1 h after start of the complex formation by the addition of control (scr)-siRNA, ibrutinib-Cy3.5 or both of them, respectively (FIG. 73A, B, C, D). The use of equal amounts of antibody-free protamine-SMCC (=1800 nM=30×molar ratio as used for the coupling of 60 nm anti-CD20-mAB) formed constantly smaller particles (FIG. 73A, lower panel and FIG. 73 E). Interestingly, the formation of the very large mixed particles, as seen in FIG. 72 was not observed in Zetaview data, because of technical limitations.

### Example 23: Complexation of Anionic Small Molecular Drugs by Carrier-Antibody-Protamine Fusions or Antibody-Protamine Conjugates

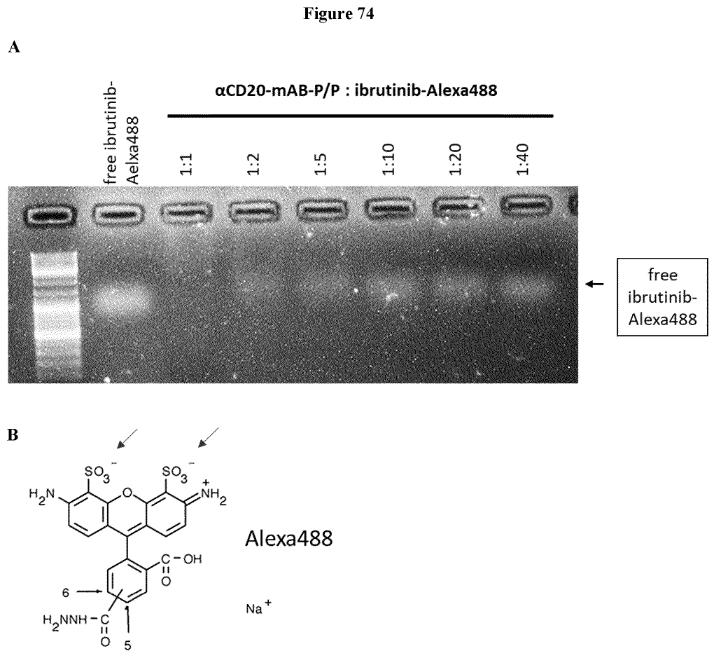

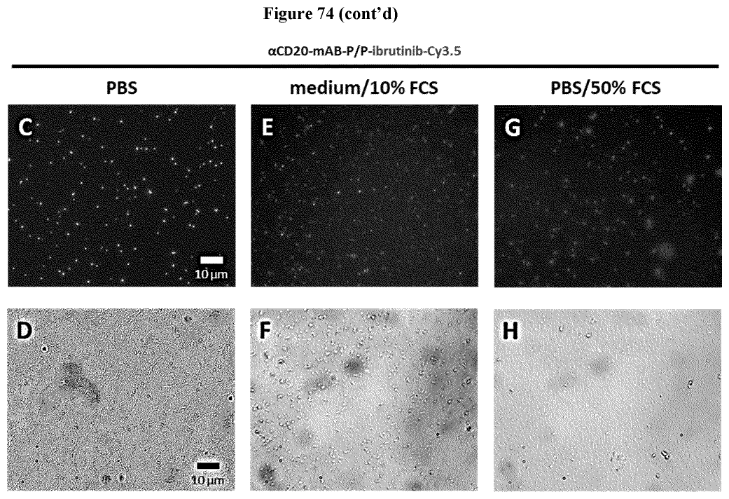

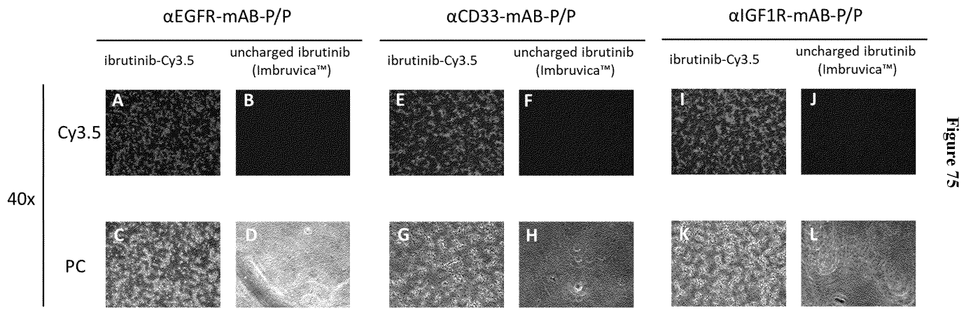

Concerning the complexation of anionic small molecules, we found that αCD20-mAB rituximab-protamine/free protamine-SMCC (αCD20-mAB-P/P) conjugates bind ibrutinib-Alexa488 in a Bandshift assay using αCD20-mAB-protamine using different ratios of ibrutinib-Alexa488 up to 1:2. α, anti. (FIG. 74, A): Due to the limited anionic charge of the Alexa488 molecule of −2 (FIG. 74, B), the interactions between the polycationic protamine fusions and Alexa488 were found to be less intense than those with Cy3.5 (next example), which has a net charge of −4. Consequently, with Alexa488-conjugated ibrutinib and protamine conjugates, coupling ratios of only 2:1 were realized. However, complexation of ibrutinib-Alexa488 with αCD20-mAB rituximab-protamine/free protamine-SMCC (αCD20-mAB-P/P) was still successful.

The building of an antibody-inhibitor-complex in form of a stable nanoparticle could be detected in fluorescence microscopy (FIG. 74 C—H), which are stable in serum (FIG. 74 E, G, F, H) under conditions as published for other nanoparticles. Importantly as ibrutinib-Cy3.5 is detectable by fluorescence, this brings along excellent tracing abilities for all downstream applications.

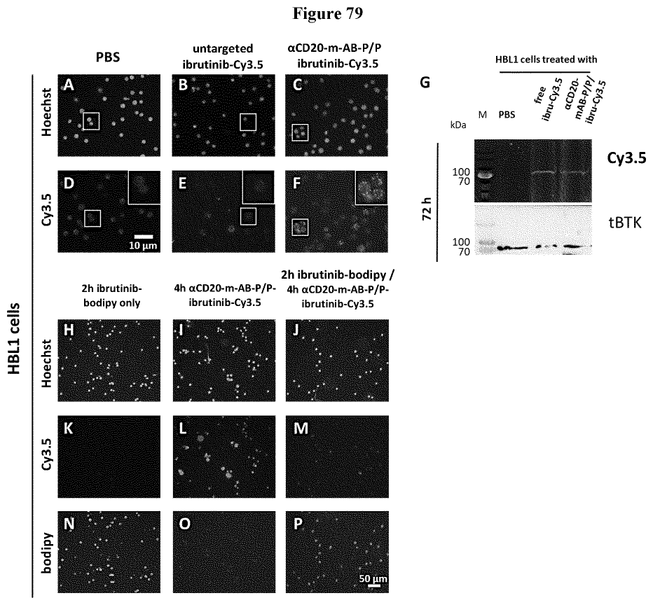

When incubated in vitro, αCD20-mAB-P/P loaded with ibrutinib-Cy3.5 led to the assembly of electrostatically stabilized nanoparticles exposing red Cy3.5 fluorescence (FIG. 78). In fluorescence microscopy, first regular shaped vesicular structures (FIG. 74 C, D), later irregular shaped aggregates larger than 2 μm plus smaller particles were detected, this process was not seen, if unmodified αCD20-mAB was used to complex ibrutinib-Cy 3.5, or modified αCD20-mAB-P/free protamine was used to complex hydrophobic ibrutinib (trade name: Imbruvica) (not shown). The electrostatic particles seen in light microscopy (FIGS. 78 A and B) were also validated in electron microscopy (FIG. 78 C), where a multitude of smaller particles ranging <100-200 nm were detected (FIG. 78 C), which induced us to choose the term “nano”carrier.

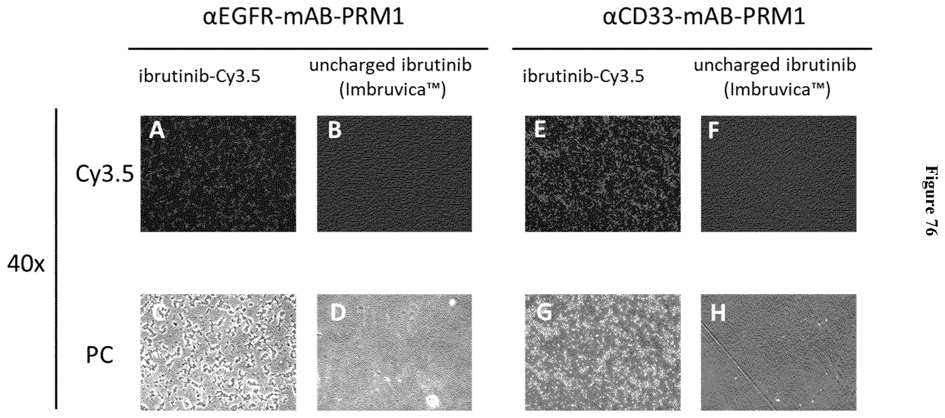

Concerning the comparison of anionic charged small molecules versus uncharged small molecules in terms of complexation t protamine conjugates or fusions, we found that charged ibrutinib-Cy3.5, but not uncharged ibrutinib (trade name: imbruvica) forms stable nanoparticles with protamine-conjugated mABs. The respective antibody carriers, conjugated to protamine were loaded with charged ibrutinib-Cy3.5 versus uncharged ibrutinib. Of note, only those ibrutinib samples conjugated with Cy3.5 showed a dense formation of nanoparticles, but not uncharged ibrutinib (FIG. 75), indicating that a poly-aninonic net charge as well as a certain structure of the poly-anion is important for the proper electrostatic interaction to protamine.

Next, we tested the complexation ability of protamine (“hPRM1”)-fused antibody constructs concerning the coordination of charged ibrutinib-Cy3.5 versus uncharged ibrutinib (trade name: imbruvica). Only Cy3.5-conjugated ibrutinib forms stable nanoparticles with two protamine-fused mABs (FIG. 76). Here, we used hPRM1-protamine-fusions of anti-EGFR (A-D) as well as hPRM1-fusions with anti-CD33 to complex charged ibrutinib-Cy3.5. Stable nanoparticles were formed with ibrutinib-Cy3.5, but not with uncharged ibrutinib.

### Example 24: Proposed Model

Taken together, the principle of binding anionic cargo molecules by a carrier consisting of antibody-protamine plus unbound protamine can be applied also to cargos other than siRNA-nucleic acids, such as small molecules, e.g. the kinase inhibitor ibrutinib. Here, it is important to modify the cargo molecule to give it a polyanionic character, and to leave unbound protamine-SMCC in the preparation to enable a strong electrostatic self-assembly of the components into the nano-structure.

This observation strongly supports that the new and unexpected macromolecular nanostructure is responsible for the in vitro and in vivo pharmacodynamic efficacy of our carrier system.

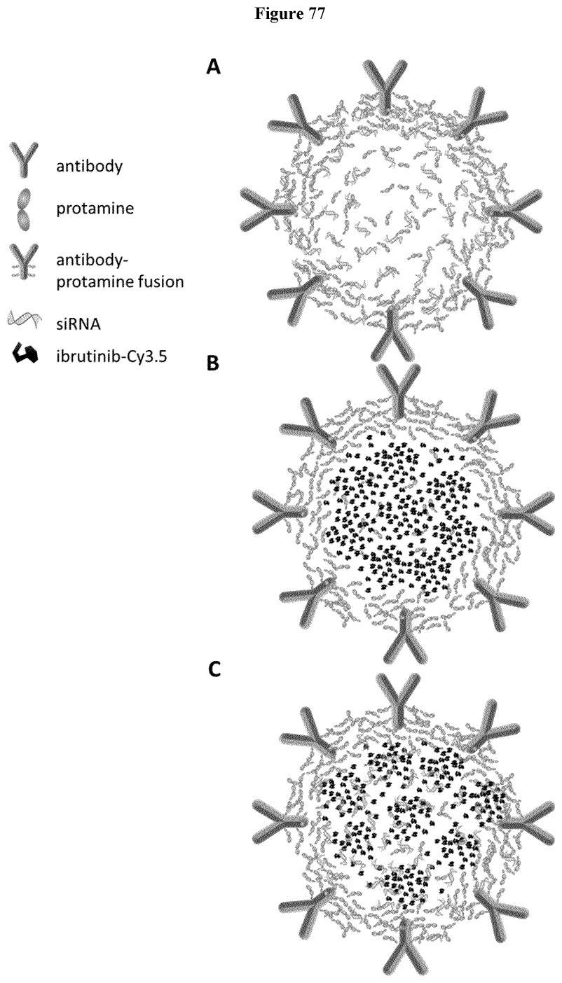

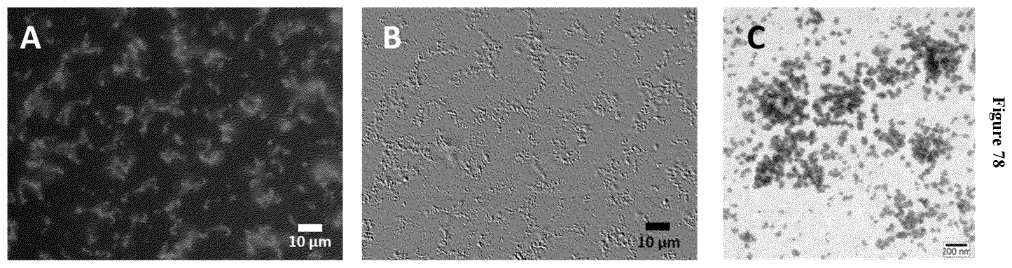

Therefore, we expect the combination of the components 1. antibody-protamine, 2. siRNA/anionic small molecule and 3. unbound protamine(-SMCC) to form a nanoparticle-like macrostructure, which is responsible for the stability of siRNA and can effectivity deliver siRNA and/or anionic small molecule inhibitors to the intended cells, which is a totally unexpected observation. An idealized model of this nanostructure assembly is shown in FIG. 77.

In conclusion, experiments using various chemically different effector pay loads with the minimal common denominator requirement of being poly-anionic and no other structural similarity, lend experimental evidence for our new and unexpected nanostructure model as being the basis for the in vitro and in vivo pharmacodynamic characteristics of our nanocarrier-siRNA carrier and our nanocarrier-ibrutinib-Cy3.5 system.

This modular nanostructure system with dual specificity, 1. for siRNA/anionic small molecule transport and specific delivery to target cells and 2. for specific intracellular oncogene inactivation or pharmacological activity can be used for various disease groups including cancer.

### Example 25: Functional Analysis of the αCD20-mAB-P/P-Ibrutinib-Cy3.5 Nanocarrier In Vitro

Next, the efficacy of this αCD20-mAB-P/P-ibrutinib-Cy3.5 nanocarrier in different cellular model systems was investigated.

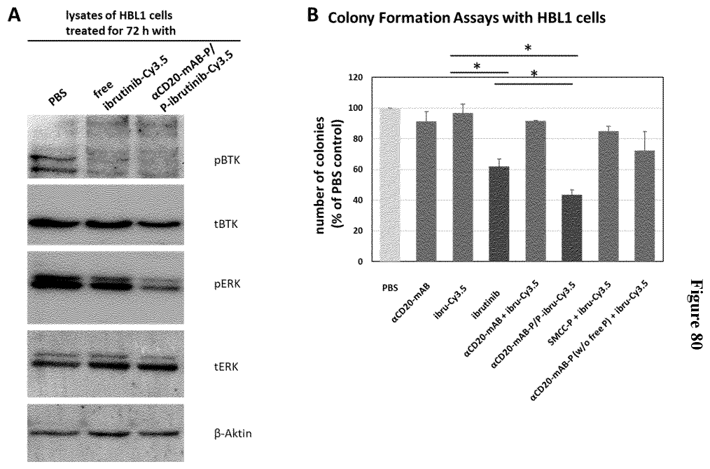

First, the internalisation into CD20-positive DLBCL cells via Cy3.5 fluorescence was examined. HBL1 and TMD-8 lymphoma cells treated overnight with uncoupled ibrutinib-Cy3.5 show decent red fluorescence marking of cells (white in FIG. 79 E), which was intensified, when ibrutinib-Cy3.5 was complexed and transported with αCD20-mAB-P/P (FIG. 79 F). This indicated a beneficial process of internalization by the CD20 receptor over the untargeted uptake mechanisms for ibrutinib-Cy3.5 anion without carrier antibody implementation (FIG. 79 E). Next, a 72 hrs treatment of cells with the conjugates show a singular band of covalent Cy3.5 marking of a 70 kDa protein in an SDS PAGE electrophoresis, indicating binding and functionality of the modified ibrutinib-Cy3.5 compound (FIG. 79 G). For fluorescence detection of BTK, the gel had to be considerable overloaded, in order to show equal loading of lanes and identification of BTK, so next we blotted the gel for immunodetection of BTK after fluorescence detection. Indeed, a band representing BTK appeared at the same position as seen in the Cy3.5 fluorescence, indicating that ibrutinib-Cy3.5 had covalently bound exclusively to BTK, as anticipated (FIG. 79 G).

Moreover, HBL1 cells were incubated with ibrutinib-bodipy for 2 h, washed and treated with αCD20-mAB-P/P-ibrutinib-Cy3.5. Cells incorporate ibrutinib-bodipy (FIGS. 79 N and P), but Cy3.5 fluorescence only appears in non-pretreated cells (FIG. 79 L) and not in cells pre-treated with ibrutinib-bodipy (FIG. 79 P). Some subcellular red vesicles indicate CD20-mediated internalization of ibrutinib-Cy3.5 (FIG. 79 P), but a pattern that hints at BTK binding (see FIG. 79 L for ibrutinib-Cy3.5 and FIGS. 79 N and P for ibrutinib-bodipy) does not occur. This is also true after 24 h of αCD20-mAB-P/P-ibrutinib-Cy3.5 treatment and after pre-incubation with and washout of non-fluorescent ibrutinib.

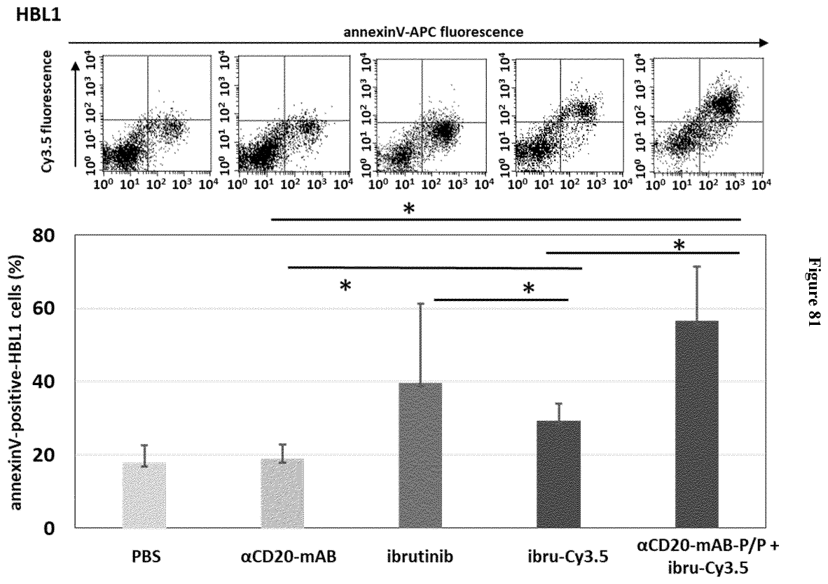

The functional effect of covalent targeting of BTK by ibrutinib is the inhibition of BTK autophosphorylation ability. Therefore, the phosphorylation status of BTK in DLBCL cells after ibrutinib-Cy3.5 treatment with and without complexation in the αCD20-mAB/P/P nanocarrier was analysed (FIG. 80A). Cells were treated for 72 hrs with PBS, uncomplexed ibrutinib-Cy3.5 and with the αCD20-mAB-P/P/ibrutinib-Cy3.5 complex, lysed and subjected to Western blot analysis. We found that phosphorylation of BTK at tyrosine 223, detected by a specific phospho-BTK-antibody was significantly decreased in HBL1 (FIG. 80A, left panel) and TMD8 cells (Data not shown) upon treatment with ibrutinib-Cy3.5, irrespective if it was complexed or not. This was in accordance with its binding to BTK as depicted in FIG. 79 G. Expression of total BTK was mildly influenced (FIG. 80A). We concluded that the synthesized ibrutinib-Cy3.5 conjugate retains full functionality in terms of binding the target molecule BTK as well as inactivation of BTK autophosphorylation.

Interestingly, in all tested lymphoma cell lines, the lymphoma-specific αCD20-mAB-P/P/ibru-Cy3.5 nanocarrier system significantly inhibited colony growth in soft agar cultures. This was observed to a much lesser degree for ibrutinib or ibrutinib-Cy3.5 as single agents, and not if unmodified rituximab (αCD20-mAB) was used (HBL1: FIG. 80 B). This colony-assay is used for quantification of anchorage-independent clonal cell growth and is a standard in vitro surrogate for tumorigenicity in vivo. We therefore argue that a robust therapeutic effect of ibrutinib-Cy3.5 is only seen, when the anionic compound is assembled into a stable electrostatic nanoparticle composed of the cationic αCD20-mAB-protamine/free protamine carrier complex and the anionic cargo effector.

Next, the functional consequences of BTK inactivation by αCD20-mAB-P/P-ibrutinib-Cy3.5 on DLBCL cell lines in terms of induction of apoptosis was explored. Here, in HBL1 (FIG. 81) as well as in TMD8 cells (data not shown), αCD20-mAB-P/P-ibrutinib-Cy3.5 treatment offered superior induction of apoptosis signals (FIG. 81, rightmost bar), whereas the uncomplexed ibrutinib-Cy3.5 treatment showed only mild effects in comparison to the targeted treatment as well as the free ibrutinib treatment. It is therefore assumed that the targeted treatment of αCD20-mAB-P/P-ibrutinib-Cy3.5 leads to an accumulation of active ibrutinib-Cy3.5 in the cells and hence to a more severe induction of apoptosis than the uncomplexed ibrutinib-Cy3.5. It also seems that the anionic molecule ibrutinib-Cy3.5, if uncomplexed is less accessible or at least less effective to the cells as the hydrophobic free ibrutinib, judged by the lower induction of apoptosis as compared to free ibrutinib.

### Example 26: Ewing Sarcoma Xenograft Tumor Growth is Inhibited Upon Knockdown of Oncogenic EWS-FLI1 Translocation Product Through Systemic Therapy with αIGF1R-mAB-Protamine-siRNA-Protamine Nano-Carriers

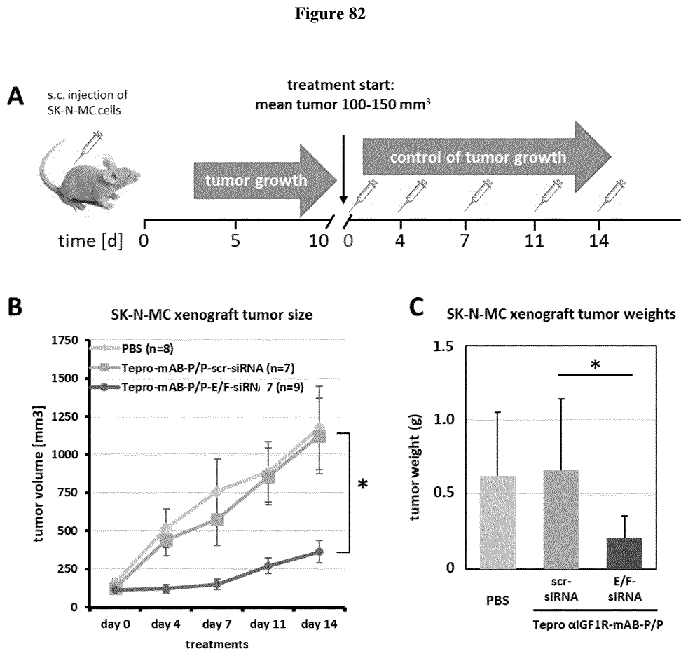

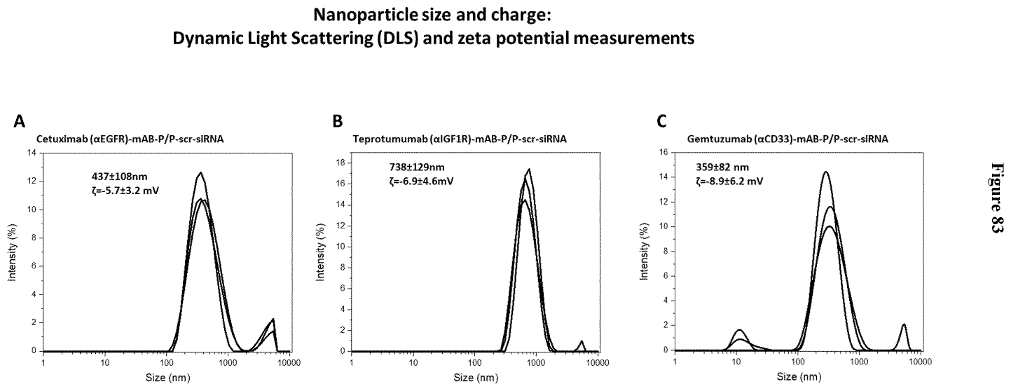

To test the in vivo-efficacy of teprotumumab-protamine nanocarriers, 107 human SK-N-MC cells were subcutaneously (s.c.) xenotransplanted into the flank of CD1-nude mice and treated cohorts of at least 7 mice with either PBS or αIGF1R-mAB-P/P in complex with scrambled control-siRNA, or in complex with the above mentioned EWS-FLI1-siRNA i.p. (FIG. 82A-C). Treatment was started when tumors had reached an average size of 100-150 mm3. Tumors in the treatment group that obtained Tepro-mAB-P/EWS-FLI1-siRNA/P nanoparticles showed a significant and almost complete growth inhibition when compared to both control groups (FIGS. 82 B and C). This suggested that the knockdown of EWS-FLI1 via Tepro-mAB-P/siRNA/P nanoparticles was successful after systemic in vivo application.

### Example 27: Nanoparticles Formed by Carrier Antibodies-Protamine/Free Protamine and siRNA Expose an Almost Neutral Surface Charge

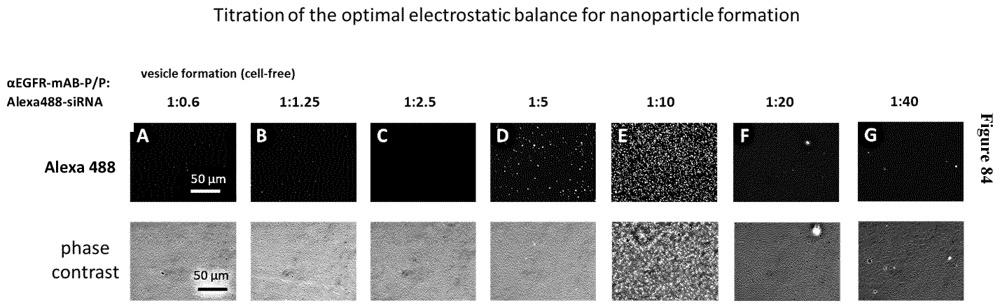

The formation of nanoparticles from antibody-protamine/free protamine plus siRNA was found to be rapid and reproducible, but depending on the antibody preparation. For instance, different α-IGFR-protamine preparations tended towards larger particles than those formed by αEGFR-protamine preparations or αCD33-preparations, seen by DLS analysis (FIG. 83) and microscopic analysis. Next, the surface charge varied only slightly in the weak anionic range, exposing nearly neutrally charged particles. It is concluded that the nature of the antibody itself as well as the electrostatic balance of anionic and cationic components defines the attributes of the nanoparticle in size and surface charge.

### Example 28: Deciphering Preconditions for Effective Nanoparticle Formation Between Anti-EGFR-mAB-SMCC-Protamine Conjugate, Free SMCC-Protamine and siRNA

Moreover, it was assessed if siRNA is needed to form vesicles. A constant amount of αEGFR-mAB-P with constant 32× free SMCC-protamine was incubated with different amounts of Alexa488-control-siRNA (FIG. 84A-G, green fluorescence in upper panels, phase contrast in lower panels). Remarkably, nanoparticles are efficiently formed with an optimal molar excess of siRNA of 5-10 times over the antibody (FIG. 84 D-E).

### Example 29: Nanoparticles Formed by αEGFR-Protamine/Free Protamine-Alexa488-siRNA are Stable in Serum-Containing Conditions

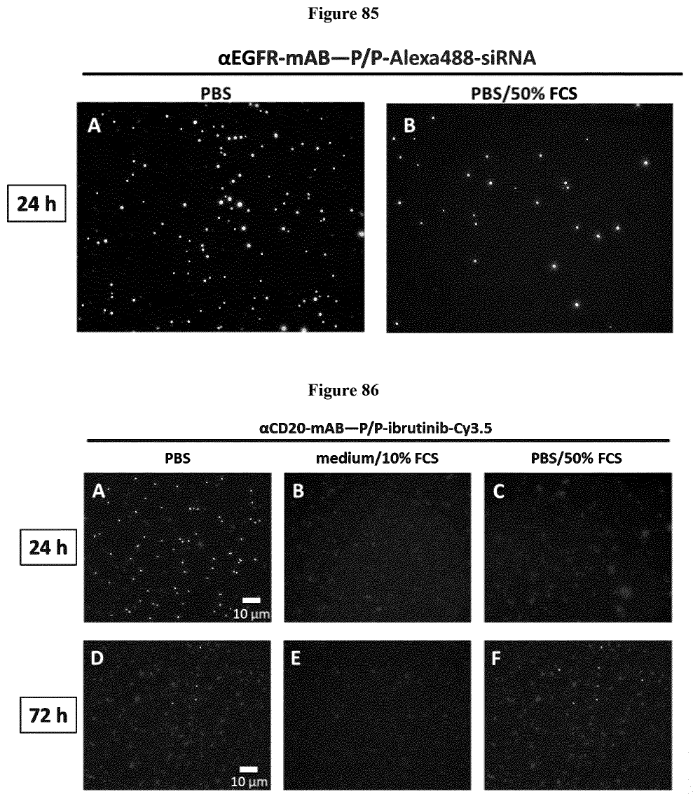

For a systemic therapeutic application of a targeted nanoparticle, its stability in various challenging conditions is of highest importance, otherwise the active substance would be separated from the nanocarrier by disintegration. Here, the αEGFR-mAB-protamine, free protamine and Alexa488-siRNA were tested for stability in a high concentration of bovine serum albumin and the nanocarrier proved to be stable even after 24 hrs (FIG. 85 B).

### Example 30: Serum Stability of the αCD20-mAB-Protamine/Free P-Ibrutinib-Cy3.5 Nanocarrier

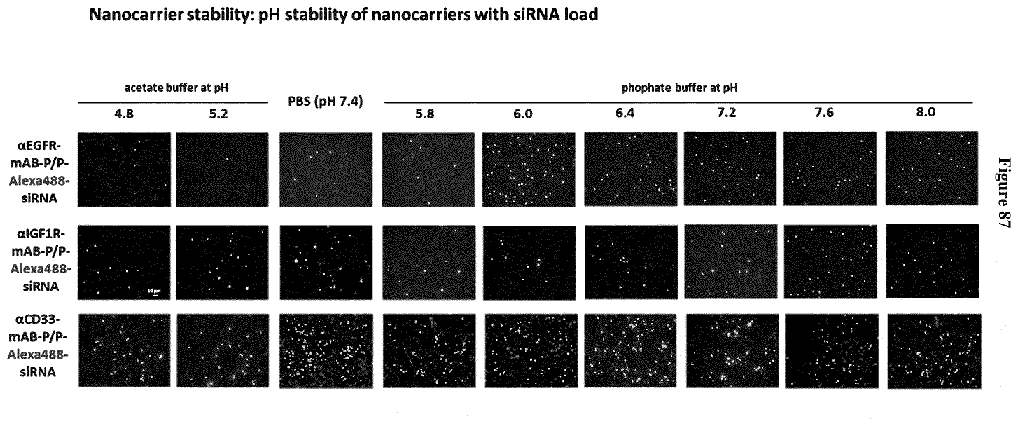

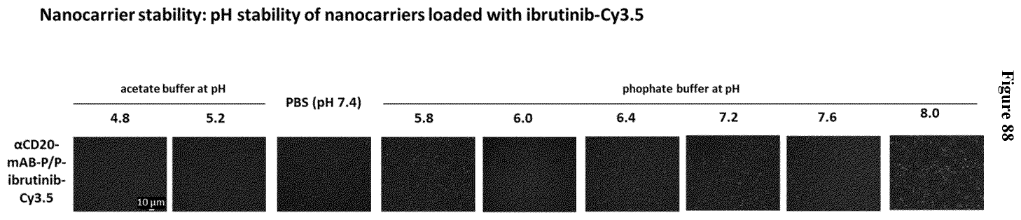

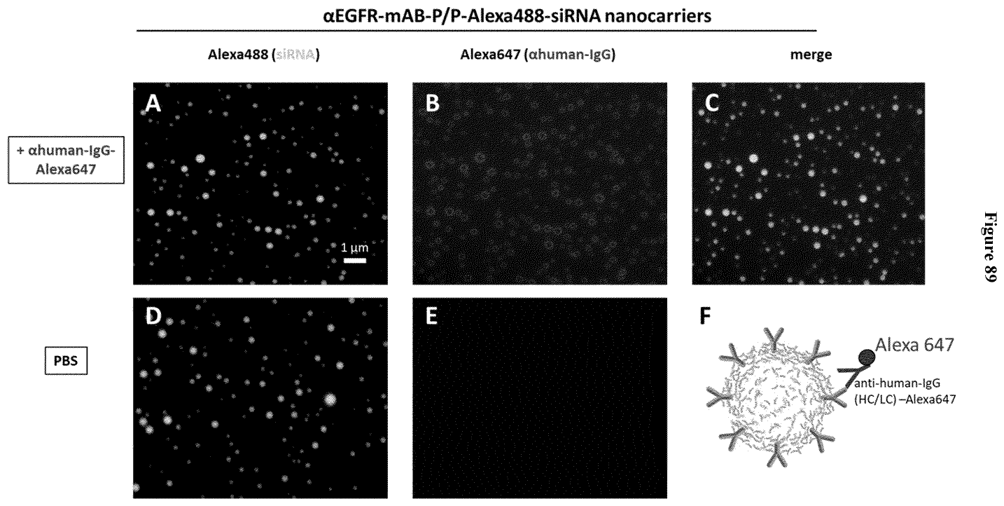

The building of an αCD20-mAB-protamine/free P-ibrutinib-Cy3.5 antibody-inhibitor-complex in form of a stable nanoparticle could be detected in fluorescence microscopy (FIG. 86A-F), which are stable in serum for 24 h (FIG. 86 B-C) and even 72 h (FIG. 86 E-F).

### Example 31: pH Stability of siRNA Nanocarriers Constructed with Three Different Targeting Antibodies

For a systemic application of the nanocarriers, it is important under which pH conditions the structures are stable, in order to prevent a premature disassembly and loss of co-ordinated siRNA effector molecule. Here, we formed siRNA nanocarriers with three different targeting antibodies and siRNA under standard conditions and tested them for integrity under pH conditions ranging between pH 4.8 and 8.0 (FIG. 74), thus covering all pH conditions the nanocarrier may be challenged with during therapeutic application. It turned out that the nanocarriers, judged by Alexa488 fluorescence of the complexed siRNA, were stable in pH conditions between 5.2 and 8.0, with a tendency of the structured to form larger superstructures at lower pH.

### Example 32: pH Stability of Nanocarriers Constructed with αCD20-mAB-Protamine/Free Protamine and Ibrutinib-Cy3.5

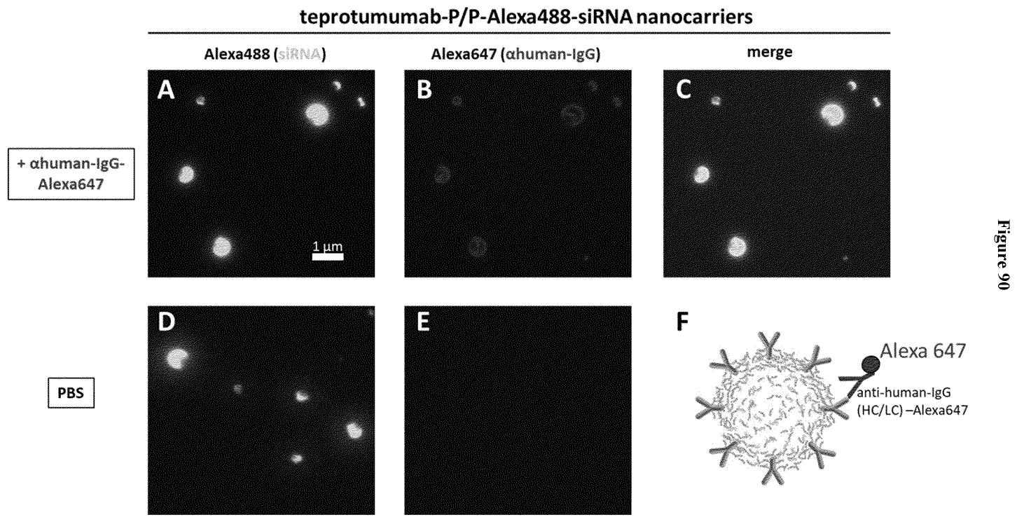

Here, ibrutinib-Cy3.5 nanocarriers were formed with αCD20-mAB-protamine/free protamine under standard conditions and tested them for integrity under pH conditions ranging between pH 4.8 and 8.0, thus covering all pH conditions the nanocarrier may be challenged with during therapeutic application (FIG. 88). It turned out that the nanocarriers, judged by Cy3.5 fluorescence of the complexed ibrutinib-Cy3.5, was stable in pH conditions between 5.8 and 8.0, with a tendency of the structured to disintegrate at lower pH.

### Example 33: Immunolabeling of Targeting IgG Antibodies in αEGFR-mAB-P/Free Protamine-siRNA Nanocarriers and in αIGF1R-mAB-P/Free Protamine siRNA Nanocarriers

The auto-assembly process of the cationic antibody-protamine/free protamine preparation and the siRNA leads to a nanoparticle structure with defined architecture: Here, αEGFR-mAB-protamine/free protamine-siRNA nanoparticles (FIG. 76) and αIGF1R (teprotumumab)mAB-protamine/free protamine/siRNA nanoparticles (FIG. 77) were subjected to an immune-detection of human IgG signals. A a-human IgG-Alexa647 was used to visualize human IgG position and orientation in the nanocarrier, which exposed signals only in the outer rim of the nanoparticle micellar structure (FIGS. 76 B and 77 B), but not in the lumen. Instead, signals for fluorescently labelled siRNA were found in the lumen of the structure (FIGS. 76A and 77 A). Therefore, it was concluded that the bulky IgG molecules were oriented facing outward of the nanoparticle micelle and thus must be definitively accessible to their protein targets, the extracellular domains of cell surface molecules and receptor tyrosine kinases.

### Example 34: Visualisation of the Free Protamine in the Nanocarrier Complex

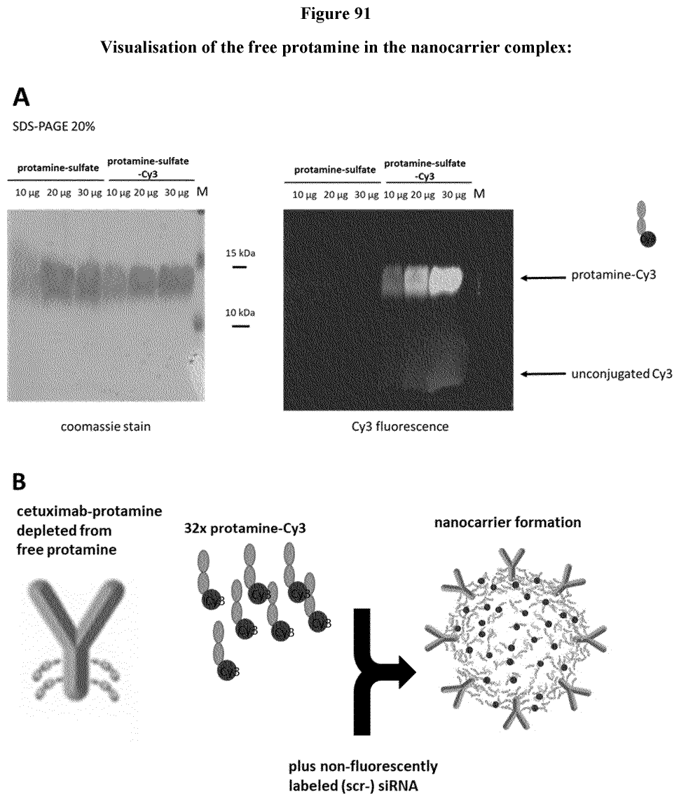

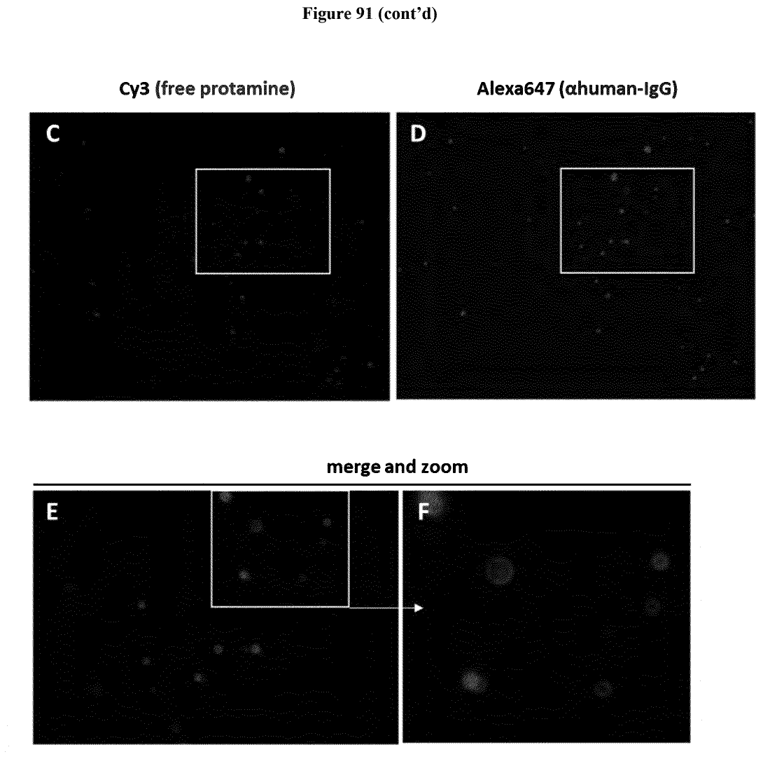

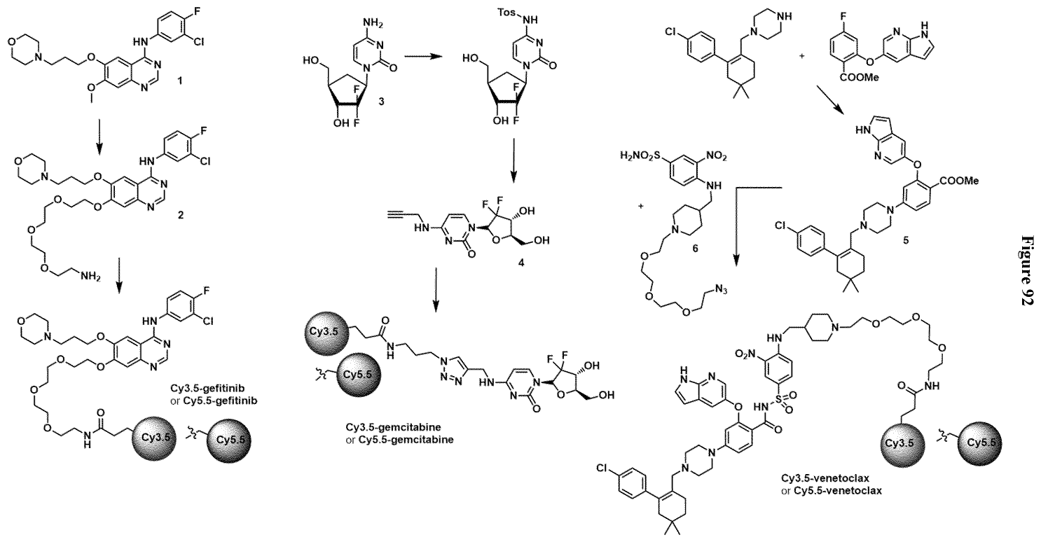

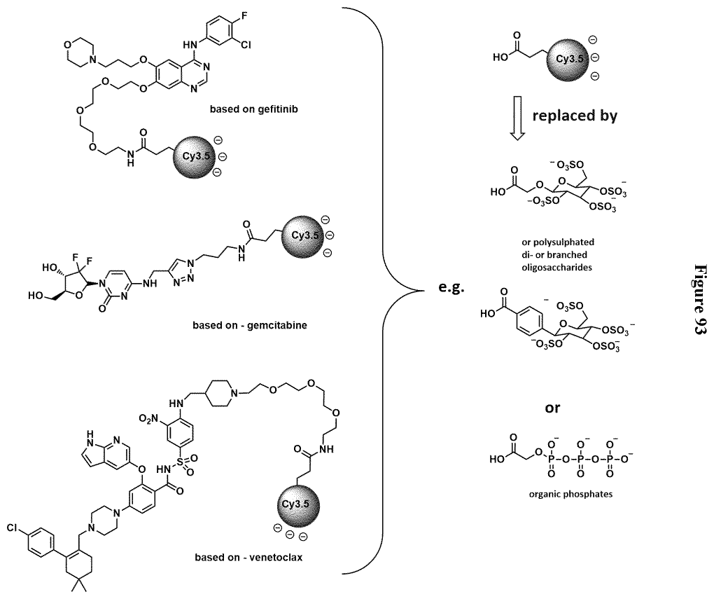

The position of the important free protamine in the nanocarrier remained unclear so far, so we turned towards this point and replaced the free protamine of a give αEGFR-protamine preparation with protamine that was conjugated to Cy3-NHS ester (FIG. 91 A) and formed a nanocarrier structure with this in combination with non-fluorescent siRNA (FIG. 91 B). The nanocarriers were then subjected to fluorescence microscopy and revealed a staining pattern, where the protamine-Cy3 was located in the lumen of the nanocarriers (FIG. 91 C-E), whereas the IgG portion was stained with the anti-human IgG-Alexa647 was located at the rim sections of the nanocarriers (FIG. 91 E-F).

### Example 35: Synthesis of Cyanine-Dye Conjugated Inhibitors Gefitinib, Gemcitabine and Venetoclax

To this end, the synthesis of the three new compounds are to be conducted, each connected to two different cyanine dyes, sulfo-Cy3.5™ (excitation 591 nm/emission 604 nm) and sulfo-Cy5.5™ (ex 684 nm, em 710 nm). Both cyanine dyes share the identical core fluorophore structure exhibiting the four strongly anionic sulfonyl groups necessary for the protamine cationic peptide coordination, but differ only in the number of conjugated double bonds, leading to discriminable fluorochrome attributes. Analogously to ibrutinib, three different drug-dye-conjugates are to be synthsized with comparable overall molecular shape. As possible candidates gefitinib (EGFR inhibitor), gemcitabine (cytostatic drug) and venetoclax (BLCL-2 inhibitor) were chosen because in all cases, they retained their binding potency to the target molecule after conjugation to dyes and allow fluorescence imaging applications (Wu et al. 2020; Zhu et al. 2018; Gonzales et al. 2018). They are to be conjugated to the cyanine dyes by installing a PEG4-spacer and using commercially available reactive NHS-ester or azido functionalized dyes (see FIG. 79).

First, the gefitinib analog is to be synthesized starting with the commercially available gefitinib 1, which will be demethylated and the resulting phenol will be converted by nucleophilic attack of an azido-PEG4-mesylate. The resulting azide will be reduced to the amine 2 and labelled with sulfo-Cy3.5 or sulfo-Cy5.5 yielding the gefitinib-conjugates for further complexation into the nanocarrier.

For gemcitabine 3, the hydroxyl groups will be protected and a leaving group will be installed on the cytosine to get after nucleophilic attack of propargyl amine the alkyne 4. (Solanki et al. 2020). After labelling with the corresponding azido functionalized cyanine dyes by click-reaction, the needed conjugates will be obtained.

In terms of the third example venetoclax, it will be started with the synthesis of the known venetoclax core-structure 5. (Giedt et al. 2014) The sulfonamide 6 will be reached in three steps using the already mentioned mesyl-PEG4-azide and after connection to 5, reduction of the azide and subsequent labelling with the cyanine dyes (NHS-ester) yield the corresponding venetoclax conjugates for further evaluation.

### Example 36: Expanding the Concept to Easier and Cheaper Polyanionic Molecular Moieties and Also to Other Therapeutic Interventions Like PDT and Radiotherapy

After the conversion and evaluation of the electrostatic binding principle to other anticancer drugs with different binding motifs and targets it is intended to change the necessary anionic character from the cyanine dyes used here (important for initial optical characterization and fluorescence imaging) to (1) easier accessible electrostatic connectors in terms of easier and cheaper synthesis, to facilitate a translation into the clinical evaluation. Candidates for this are poly-sulfated mono-, di- and branched oligosaccharides or mono-, di- and triphosphates which could pave the way for large scale synthesis (FIG. 93).

The invention illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims.

### NON-PATENT REFERENCES

Arvand, A., and Denny, C. T. (2001). Biology of EWS/ETS fusions in Ewing's family tumors. Oncogene 20, 5747-5754.
Bäumer, N., Appel, N., Terheyden, L., Buchholz, F., Rossig, C., Müller-Tidow, C., Berdel, W. E., and Bäumer, S. (2016). Antibody-coupled siRNA as an efficient method for in vivo mRNA knockdown. Nat. Protoc. 11, 22-36.
Bäumer, N., Berdel, W. E., and Bäumer, S. (2017). Immunoprotein-Mediated siRNA Delivery. Mol. Pharm. 14, 1339-1351.
Bäumer, N., Rehkämper, J., Appel, N., Terheyden, L., Hartmann, W., Wardelmann, E., Buchholz, F., Müller-Tidow, C., Berdel, W. E., and Bäumer, S. (2018). Downregulation of PIK3CA via antibody-esiRNA-complexes suppresses human xenograft tumor growth. PLOS One 13, e0200163.
Bäumer, S., Bäumer, N., Appel, N., Terheyden, L., Fremerey, J., Schelhaas, S., Wardelmann, E., Buchholz, F., Berdel, W. E., and Müller-Tidow, C. (2015). Antibody-Mediated Delivery of Anti-KRAS-siRNA In Vivo Overcomes Therapy Resistance in Colon Cancer. Clin Cancer Res 21, 1383-94.
Bose, P., and Grant, S. (2013). Mcl-1 as a Therapeutic Target in Acute Myelogenous Leukemia (AML). Leuk Res. Rep. 2, 12-14.
Byrd, J. C.; Furman, R. R.; Coutre, S. E.; Flinn, I. W.; Burger, J. A.; Blum, K. A. et al. (2013): Targeting BTK with ibrutinib in relapsed chronic lymphocytic leukemia. In: N. Engl. J. Med. 369 (1), S. 32-42. DOI: 10.1056/NEJMoa1215637.
Chen, J.; Kinoshita, T.; Sukbuntherng, J.; Chang, B. Y.; Elias, L. (2016): Ibrutinib Inhibits ERBB Receptor Tyrosine Kinases and HER2-Amplified Breast Cancer Cell Growth. In: Mol. Cancer. Ther. 15 (12), S. 2835-2844.
Chen, X., Stewart, E., Shelat, A. A., Qu, C., Bahrami, A., Hatley, M., Wu, G., Bradley, C., McEvoy, J., Pappo, A., et al. (2013). Targeting oxidative stress in embryonal rhabdomyosarcoma. Cancer. Cell. 24, 710-724.
Choi, Y. S., Lee, J. Y., Suh, J. S., Kwon, Y. M., Lee, S. J., Chung, J. K., Lee, D. S., Yang, V. C., Chung, C. P., and Park, Y. J. (2009). The systemic delivery of siRNAs by a cell penetrating peptide, low molecular weight protamine. Biomaterials 31, 1429-43.
Chono, S., Li, S. D., Conwell, C. C., and Huang, L. (2008). An efficient and low immunostimulatory nanoparticle formulation for systemic siRNA delivery to the tumor. J. Control. Release 131, 64-69.
Fire, A., Xu, S., Montgomery, M. K., Kostas, S. A., Driver, S. E., and Mello, C. C. (1998). Potent and specific genetic interference by double-stranded RNA in Caenorhabditis elegans. Nature 391, 806-11.
Giedt, R. J., Sprachman, M. M., Yang, K. S. & Weissleder, R. Imaging cellular distribution of Bcl inhibitors using small molecule drug conjugates. Bioconjug. Chem. 25, 2081-2085; 10.1021/bc500433k (2014).
Gonzales, J. et al. Nanoemulsion-Based Delivery of Fluorescent PARP Inhibitors in Mouse Models of Small Cell Lung Cancer. Bioconjug. Chem. 29, 3776-3782; 10.1021/acs.bioconjchem.8b00640 (2018).
Ha, H. T., Griffith, K. A., Zalupski, M. M., Schuetze, S. M., Thomas, D. G., Lucas, D. R., Baker, L. H., and Chugh, R. (2013). Phase II trial of cetuximab in patients with metastatic or locally advanced soft tissue or bone sarcoma. Am. J. Clin. Oncol. 36, 77-82.
Hansen, B., Linde, S., Kolendorf, K., and Jensen, F. (1979). Absorption of protamine-insulin in diabetic patients. I. Preparation and characterization of protamine-125I-insulin. Horm. Metab. Res. 11, 85-90.
Hauser, P. V., Pippin, J. W., Kaiser, C., Krofft, R. D., Brinkkoetter, P. T., Hudkins, K. L., Kerjaschki, D., Reiser, J., Alpers, C. E., and Shankland, S. J. (2010). Novel siRNA delivery system to target podocytes in vivo. PLOS One 5, e9463.
He, H., Ye, J., Liu, E., Liang, Q., Liu, Q., and Yang, V. C. (2014). Low molecular weight protamine (LMWP): a nontoxic protamine substitute and an effective cell-penetrating peptide. J. Control. Release 193, 63-73.
Herrmann, D., Seitz, G., Warmann, S. W., Bonin, M., Fuchs, J., and Armeanu-Ebinger, S. (2010). Cetuximab promotes immunotoxicity against rhabdomyosarcoma in vitro. J. Immunother. 33, 279-286.
Kikuchi, K., Tsuchiya, K., Otabe, O., Gotoh, T., Tamura, S., Katsumi, Y., Yagyu, S., Tsubai-Shimizu, S., Miyachi, M., Iehara, T., and Hosoi, H. (2008). Effects of PAX3-FKHR on malignant phenotypes in alveolar rhabdomyosarcoma. Biochem. Biophys. Res. Commun. 365, 568-574.
Kim, E.; Yang, K. S.; Kohler, R. H.; Dubach, J. M.; Mikula, H.; Weissleder, R. (2015): Optimized Near-IR Fluorescent Agents for in Vivo Imaging of Btk Expression. In: Bioconjug. Chem. 26 (8), S. 1513-1518. DOI: 10.1021/acs.bioconjchem.5b00152.
Lenz, G. (2017): Deciphering Ibrutinib Resistance in Chronic Lymphocytic Leukemia. In: J. Clin. Oncol. 35 (13), S. 1451-1452. DOI: 10.1200/JCO.2016.72.0102.
Liu, B. (2007). Exploring cell type-specific internalizing antibodies for targeted delivery of siRNA. Brief Funct. Genomic Proteomic 6, 112-119.
Liu, L., Wang, Y. D., Wu, J., Cui, J., and Chen, T. (2012). Carnitine palmitoyltransferase 1A (CPT1A): a transcriptional target of PAX3-FKHR and mediates PAX3-FKHR-dependent motility in alveolar rhabdomyosarcoma cells. BMC Cancer 12, 154-2407-12-154.
Paulussen, M., Ahrens, S., Craft, A. W., Dunst, J., Frohlich, B., Jabar, S., Rube, C., Winkelmann, W., Wissing, S., Zoubek, A., and Jurgens, H. (1998). Ewing's tumors with primary lung metastases: survival analysis of 114 (European Intergroup) Cooperative Ewing's Sarcoma Studies patients. J. Clin. Oncol. 16, 3044-3052.
Paulussen, M., Bielack, S., Jurgens, H., Jost, L., and ESMO Guidelines Working Group. (2008). Ewing's sarcoma of the bone: ESMO clinical recommendations for diagnosis, treatment and follow-up. Ann. Oncol. 19 Suppl 2, ii97-8.
Peer, D.; Zhu, P.; Carman, C. V.; Lieberman, J.; Shimaoka, M. (2007): Selective gene silencing in activated leukocytes by targeting siRNAs to the integrin lymphocyte function-associated antigen-1. In: Proc. Natl. Acad. Sci. U.S.A 104 (10), S. 4095-4100.
Peer, D.; Park, E. J.; Morishita, Y.; Carman, C. V.; Shimaoka, M. (2008): Systemic leukocyte-directed siRNA delivery revealing cyclin DI as an anti-inflammatory target. In: Science 319 (5863), S. 627-630. DOI: 10.1126/science.1149859
Smith, C. G., Fisher, D., Claes, B., Maughan, T. S., Idziaszczyk, S., Peuteman, G., Harris, R., James, M. D., Meade, A., Jasani, B., et al. (2013). Somatic profiling of the epidermal growth factor receptor pathway in tumors from patients with advanced colorectal cancer treated with chemotherapy+/−cetuximab. Clin. Cancer Res. 19, 4104-4113.
Solanki, A., King, D., Thibault, G., Wang, L. & Gibbs, S. L. Quantification of fluorophore distribution and therapeutic response in matched in vivo and ex vivo pancreatic cancer model systems. PLOS One 15, e0229407; 10.1371/journal.pone.0229407 (2020).
Song, E., Zhu, P., Lee, S. K., Chowdhury, D., Kussman, S., Dykxhoorn, D. M., Feng, Y., Palliser, D., Weiner, D. B., Shankar, P., Marasco, W. A., and Lieberman, J. (2005a). Antibody mediated in vivo delivery of small interfering RNAs via cell-surface receptors. Nat Biotechnol 23, 709-17.
Song, E., Zhu, P., Lee, S. K., Chowdhury, D., Kussman, S., Dykxhoorn, D. M., Feng, Y., Palliser, D., Weiner, D. B., Shankar, P., Marasco, W. A., and Lieberman, J. (2005b). Antibody mediated in vivo delivery of small interfering RNAs via cell-surface receptors. Nat Biotechnol 23, 709-17.
Stevens, M. C. (2005). Treatment for childhood rhabdomyosarcoma: the cost of cure. Lancet Oncol. 6, 77-84.
Turetsky, A.; Kim, E.; Kohler, R. H.; Miller, M. A.; Weissleder, R. (2014): Single cell imaging of Bruton's tyrosine kinase using an irreversible inhibitor. In: Sci. Rep. 4, S. 4782. DOI: 10.1038/srep04782.
Verdine, G. L., and Walensky, L. D. (2007). The challenge of drugging undruggable targets in cancer: lessons learned from targeting BCL-2 family members. Clin. Cancer Res. 13, 7264-7270.
Wilson, W. H.; Young, R. M.; Schmitz, R.; Yang, Y.; Pittaluga, S.; Wright, G. et al. (2015): Targeting B cell receptor signaling with ibrutinib in diffuse large B cell lymphoma. In: Nat. Med. 21 (8), S. 922-926. DOI: 10.1038/nm.3884.
Wu, Q. et al. Development of small molecule inhibitor-based fluorescent probes for highly specific super-resolution imaging. Nanoscale 12, 21591-21598; 10.1039/d0nr05188 h (2020).
Yao, Y. D.; Sun, T. M.; Huang, S. Y.; Dou, S.; Lin, L.; Chen, J. N. et al. (2012): Targeted delivery of PLK1-siRNA by ScFv suppresses Her2+ breast cancer growth and metastasis. In: Sci. Transl. Med. 4 (130), 130ra48. DOI: 10.1126/scitranslmed.3003601.
Zhang, Hui; Patel, Atish; Wang, Yi-Jun; Zhang, Yun-Kai; Kathawala, Rishil J.; Qiu, Long-Hui et al. (2017): The BTK Inhibitor Ibrutinib (PCI-32765) Overcomes Paclitaxel Resistance in ABCB1- and ABCC10-Overexpressing Cells and Tumors. In: Mol. Cancer. Ther. 16 (6), S. 1021-1030. DOI: 10.1158/1535-7163.MCT-16-0511.
Zhu, D. et al. Cell- and Tissue-Based Proteome Profiling and Dual Imaging of Apoptosis Markers with Probes Derived from Venetoclax and Idasanutlin. Angewandte Chemie (International ed. in English) 57, 9284-9289; 10.1002/anie.201802003 (2018).

1. A method of generating a nanoparticle comprising contacting
a) a fusion protein (A), said fusion protein (A) comprising an antibody (A1) and a positively charged polypeptide (A2);
b) a positively charged polypeptide (B); and
c) a negatively charged molecule (C);
thereby forming a nanoparticle.

2. The method of claim 1, wherein the molar ratio between the positively charged polypeptide (B) and the fusion protein (A) is at least about 10:1.

3. The method of claim 1, wherein the antibody (A1) comprises a heavy chain and a light chain.

4. The method of claim 1, wherein in the fusion protein (A) the positively charged polypeptide (A2) is fused to the C terminus of a heavy chain of the antibody (A1) and/or the C terminus of a light chain of the antibody (A1).

5. The method of claim 1, wherein the antibody (A1) is specific for a cell surface molecule.

6. The method of claim 1, wherein the negatively charged molecule (C) is a nucleic acid.

7. The method of claim 1, wherein the negatively charged molecule (C) has a molecular weight of about 20 kDa or less.

8. The method of claim 1, wherein the positively charged polypeptide (B) is a protamine or histone.

9. A nanoparticle obtainable by a method of claim 1.

10. A nanoparticle comprising:
a) a fusion protein (A), said fusion protein (A) comprising an antibody (A1) and a positively charged polypeptide (A2);
b) a positively charged polypeptide (B); and
c) one or more negatively charged molecule(s) (C).

11. The nanoparticle of claim 10, wherein the fusion protein (A) is enriched in the outer portion of the nanoparticle.

12. The nanoparticle of claim 10, wherein the nanoparticle has a mean diameter of about 0.05 μm to about 10 μm.

13. A composition comprising a nanoparticle of claim 10.

14. A method of treating a disease comprising administering to a subject in need thereof a nanoparticle of claim 10.

15. A kit comprising a nanoparticle of claim 10.

16. A method of treating a disease comprising administering to a subject in need thereof a composition of claim 13.

17. A kit comprising a composition of claim 13.

18. The method of claim 1, wherein the positively charged polypeptide (A2) has a net charge of at least +5.

19. The method of claim 1, wherein the positively charged polypeptide (B) has a net charge of at least +5.

20. The method of claim 1, wherein the positively charged polypeptide (A2) or the or the positively charged polypeptide (B) has a length of 10-300 amino acid residues.


SEQUENCE LISTING (82 sequences)

SEQ ID NO: 1 - protein, 8 aa, Artificial Sequence
  Cetuximab CDR-H1
GFSLTNYG

SEQ ID NO: 2 - protein, 7 aa, Artificial Sequence
  Cetuximab CDR-H2
IWSGGNT

SEQ ID NO: 3 - protein, 13 aa, Artificial Sequence
  Cetuximab CDR-H3
ARALTYYDYEFAY

SEQ ID NO: 4 - protein, 6 aa, Artificial Sequence
  Cetuximab CDR-L1
QSIGTN

SEQ ID NO: 5 - protein, 9 aa, Artificial Sequence
  Cetuximab CDR-L3
QQNNNWPTT

SEQ ID NO: 6 - protein, 119 aa, Artificial Sequence
  Cetuximab VH
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 7 - protein, 107 aa, Artificial Sequence
  Cetuximab VL
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 8 - protein, 449 aa, Artificial Sequence
  Cetuximab heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, Artificial Sequence
  Cetuximab light chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 8 aa, Artificial Sequence
  Gemtuzumab CDR-H1
GYTITDSN

SEQ ID NO: 11 - protein, 8 aa, Artificial Sequence
  Gemtuzumab CDR-H2
IYPYNGGT

SEQ ID NO: 12 - protein, 9 aa, Artificial Sequence
  Gemtuzumab CDR-H3
VNGNPWLAY

SEQ ID NO: 13 - protein, 10 aa, Artificial Sequence
  Gemtuzumab CDR-L1
ESLDNYGIRF

SEQ ID NO: 14 - protein, 9 aa, Artificial Sequence
  Gemtuzumab CDR-L3
QQTKEVPWS

SEQ ID NO: 15 - protein, 116 aa, Artificial Sequence [sequence omitted]

SEQ ID NO: 16 - protein, 111 aa, Artificial Sequence [sequence omitted]

SEQ ID NO: 17 - protein, 443 aa, Artificial Sequence
  Gemtuzumab Heavy chain
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 218 aa, Artificial Sequence
  Gemtuzumab Light chain
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 10 aa, Artificial Sequence
  Cixutumumab CDR-H1
GGTFSSYAIS

SEQ ID NO: 20 - protein, 16 aa, Artificial Sequence
  Cixutumumab CDR-H2
GIIPIFGTANYAQKFQ

SEQ ID NO: 21 - protein, 21 aa, Artificial Sequence
  Cixutumumab CDR-H3
APLRFLEWSTQDHYYYYYMDV

SEQ ID NO: 22 - protein, 11 aa, Artificial Sequence
  Cixutumumab CDR-L1
QGDSLRSYYAT

SEQ ID NO: 23 - protein, 7 aa, Artificial Sequence
  Cixutumumab CDR-L2
GENKRPS

SEQ ID NO: 24 - protein, 11 aa, Artificial Sequence
  Cixutumumab CDR-L3
KSRDGSGQHLV

SEQ ID NO: 25 - protein, 130 aa, Artificial Sequence
  Cixutumumab VH
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSS

SEQ ID NO: 26 - protein, 108 aa, Artificial Sequence
  Cixutumumab VL
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVL

SEQ ID NO: 27 - protein, 479 aa, Artificial Sequence
  Cixutumumab heavy chain
MGWSCIILFLVATATGVHSEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 233 aa, Artificial Sequence
  Cixutumumab light chain
MGWSCIILFLVATATGVHSSSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 29 - protein, 8 aa, Artificial Sequence
  Teprotumumab CDR-H1
GFTFSSYG

SEQ ID NO: 30 - protein, 8 aa, Artificial Sequence
  Teprotumumab CDR-H2
IWFDGSST

SEQ ID NO: 31 - protein, 11 aa, Artificial Sequence
  Teprotumumab CDR-H3
ARELGRRYFDL

SEQ ID NO: 32 - protein, 6 aa, Artificial Sequence
  Teprotumumab CDR-L1
QSVSSY

SEQ ID NO: 33 - protein, 8 aa, Artificial Sequence
  Teprotumumab CDR-L2
IWFDGSST

SEQ ID NO: 34 - protein, 10 aa, Artificial Sequence
  Teprotumumab CDR-L3
QQRSKWPPWT

SEQ ID NO: 35 - protein, 118 aa, Artificial Sequence
  Teprotumumab VH
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 36 - protein, 108 aa, Artificial Sequence
  Teprotumumab VL
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 37 - protein, 448 aa, Artificial Sequence
  Teprotumumab heavy chain
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 215 aa, Artificial Sequence
  Teprotumumab light chain
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 8 aa, Artificial Sequence
  Rituximab CDR-H1
GYTFTSYN

SEQ ID NO: 40 - protein, 8 aa, Artificial Sequence
  Rituximab CDR-H2
IYPGNGDT

SEQ ID NO: 41 - protein, 15 aa, Artificial Sequence
  Rituximab CDR-H3
CARSTYYGGDWYFNV

SEQ ID NO: 42 - protein, 6 aa, Artificial Sequence
  Rituximab CDR-L1
SSVSYI

SEQ ID NO: 43 - protein, 9 aa, Artificial Sequence
  Rituximab CDR-L3
QQWTSNPPT

SEQ ID NO: 44 - protein, 121 aa, Artificial Sequence [sequence omitted]

SEQ ID NO: 45 - protein, 106 aa, Artificial Sequence [sequence omitted]

SEQ ID NO: 46 - protein, 451 aa, Artificial Sequence
  Rituximab heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 213 aa, Artificial Sequence
  Rituximab light chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - DNA, 29 bp, Artificial Sequence
  fusion oncogene PAX3-Fork head
tggcctctcacctcagaattcaattcgtc

SEQ ID NO: 49 - DNA, 20 bp, Artificial Sequence
  siRNA
ggcctctcacctcagaattc

SEQ ID NO: 50 - DNA, 20 bp, Artificial Sequence
  siRNA
gcctctcacctcagaattca

SEQ ID NO: 51 - DNA, 20 bp, Artificial Sequence
  siRNA
cctctcacctcagaattcaa

SEQ ID NO: 52 - DNA, 102 bp, chum salmon
atgcccagaagacgcagatcctccagccgacctgtccgcaggcgccgccgccctagggtgtcccgacgtcgtcgcaggagaggaggccgcaggaggcgttag

SEQ ID NO: 53 - protein, 33 aa, chum salmon
MPRRRRSSSRPVRRRRRPRVSRRRRRRGGRRRR

SEQ ID NO: 54 - DNA, 153 bp, human
atggccaggtacagatgctgtcgcagccagagccggagcagatattaccgccagagacaaagaagtcgcagacgaaggaggcggagctgccagacacggaggagagccatgaggtgctgccgccccaggtacagaccgagatgtagaagacac

SEQ ID NO: 55 - protein, 51 aa, human
MARYRCCRSQSRSRYYRQRQRSRRRRRRSCQTRRRAMRCCRPRYRPRCRRH

SEQ ID NO: 56 - protein, 130 aa, human
MSGRGKQGGKARAKAKTRSSRAGLQFPVGRVRRLLRKGNYAERVGAGAPVYLAAVLEYLTAEILELAGNAARDNKKTRIIPRHLQLAIRNDEELNKLLGKVTIAQGGVLPNIQAVLLPKKTESHHKAKGK

SEQ ID NO: 57 - protein, 36 aa, Artificial Sequence
  human histone H2 derived peptide
GRGKQGGKARAKAKTRSSRAGLQFPVGRVHRLLRKG

SEQ ID NO: 58 - protein, 36 aa, Artificial Sequence
  human histone H2 derived peptide
GRGKQGGKARAKAKTRSSRAGLQFPVGRVHRLLRKW

SEQ ID NO: 59 - DNA, 21 bp, Artificial Sequence
  siRNA
uucugcuugugacauuaaaaa

SEQ ID NO: 60 - DNA, 21 bp, Artificial Sequence
  siRNA
aaacuuggcugaaguuuaaaa

SEQ ID NO: 61 - DNA, 22 bp, Artificial Sequence
  siRNA
ugaauucugaggugagaggctt

SEQ ID NO: 62 - DNA, 21 bp, Artificial Sequence
  siRNA
ggcagcagaacccuucuuauu

SEQ ID NO: 63 - DNA, 21 bp, Artificial Sequence
  siRNA
acacaaacuugaacagcuatt

SEQ ID NO: 64 - DNA, 21 bp, Artificial Sequence
  siRNA
gaaauguucuugcaguuaatt

SEQ ID NO: 65 - DNA, 9901 bp, Artificial Sequence
  Expression vector for Gemtuzumab-protamine fusion protein
cgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgaccctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacaactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctgcctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtcagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctaggcctccccgagcgccctgcctccgagggccggcgcaccataaaagaagccgccctagccacgtcccctcgcagttcggcggtcccgcgggtctgtctcaagcttgccgccagaacacaggtaagtgccgtgtgtggttcccgcgggcctggcctctttacgggttatggcccttgcgtgccttgaattacttccatgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgctaatctggtggcaccttcgcgcctgtctcgctgctttcgctaagtctctagccatttaaaatttttgataaccagctgcgacgctttttttctggcgagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaaactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgcccgggagagcgggcgggtgagtcacccacacaaaggaaaagggcctttccttcctcatccgtcgcttcatgtgactccacggagtaccgggcgccgtccaggcacctcgattagttgtcgagcttttggagtacgtcgtctttaggttggggggaggggttttatgcgatggagtttccccacactgagtgggtggagactgaagagttaggccagcttggcacttgatgtaattctccttggaatttgccctttttgagtttggatcttgcctcattctcaagcctcagacagtggttcaaagtttttttcttccatttcaggtgtcgtgaaaactacccctaaaagccaccggcgctcgagatgggctggagcggagtttttatcttcctgctgtctggcacagccggcgtgcactctgaagttcagctggttcagtctggcgccgaagtgaagaaacctggcagcagcgtgaaggtgtcctgcaaggccagcggctacaccatcaccgacagcaacatccactgggtccgacaggctccaggccagtctcttgagtggatcggctacatctacccctacaacggcggcaccgactacaaccagaagttcaagaaccgggccacactgaccgtggacaaccctaccaataccgcctacatggaactgagcagcctgcggagcgaggacaccgccttttactactgcgtgaacggcaacccctggctggcctattggggacagggaacactggtcaccgtgtccagcgcctccaccaagggcccatcggtcttccccctggcaccctcctccaagagtacctctgggggcacagcggccctgggctgcctggtcaaggactacttccccgaaccggtgacggtgtcgtggaactcaggcgccctgaccagtggcgtgcacaccttcccggctgtcctacagtcctcaggactctactccctcagtagtgtggtgaccgtgccctccagtagtttgggcacccagacctacatctgcaacgtgaatcacaagcccagtaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagtcacgaagaccctgaggtcaagttcaactggtacgtggacggcgtggaggtgcataatgccaagacaaagccgcgggaggagcagtacaacagtacgtaccgtgtggtcagtgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagtctgacctgcctggtcaaaggcttctatcccagtgacatcgccgtggagtgggagagtaatgggcagccggagaacaactacaagaccacgcctcccgtgctggactccgacggctccttcttcctctacagtaagctcaccgtggacaagagtaggtggcagcaggggaacgtcttctcatgctccgtgatgcatgaggctctgcacaaccactacacgcagaagagtctctccctgtctccgggtaaaggcggcagcggcggcagcggcggcagcggcggcagcatggccaggtacagatgctgtcgcagccagagccggagcagatattaccgccagagacaaagaagtcgcagacgaaggaggcggagctgccagacacggaggagagccatgaggtgctgccgccccaggtacagaccgagatgtagaagacacacgcgtacgcggccgctcgagcagaaactcatctcagaagaggatctggcagcaaatgatatcctggattacaaggatgacgacgataagggcggcagcggcggcagcggcggcagcggcggcagcggcggcagcggcggcagcggcggcagcggcggcagcggcggcagcagcggcttctggttcggctgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagttccgccagagcgcgcgagggcctccagcggccgcccctcccccacagcaggggcggggtcccgcgcccaccggaaggagcgggctcggggcgggcggcgctgattggccggggcgggcctgacgccgacgcggctataagagaccacaagcgacccgcagggccagacgttcttcgccgaagcttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtgatatcggatccacgcgtgccgccaccatggtttctacagcccagttcctgttcctgctggtgctgtggatccaagagacaaacggcgacatccagctgacacagagcccctctacactgtctgccagcgtgggcgacagagtgaccatcacatgtagagccagcgagagcctggacaactacggcatcagatttctgacctggttccagcagaagcccggcaaggctcctaagctgctgatgtacgccgccagcaatcaaggcagcggagtgcctagcagattttccggctctggcagcggcaccgagttcaccctgaccatctctagcctgcagcctgacgacttcgccacctactactgccagcagaccaaagaggtgccctggtcttttggccagggcacaaaggtggaagtgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagtaaggacagtacctacagtctcagtagtaccctgacgctgagtaaagcagactacgagaaacacaaagtctacgcctgcgaagtcacccatcagggcctgagttcgcccgtcacaaagagtttcaacaggggagagtgttagggaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaagga

SEQ ID NO: 66 - protein, 465 aa, Artificial Sequence
  Gemtuzumab heavy chain 2
MGWSGVFIFLLSGTAGVHSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 12 aa, Artificial Sequence
  linker
GGSGGSGGSGGS

SEQ ID NO: 68 - protein, 30 aa, Artificial sequence
  DKK tag
TRTRPLEQKLISEEDLAANDILDYKDDDDK

SEQ ID NO: 69 - protein, 33 aa, Artificial Sequence
  Linker with endosomal escape domain
GGSGGSGGSGGSGGSGGSGGSGGSGGSSGFWFG

SEQ ID NO: 70 - DNA, 9723 bp, Artificial Sequence
  Expression vector for Cetuximab-protamine fusion protein
cctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtcagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctaggcctccccgagcgccctgcctccgagggccggcgcaccataaaagaagccgccctagccacgtcccctcgcagttcggcggtcccgcgggtctgtctcaagcttgccgccagaacacaggtaagtgccgtgtgtggttcccgcgggcctggcctctttacgggttatggcccttgcgtgccttgaattacttccatgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgctaatctggtggcaccttcgcgcctgtctcgctgctttcgctaagtctctagccatttaaaatttttgataaccagctgcgacgctttttttctggcgagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaaactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgcccgggagagcgggcgggtgagtcacccacacaaaggaaaagggcctttccttcctcatccgtcgcttcatgtgactccacggagtaccgggcgccgtccaggcacctcgattagttgtcgagcttttggagtacgtcgtctttaggttggggggaggggttttatgcgatggagtttccccacactgagtgggtggagactgaagagttaggccagcttggcacttgatgtaattctccttggaatttgccctttttgagtttggatcttgcctcattctcaagcctcagacagtggttcaaagtttttttcttccatttcaggtgtcgtgaaaactacccctaaaagccaccggcgagatctgccaccatgggttggagcctcatcttgctcttccttgtcgctgttgctacgcgtgtcctgtcccaggtgcagctgaagcagagcggccccggcctggtgcagcccagccagagcctgagcatcacctgcaccgtgagcggcttcagcctgaccaactacggcgtgcactgggtgaggcagagccccggcaagggcctggagtggctgggcgtgatctggagcggcggcaacaccgactacaacacccccttcaccagcaggctgagcatcaacaaggacaacagcaagagccaggtgttcttcaagatgaacagcctgcagagcaacgacaccgccatctactactgcgccagggccctgacctactacgactacgagttcgcctactggggccagggcaccctggtgaccgtgagcgccgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagggcggcagcggcggcagcggcggcagcggcggcagcatggccaggtacagatgctgtcgcagccagagccggagcagatattaccgccagagacaaagaagtcgcagacgaaggaggcggagctgccagacacggaggagagccatgaggtgctgccgccccaggtacagaccgagatgtagaagacactgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagttccgccagagcgcgcgagggcctccagcggccgcccctcccccacagcaggggcggggtcccgcgcccaccggaaggagcgggctcggggcgggcggcgctgattggccggggcgggcctgacgccgacgcggctataagagaccacaagcgacccgcagggccagacgttcttcgccgaagcttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtgccaccatggattttcaggtgcagattatcagcttcctgctaatcagtgcttcagtcataatgtccagaggagacatcctgctgacccagagccccgtgatcctgagcgtgagccccggcgagagggtgagcttcagctgcagggccagccagagcatcggcaccaacatccactggtaccagcagaggaccaacggcagccccaggctgctgatcaagtacgccagcgagagcatcagcggcatccccagcaggttcagcggcagcggcagcggcaccgacttcaccctgagcatcaacagcgtggagagcgaggacatcgccgactactactgccagcagaacaacaactggcccaccaccttcggcgccggcaccaagctggagctgaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgctgagatatcggatctctcgaggttaacgaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgaccctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacaactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctg

SEQ ID NO: 71 - protein, 531 aa, Artificial Sequence
  Cetuximab-protamine fusion protein
MGWSLILLFLVAVATRVLSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSMARYRCCRSQSRSRYYRQRQRSRRRRRRSCQTRRRAMRCCRPRYRPRCRRH

SEQ ID NO: 72 - DNA, 9912 bp, Artificial Sequence
  Expression vector for Cetuximab-protamine fusion protein
cctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtcagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctaggcctccccgagcgccctgcctccgagggccggcgcaccataaaagaagccgccctagccacgtcccctcgcagttcggcggtcccgcgggtctgtctcaagcttgccgccagaacacaggtaagtgccgtgtgtggttcccgcgggcctggcctctttacgggttatggcccttgcgtgccttgaattacttccatgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgctaatctggtggcaccttcgcgcctgtctcgctgctttcgctaagtctctagccatttaaaatttttgataaccagctgcgacgctttttttctggcgagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaaactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgcccgggagagcgggcgggtgagtcacccacacaaaggaaaagggcctttccttcctcatccgtcgcttcatgtgactccacggagtaccgggcgccgtccaggcacctcgattagttgtcgagcttttggagtacgtcgtctttaggttggggggaggggttttatgcgatggagtttccccacactgagtgggtggagactgaagagttaggccagcttggcacttgatgtaattctccttggaatttgccctttttgagtttggatcttgcctcattctcaagcctcagacagtggttcaaagtttttttcttccatttcaggtgtcgtgaaaactacccctaaaagccaccggcgagatctgccaccatgggttggagcctcatcttgctcttccttgtcgctgttgctacgcgtgtcctgtcccaggtgcagctgaagcagagcggccccggcctggtgcagcccagccagagcctgagcatcacctgcaccgtgagcggcttcagcctgaccaactacggcgtgcactgggtgaggcagagccccggcaagggcctggagtggctgggcgtgatctggagcggcggcaacaccgactacaacacccccttcaccagcaggctgagcatcaacaaggacaacagcaagagccaggtgttcttcaagatgaacagcctgcagagcaacgacaccgccatctactactgcgccagggccctgacctactacgactacgagttcgcctactggggccagggcaccctggtgaccgtgagcgccgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagggcggcagcggcggcagcggcggcagcggcggcagcatggccaggtacagatgctgtcgcagccagagccggagcagatattaccgccagagacaaagaagtcgcagacgaaggaggcggagctgccagacacggaggagagccatgaggtgctgccgccccaggtacagaccgagatgtagaagacactgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagttccgccagagcgcgcgagggcctccagcggccgcccctcccccacagcaggggcggggtcccgcgcccaccggaaggagcgggctcggggcgggcggcgctgattggccggggcgggcctgacgccgacgcggctataagagaccacaagcgacccgcagggccagacgttcttcgccgaagcttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtgccaccatggattttcaggtgcagattatcagcttcctgctaatcagtgcttcagtcataatgtccagaggagacatcctgctgacccagagccccgtgatcctgagcgtgagccccggcgagagggtgagcttcagctgcagggccagccagagcatcggcaccaacatccactggtaccagcagaggaccaacggcagccccaggctgctgatcaagtacgccagcgagagcatcagcggcatccccagcaggttcagcggcagcggcagcggcaccgacttcaccctgagcatcaacagcgtggagagcgaggacatcgccgactactactgccagcagaacaacaactggcccaccaccttcggcgccggcaccaagctggagctgaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgcggcggcagcggcggcagcggcggcagcggcggcagcatggccaggtacagatgctgtcgcagccagagccggagcagatattaccgccagagacaaagaagtcgcagacgaaggaggcggagctgccagacacggaggagagccatgaggtgctgccgccccaggtacagaccgagatgtagaagacactgagatatcggatctctcgaggttaacgaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgaccctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacaactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctg

SEQ ID NO: 73 - protein, 299 aa, Artificial Sequence
  Cetuximab-protamine fusion protein
MDFQVQIISFLLISASVIMSRGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSGGSGGSMARYRCCRSQSRSRYYRQRQRSRRRRRRSCQTRRRAMRCCRPRYRPRCRRH

SEQ ID NO: 74 - DNA, 10065 bp, Artificial Sequence
  Expression vector for Cetuximab-protamine fusion protein
cctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtcagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctaggcctccccgagcgccctgcctccgagggccggcgcaccataaaagaagccgccctagccacgtcccctcgcagttcggcggtcccgcgggtctgtctcaagcttgccgccagaacacaggtaagtgccgtgtgtggttcccgcgggcctggcctctttacgggttatggcccttgcgtgccttgaattacttccatgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgctaatctggtggcaccttcgcgcctgtctcgctgctttcgctaagtctctagccatttaaaatttttgataaccagctgcgacgctttttttctggcgagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaaactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgcccgggagagcgggcgggtgagtcacccacacaaaggaaaagggcctttccttcctcatccgtcgcttcatgtgactccacggagtaccgggcgccgtccaggcacctcgattagttgtcgagcttttggagtacgtcgtctttaggttggggggaggggttttatgcgatggagtttccccacactgagtgggtggagactgaagagttaggccagcttggcacttgatgtaattctccttggaatttgccctttttgagtttggatcttgcctcattctcaagcctcagacagtggttcaaagtttttttcttccatttcaggtgtcgtgaaaactacccctaaaagccaccggcgagatctgccaccatgggttggagcctcatcttgctcttccttgtcgctgttgctacgcgtgtcctgtcccaggtgcagctgaagcagagcggccccggcctggtgcagcccagccagagcctgagcatcacctgcaccgtgagcggcttcagcctgaccaactacggcgtgcactgggtgaggcagagccccggcaagggcctggagtggctgggcgtgatctggagcggcggcaacaccgactacaacacccccttcaccagcaggctgagcatcaacaaggacaacagcaagagccaggtgttcttcaagatgaacagcctgcagagcaacgacaccgccatctactactgcgccagggccctgacctactacgactacgagttcgcctactggggccagggcaccctggtgaccgtgagcgccgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagggcggcagcggcggcagcggcggcagcggcggcagcatggccaggtacagatgctgtcgcagccagagccggagcagatattaccgccagagacaaagaagtcgcagacgaaggaggcggagctgccagacacggaggagagccatgaggtgctgccgccccaggtacagaccgagatgtagaagacactgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagttccgccagagcgcgcgagggcctccagcggccgcccctcccccacagcaggggcggggtcccgcgcccaccggaaggagcgggctcggggcgggcggcgctgattggccggggcgggcctgacgccgacgcggctataagagaccacaagcgacccgcagggccagacgttcttcgccgaagcttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtgccaccatggattttcaggtgcagattatcagcttcctgctaatcagtgcttcagtcataatgtccagaggagacatcctgctgacccagagccccgtgatcctgagcgtgagccccggcgagagggtgagcttcagctgcagggccagccagagcatcggcaccaacatccactggtaccagcagaggaccaacggcagccccaggctgctgatcaagtacgccagcgagagcatcagcggcatccccagcaggttcagcggcagcggcagcggcaccgacttcaccctgagcatcaacagcgtggagagcgaggacatcgccgactactactgccagcagaacaacaactggcccaccaccttcggcgccggcaccaagctggagctgaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgcggcggcagcggcggcagcggcggcagcggcggcagcatggtccgataccgcgtgaggagcctgagcgaacgctcgcacgaggtgtacaggcagcagttgcatgggcaagagcaaggacaccacggccaagaggagcaagggctgagcccggagcacgtcgaggtctacgagaggacccatggccagtctcactataggcgcagacactgctctcgaaggaggctgcaccggatccacaggcggcagcatcgctcctgcagaaggcgcaaaagacgctcctgcaggcaccggaggaggcatcgcagaggctgcagaaccaggaagagaacatgcagaaggcactgagatatcggatctctcgaggttaacgaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgaccctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacaactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctg

SEQ ID NO: 75 - protein, 350 aa, Artificial Sequence
  Cetuximab-protamine fusion protein
MDFQVQIISFLLISASVIMSRGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSGGSGGSMVRYRVRSLSERSHEVYRQQLHGQEQGHHGQEEQGLSPEHVEVYERTHGQSHYRRRHCSRRRLHRIHRRQHRSCRRRKRRSCRHRRRHRRGCRTRKRTCRRH

SEQ ID NO: 76 - DNA, 9723 bp, Artificial Sequence
  Expression vector for Cetuximab-protamine fusion protein
cctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtcagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctaggcctccccgagcgccctgcctccgagggccggcgcaccataaaagaagccgccctagccacgtcccctcgcagttcggcggtcccgcgggtctgtctcaagcttgccgccagaacacaggtaagtgccgtgtgtggttcccgcgggcctggcctctttacgggttatggcccttgcgtgccttgaattacttccatgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgctaatctggtggcaccttcgcgcctgtctcgctgctttcgctaagtctctagccatttaaaatttttgataaccagctgcgacgctttttttctggcgagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaaactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgcccgggagagcgggcgggtgagtcacccacacaaaggaaaagggcctttccttcctcatccgtcgcttcatgtgactccacggagtaccgggcgccgtccaggcacctcgattagttgtcgagcttttggagtacgtcgtctttaggttggggggaggggttttatgcgatggagtttccccacactgagtgggtggagactgaagagttaggccagcttggcacttgatgtaattctccttggaatttgccctttttgagtttggatcttgcctcattctcaagcctcagacagtggttcaaagtttttttcttccatttcaggtgtcgtgaaaactacccctaaaagccaccggcgagatctgccaccatgggttggagcctcatcttgctcttccttgtcgctgttgctacgcgtgtcctgtcccaggtgcagctgaagcagagcggccccggcctggtgcagcccagccagagcctgagcatcacctgcaccgtgagcggcttcagcctgaccaactacggcgtgcactgggtgaggcagagccccggcaagggcctggagtggctgggcgtgatctggagcggcggcaacaccgactacaacacccccttcaccagcaggctgagcatcaacaaggacaacagcaagagccaggtgttcttcaagatgaacagcctgcagagcaacgacaccgccatctactactgcgccagggccctgacctactacgactacgagttcgcctactggggccagggcaccctggtgaccgtgagcgccgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagtgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagttccgccagagcgcgcgagggcctccagcggccgcccctcccccacagcaggggcggggtcccgcgcccaccggaaggagcgggctcggggcgggcggcgctgattggccggggcgggcctgacgccgacgcggctataagagaccacaagcgacccgcagggccagacgttcttcgccgaagcttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtgccaccatggattttcaggtgcagattatcagcttcctgctaatcagtgcttcagtcataatgtccagaggagacatcctgctgacccagagccccgtgatcctgagcgtgagccccggcgagagggtgagcttcagctgcagggccagccagagcatcggcaccaacatccactggtaccagcagaggaccaacggcagccccaggctgctgatcaagtacgccagcgagagcatcagcggcatccccagcaggttcagcggcagcggcagcggcaccgacttcaccctgagcatcaacagcgtggagagcgaggacatcgccgactactactgccagcagaacaacaactggcccaccaccttcggcgccggcaccaagctggagctgaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgcggcggcagcggcggcagcggcggcagcggcggcagcatggccaggtacagatgctgtcgcagccagagccggagcagatattaccgccagagacaaagaagtcgcagacgaaggaggcggagctgccagacacggaggagagccatgaggtgctgccgccccaggtacagaccgagatgtagaagacactgagatatcggatctctcgaggttaacgaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgaccctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacaactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctg

SEQ ID NO: 77 - DNA, 9876 bp, Artificial Sequence
  Expression vector for Cetuximab-protamine fusion protein
cctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtcagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctaggcctccccgagcgccctgcctccgagggccggcgcaccataaaagaagccgccctagccacgtcccctcgcagttcggcggtcccgcgggtctgtctcaagcttgccgccagaacacaggtaagtgccgtgtgtggttcccgcgggcctggcctctttacgggttatggcccttgcgtgccttgaattacttccatgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgctaatctggtggcaccttcgcgcctgtctcgctgctttcgctaagtctctagccatttaaaatttttgataaccagctgcgacgctttttttctggcgagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaaactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgcccgggagagcgggcgggtgagtcacccacacaaaggaaaagggcctttccttcctcatccgtcgcttcatgtgactccacggagtaccgggcgccgtccaggcacctcgattagttgtcgagcttttggagtacgtcgtctttaggttggggggaggggttttatgcgatggagtttccccacactgagtgggtggagactgaagagttaggccagcttggcacttgatgtaattctccttggaatttgccctttttgagtttggatcttgcctcattctcaagcctcagacagtggttcaaagtttttttcttccatttcaggtgtcgtgaaaactacccctaaaagccaccggcgagatctgccaccatgggttggagcctcatcttgctcttccttgtcgctgttgctacgcgtgtcctgtcccaggtgcagctgaagcagagcggccccggcctggtgcagcccagccagagcctgagcatcacctgcaccgtgagcggcttcagcctgaccaactacggcgtgcactgggtgaggcagagccccggcaagggcctggagtggctgggcgtgatctggagcggcggcaacaccgactacaacacccccttcaccagcaggctgagcatcaacaaggacaacagcaagagccaggtgttcttcaagatgaacagcctgcagagcaacgacaccgccatctactactgcgccagggccctgacctactacgactacgagttcgcctactggggccagggcaccctggtgaccgtgagcgccgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagtgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagttccgccagagcgcgcgagggcctccagcggccgcccctcccccacagcaggggcggggtcccgcgcccaccggaaggagcgggctcggggcgggcggcgctgattggccggggcgggcctgacgccgacgcggctataagagaccacaagcgacccgcagggccagacgttcttcgccgaagcttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtgccaccatggattttcaggtgcagattatcagcttcctgctaatcagtgcttcagtcataatgtccagaggagacatcctgctgacccagagccccgtgatcctgagcgtgagccccggcgagagggtgagcttcagctgcagggccagccagagcatcggcaccaacatccactggtaccagcagaggaccaacggcagccccaggctgctgatcaagtacgccagcgagagcatcagcggcatccccagcaggttcagcggcagcggcagcggcaccgacttcaccctgagcatcaacagcgtggagagcgaggacatcgccgactactactgccagcagaacaacaactggcccaccaccttcggcgccggcaccaagctggagctgaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgcggcggcagcggcggcagcggcggcagcggcggcagcatggtccgataccgcgtgaggagcctgagcgaacgctcgcacgaggtgtacaggcagcagttgcatgggcaagagcaaggacaccacggccaagaggagcaagggctgagcccggagcacgtcgaggtctacgagaggacccatggccagtctcactataggcgcagacactgctctcgaaggaggctgcaccggatccacaggcggcagcatcgctcctgcagaaggcgcaaaagacgctcctgcaggcaccggaggaggcatcgcagaggctgcagaaccaggaagagaacatgcagaaggcactgagatatcggatctctcgaggttaacgaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgaccctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacaactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctg

SEQ ID NO: 78 - DNA, 9723 bp, Artificial Sequence
  Expression vector for Teprotumumab-protamine fusion protein
cctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtcagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctaggcctccccgagcgccctgcctccgagggccggcgcaccataaaagaagccgccctagccacgtcccctcgcagttcggcggtcccgcgggtctgtctcaagcttgccgccagaacacaggtaagtgccgtgtgtggttcccgcgggcctggcctctttacgggttatggcccttgcgtgccttgaattacttccatgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgctaatctggtggcaccttcgcgcctgtctcgctgctttcgctaagtctctagccatttaaaatttttgataaccagctgcgacgctttttttctggcgagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaaactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgcccgggagagcgggcgggtgagtcacccacacaaaggaaaagggcctttccttcctcatccgtcgcttcatgtgactccacggagtaccgggcgccgtccaggcacctcgattagttgtcgagcttttggagtacgtcgtctttaggttggggggaggggttttatgcgatggagtttccccacactgagtgggtggagactgaagagttaggccagcttggcacttgatgtaattctccttggaatttgccctttttgagtttggatcttgcctcattctcaagcctcagacagtggttcaaagtttttttcttccatttcaggtgtcgtgaaaactacccctaaaagccaccggcgagatctgccaccatgggttggagcctcatcttgctcttccttgtcgctgttgctacgcgtgtcctgtcccaggtggagctggtggagagcggcggcggcgtggtgcagcccggcaggagccagaggctgagctgcgccgccagcggcttcaccttcagcagctacggcatgcactgggtgaggcaggcccccggcaagggcctggagtgggtggccatcatctggttcgacggcagcagcacctactacgccgacagcgtgaggggcaggttcaccatcagcagggacaacagcaagaacaccctgtacctgcagatgaacagcctgagggccgaggacaccgccgtgtacttctgcgccagggagctgggcaggaggtacttcgacctgtggggcaggggcaccctggtgagcgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagggcggcagcggcggcagcggcggcagcggcggcagcatggccaggtacagatgctgtcgcagccagagccggagcagatattaccgccagagacaaagaagtcgcagacgaaggaggcggagctgccagacacggaggagagccatgaggtgctgccgccccaggtacagaccgagatgtagaagacactgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagttccgccagagcgcgcgagggcctccagcggccgcccctcccccacagcaggggcggggtcccgcgcccaccggaaggagcgggctcggggcgggcggcgctgattggccggggcgggcctgacgccgacgcggctataagagaccacaagcgacccgcagggccagacgttcttcgccgaagcttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtgccaccatggattttcaggtgcagattatcagcttcctgctaatcagtgcttcagtcataatgtccagaggagagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaagagggccaccggcatccccgccaggttcagcggcagcggcagcggcaccgacttcaccctgaccatcagcagcctggagcccgaggacttcgccgtgtactactgccagcagaggagcaagtggcccccctggaccttcggccagggcaccaaggtggagagcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgctgagatatcggatctctcgaggttaacgaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgaccctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacaactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctg

SEQ ID NO: 79 - protein, 530 aa, Artificial Sequence
  Teprotumumab-protamine fusion protein
MGWSLILLFLVAVATRVLSQVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSMARYRCCRSQSRSRYYRQRQRSRRRRRRSCQTRRRAMRCCRPRYRPRCRRH

SEQ ID NO: 80 - DNA, 9912 bp, Artificial Sequence
  Expression vector for Teprotumumab-protamine fusion protein
cctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtcagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctaggcctccccgagcgccctgcctccgagggccggcgcaccataaaagaagccgccctagccacgtcccctcgcagttcggcggtcccgcgggtctgtctcaagcttgccgccagaacacaggtaagtgccgtgtgtggttcccgcgggcctggcctctttacgggttatggcccttgcgtgccttgaattacttccatgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgctaatctggtggcaccttcgcgcctgtctcgctgctttcgctaagtctctagccatttaaaatttttgataaccagctgcgacgctttttttctggcgagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaaactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgcccgggagagcgggcgggtgagtcacccacacaaaggaaaagggcctttccttcctcatccgtcgcttcatgtgactccacggagtaccgggcgccgtccaggcacctcgattagttgtcgagcttttggagtacgtcgtctttaggttggggggaggggttttatgcgatggagtttccccacactgagtgggtggagactgaagagttaggccagcttggcacttgatgtaattctccttggaatttgccctttttgagtttggatcttgcctcattctcaagcctcagacagtggttcaaagtttttttcttccatttcaggtgtcgtgaaaactacccctaaaagccaccggcgagatctgccaccatgggttggagcctcatcttgctcttccttgtcgctgttgctacgcgtgtcctgtcccaggtggagctggtggagagcggcggcggcgtggtgcagcccggcaggagccagaggctgagctgcgccgccagcggcttcaccttcagcagctacggcatgcactgggtgaggcaggcccccggcaagggcctggagtgggtggccatcatctggttcgacggcagcagcacctactacgccgacagcgtgaggggcaggttcaccatcagcagggacaacagcaagaacaccctgtacctgcagatgaacagcctgagggccgaggacaccgccgtgtacttctgcgccagggagctgggcaggaggtacttcgacctgtggggcaggggcaccctggtgagcgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagggcggcagcggcggcagcggcggcagcggcggcagcatggccaggtacagatgctgtcgcagccagagccggagcagatattaccgccagagacaaagaagtcgcagacgaaggaggcggagctgccagacacggaggagagccatgaggtgctgccgccccaggtacagaccgagatgtagaagacactgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagttccgccagagcgcgcgagggcctccagcggccgcccctcccccacagcaggggcggggtcccgcgcccaccggaaggagcgggctcggggcgggcggcgctgattggccggggcgggcctgacgccgacgcggctataagagaccacaagcgacccgcagggccagacgttcttcgccgaagcttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtgccaccatggattttcaggtgcagattatcagcttcctgctaatcagtgcttcagtcataatgtccagaggagagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaagagggccaccggcatccccgccaggttcagcggcagcggcagcggcaccgacttcaccctgaccatcagcagcctggagcccgaggacttcgccgtgtactactgccagcagaggagcaagtggcccccctggaccttcggccagggcaccaaggtggagagcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgcggcggcagcggcggcagcggcggcagcggcggcagcatggccaggtacagatgctgtcgcagccagagccggagcagatattaccgccagagacaaagaagtcgcagacgaaggaggcggagctgccagacacggaggagagccatgaggtgctgccgccccaggtacagaccgagatgtagaagacactgagatatcggatctctcgaggttaacgaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgaccctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacaactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctg

SEQ ID NO: 81 - protein, 300 aa, Artificial Sequence
  Teprotumumab-protamine fusion protein
MDFQVQIISFLLISASVIMSRGEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSGGSGGSMARYRCCRSQSRSRYYRQRQRSRRRRRRSCQTRRRAMRCCRPRYRPRCRRH

SEQ ID NO: 82 - DNA, 9723 bp, Artificial Sequence
  Expression vector for Teprotumumab-protamine fusion protein
cctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtcagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctaggcctccccgagcgccctgcctccgagggccggcgcaccataaaagaagccgccctagccacgtcccctcgcagttcggcggtcccgcgggtctgtctcaagcttgccgccagaacacaggtaagtgccgtgtgtggttcccgcgggcctggcctctttacgggttatggcccttgcgtgccttgaattacttccatgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgctaatctggtggcaccttcgcgcctgtctcgctgctttcgctaagtctctagccatttaaaatttttgataaccagctgcgacgctttttttctggcgagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaaactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgcccgggagagcgggcgggtgagtcacccacacaaaggaaaagggcctttccttcctcatccgtcgcttcatgtgactccacggagtaccgggcgccgtccaggcacctcgattagttgtcgagcttttggagtacgtcgtctttaggttggggggaggggttttatgcgatggagtttccccacactgagtgggtggagactgaagagttaggccagcttggcacttgatgtaattctccttggaatttgccctttttgagtttggatcttgcctcattctcaagcctcagacagtggttcaaagtttttttcttccatttcaggtgtcgtgaaaactacccctaaaagccaccggcgagatctgccaccatgggttggagcctcatcttgctcttccttgtcgctgttgctacgcgtgtcctgtcccaggtggagctggtggagagcggcggcggcgtggtgcagcccggcaggagccagaggctgagctgcgccgccagcggcttcaccttcagcagctacggcatgcactgggtgaggcaggcccccggcaagggcctggagtgggtggccatcatctggttcgacggcagcagcacctactacgccgacagcgtgaggggcaggttcaccatcagcagggacaacagcaagaacaccctgtacctgcagatgaacagcctgagggccgaggacaccgccgtgtacttctgcgccagggagctgggcaggaggtacttcgacctgtggggcaggggcaccctggtgagcgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagtgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagttccgccagagcgcgcgagggcctccagcggccgcccctcccccacagcaggggcggggtcccgcgcccaccggaaggagcgggctcggggcgggcggcgctgattggccggggcgggcctgacgccgacgcggctataagagaccacaagcgacccgcagggccagacgttcttcgccgaagcttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtgccaccatggattttcaggtgcagattatcagcttcctgctaatcagtgcttcagtcataatgtccagaggagagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaagagggccaccggcatccccgccaggttcagcggcagcggcagcggcaccgacttcaccctgaccatcagcagcctggagcccgaggacttcgccgtgtactactgccagcagaggagcaagtggcccccctggaccttcggccagggcaccaaggtggagagcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgcggcggcagcggcggcagcggcggcagcggcggcagcatggccaggtacagatgctgtcgcagccagagccggagcagatattaccgccagagacaaagaagtcgcagacgaaggaggcggagctgccagacacggaggagagccatgaggtgctgccgccccaggtacagaccgagatgtagaagacactgagatatcggatctctcgaggttaacgaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgaccctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacaactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctg